(19) United States
(12) Patent Application Publication
(10) Pub. No.: US 2023/0363270 A1
(43) Pub. Date: Nov. 9, 2023

(54) ORGANIC COMPOUNDS, ORGANIC MIXTURES AND THE USES THEREOF

(71) Applicant: Zhejiang Brilliant Optoelectronic Technology Co.,Ltd., Taizhou (CN)

(72) Inventors: Junyou PAN, Taizhou (CN); Hong HUANG, Taizhou (CN); Kun WANG, Taizhou (CN)

(73) Assignee: Zhejiang Brilliant Optoelectronic Technology Co.,Ltd., Taizhou (CN)

(21) Appl. No.: 18/351,686

(22) Filed: Jul. 13, 2023

Related U.S. Application Data
(63) Continuation of application No. PCT/CN2022/071817, filed on Jan. 13, 2022.

(30) Foreign Application Priority Data
Jan. 13, 2021 (CN) 202110041372.9

(51) Int. Cl. H10K 85/60; C07D 471/04; C09K 11/06; C07D 209/86; C07F 5/04

(52) U.S. Cl. CPC H10K 85/6572; C07D 471/04; C09K 11/06; C07D 209/86; C07F 5/04; H10K 85/658; H10K 85/654; H10K 2101/90

(57) ABSTRACT

Provided are organic compounds, including a structure as represented by chemical formula (I). Also provided are organic mixtures including at least one first compound and at least one second compound, wherein the first compound contains the organic compounds. Further provided are organic electronic devices containing the organic compounds or the organic mixtures.

### CROSS-REFERENCE TO RELATED APPLICATIONS

The present application is a continuation of International Application No. PCT/CN2022/071817, filed on Jan. 13, 2022, which claims priority to Chinese Patent Application No. 202110041372.9, filed on Jan. 13, 2021. All of the aforementioned applications are incorporated herein by reference in their entireties.

### TECHNICAL FIELD

The present disclosure relates to the field of electroluminescent material, and in particularly to an organic compound, an organic mixture, and the applications thereof in organic electronic devices, particularly in organic electroluminescent devices.

### BACKGROUND

Because of the synthetic diversity and feasibility of organic electroluminescent materials, the possibility of achieving large-area noval display devices has a solid foundation. In order to improve the luminescence efficiency of OLEDs, a variety of emitting material systems base on fluorescent and phosphorescent materials have been developed so far. OLEDs using fluorescent materials present high reliability, but their internal electroluminescence quantum efficiency is limited to 25% due to the 1:3 branching ratio between the single and triple excited states of the exciton. In contrast, OLEDs using phosphorescent materials have achieved almost an internal luminescence quantum efficiency of 100%. However, the stability of phosphorescent OLEDs still needs to be improved, where the key factor is the host material, in addition to the light emitter.

In order to simplify the device preparation process, the red/green/phosphorescent light-emitting devices are generally prepared by the single-host materials. However, the single-host materials usually result in imbalance in carrier transport, which leads to serious roll-off of device efficiency at high luminance and short lifetime. Although co-evaporation of two single-host materials with different polarities can attenuate the carrier imbalance caused by the single-host, the evaporation process is complex and not conducive to mass production.

In order to further attenuate the efficiency roll-off of the device at high luminance while simplifying the evaporation process of the material, a co-host material (i.e., organic mixture material) is commonly used to prepare a light-emitting layer in the industry. As a result, it makes the carrier transport relatively balanced, thus improving the device luminescence efficiency as well as lifetime, reducing the display production cost, and improving the production yield. However, the lifetime of the device still needs to be improved.

In order to meet the ever-increasing requirements on material stability, device simplicity and device lifetime, materials with more stable photoelectric performance are needed. Existing technologies, especially material solutions, still need to be improved and developed.

### SUMMARY

In one aspect, the present disclosure provides an organic compound comprising a structure of formula (I):

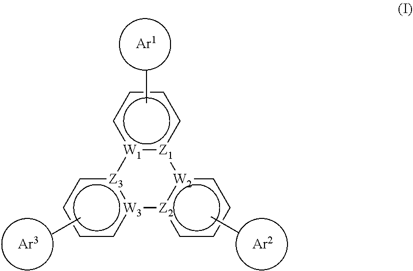

Where each of Z₁ to Z₃ is independently N or C, at least one of Z₁ to Z₃ is N; each of W₁ to W₃ is independently B or C, at least one of W₁ to W₃ is B; Ar¹ to Ar³ at multiple occurrences, are independently selected from the group consisting of null, a C₅-C₃₀ aromatic group, a C₅-C₃₀ heteroaromatic group, a C₅-C₃₀ non-aromatic ring system, and can be substituted with one or more R⁸s.

R⁸ at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic group containing 6 to 40 ring atoms, a substituted/unsubstituted heteroaromatic group containing 5 to 40 ring atoms, an aryloxy or heteroaryloxy group containing 6 to 40 ring atoms, and any combination thereof, where one or more R⁸s may form a monocyclic or polycyclic aliphatic or aromatic ring system with each other and/or with the rings bonded thereto.

In another aspect, the present disclosure also provides an organic mixture comprising a first compound H1 and a second compound H2, the first compound H1 comprises an organic compound as described herein, and min(LUMO(H1)−HOMO(H2), LUMO(H2)−HOMO(H1))≤min(E_{T}(H1), E_{T}(H2))+0.1 eV.

In yet another aspect, the present disclosure further provides an organic electronic device comprising an organic compound, or an organic mixture as described herein.

Beneficial effect: the organic compound as described herein may be used as a phosphorescent host material to obtain good device result. In particular, the organic mixture includes two host materials (i.e., a first compound H1 and a second compound H2) capable of forming an exiplex, and the first compound H1 and the second compound H2 can form type II heterojunction energy structure, and have high stability. The organic compound or organic mixture as described herein can be used as an electrophosphorescent host material or co-host material, which can improve the device luminescence efficiency and lifetime by matching a suitable dopant material. The organic mixture as described herein can also be used as a fluorescent co-host material or an emitting material, and the efficiency and lifetime of the electroluminescent device can be improved by combining a suitable fluorescent host material or guest material, thus providing a solution for manufacturing low-cost, high-efficiency and long-lifetime light-emitting devices.

### DETAILED DESCRIPTION OF THE EMBODIMENTS

The present disclosure provides an organic compound, an organic mixture, and the applications thereof in organic electroluminescent devices, aiming to solve the existing problems of poor organic host material stability, short device lifetime, and serious efficiency roll-off at high luminance.

In order to make the objects, technical solutions, and effects of the present disclosure more clear and definite, the present disclosure is further described in detail below. It should be understood that the embodiments described herein are only intended to explain the present disclosure and are not intended to limit the present disclosure.

As used herein, the terms “formulation”, “printing ink” and “ink” have the same meaning, and they are interchangeable with each other.

As used herein, the terms “host material”, “matrix material” have the same meaning, and they are interchangeable with each other.

In the present disclosure, the heterojunction refers to an interface region formed by contacting two different semiconductors. According to the alignment of the conduction band (LUMO) and the valence band (HOMO) of the two materials in the heterojunction, the heterojunction can be classified into type I heterojunction and type II heterojunction, while the basic features of the type II heterojunction are the separation of electrons and hole spatially near the intersection and the localization in the self-consistent quantum well. In addition, the overlap of wave functions near the interface leads to a reduction of the optical matrix elements, resulting in longer radiative lifetime and less exciton binding energy.

In one aspect, the present disclosure provides an organic compound comprising a structure of formula (I):

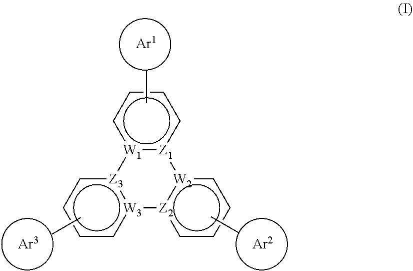

Where each of Z₁ to Z₃ is independently N or C, at least one of Z₁ to Z₃ is N; each of W₁ to W₃ is independently B or C, at least one of W₁ to W₃ is B; Ar¹ to Ar³ at multiple occurrences, are independently selected from the group consisting of null, a C₅-C₃₀ aromatic group, a C₅-C₃₀ heteroaromatic group, a C₅-C₃₀ non-aromatic ring system, and can be substituted with one or more R⁸s.

R⁸ at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic group containing 6 to 40 ring atoms, a substituted/unsubstituted heteroaromatic group containing 5 to 40 ring atoms, an aryloxy or heteroaryloxy group containing 6 to 40 ring atoms, and any combination thereof, where one or more R⁸s may form a monocyclic or polycyclic aliphatic or aromatic ring system with each other and/or with the rings bonded thereto.

Preferably, R⁸ at each occurrence is independently selected from the group consisting of F, Cl, Br, I, D, CN, NO₂, CF₃, B(OR⁹)₂, Si(R⁹)₃, a linear alkane, an alkane ether, a C₁-C₁₀ alkane thioether, a branched alkane, a cycloalkane, a C₃-C₁₀ alkane ether group; where R⁹ at each occurrence is independently selected from the group consisting of H, D, a C₁-C₁₀ aliphatic alkane, a aromatic hydrocarbon, and a C₅-C₁₀ substituted/unsubstituted aromatic or hetero-aromatic group.

In some embodiments, Ar¹ to Ar³ at multiple occurrences, are independently selected from the group consisting of an aromatic or a heteroaromatic ring containing 5 to 30 ring atoms. In some embodiments, the aromatic ring system contains 5 to 25 ring atoms in the ring system, the heteroaromatic ring system contains 5 to 25 ring atoms, together with at least one heteroatom, while the total number of the carbon atoms and the heteroatoms is at least 5. In some embodiments, the aromatic ring system contains 5 to 20 ring atoms in the ring system, the heteroaromatic ring system contains 5 to 20 ring atoms in the ring system, together with at least one heteroatom. In some embodiments, the aromatic ring system contains 5 to 20 ring atoms in the ring system, the heteroaromatic ring system contains 5 to 20 ring atoms in the ring system, together with at least one heteroatom. In some embodiments, the aromatic ring system contains 5 to 16 ring atoms in the ring system, the heteroaromatic ring system contains 5 to 16 ring atoms in the ring system, together with at least one heteroatom. The heteroatom is preferably selected from Si, N, P, O, S, and/or Ge, and particularly preferably selected from Si, N, P, O, and/or S.

The term “aromatic ring system” or “aromatic group” refers to a hydrocarbon group consisting of an aromatic ring, including monocyclic groups and polycyclic systems. The term “heteroaromatic ring system” or “heteroaromatic group” refers to a hydrocarbon group (containing a heteroatom) consisting of at least one heteroaromatic ring, including monocyclic groups and polycyclic systems. The polycyclic systems contain two or more rings, in which two carbon atoms are shared by two adjacent rings, i. e., fused rings. Specifically, at least one of the rings in the polycyclic rings are aromatic or heteroaromatic. For the purposes of the present disclosure, the aromatic ring groups or heteroaromatic groups comprise not only aromatic or heteroaromatic systems, but also a plurality of aromatic or heteroaromatic groups are interconnected by short non-aromatic units (for example by <10% of non-H atoms, more specifically 5% of non-H atoms, such as C, N or O atoms). Therefore, systems such as 9,9′-spirobifluorene, 9,9-diaryl fluorene, triarylamine, diaryl ethers, and other systems, should also be considered as aromatic groups for the purpose of this present disclosure.

Specifically, examples of the aromatic groups include benzene, naphthalene, anthracene, phenanthrene, perylene, naphthacene, pyrene, benzpyrene, triphenylene, acenaphthene, fluorene, and derivatives thereof.

Specifically, examples of heteroaromatic groups include furan, benzofuran, thiophene, benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzoisothiazole, benzimidazole, pyridine, pyrazine, pyridazine, pyrimidine, triazine, quinoline, isoquinoline, phenanthroline, quinoxaline, phenanthridine, primidine, quinazoline, quinazolinone, and derivatives thereof.

In some embodiments, at least one of Ar¹ to Ar³ comprises a non-aromatic ring system containing 2 to 20 carbon atoms which is either unsubstituted or substituted with R⁸.

For one of the purposes of the present disclosure, the non-aromatic ring system contains 1 to 10 carbon atoms in the ring system, preferably contains 1 to 6 carbon atoms, including both the saturated ring system and the partially unsaturated ring system, which may be either unsubstituted or substituted with one or more R²¹s, R²¹s may be the same or different at each occurrence and may also comprise one or more heteroatoms, preferably selected from Si, N, P, O, S and/or Ge, particularly preferably from Si, N, P, O and/or S. For example, cyclohexyl-like, piperidine-like systems, or cyclooctadiene-like ring systems. Meanwhile, the term also applies to fused non-aromatic ring systems.

In some embodiments, Ar¹ to Ar³ at multiple occurrences, are independently selected from null, one or combinations of more than one of the following groups:

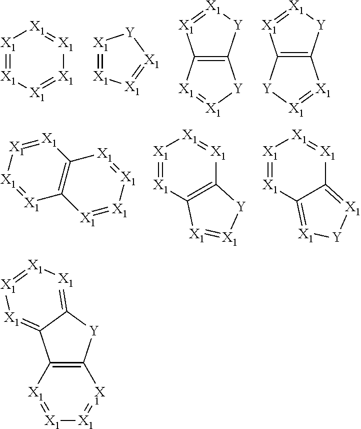

Where each X₁ is CR¹⁰ or N; each Y is selected from CR¹¹R¹², SiR¹³R¹⁴, NR¹⁵, C(═O), 5, or O; each of R¹⁰ to R¹⁵ is independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R¹⁰-R¹⁵ may form a monocyclic or polycyclic aliphatic or aromatic ring system with each other and/or with the rings bonded thereto.

In some embodiments, Ar¹ to Ar³ comprise the following structural formulas, which may be further substituted with one or more R¹⁰s, each R¹⁰ is defined as described above.

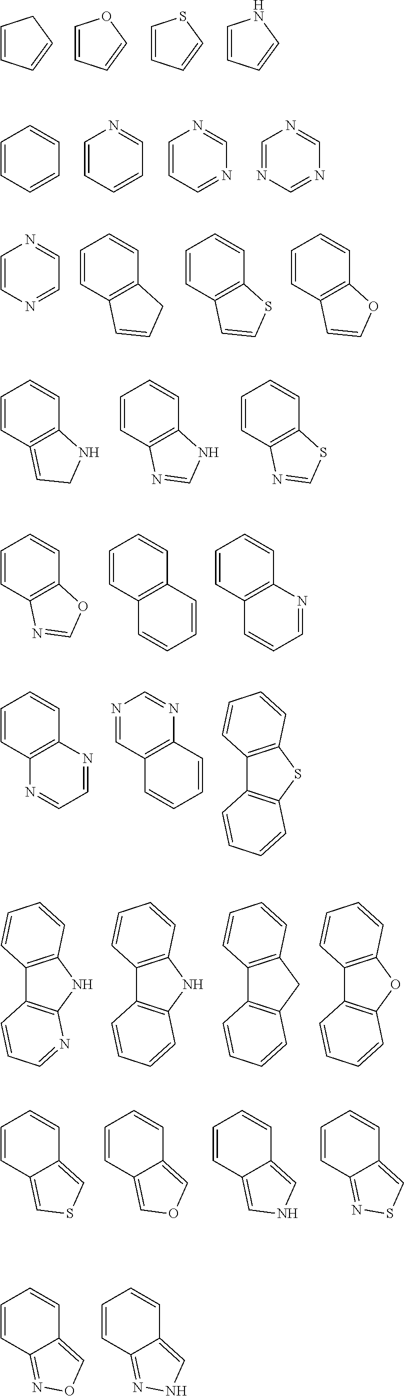

In some embodiments, Ar¹ to Ar³ at multiple occurrences, are independently selected from one or combinations of more than one of the following groups:

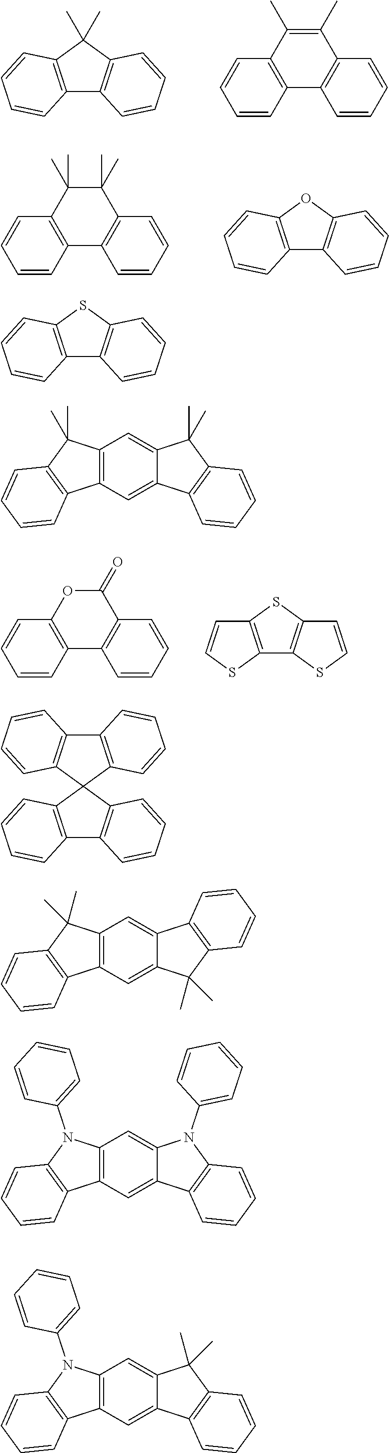

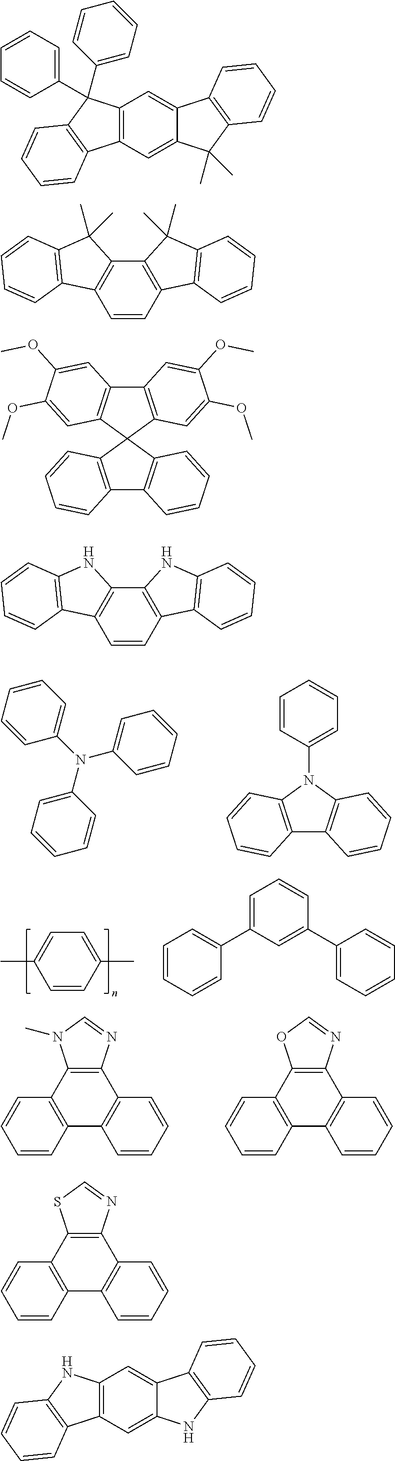

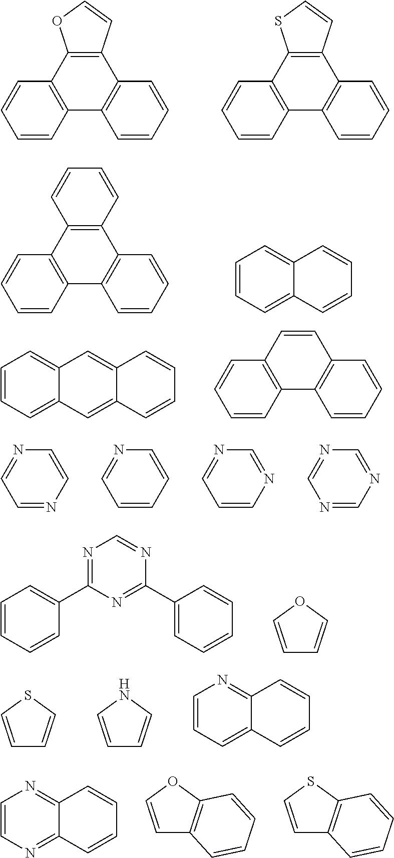

Where u is 1, or 2, or 3, or 4.

In some embodiments, the organic compound as described herein is selected from one of formulas (I-1)-(I-3):

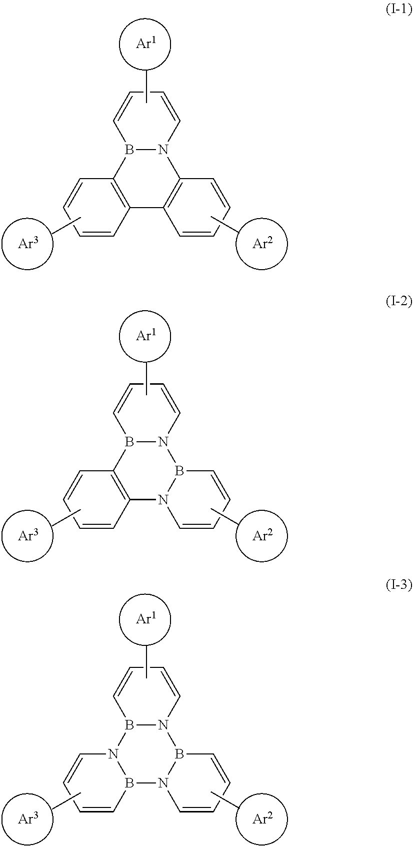

Where Ar¹ to Ar³ are identically defined as described above.

In some embodiments, the organic compound as described herein has a high triplet energy level T₁, generally ≥2.2 eV, preferably ≥2.4 eV, more preferably ≥2.5 eV, further preferably ≥2.6 eV, and most preferably ≥2.8 eV.

In some embodiments, the organic compound as described herein is partially deuterated; preferably 10% or more of total H, more preferably 20% or more of total H, further preferably 30% or more of total H, and most preferably 40% or more of total H, are deuterated.

In some embodiments, the organic compound as described herein facilitates a thermally activated delayed fluorescence effect. And the triplet exciton of the organic compound can be converted to a singlet exciton by reverse internal conversion when ΔE_{ST} is sufficiently small, thus achieving high luminescence efficiency and improving the material stability. In general, such material is obtained by connecting electron-donating group (Donor) to electron-deficient or electron-accepting (Acceptor) groups, having obvious D-A structures.

In some embodiments, in the organic compound of formulas (I), (I-1), (I-2), and (I-3), Ar¹ to Ar³ at multiple occurrences, at least one of them comprises an electron-donating group.

In some embodiments, the electron-donating group can be selected from formulas D1-D10:

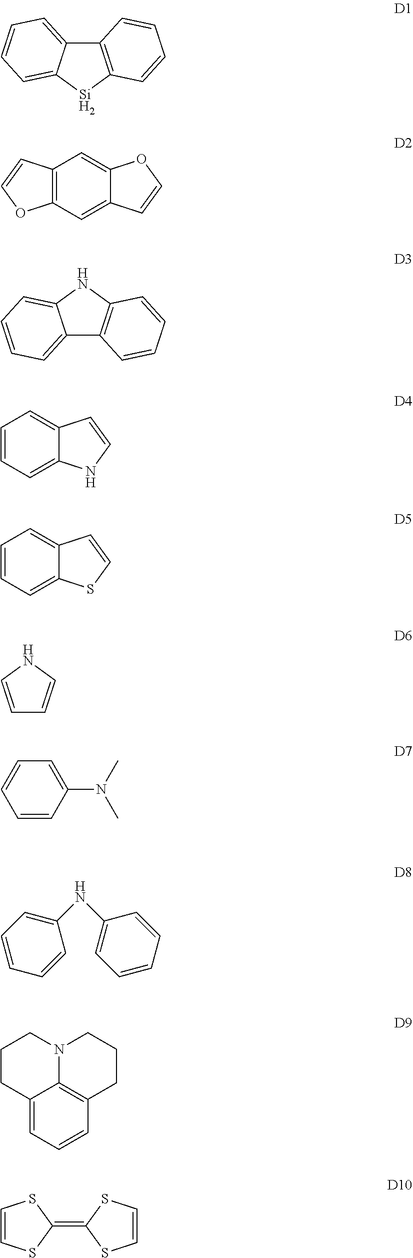

In some embodiments, in the organic compound of formulas (I), (I-1), (I-2), and (I-3), Ar¹ to Ar³ at multiple occurrences, at least one of them comprises an electron-accepting group.

In some embodiments, the electron-accepting group can be selected from F, a cyano group, or comprise the following groups:

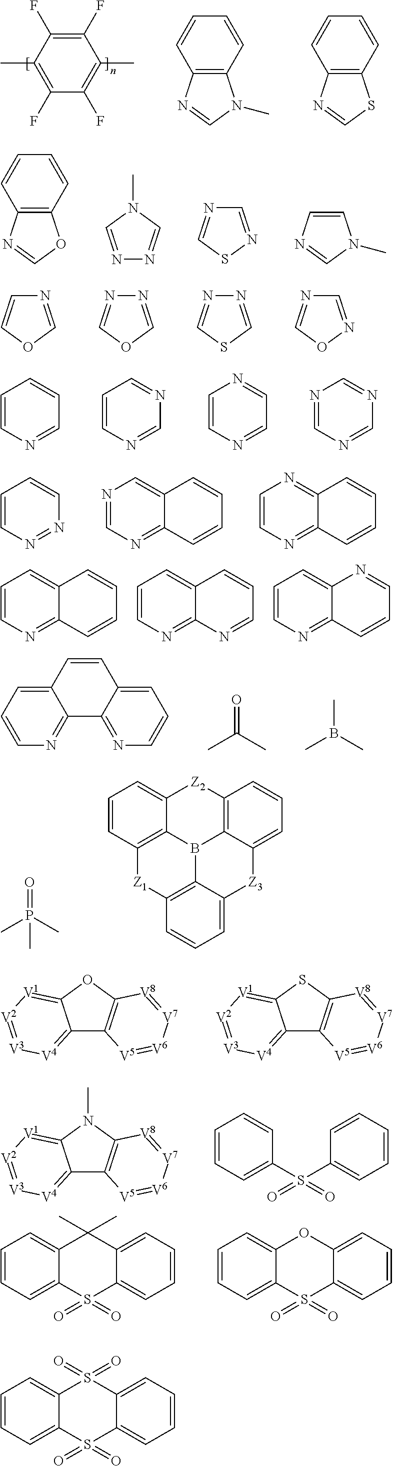

Where n is an integer from 1 to 3; each of V¹ to V⁸ is CR¹⁶ or N, and at least one of them is N; each of Z₁ to Z₃ is a single bond, or C(R¹⁶)₂, or O, or S; R¹⁶ at multiple occurrences, is independently selected from the following groups: H, alkyl, alkoxy, amino, alkene, alkyne, aralkyl, heteroalkyl, aryl, or heteroaryl.

Examples of the preferred organic compounds according to formulas (I), (I-1) to (I-3) are shown in the following structures. And these structures can be substituted arbitrarily.

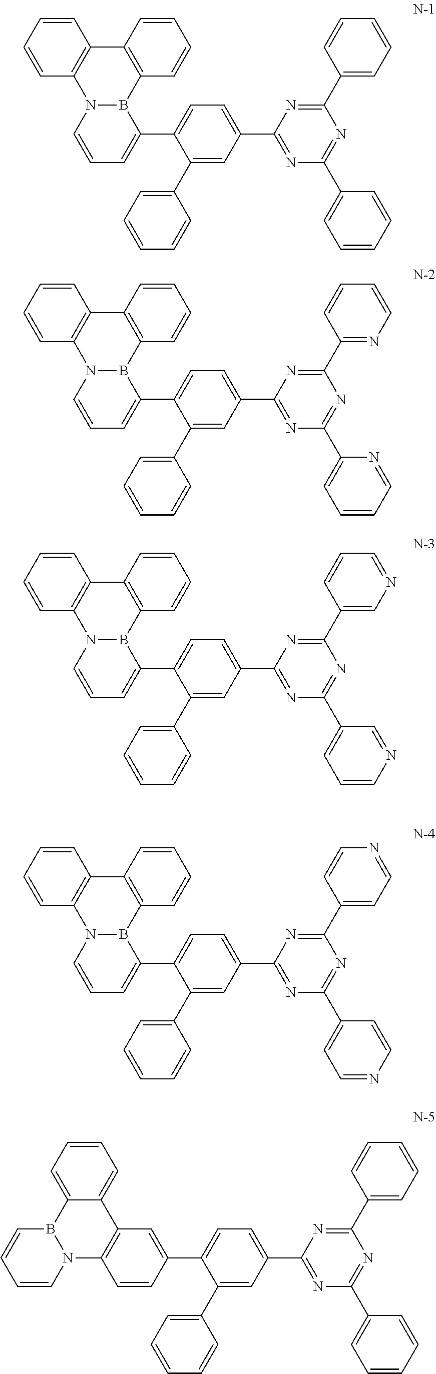

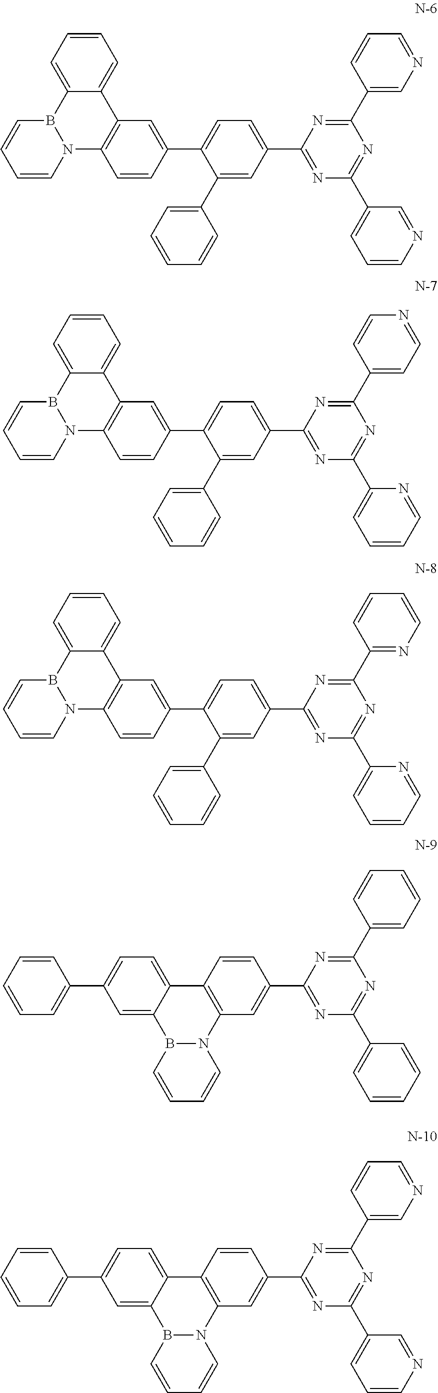

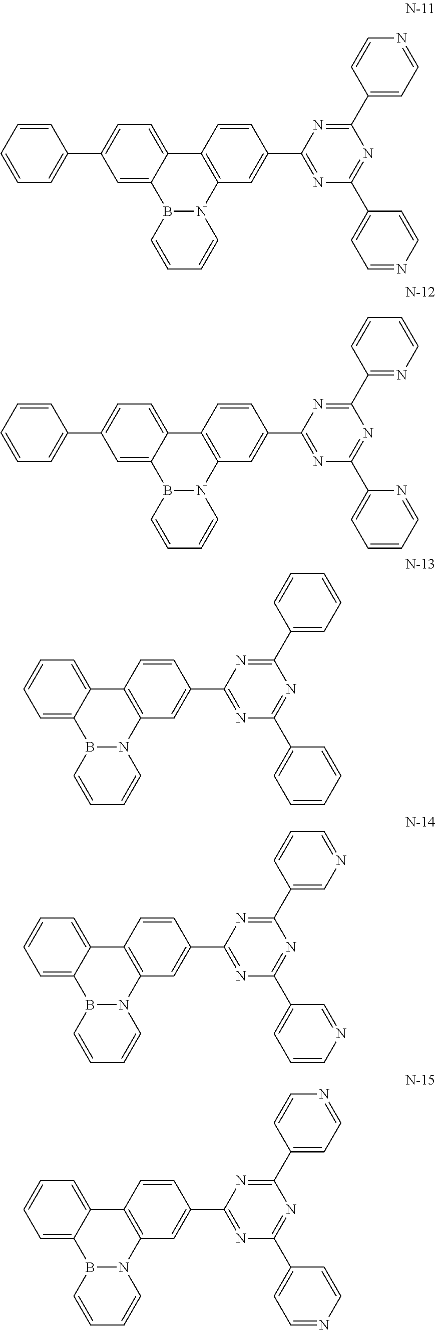

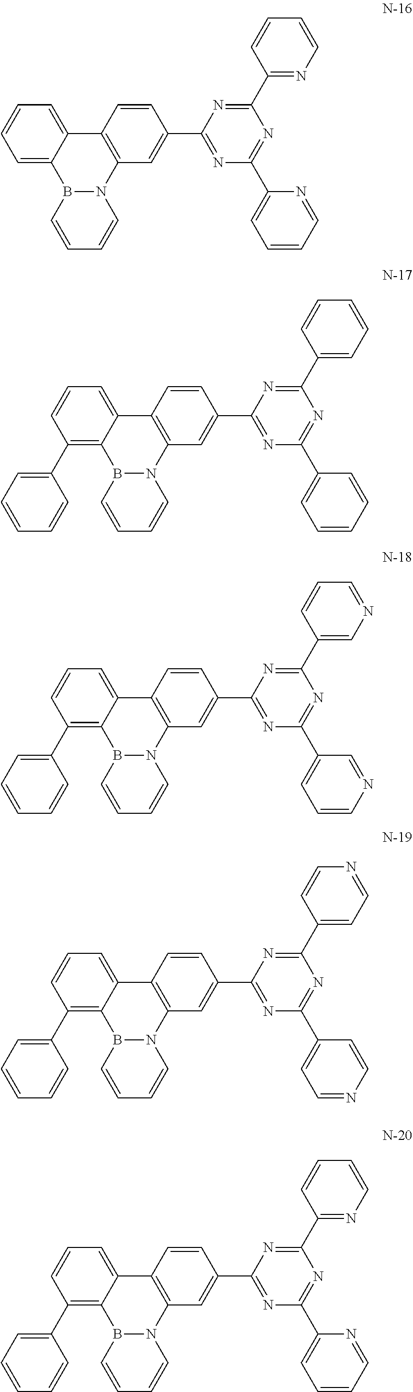 [SMILES: C1=CB2c3ccccc3-c3ccc(-c4nc(-c5ccccn5)nc(-c5ccccn5)n4)cc3N2C=C1 ; C1=CB2c3c(-c4ccccc4)cccc3-c3ccc(-c4nc(-c5ccccc5)nc(-c5ccccc5)n4)cc3N2C=C1 ; C1=CB2c3c(-c4ccccc4)cccc3-c3ccc(-c4nc(-c5cccnc5)nc(-c5cccnc5)n4)cc3N2C=C1 ; C1=CB2c3c(-c4ccccc4)cccc3-c3ccc(-c4nc(-c5ccncc5)nc(-c5ccncc5)n4)cc3N2C=C1 ; C1=CB2c3c(-c4ccccc4)cccc3-c3ccc(-c4nc(-c5ccccn5)nc(-c5ccccn5)n4)cc3N2C=C1]

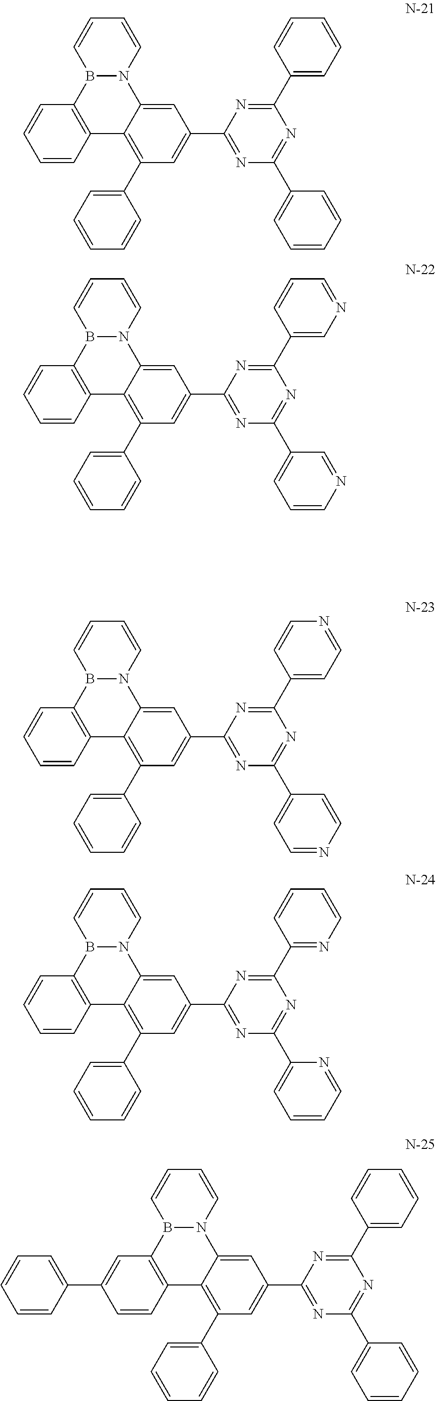

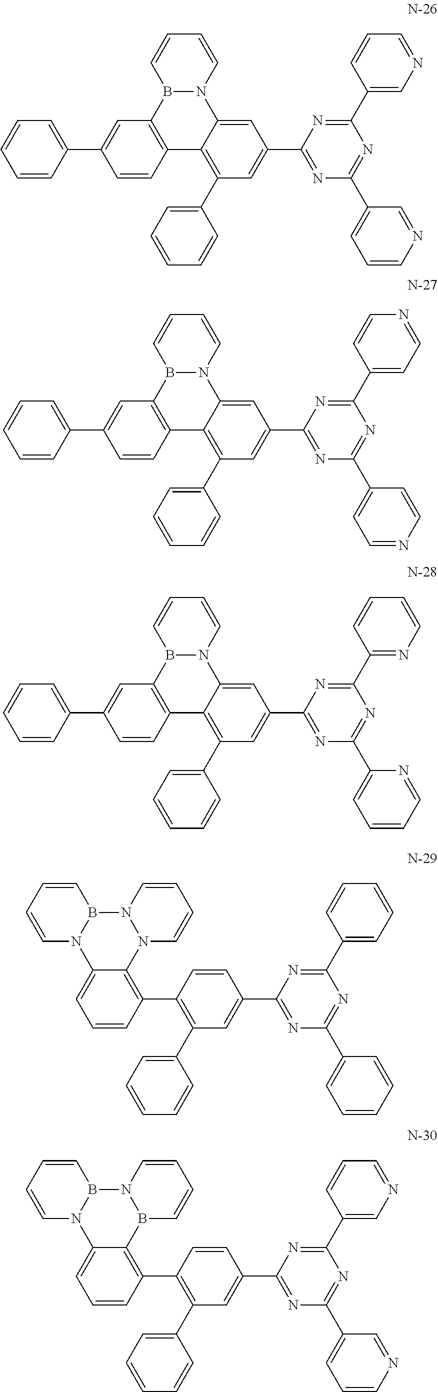

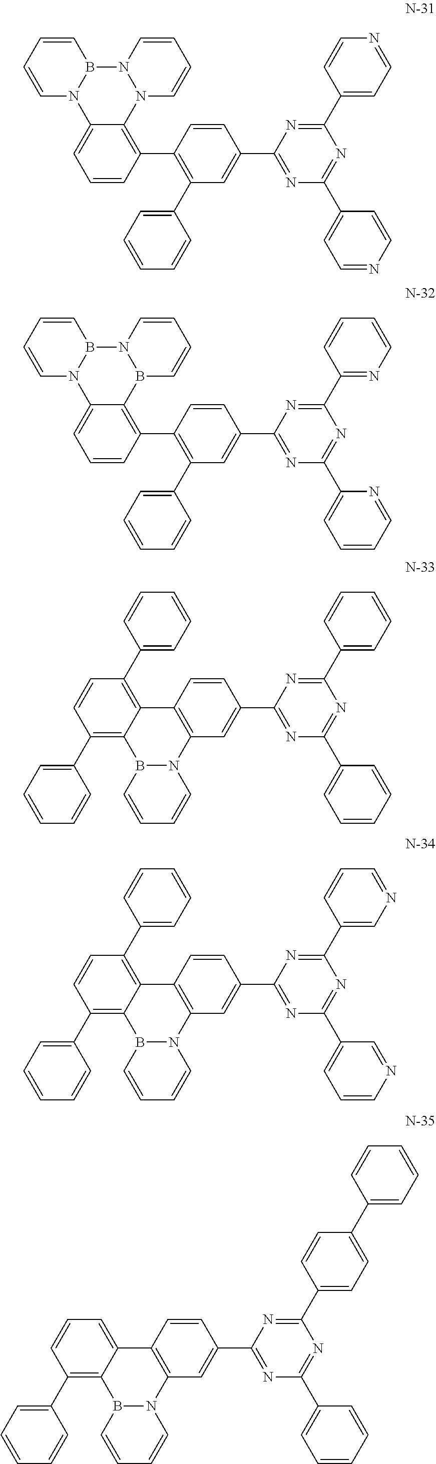

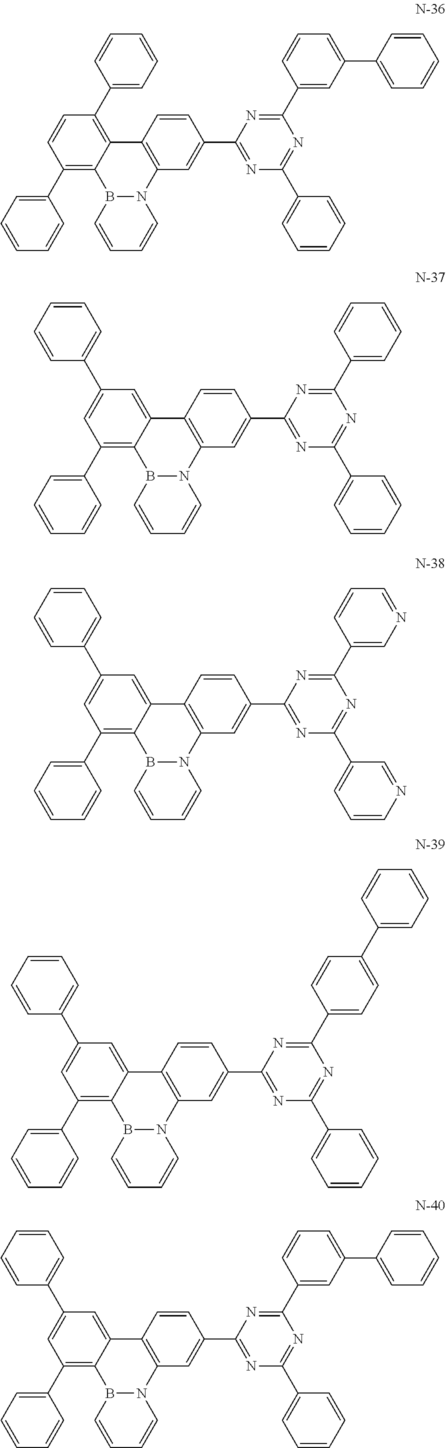

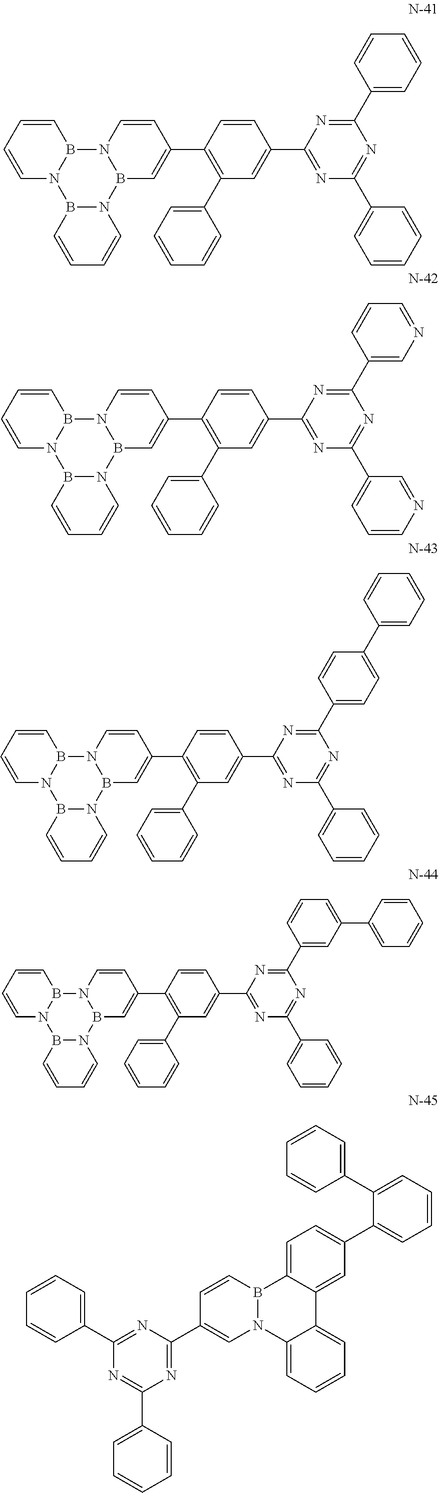

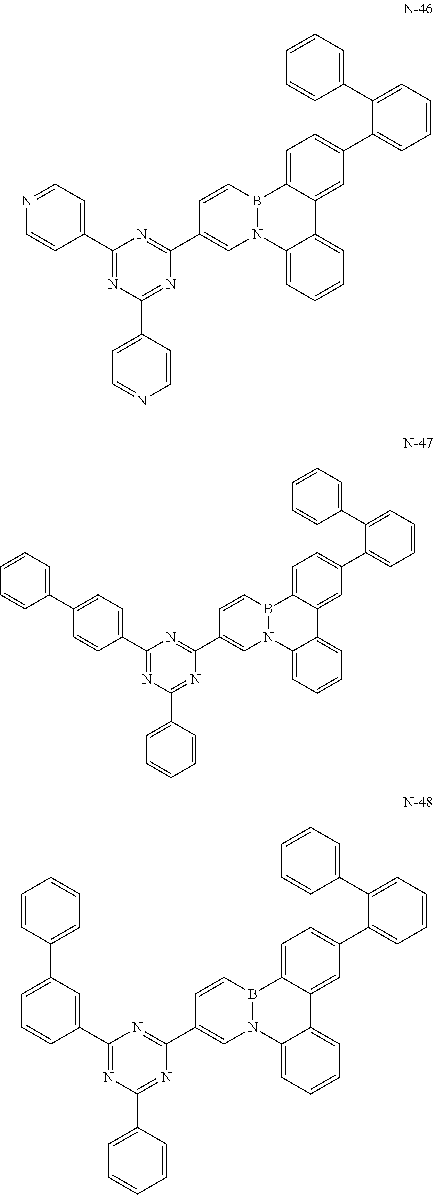 [SMILES: C1=CC(c2nc(-c3ccncc3)nc(-c3ccncc3)n2)=CN2B1c1ccc(-c3ccccc3-c3ccccc3)cc1-c1ccccc12 ; C1=CC(c2nc(-c3ccccc3)nc(-c3ccc(-c4ccccc4)cc3)n2)=CN2B1c1ccc(-c3ccccc3-c3ccccc3)cc1-c1ccccc12 ; C1=CC(c2nc(-c3ccccc3)nc(-c3cccc(-c4ccccc4)c3)n2)=CN2B1c1ccc(-c3ccccc3-c3ccccc3)cc1-c1ccccc12]

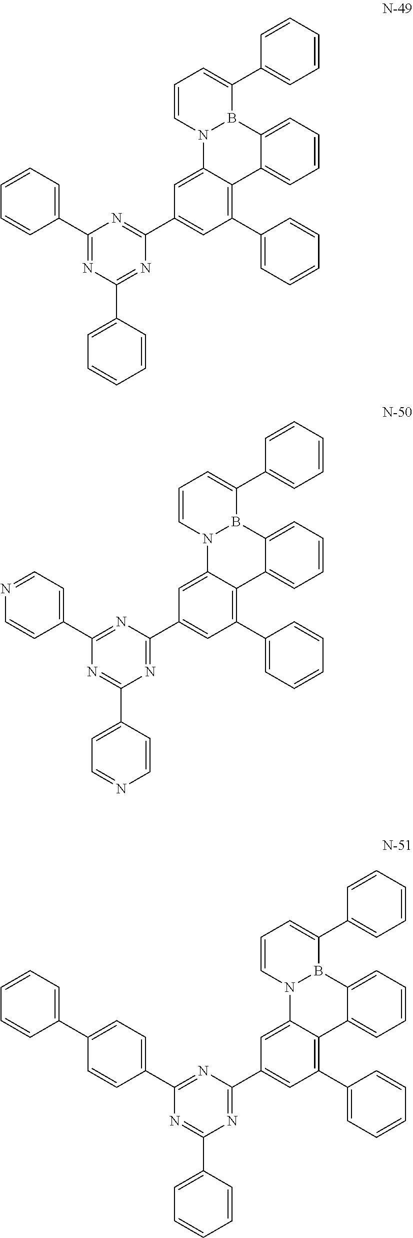 [SMILES: C1=CN2B(C(c3ccccc3)=C1)c1ccccc1-c1c(-c3ccccc3)cc(-c3nc(-c4ccccc4)nc(-c4ccccc4)n3)cc12 ; C1=CN2B(C(c3ccccc3)=C1)c1ccccc1-c1c(-c3ccccc3)cc(-c3nc(-c4ccncc4)nc(-c4ccncc4)n3)cc12 ; C1=CN2B(C(c3ccccc3)=C1)c1ccccc1-c1c(-c3ccccc3)cc(-c3nc(-c4ccccc4)nc(-c4ccc(-c5ccccc5)cc4)n3)cc12]

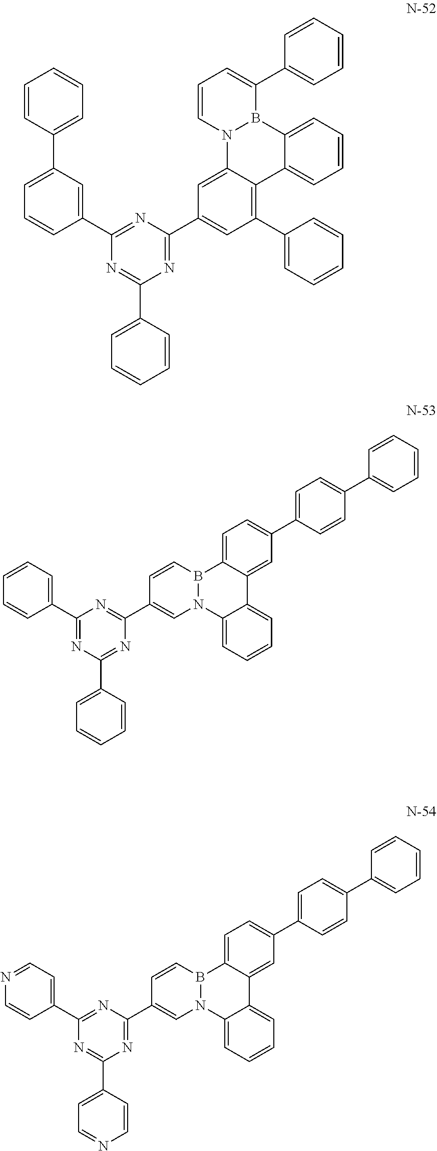 [SMILES: C1=CN2B(C(c3ccccc3)=C1)c1ccccc1-c1c(-c3ccccc3)cc(-c3nc(-c4ccccc4)nc(-c4cccc(-c5ccccc5)c4)n3)cc12 ; C1=CC(c2nc(-c3ccccc3)nc(-c3ccccc3)n2)=CN2B1c1ccc(-c3ccc(-c4ccccc4)cc3)cc1-c1ccccc12 ; C1=CC(c2nc(-c3ccncc3)nc(-c3ccncc3)n2)=CN2B1c1ccc(-c3ccc(-c4ccccc4)cc3)cc1-c1ccccc12]

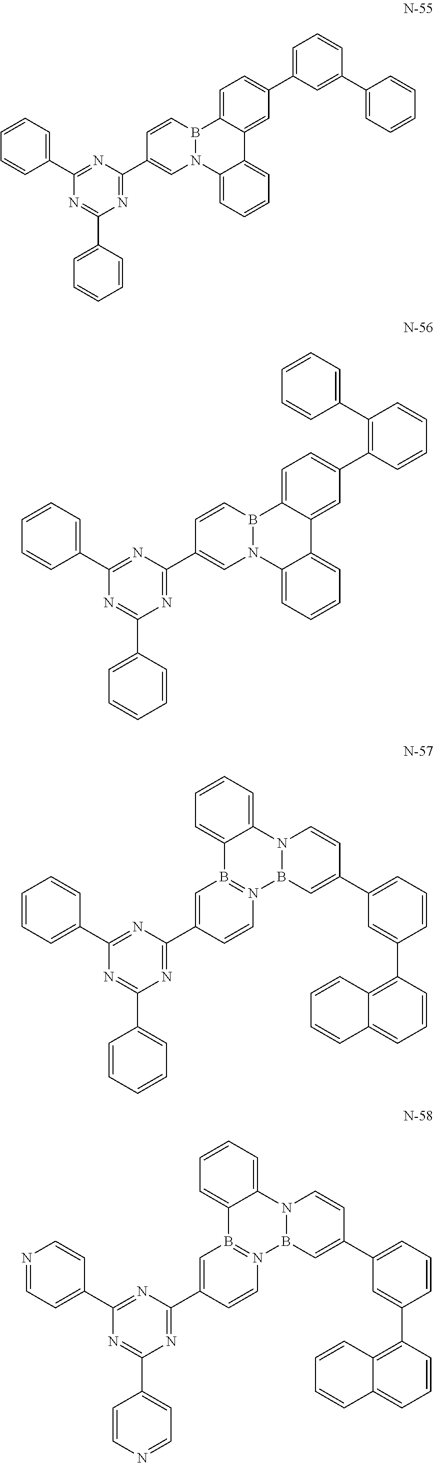

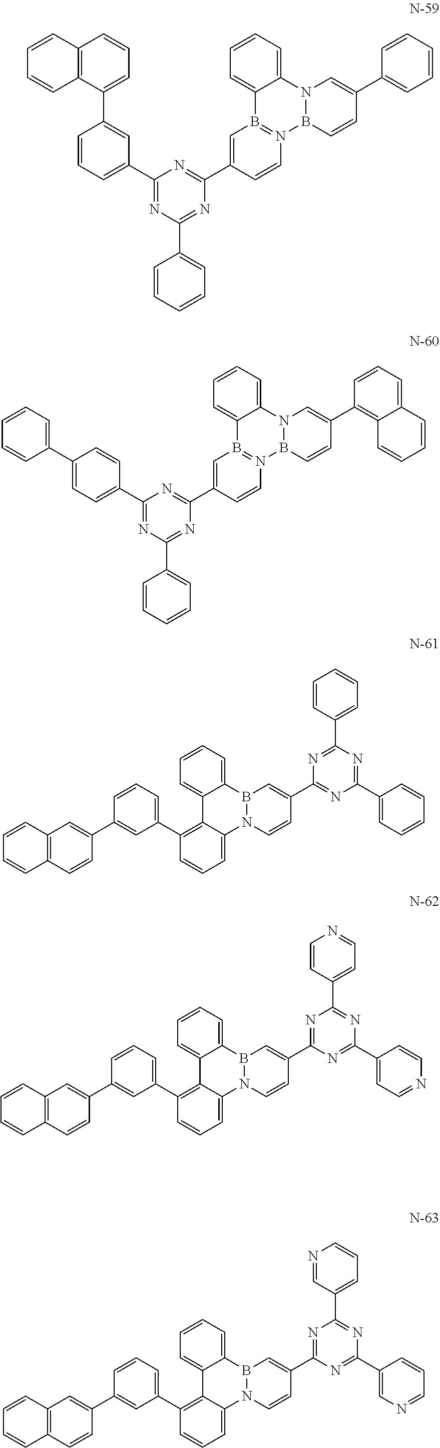

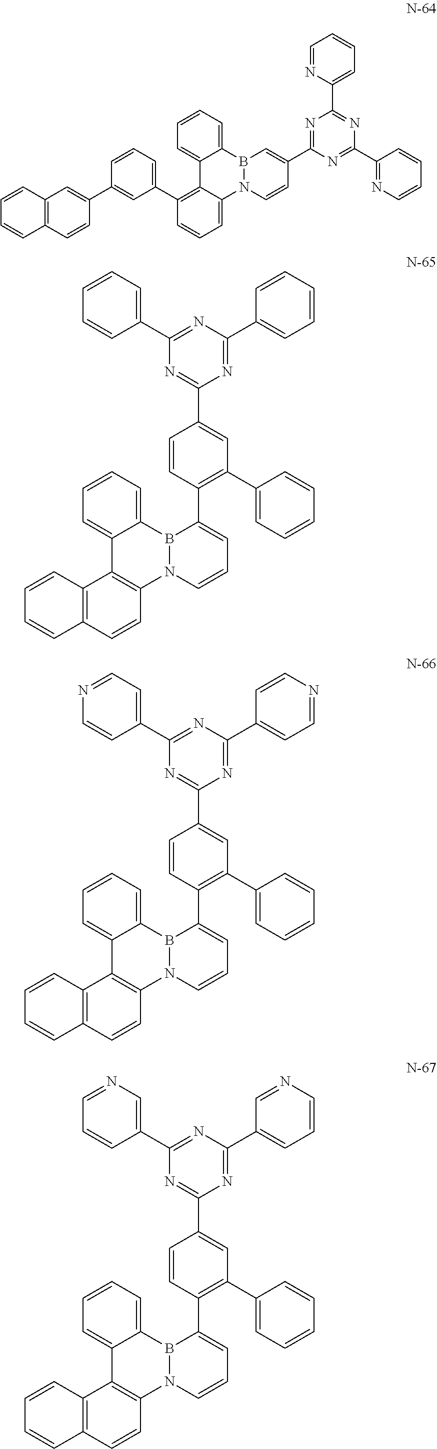

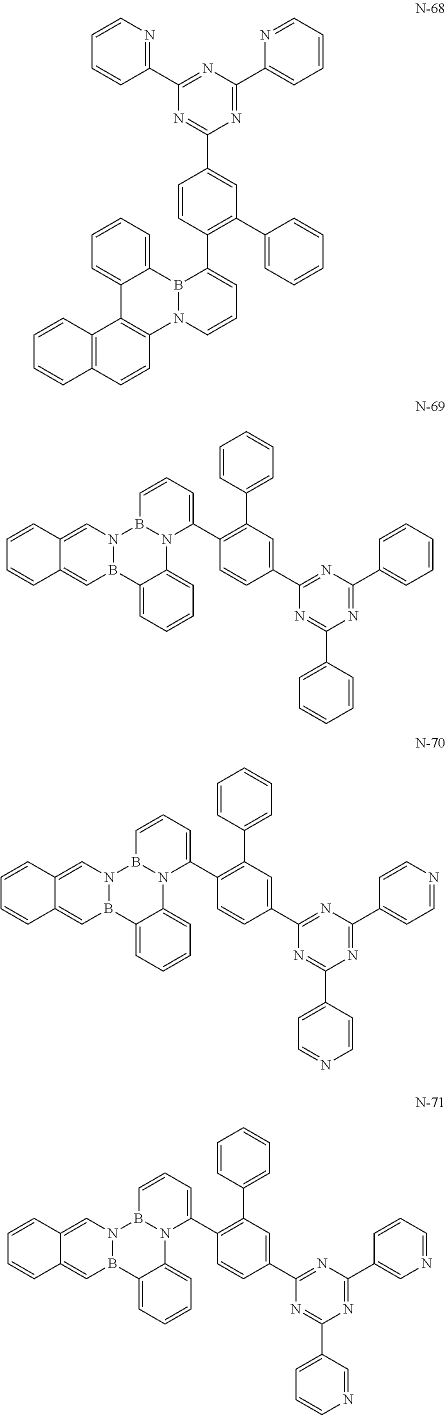 [SMILES: C1=CN2B(C(c3ccc(-c4nc(-c5ccccn5)nc(-c5ccccn5)n4)cc3-c3ccccc3)=C1)c1ccccc1-c1c2ccc2ccccc12 ; C1=CB2N3C=c4ccccc4=CB3c3ccccc3N2C(c2ccc(-c3nc(-c4ccccc4)nc(-c4ccccc4)n3)cc2-c2ccccc2)=C1 ; C1=CB2N3C=c4ccccc4=CB3c3ccccc3N2C(c2ccc(-c3nc(-c4ccncc4)nc(-c4ccncc4)n3)cc2-c2ccccc2)=C1 ; C1=CB2N3C=c4ccccc4=CB3c3ccccc3N2C(c2ccc(-c3nc(-c4cccnc4)nc(-c4cccnc4)n3)cc2-c2ccccc2)=C1]

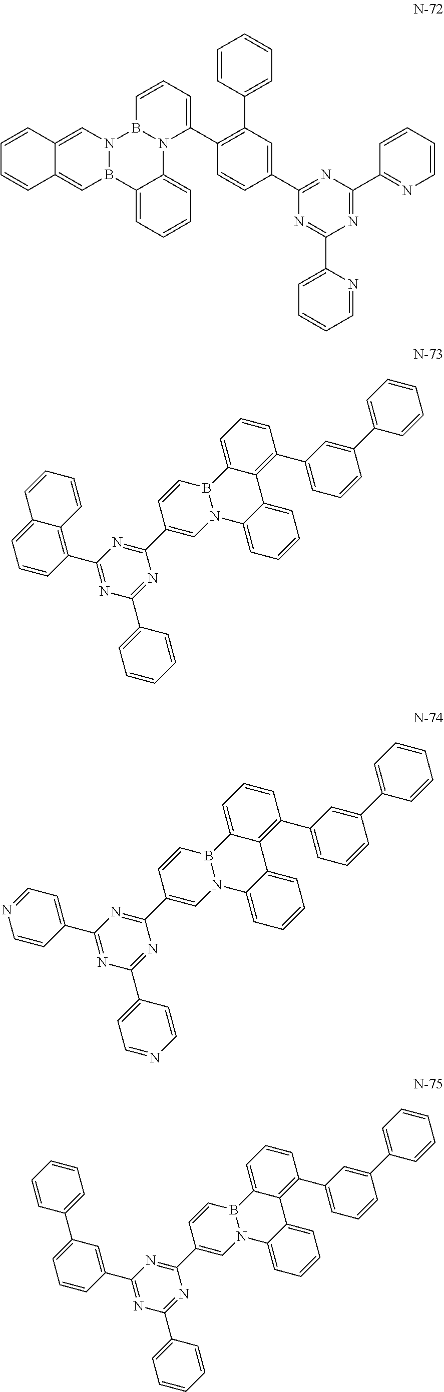 [SMILES: C1=CB2N3C=c4ccccc4=CB3c3ccccc3N2C(c2ccc(-c3nc(-c4ccccn4)nc(-c4ccccn4)n3)cc2-c2ccccc2)=C1 ; C1=CC(c2nc(-c3ccccc3)nc(-c3cccc4ccccc34)n2)=CN2B1c1cccc(-c3cccc(-c4ccccc4)c3)c1-c1ccccc12 ; C1=CC(c2nc(-c3ccncc3)nc(-c3ccncc3)n2)=CN2B1c1cccc(-c3cccc(-c4ccccc4)c3)c1-c1ccccc12 ; C1=CC(c2nc(-c3ccccc3)nc(-c3cccc(-c4ccccc4)c3)n2)=CN2B1c1cccc(-c3cccc(-c4ccccc4)c3)c1-c1ccccc12]

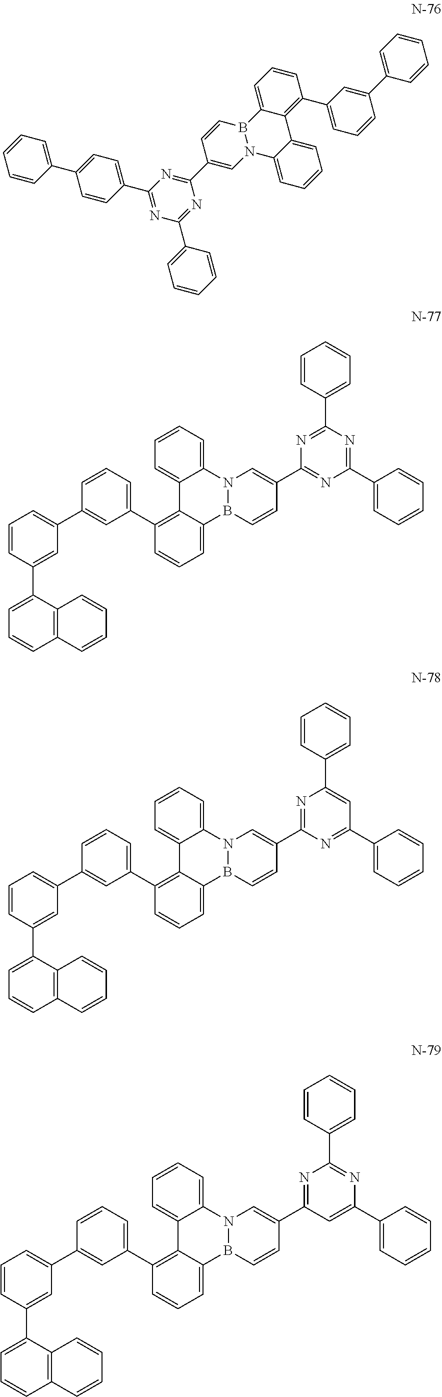 [SMILES: C1=CC(c2nc(-c3ccccc3)nc(-c3ccc(-c4ccccc4)cc3)n2)=CN2B1c1cccc(-c3cccc(-c4ccccc4)c3)c1-c1ccccc12 ; C1=CC(c2nc(-c3ccccc3)nc(-c3ccccc3)n2)=CN2B1c1cccc(-c3cccc(-c4cccc(-c5cccc6ccccc56)c4)c3)c1-c1ccccc12 ; C1=CC(c2nc(-c3ccccc3)cc(-c3ccccc3)n2)=CN2B1c1cccc(-c3cccc(-c4cccc(-c5cccc6ccccc56)c4)c3)c1-c1ccccc12 ; C1=CC(c2cc(-c3ccccc3)nc(-c3ccccc3)n2)=CN2B1c1cccc(-c3cccc(-c4cccc(-c5cccc6ccccc56)c4)c3)c1-c1ccccc12]

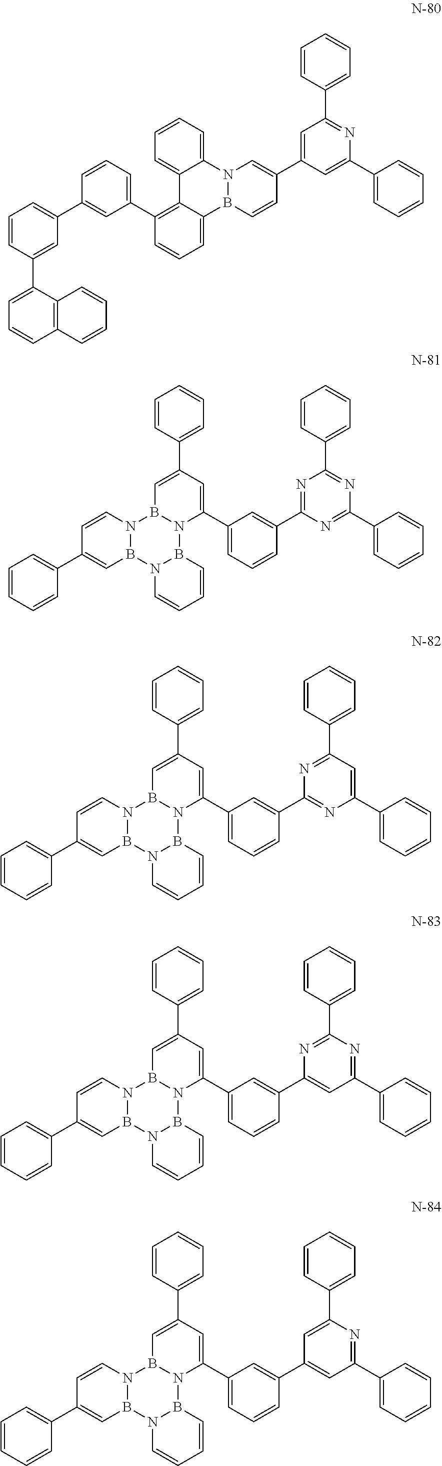 [SMILES: C1=CC(c2cc(-c3ccccc3)nc(-c3ccccc3)c2)=CN2B1c1cccc(-c3cccc(-c4cccc(-c5cccc6ccccc56)c4)c3)c1-c1ccccc12 ; C1=CB2N(C=C1)B1C=C(c3ccccc3)C=CN1B1C=C(c3ccccc3)C=C(c3cccc(-c4nc(-c5ccccc5)nc(-c5ccccc5)n4)c3)N21 ; C1=CB2N(C=C1)B1C=C(c3ccccc3)C=CN1B1C=C(c3ccccc3)C=C(c3cccc(-c4nc(-c5ccccc5)cc(-c5ccccc5)n4)c3)N21 ; C1=CB2N(C=C1)B1C=C(c3ccccc3)C=CN1B1C=C(c3ccccc3)C=C(c3cccc(-c4cc(-c5ccccc5)nc(-c5ccccc5)n4)c3)N21 ; C1=CB2N(C=C1)B1C=C(c3ccccc3)C=CN1B1C=C(c3ccccc3)C=C(c3cccc(-c4cc(-c5ccccc5)nc(-c5ccccc5)c4)c3)N21]

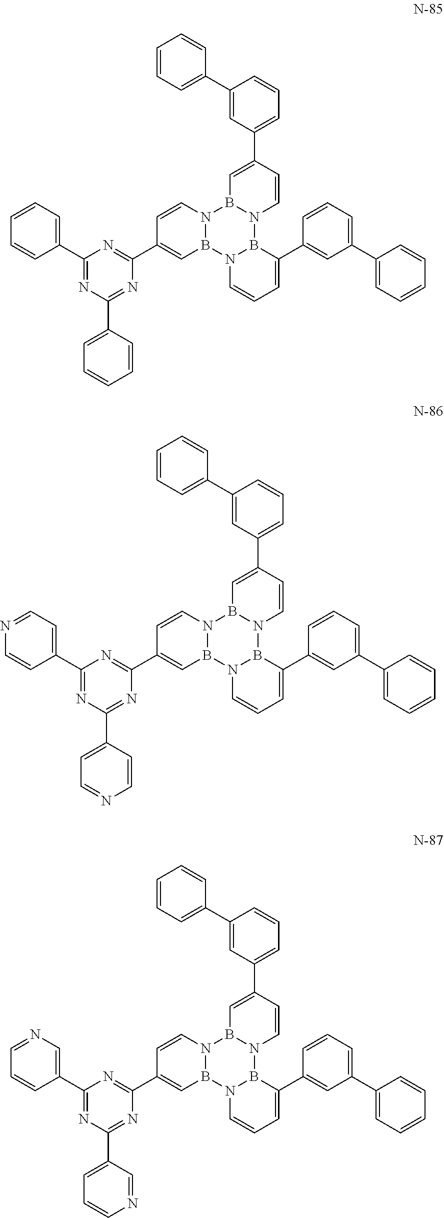 [SMILES: C1=CN2B3C=C(c4nc(-c5ccccc5)nc(-c5ccccc5)n4)C=CN3B3C=C(c4cccc(-c5ccccc5)c4)C=CN3B2C(c2cccc(-c3ccccc3)c2)=C1 ; C1=CN2B3C=C(c4nc(-c5ccncc5)nc(-c5ccncc5)n4)C=CN3B3C=C(c4cccc(-c5ccccc5)c4)C=CN3B2C(c2cccc(-c3ccccc3)c2)=C1 ; C1=CN2B3C=C(c4nc(-c5cccnc5)nc(-c5cccnc5)n4)C=CN3B3C=C(c4cccc(-c5ccccc5)c4)C=CN3B2C(c2cccc(-c3ccccc3)c2)=C1]

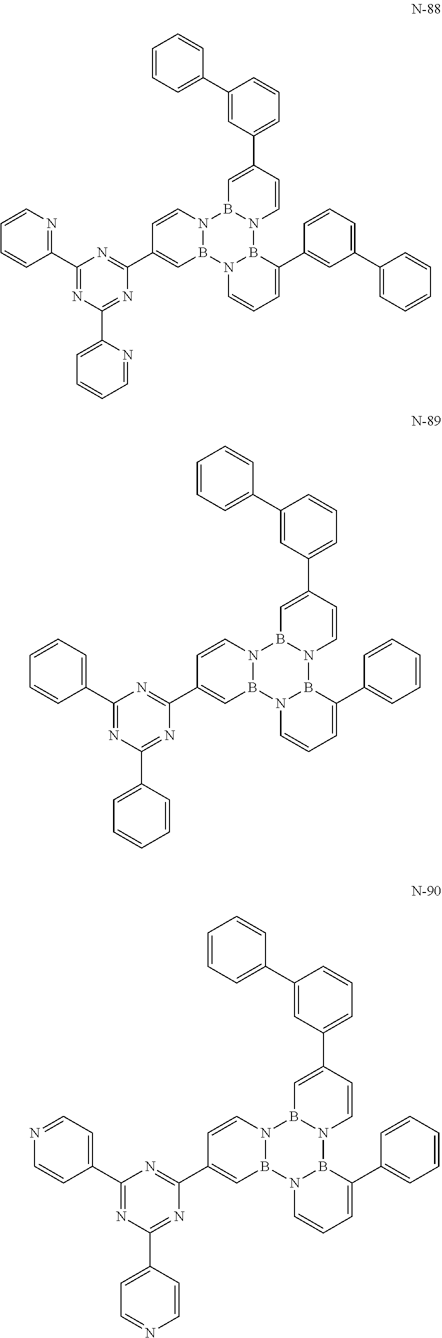 [SMILES: C1=CN2B3C=C(c4nc(-c5ccccn5)nc(-c5ccccn5)n4)C=CN3B3C=C(c4cccc(-c5ccccc5)c4)C=CN3B2C(c2cccc(-c3ccccc3)c2)=C1 ; C1=CN2B3C=C(c4nc(-c5ccccc5)nc(-c5ccccc5)n4)C=CN3B3C=C(c4cccc(-c5ccccc5)c4)C=CN3B2C(c2ccccc2)=C1 ; C1=CN2B3C=C(c4nc(-c5ccncc5)nc(-c5ccncc5)n4)C=CN3B3C=C(c4cccc(-c5ccccc5)c4)C=CN3B2C(c2ccccc2)=C1]

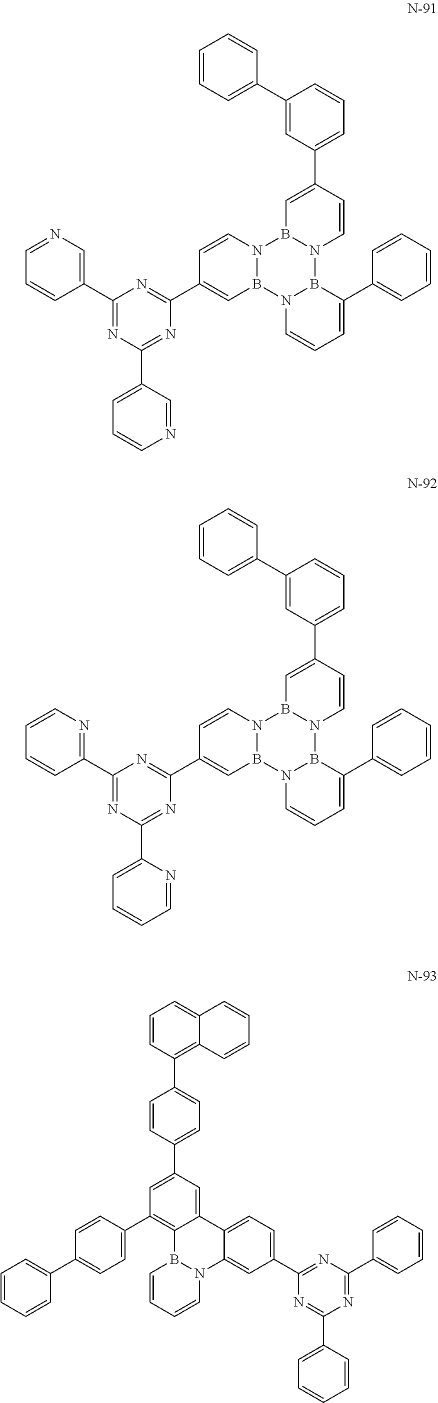 [SMILES: C1=CN2B3C=C(c4nc(-c5cccnc5)nc(-c5cccnc5)n4)C=CN3B3C=C(c4cccc(-c5ccccc5)c4)C=CN3B2C(c2ccccc2)=C1 ; C1=CN2B3C=C(c4nc(-c5ccccn5)nc(-c5ccccn5)n4)C=CN3B3C=C(c4cccc(-c5ccccc5)c4)C=CN3B2C(c2ccccc2)=C1 ; C1=CB2c3c(-c4ccc(-c5ccccc5)cc4)cc(-c4ccc(-c5cccc6ccccc56)cc4)cc3-c3ccc(-c4nc(-c5ccccc5)nc(-c5ccccc5)n4)cc3N2C=C1]

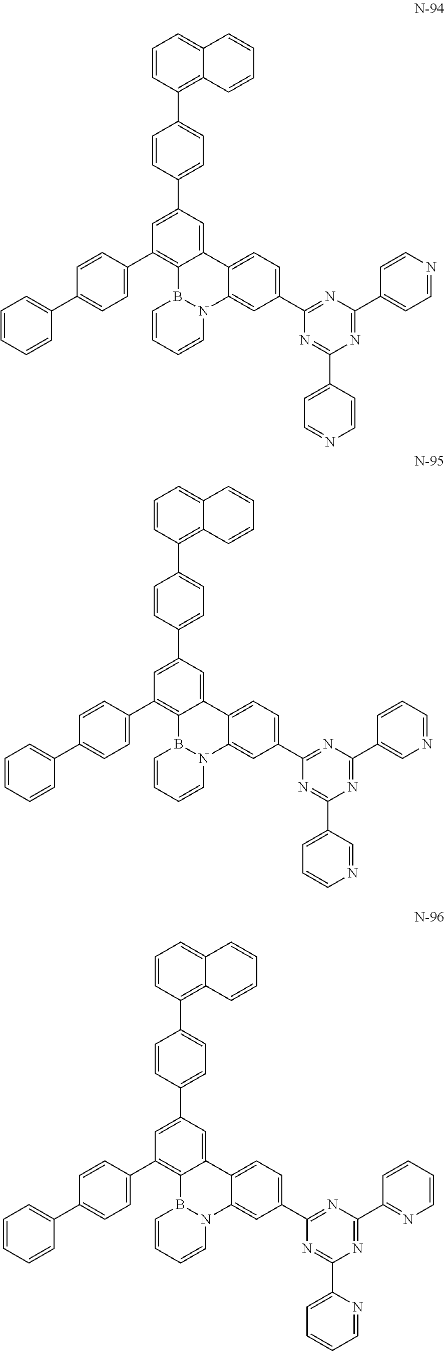 [SMILES: C1=CB2c3c(-c4ccc(-c5ccccc5)cc4)cc(-c4ccc(-c5cccc6ccccc56)cc4)cc3-c3ccc(-c4nc(-c5ccncc5)nc(-c5ccncc5)n4)cc3N2C=C1 ; C1=CB2c3c(-c4ccc(-c5ccccc5)cc4)cc(-c4ccc(-c5cccc6ccccc56)cc4)cc3-c3ccc(-c4nc(-c5cccnc5)nc(-c5cccnc5)n4)cc3N2C=C1 ; C1=CB2c3c(-c4ccc(-c5ccccc5)cc4)cc(-c4ccc(-c5cccc6ccccc56)cc4)cc3-c3ccc(-c4nc(-c5ccccn5)nc(-c5ccccn5)n4)cc3N2C=C1]

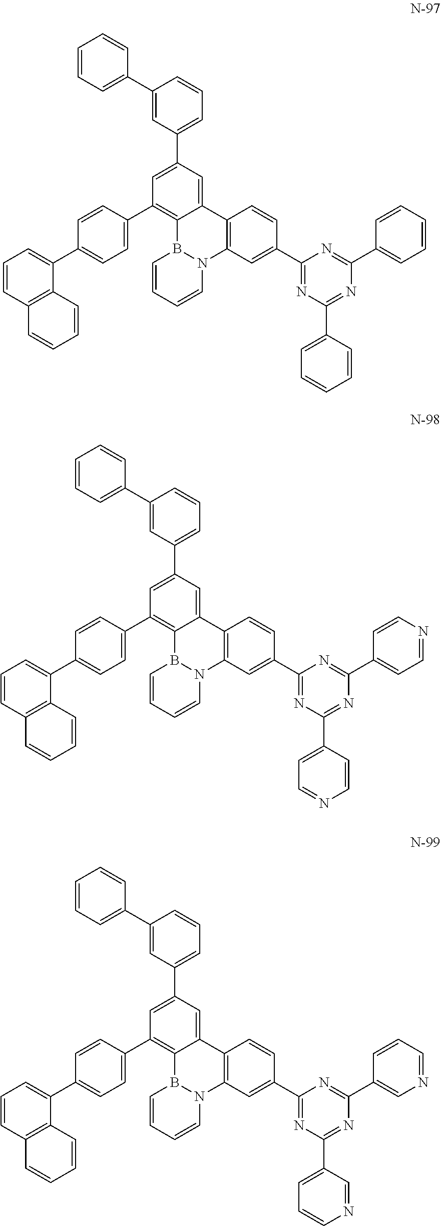 [SMILES: C1=CB2c3c(-c4ccc(-c5cccc6ccccc56)cc4)cc(-c4cccc(-c5ccccc5)c4)cc3-c3ccc(-c4nc(-c5ccccc5)nc(-c5ccccc5)n4)cc3N2C=C1 ; C1=CB2c3c(-c4ccc(-c5cccc6ccccc56)cc4)cc(-c4cccc(-c5ccccc5)c4)cc3-c3ccc(-c4nc(-c5ccncc5)nc(-c5ccncc5)n4)cc3N2C=C1 ; C1=CB2c3c(-c4ccc(-c5cccc6ccccc56)cc4)cc(-c4cccc(-c5ccccc5)c4)cc3-c3ccc(-c4nc(-c5cccnc5)nc(-c5cccnc5)n4)cc3N2C=C1]

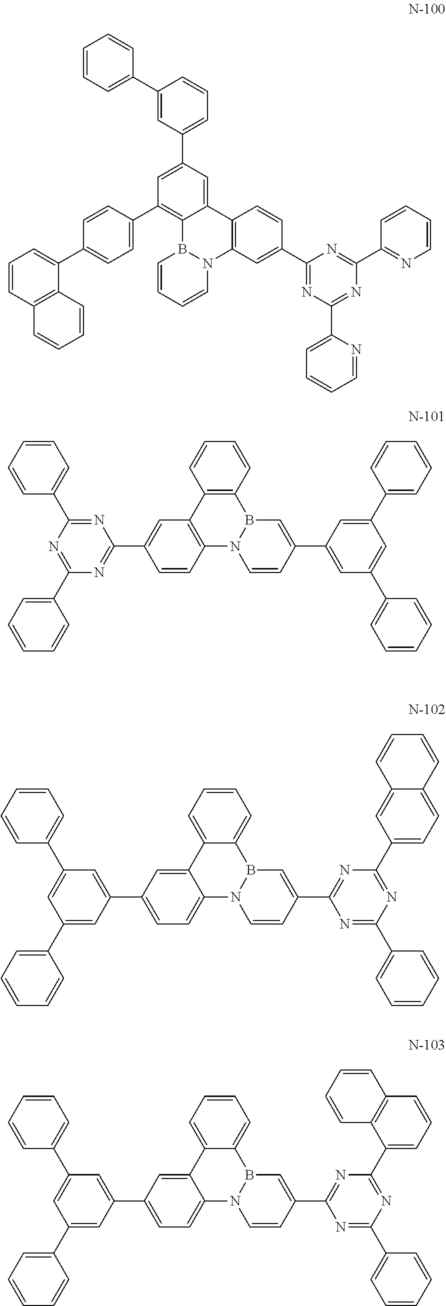 [SMILES: C1=CB2c3c(-c4ccc(-c5cccc6ccccc56)cc4)cc(-c4cccc(-c5ccccc5)c4)cc3-c3ccc(-c4nc(-c5ccccn5)nc(-c5ccccn5)n4)cc3N2C=C1 ; C1=CN2B(C=C1c1cc(-c3ccccc3)cc(-c3ccccc3)c1)c1ccccc1-c1cc(-c3nc(-c4ccccc4)nc(-c4ccccc4)n3)ccc12 ; C1=CN2B(C=C1c1nc(-c3ccccc3)nc(-c3ccc4ccccc4c3)n1)c1ccccc1-c1cc(-c3cc(-c4ccccc4)cc(-c4ccccc4)c3)ccc12 ; C1=CN2B(C=C1c1nc(-c3ccccc3)nc(-c3cccc4ccccc34)n1)c1ccccc1-c1cc(-c3cc(-c4ccccc4)cc(-c4ccccc4)c3)ccc12]

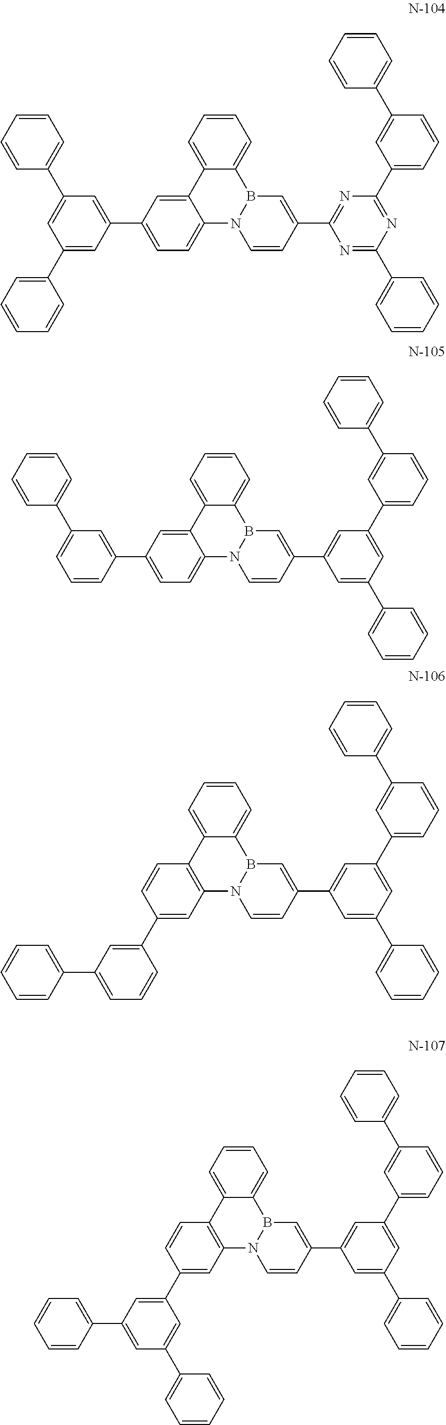 [SMILES: C1=CN2B(C=C1c1nc(-c3ccccc3)nc(-c3cccc(-c4ccccc4)c3)n1)c1ccccc1-c1cc(-c3cc(-c4ccccc4)cc(-c4ccccc4)c3)ccc12 ; C1=CN2B(C=C1c1cc(-c3ccccc3)cc(-c3cccc(-c4ccccc4)c3)c1)c1ccccc1-c1cc(-c3cccc(-c4ccccc4)c3)ccc12 ; C1=CN2B(C=C1c1cc(-c3ccccc3)cc(-c3cccc(-c4ccccc4)c3)c1)c1ccccc1-c1ccc(-c3cccc(-c4ccccc4)c3)cc12 ; C1=CN2B(C=C1c1cc(-c3ccccc3)cc(-c3cccc(-c4ccccc4)c3)c1)c1ccccc1-c1ccc(-c3cc(-c4ccccc4)cc(-c4ccccc4)c3)cc12]

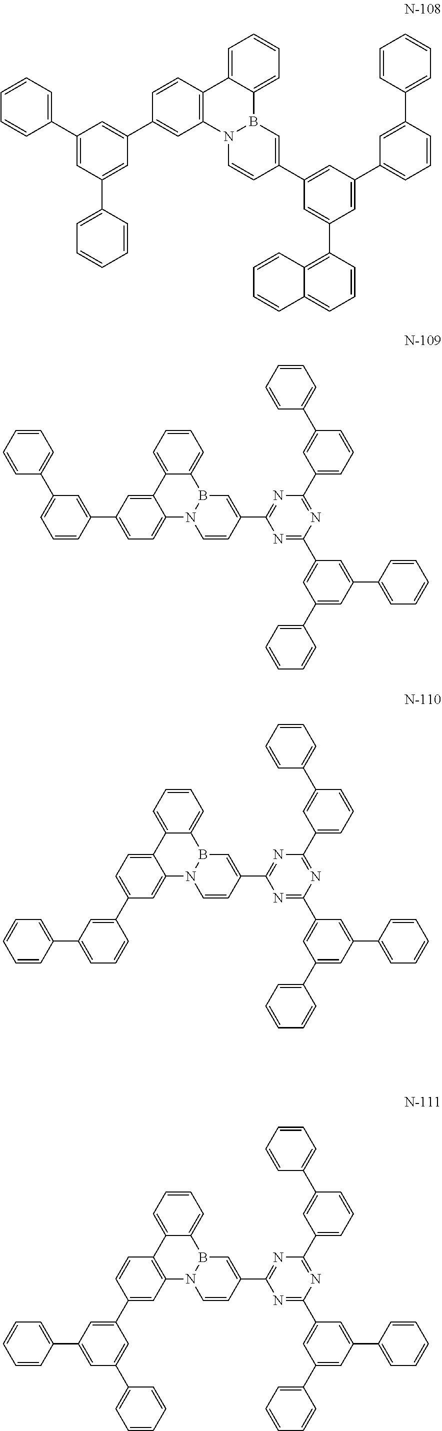 [SMILES: C1=CN2B(C=C1c1cc(-c3cccc(-c4ccccc4)c3)cc(-c3cccc4ccccc34)c1)c1ccccc1-c1ccc(-c3cc(-c4ccccc4)cc(-c4ccccc4)c3)cc12 ; C1=CN2B(C=C1c1nc(-c3cccc(-c4ccccc4)c3)nc(-c3cc(-c4ccccc4)cc(-c4ccccc4)c3)n1)c1ccccc1-c1cc(-c3cccc(-c4ccccc4)c3)ccc12 ; C1=CN2B(C=C1c1nc(-c3cccc(-c4ccccc4)c3)nc(-c3cc(-c4ccccc4)cc(-c4ccccc4)c3)n1)c1ccccc1-c1ccc(-c3cccc(-c4ccccc4)c3)cc12 ; C1=CN2B(C=C1c1nc(-c3cccc(-c4ccccc4)c3)nc(-c3cc(-c4ccccc4)cc(-c4ccccc4)c3)n1)c1ccccc1-c1ccc(-c3cc(-c4ccccc4)cc(-c4ccccc4)c3)cc12]

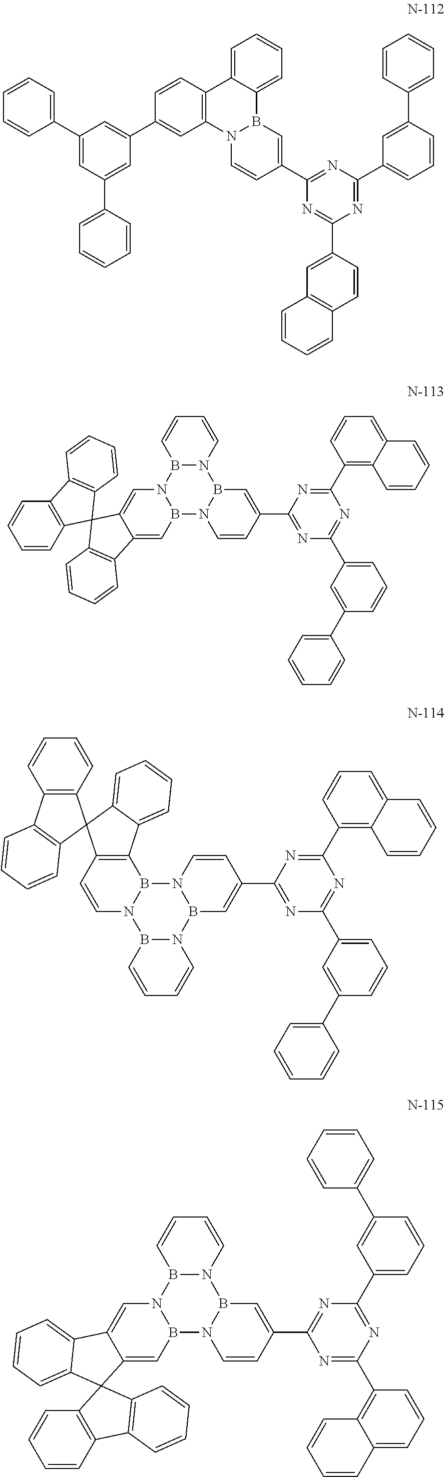 [SMILES: C1=CN2B(C=C1c1nc(-c3cccc(-c4ccccc4)c3)nc(-c3ccc4ccccc4c3)n1)c1ccccc1-c1ccc(-c3cc(-c4ccccc4)cc(-c4ccccc4)c3)cc12 ; C1=CB2N(C=C1)B1C=C(c3nc(-c4cccc(-c5ccccc5)c4)nc(-c4cccc5ccccc45)n3)C=CN1B1C=C3C(=CN21)C1(c2ccccc23)c2ccccc2-c2ccccc21 ; C1=CB2N(C=C1)B1C=C(c3nc(-c4cccc(-c5ccccc5)c4)nc(-c4cccc5ccccc45)n3)C=CN1B1C3=C(C=CN21)C1(c2ccccc23)c2ccccc2-c2ccccc21 ; C1=CB2N(C=C1)B1C=C(c3nc(-c4cccc(-c5ccccc5)c4)nc(-c4cccc5ccccc45)n3)C=CN1B1C=C3C(=CN21)c1ccccc1C31c2ccccc2-c2ccccc21]

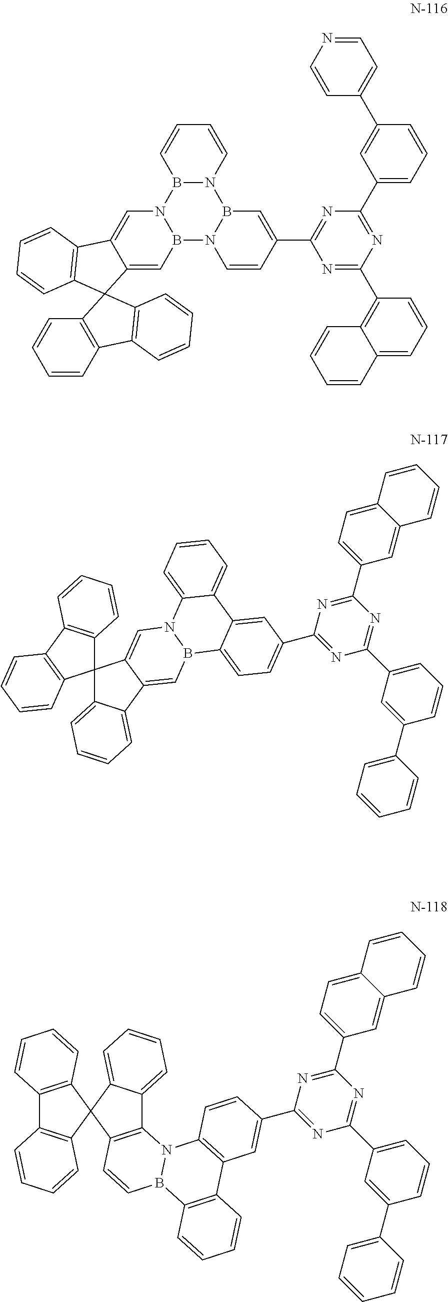 [SMILES: C1=CB2N(C=C1)B1C=C(c3nc(-c4cccc(-c5ccncc5)c4)nc(-c4cccc5ccccc45)n3)C=CN1B1C=C3C(=CN21)c1ccccc1C31c2ccccc2-c2ccccc21 ; C1=C2C(=CN3B1c1ccc(-c4nc(-c5cccc(-c6ccccc6)c5)nc(-c5ccc6ccccc6c5)n4)cc1-c1ccccc13)C1(c3ccccc32)c2ccccc2-c2ccccc21 ; C1=CC2=C(c3ccccc3C23c2ccccc2-c2ccccc23)N2B1c1ccccc1-c1cc(-c3nc(-c4cccc(-c5ccccc5)c4)nc(-c4ccc5ccccc5c4)n3)ccc12]

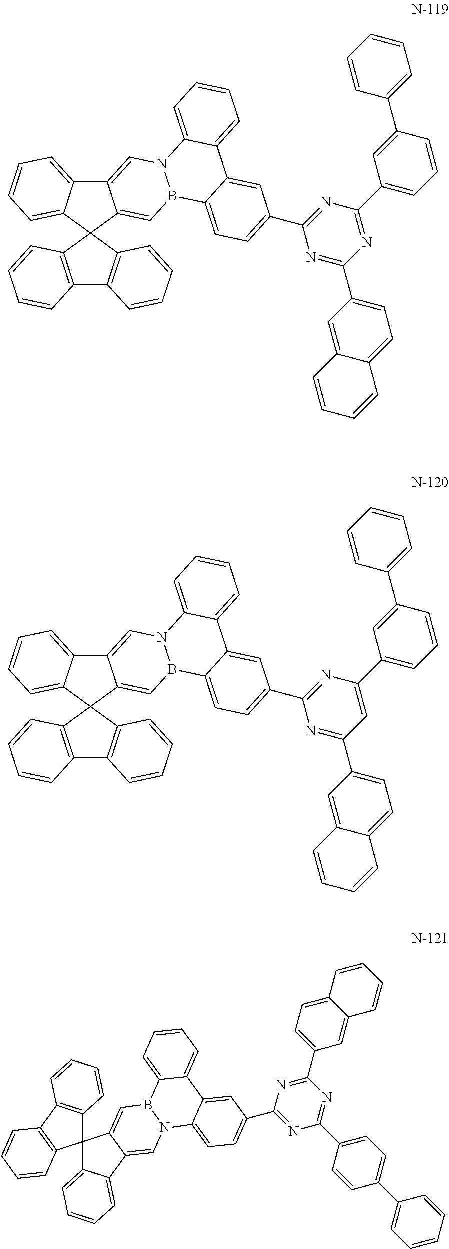 [SMILES: C1=C2C(=CB3c4ccc(-c5nc(-c6cccc(-c7ccccc7)c6)nc(-c6ccc7ccccc7c6)n5)cc4-c4ccccc4N13)C1(c3ccccc32)c2ccccc2-c2ccccc21 ; C1=C2C(=CB3c4ccc(-c5nc(-c6cccc(-c7ccccc7)c6)cc(-c6ccc7ccccc7c6)n5)cc4-c4ccccc4N13)C1(c3ccccc32)c2ccccc2-c2ccccc21 ; C1=C2C(=CB3c4ccccc4-c4cc(-c5nc(-c6ccc(-c7ccccc7)cc6)nc(-c6ccc7ccccc7c6)n5)ccc4N13)C1(c3ccccc32)c2ccccc2-c2ccccc21]

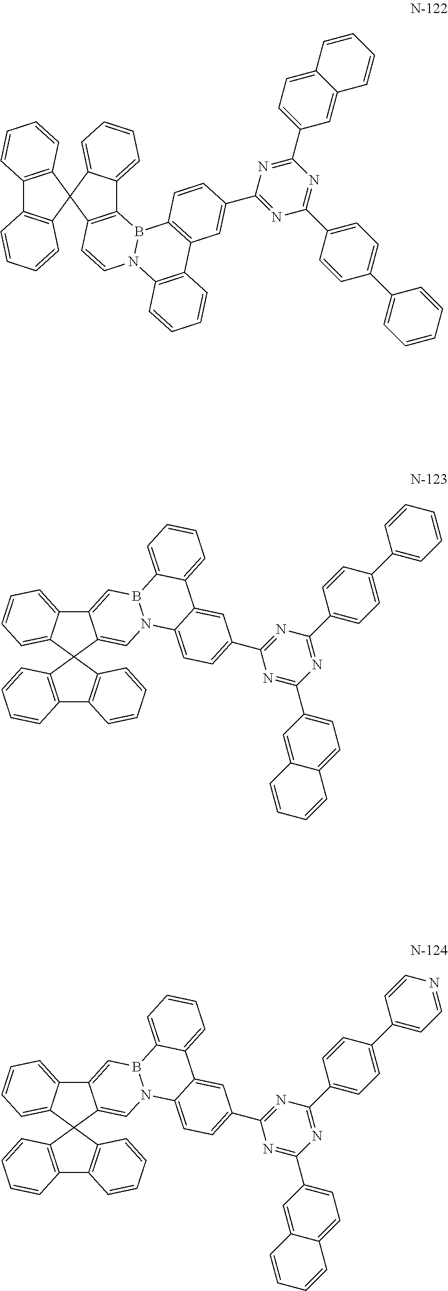 [SMILES: C1=CN2B(C3=C1C1(c4ccccc43)c3ccccc3-c3ccccc31)c1ccc(-c3nc(-c4ccc(-c5ccccc5)cc4)nc(-c4ccc5ccccc5c4)n3)cc1-c1ccccc12 ; C1=C2C(=CN3B1c1ccccc1-c1cc(-c4nc(-c5ccc(-c6ccccc6)cc5)nc(-c5ccc6ccccc6c5)n4)ccc13)C1(c3ccccc32)c2ccccc2-c2ccccc21 ; C1=C2C(=CN3B1c1ccccc1-c1cc(-c4nc(-c5ccc(-c6ccncc6)cc5)nc(-c5ccc6ccccc6c5)n4)ccc13)C1(c3ccccc32)c2ccccc2-c2ccccc21]

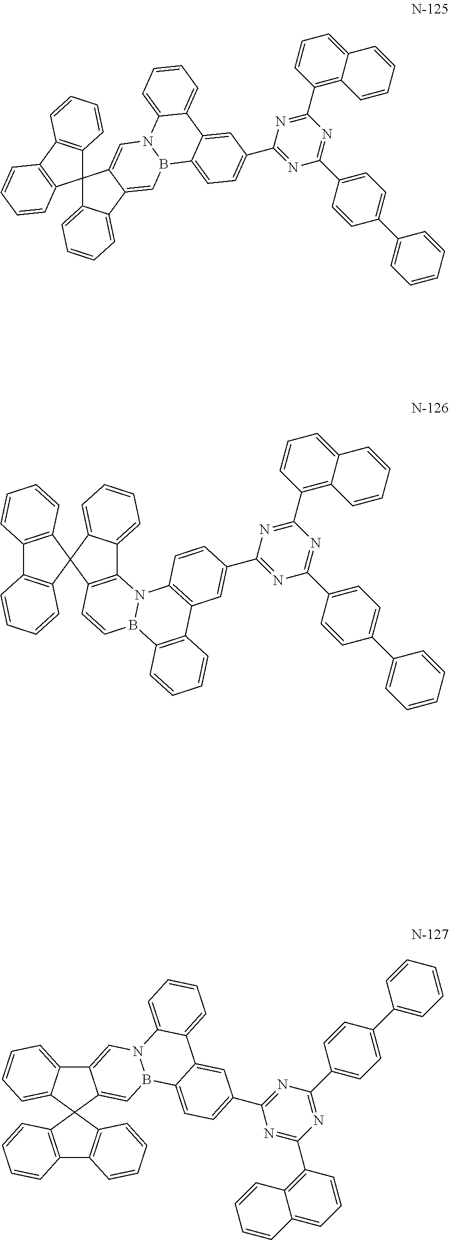 [SMILES: C1=C2C(=CN3B1c1ccc(-c4nc(-c5ccc(-c6ccccc6)cc5)nc(-c5cccc6ccccc56)n4)cc1-c1ccccc13)C1(c3ccccc32)c2ccccc2-c2ccccc21 ; C1=CC2=C(c3ccccc3C23c2ccccc2-c2ccccc23)N2B1c1ccccc1-c1cc(-c3nc(-c4ccc(-c5ccccc5)cc4)nc(-c4cccc5ccccc45)n3)ccc12 ; C1=C2C(=CB3c4ccc(-c5nc(-c6ccc(-c7ccccc7)cc6)nc(-c6cccc7ccccc67)n5)cc4-c4ccccc4N13)C1(c3ccccc32)c2ccccc2-c2ccccc21]

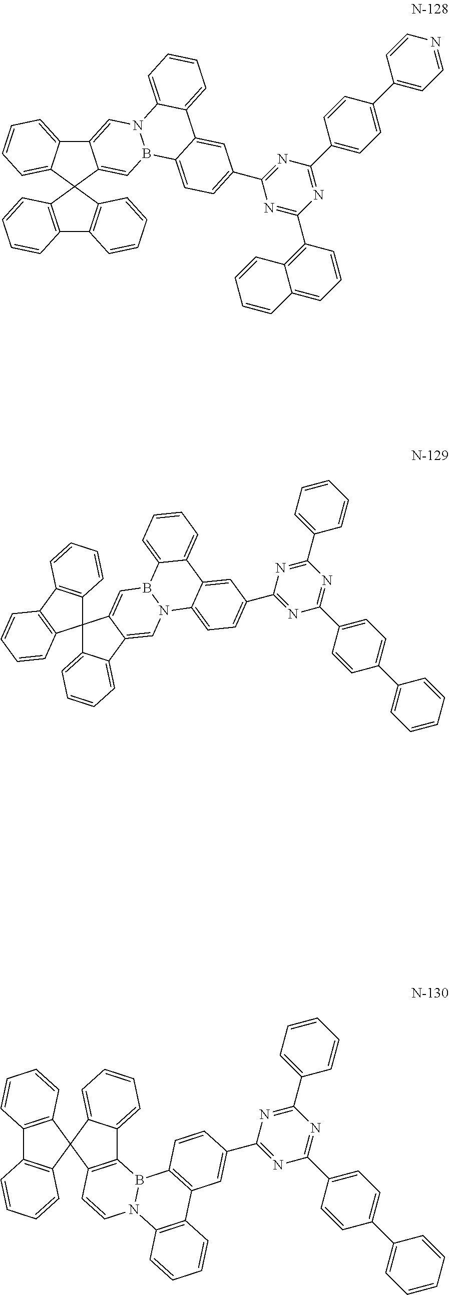 [SMILES: C1=C2C(=CB3c4ccc(-c5nc(-c6ccc(-c7ccncc7)cc6)nc(-c6cccc7ccccc67)n5)cc4-c4ccccc4N13)C1(c3ccccc32)c2ccccc2-c2ccccc21 ; C1=C2C(=CB3c4ccccc4-c4cc(-c5nc(-c6ccccc6)nc(-c6ccc(-c7ccccc7)cc6)n5)ccc4N13)C1(c3ccccc32)c2ccccc2-c2ccccc21 ; C1=CN2B(C3=C1C1(c4ccccc43)c3ccccc3-c3ccccc31)c1ccc(-c3nc(-c4ccccc4)nc(-c4ccc(-c5ccccc5)cc4)n3)cc1-c1ccccc12]

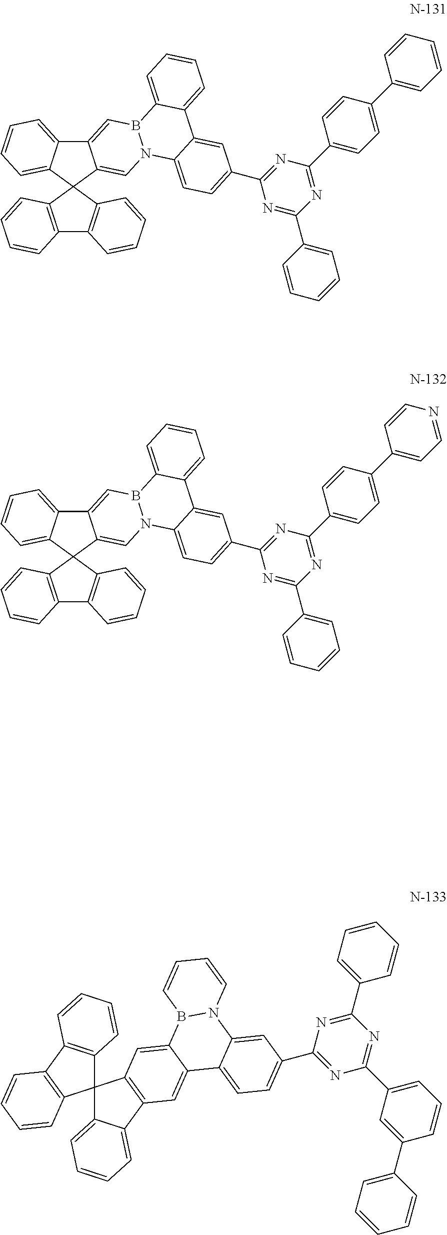 [SMILES: C1=C2C(=CN3B1c1ccccc1-c1cc(-c4nc(-c5ccccc5)nc(-c5ccc(-c6ccccc6)cc5)n4)ccc13)C1(c3ccccc32)c2ccccc2-c2ccccc21 ; C1=C2C(=CN3B1c1ccccc1-c1cc(-c4nc(-c5ccccc5)nc(-c5ccc(-c6ccncc6)cc5)n4)ccc13)C1(c3ccccc32)c2ccccc2-c2ccccc21 ; C1=CB2c3cc4c(cc3-c3ccc(-c5nc(-c6ccccc6)nc(-c6cccc(-c7ccccc7)c6)n5)cc3N2C=C1)-c1ccccc1C41c2ccccc2-c2ccccc21]

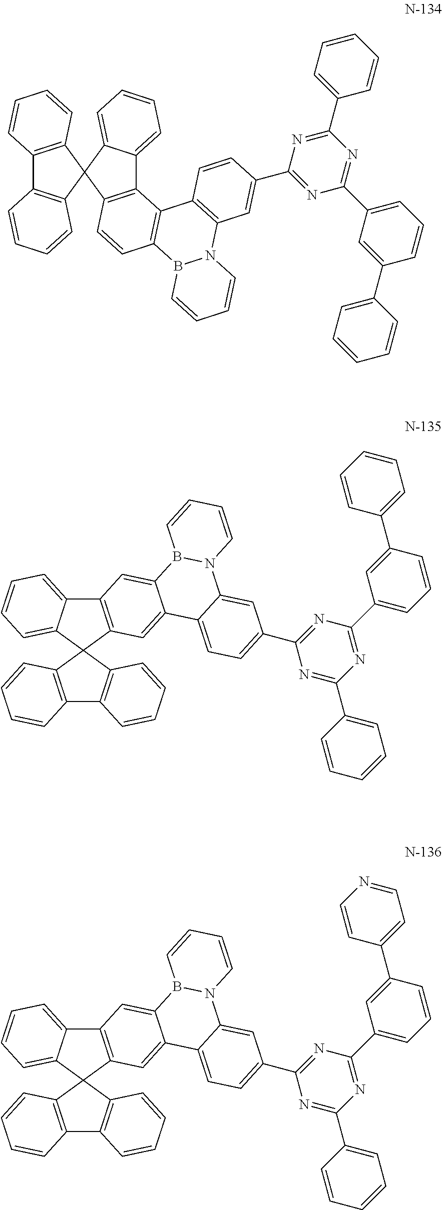 [SMILES: C1=CB2c3ccc4c(c3-c3ccc(-c5nc(-c6ccccc6)nc(-c6cccc(-c7ccccc7)c6)n5)cc3N2C=C1)-c1ccccc1C41c2ccccc2-c2ccccc21 ; C1=CB2c3cc4c(cc3-c3ccc(-c5nc(-c6ccccc6)nc(-c6cccc(-c7ccccc7)c6)n5)cc3N2C=C1)C1(c2ccccc2-c2ccccc21)c1ccccc1-4 ; C1=CB2c3cc4c(cc3-c3ccc(-c5nc(-c6ccccc6)nc(-c6cccc(-c7ccncc7)c6)n5)cc3N2C=C1)C1(c2ccccc2-c2ccccc21)c1ccccc1-4]

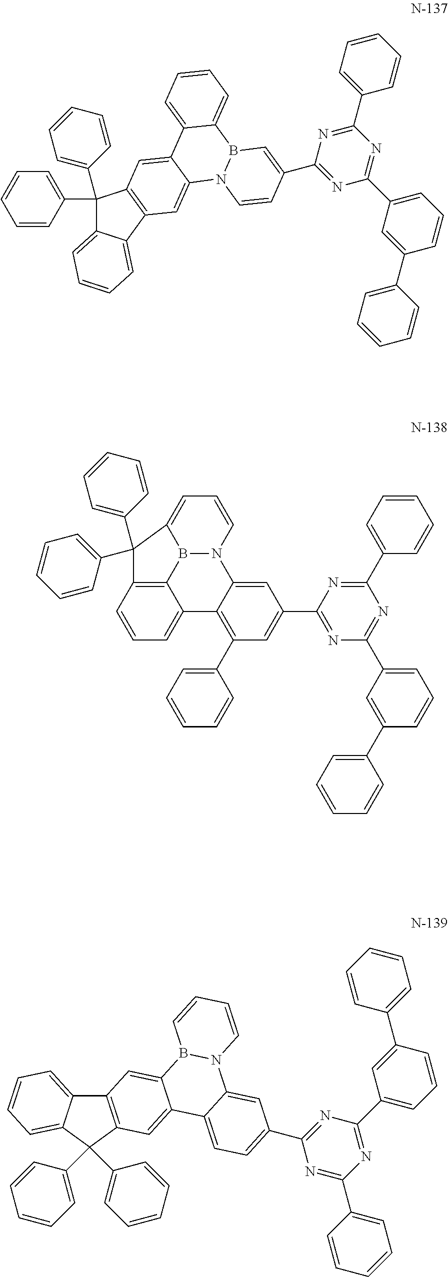 [SMILES: C1=CN2B(C=C1c1nc(-c3ccccc3)nc(-c3cccc(-c4ccccc4)c3)n1)c1ccccc1-c1cc3c(cc12)-c1ccccc1C3(c1ccccc1)c1ccccc1 ; C1=CN2B3C(=C1)C(c1ccccc1)(c1ccccc1)c1cccc(c13)-c1c(-c3ccccc3)cc(-c3nc(-c4ccccc4)nc(-c4cccc(-c5ccccc5)c4)n3)cc12 ; C1=CB2c3cc4c(cc3-c3ccc(-c5nc(-c6ccccc6)nc(-c6cccc(-c7ccccc7)c6)n5)cc3N2C=C1)C(c1ccccc1)(c1ccccc1)c1ccccc1-4]

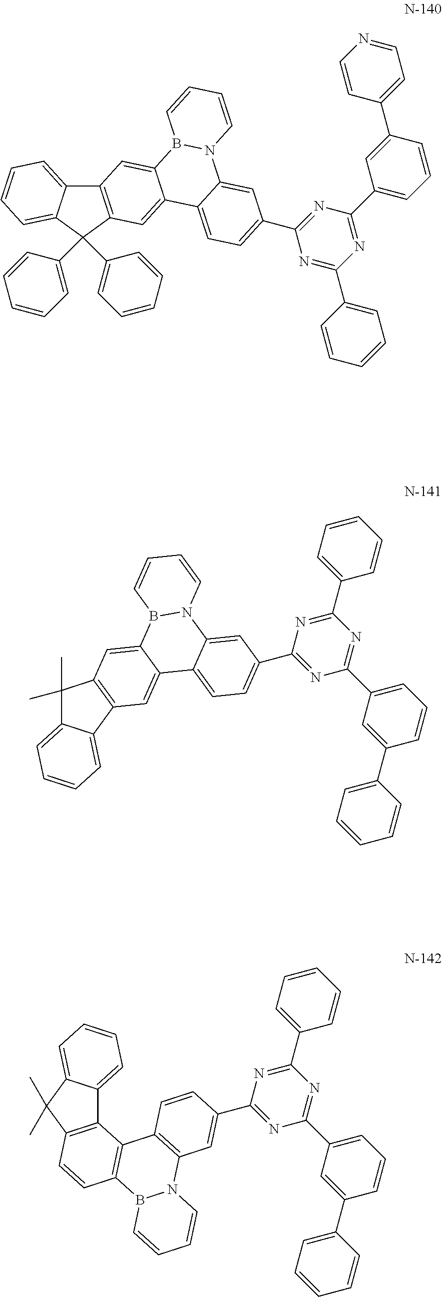 [SMILES: C1=CB2c3cc4c(cc3-c3ccc(-c5nc(-c6ccccc6)nc(-c6cccc(-c7ccncc7)c6)n5)cc3N2C=C1)C(c1ccccc1)(c1ccccc1)c1ccccc1-4 ; CC1(C)c2ccccc2-c2cc3c(cc21)B1C=CC=CN1c1cc(-c2nc(-c4ccccc4)nc(-c4cccc(-c5ccccc5)c4)n2)ccc1-3 ; CC1(C)c2ccccc2-c2c1ccc1c2-c2ccc(-c3nc(-c4ccccc4)nc(-c4cccc(-c5ccccc5)c4)n3)cc2N2C=CC=CB12]

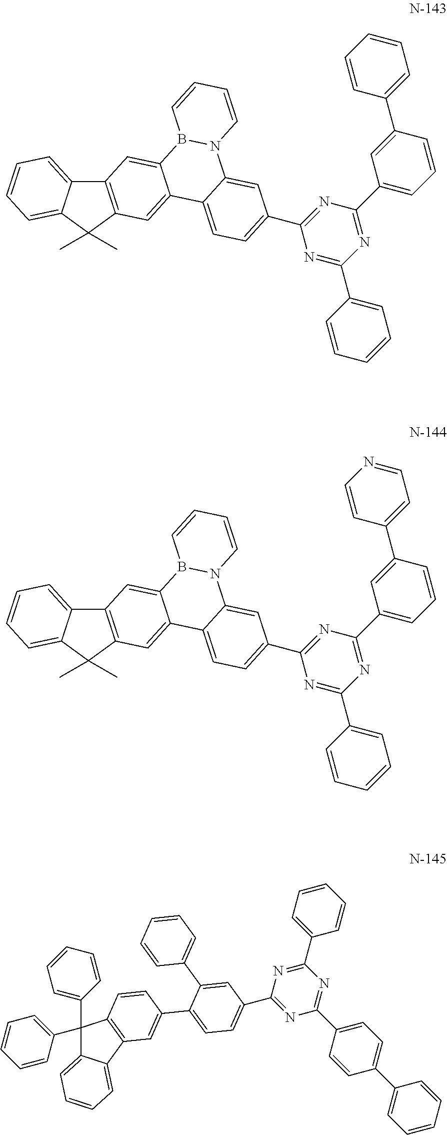 [SMILES: CC1(C)c2ccccc2-c2cc3c(cc21)-c1ccc(-c2nc(-c4ccccc4)nc(-c4cccc(-c5ccccc5)c4)n2)cc1N1C=CC=CB31 ; CC1(C)c2ccccc2-c2cc3c(cc21)-c1ccc(-c2nc(-c4ccccc4)nc(-c4cccc(-c5ccncc5)c4)n2)cc1N1C=CC=CB31 ; c1ccc(-c2ccc(-c3nc(-c4ccccc4)nc(-c4ccc(-c5ccc6c(c5)-c5ccccc5C6(c5ccccc5)c5ccccc5)c(-c5ccccc5)c4)n3)cc2)cc1]

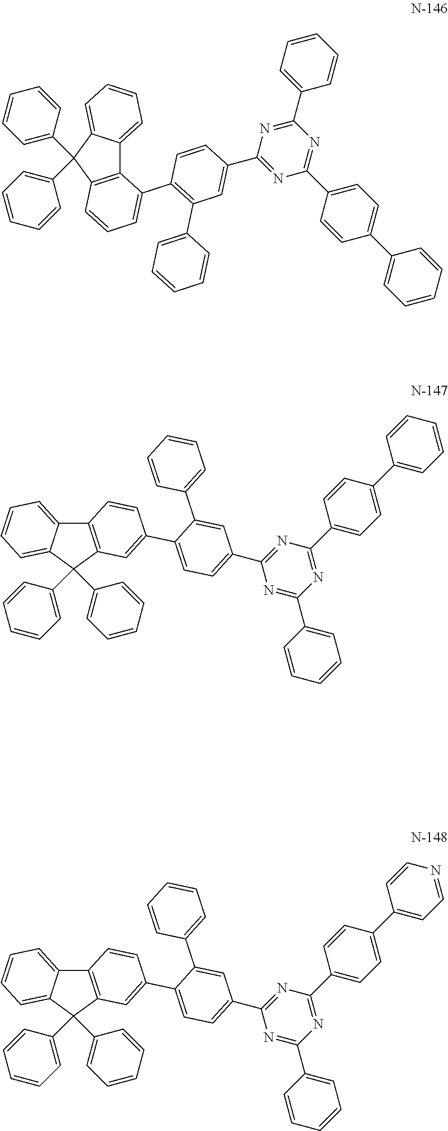 [SMILES: c1ccc(-c2ccc(-c3nc(-c4ccccc4)nc(-c4ccc(-c5cccc6c5-c5ccccc5C6(c5ccccc5)c5ccccc5)c(-c5ccccc5)c4)n3)cc2)cc1 ; c1ccc(-c2ccc(-c3nc(-c4ccccc4)nc(-c4ccc(-c5ccc6c(c5)C(c5ccccc5)(c5ccccc5)c5ccccc5-6)c(-c5ccccc5)c4)n3)cc2)cc1 ; c1ccc(-c2nc(-c3ccc(-c4ccncc4)cc3)nc(-c3ccc(-c4ccc5c(c4)C(c4ccccc4)(c4ccccc4)c4ccccc4-5)c(-c4ccccc4)c3)n2)cc1]

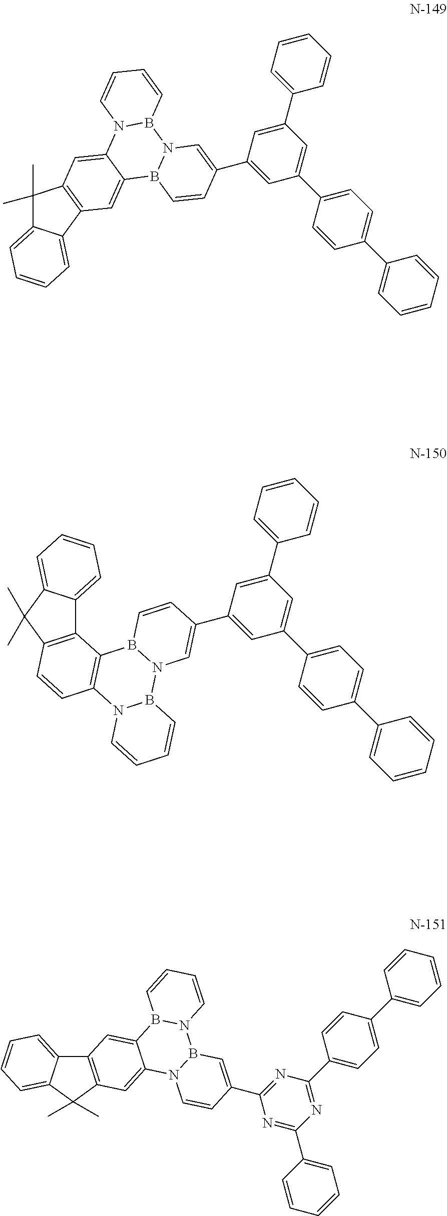 [SMILES: CC1(C)c2ccccc2-c2cc3c(cc21)N1C=CC=CB1N1C=C(c2cc(-c4ccccc4)cc(-c4ccc(-c5ccccc5)cc4)c2)C=CB31 ; CC1(C)c2ccccc2-c2c1ccc1c2B2C=CC(c3cc(-c4ccccc4)cc(-c4ccc(-c5ccccc5)cc4)c3)=CN2B2C=CC=CN21 ; CC1(C)c2ccccc2-c2cc3c(cc21)N1C=CC(c2nc(-c4ccccc4)nc(-c4ccc(-c5ccccc5)cc4)n2)=CB1N1C=CC=CB31]

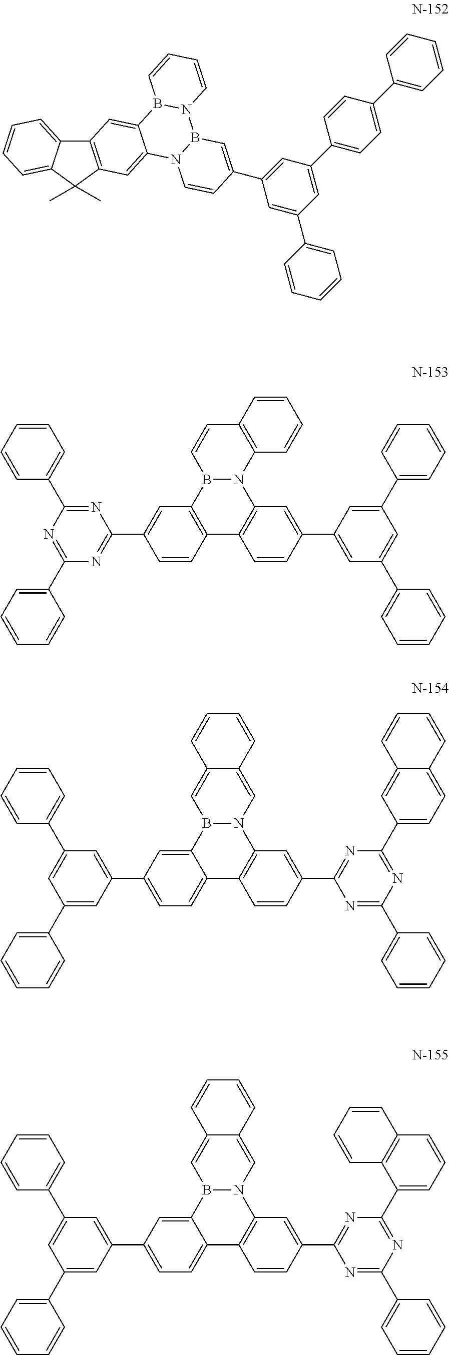 [SMILES: CC1(C)c2ccccc2-c2cc3c(cc21)N1C=CC(c2cc(-c4ccccc4)cc(-c4ccc(-c5ccccc5)cc4)c2)=CB1N1C=CC=CB31 ; C1=Cc2ccccc2N2B1c1cc(-c3nc(-c4ccccc4)nc(-c4ccccc4)n3)ccc1-c1ccc(-c3cc(-c4ccccc4)cc(-c4ccccc4)c3)cc12 ; C1=c2ccccc2=CN2B1c1cc(-c3cc(-c4ccccc4)cc(-c4ccccc4)c3)ccc1-c1ccc(-c3nc(-c4ccccc4)nc(-c4ccc5ccccc5c4)n3)cc12 ; C1=c2ccccc2=CN2B1c1cc(-c3cc(-c4ccccc4)cc(-c4ccccc4)c3)ccc1-c1ccc(-c3nc(-c4ccccc4)nc(-c4cccc5ccccc45)n3)cc12]

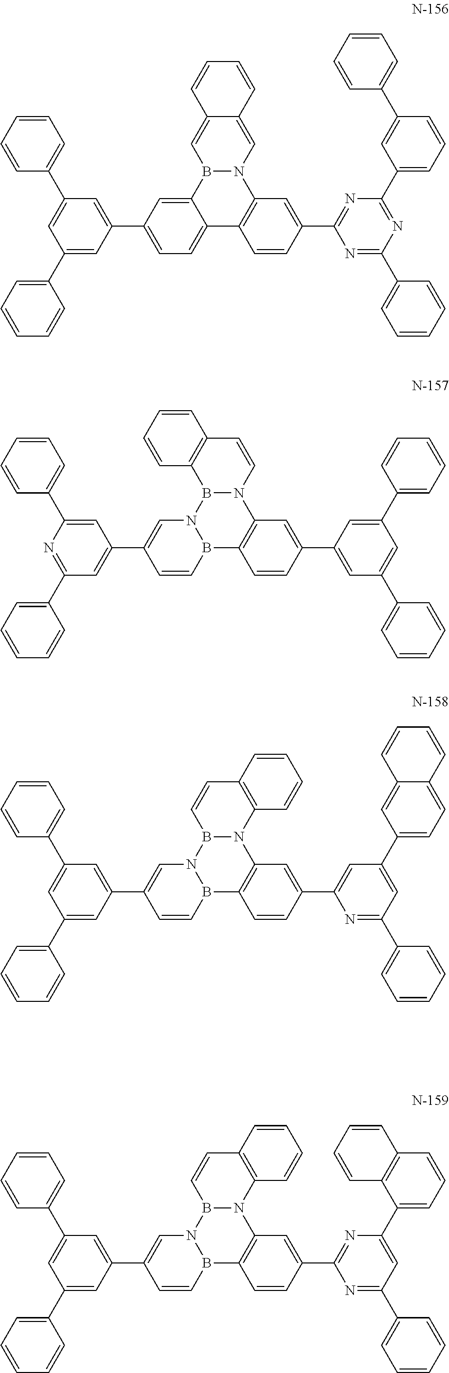 [SMILES: C1=c2ccccc2=CN2B1c1cc(-c3cc(-c4ccccc4)cc(-c4ccccc4)c3)ccc1-c1ccc(-c3nc(-c4ccccc4)nc(-c4cccc(-c5ccccc5)c4)n3)cc12 ; C1=CC(c2cc(-c3ccccc3)nc(-c3ccccc3)c2)=CN2B1c1ccc(-c3cc(-c4ccccc4)cc(-c4ccccc4)c3)cc1N1C=Cc3ccccc3B21 ; C1=CC(c2cc(-c3ccccc3)cc(-c3ccccc3)c2)=CN2B1c1ccc(-c3cc(-c4ccc5ccccc5c4)cc(-c4ccccc4)n3)cc1N1B2C=Cc2ccccc21 ; C1=CC(c2cc(-c3ccccc3)cc(-c3ccccc3)c2)=CN2B1c1ccc(-c3nc(-c4ccccc4)cc(-c4cccc5ccccc45)n3)cc1N1B2C=Cc2ccccc21]

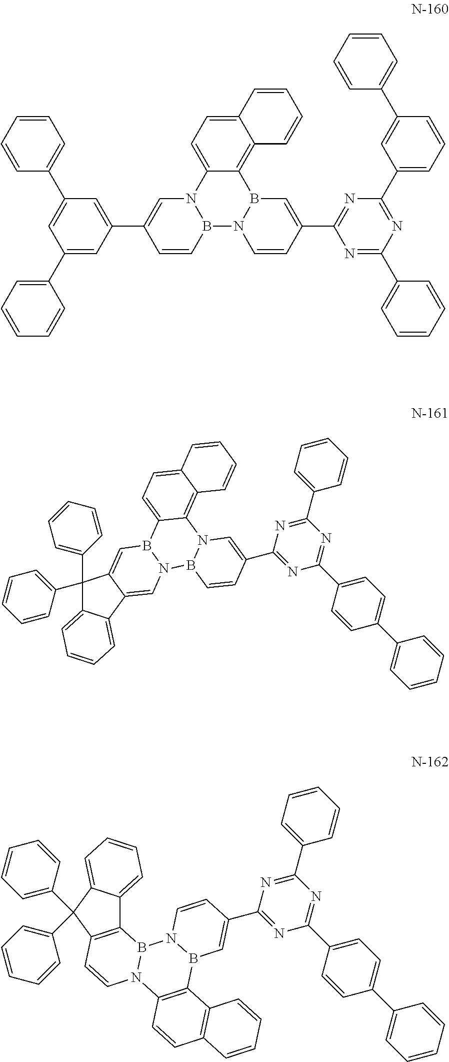 [SMILES: C1=CC(c2cc(-c3ccccc3)cc(-c3ccccc3)c2)=CN2B1N1C=CC(c3nc(-c4ccccc4)nc(-c4cccc(-c5ccccc5)c4)n3)=CB1c1c2ccc2ccccc12 ; C1=CC(c2nc(-c3ccccc3)nc(-c3ccc(-c4ccccc4)cc3)n2)=CN2B1N1C=C3C(=CB1c1ccc4ccccc4c12)C(c1ccccc1)(c1ccccc1)c1ccccc13 ; C1=CN2B(C=C1c1nc(-c3ccccc3)nc(-c3ccc(-c4ccccc4)cc3)n1)c1c(ccc3ccccc13)N1C=CC3=C(B21)c1ccccc1C3(c1ccccc1)c1ccccc1]

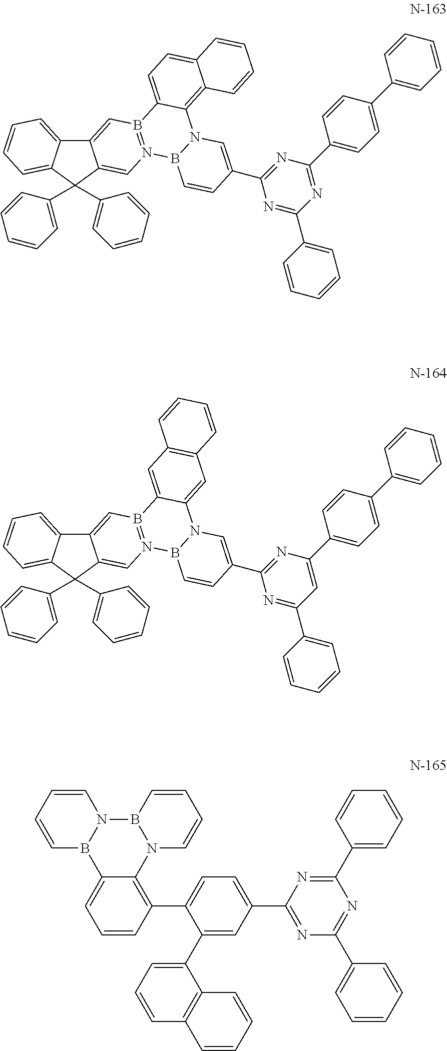

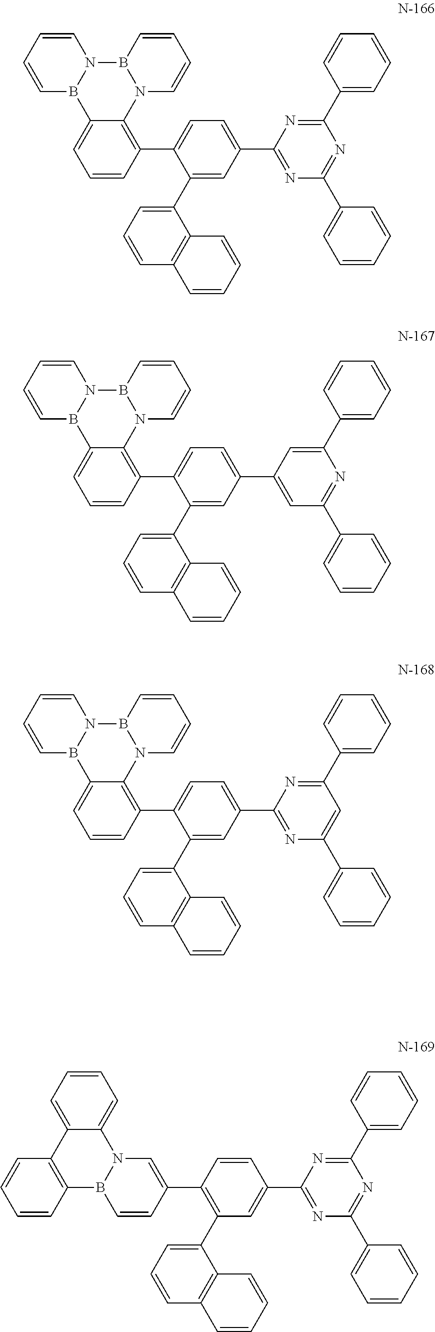 [SMILES: C1=CB2c3cccc(-c4ccc(-c5nc(-c6ccccc6)nc(-c6ccccc6)n5)cc4-c4cccc5ccccc45)c3N3C=CC=CB3N2C=C1 ; C1=CB2c3cccc(-c4ccc(-c5cc(-c6ccccc6)nc(-c6ccccc6)c5)cc4-c4cccc5ccccc45)c3N3C=CC=CB3N2C=C1 ; C1=CB2c3cccc(-c4ccc(-c5nc(-c6ccccc6)cc(-c6ccccc6)n5)cc4-c4cccc5ccccc45)c3N3C=CC=CB3N2C=C1 ; C1=CC(c2ccc(-c3nc(-c4ccccc4)nc(-c4ccccc4)n3)cc2-c2cccc3ccccc23)=CN2B1c1ccccc1-c1ccccc12]

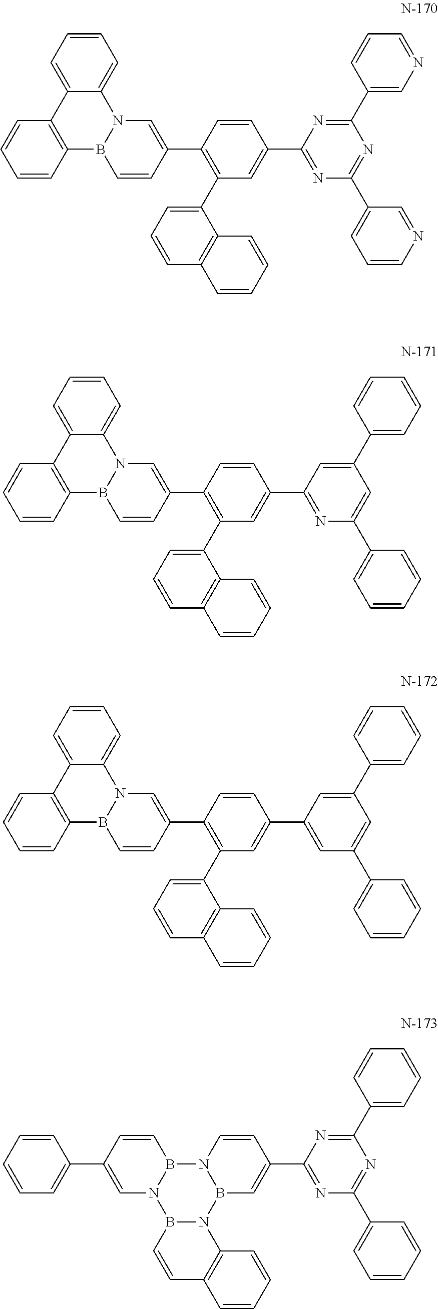 [SMILES: C1=CC(c2ccc(-c3nc(-c4cccnc4)nc(-c4cccnc4)n3)cc2-c2cccc3ccccc23)=CN2B1c1ccccc1-c1ccccc12 ; C1=CC(c2ccc(-c3cc(-c4ccccc4)cc(-c4ccccc4)n3)cc2-c2cccc3ccccc23)=CN2B1c1ccccc1-c1ccccc12 ; C1=CC(c2ccc(-c3cc(-c4ccccc4)cc(-c4ccccc4)c3)cc2-c2cccc3ccccc23)=CN2B1c1ccccc1-c1ccccc12 ; C1=CC(c2ccccc2)=CN2B1N1C=CC(c3nc(-c4ccccc4)nc(-c4ccccc4)n3)=CB1N1B2C=Cc2ccccc21]

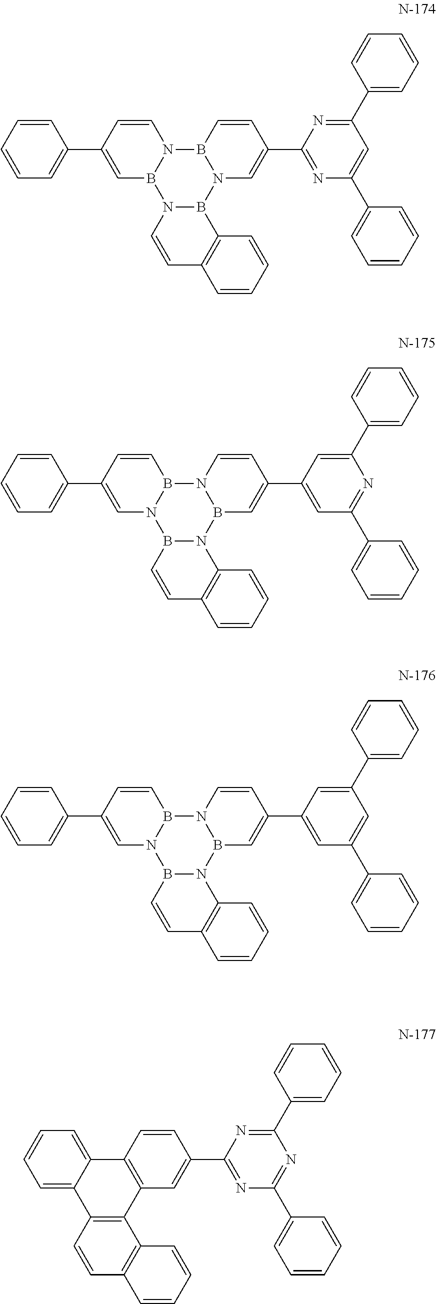 [SMILES: C1=CC(c2nc(-c3ccccc3)cc(-c3ccccc3)n2)=CN2B1N1C=CC(c3ccccc3)=CB1N1C=Cc3ccccc3B12 ; C1=CC(c2ccccc2)=CN2B1N1C=CC(c3cc(-c4ccccc4)nc(-c4ccccc4)c3)=CB1N1B2C=Cc2ccccc21 ; C1=CC(c2ccccc2)=CN2B1N1C=CC(c3cc(-c4ccccc4)cc(-c4ccccc4)c3)=CB1N1B2C=Cc2ccccc21 ; c1ccc(-c2nc(-c3ccccc3)nc(-c3ccc4c5ccccc5c5ccc6ccccc6c5c4c3)n2)cc1]

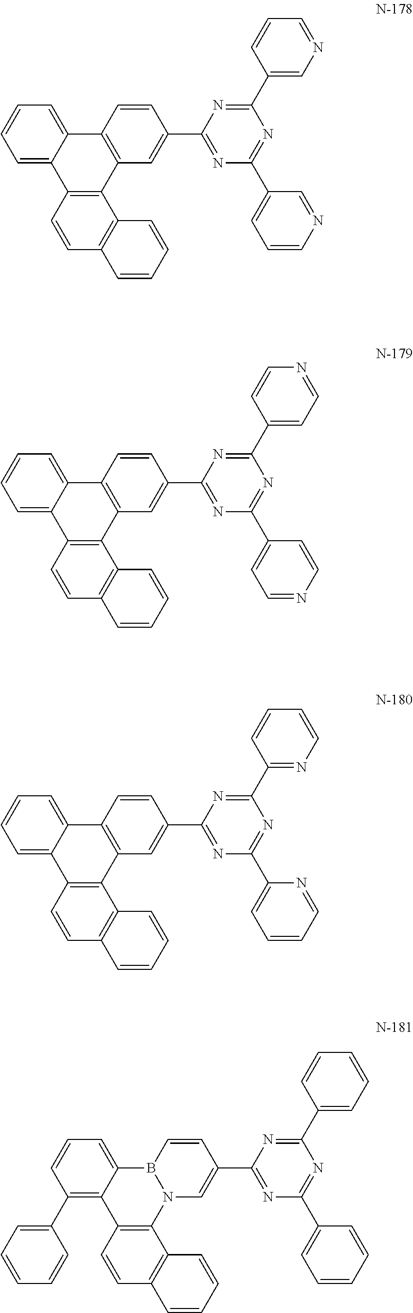 [SMILES: c1cncc(-c2nc(-c3cccnc3)nc(-c3ccc4c5ccccc5c5ccc6ccccc6c5c4c3)n2)c1 ; c1ccc2c(c1)ccc1c3ccccc3c3ccc(-c4nc(-c5ccncc5)nc(-c5ccncc5)n4)cc3c21 ; c1ccc(-c2nc(-c3ccc4c5ccccc5c5ccc6ccccc6c5c4c3)nc(-c3ccccn3)n2)nc1 ; C1=CC(c2nc(-c3ccccc3)nc(-c3ccccc3)n2)=CN2B1c1cccc(-c3ccccc3)c1-c1ccc3ccccc3c12]

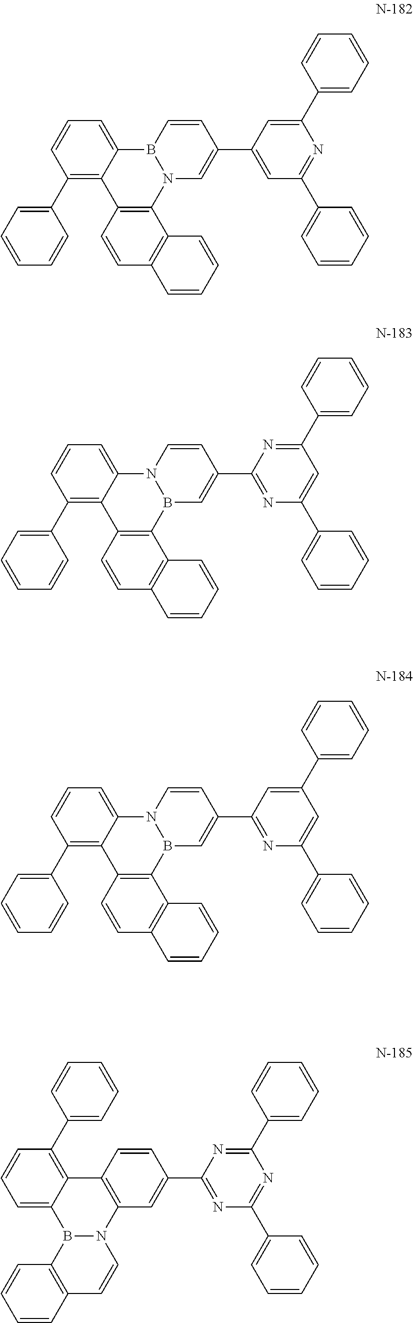 [SMILES: C1=CC(c2cc(-c3ccccc3)nc(-c3ccccc3)c2)=CN2B1c1cccc(-c3ccccc3)c1-c1ccc3ccccc3c12 ; C1=CN2B(C=C1c1nc(-c3ccccc3)cc(-c3ccccc3)n1)c1c(ccc3ccccc13)-c1c(-c3ccccc3)cccc12 ; C1=CN2B(C=C1c1cc(-c3ccccc3)cc(-c3ccccc3)n1)c1c(ccc3ccccc13)-c1c(-c3ccccc3)cccc12 ; C1=CN2B(c3ccccc31)c1cccc(-c3ccccc3)c1-c1ccc(-c3nc(-c4ccccc4)nc(-c4ccccc4)n3)cc12]

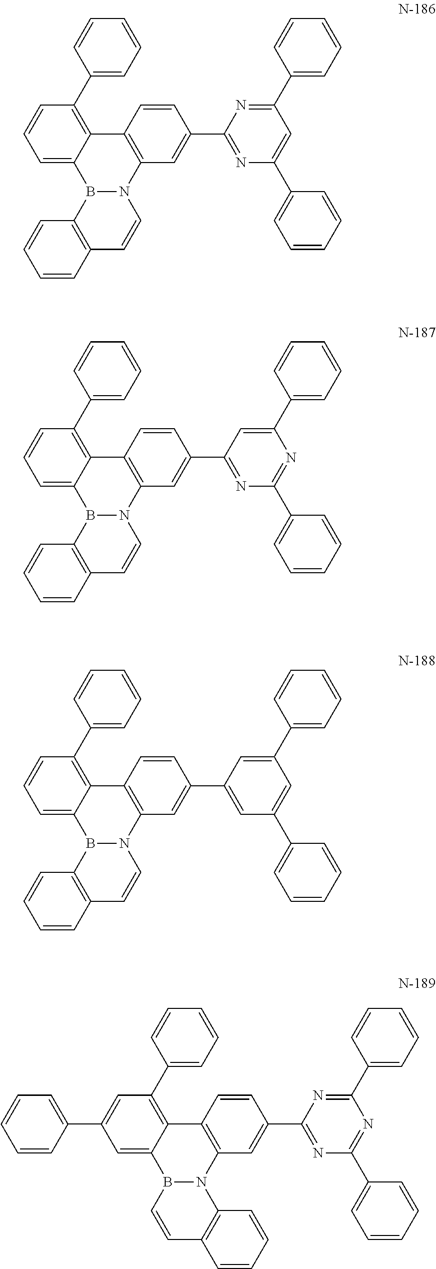 [SMILES: C1=CN2B(c3ccccc31)c1cccc(-c3ccccc3)c1-c1ccc(-c3nc(-c4ccccc4)cc(-c4ccccc4)n3)cc12 ; C1=CN2B(c3ccccc31)c1cccc(-c3ccccc3)c1-c1ccc(-c3cc(-c4ccccc4)nc(-c4ccccc4)n3)cc12 ; C1=CN2B(c3ccccc31)c1cccc(-c3ccccc3)c1-c1ccc(-c3cc(-c4ccccc4)cc(-c4ccccc4)c3)cc12 ; C1=Cc2ccccc2N2B1c1cc(-c3ccccc3)cc(-c3ccccc3)c1-c1ccc(-c3nc(-c4ccccc4)nc(-c4ccccc4)n3)cc12]

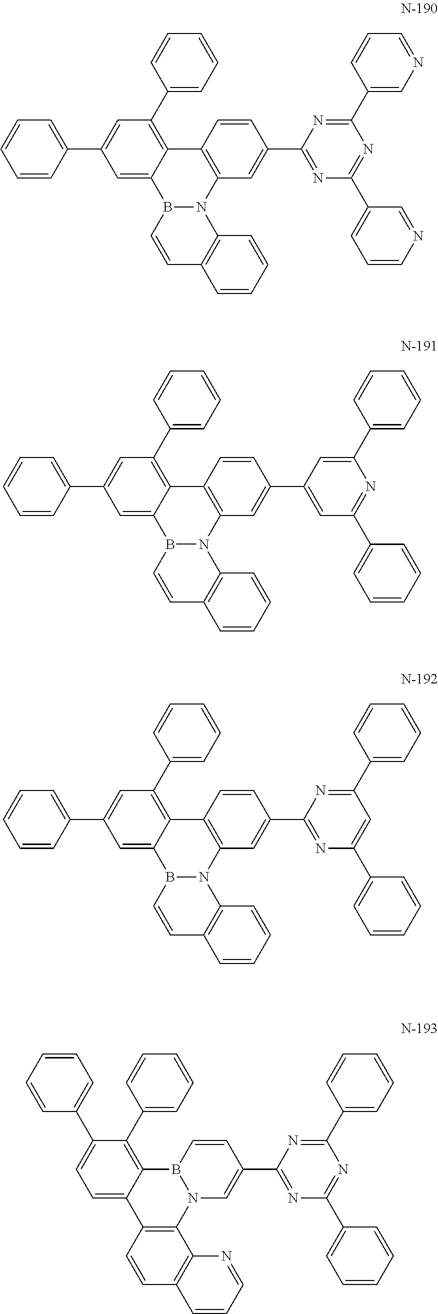 [SMILES: C1=Cc2ccccc2N2B1c1cc(-c3ccccc3)cc(-c3ccccc3)c1-c1ccc(-c3nc(-c4cccnc4)nc(-c4cccnc4)n3)cc12 ; C1=Cc2ccccc2N2B1c1cc(-c3ccccc3)cc(-c3ccccc3)c1-c1ccc(-c3cc(-c4ccccc4)nc(-c4ccccc4)c3)cc12 ; C1=Cc2ccccc2N2B1c1cc(-c3ccccc3)cc(-c3ccccc3)c1-c1ccc(-c3nc(-c4ccccc4)cc(-c4ccccc4)n3)cc12 ; C1=CC(c2nc(-c3ccccc3)nc(-c3ccccc3)n2)=CN2B1c1c(ccc(-c3ccccc3)c1-c1ccccc1)-c1ccc3cccnc3c12]

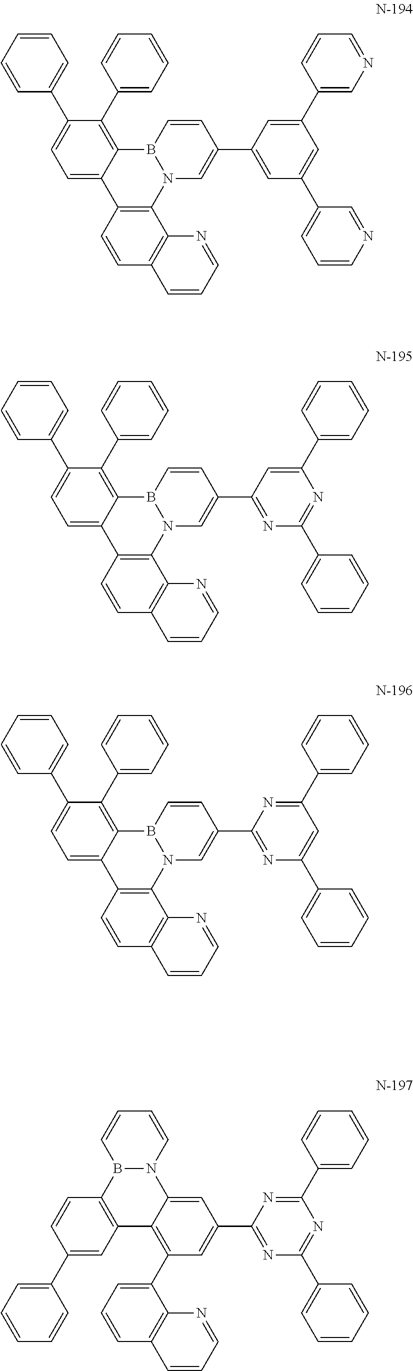 [SMILES: C1=CC(c2cc(-c3cccnc3)cc(-c3cccnc3)c2)=CN2B1c1c(ccc(-c3ccccc3)c1-c1ccccc1)-c1ccc3cccnc3c12 ; C1=CC(c2cc(-c3ccccc3)nc(-c3ccccc3)n2)=CN2B1c1c(ccc(-c3ccccc3)c1-c1ccccc1)-c1ccc3cccnc3c12 ; C1=CC(c2nc(-c3ccccc3)cc(-c3ccccc3)n2)=CN2B1c1c(ccc(-c3ccccc3)c1-c1ccccc1)-c1ccc3cccnc3c12 ; C1=CB2c3ccc(-c4ccccc4)cc3-c3c(-c4cccc5cccnc45)cc(-c4nc(-c5ccccc5)nc(-c5ccccc5)n4)cc3N2C=C1]

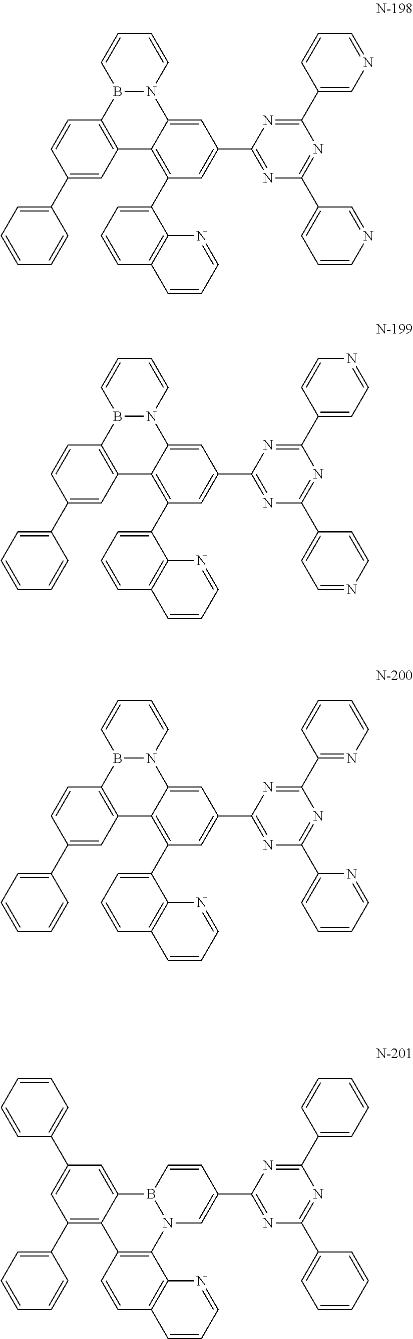 [SMILES: C1=CB2c3ccc(-c4ccccc4)cc3-c3c(-c4cccc5cccnc45)cc(-c4nc(-c5cccnc5)nc(-c5cccnc5)n4)cc3N2C=C1 ; C1=CB2c3ccc(-c4ccccc4)cc3-c3c(-c4cccc5cccnc45)cc(-c4nc(-c5ccncc5)nc(-c5ccncc5)n4)cc3N2C=C1 ; C1=CB2c3ccc(-c4ccccc4)cc3-c3c(-c4cccc5cccnc45)cc(-c4nc(-c5ccccn5)nc(-c5ccccn5)n4)cc3N2C=C1 ; C1=CC(c2nc(-c3ccccc3)nc(-c3ccccc3)n2)=CN2B1c1cc(-c3ccccc3)cc(-c3ccccc3)c1-c1ccc3cccnc3c12]

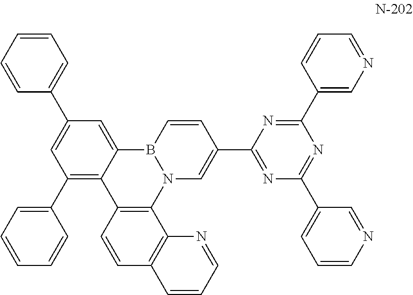 [SMILES: C1=CC(c2nc(-c3cccnc3)nc(-c3cccnc3)n2)=CN2B1c1cc(-c3ccccc3)cc(-c3ccccc3)c1-c1ccc3cccnc3c12]

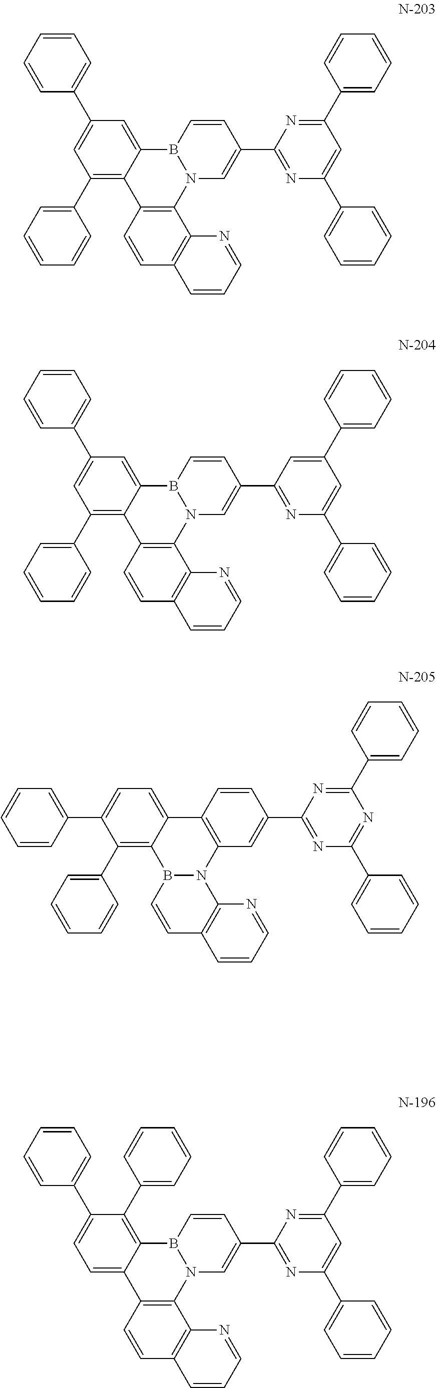 [SMILES: C1=CC(c2nc(-c3ccccc3)cc(-c3ccccc3)n2)=CN2B1c1cc(-c3ccccc3)cc(-c3ccccc3)c1-c1ccc3cccnc3c12 ; C1=CC(c2cc(-c3ccccc3)cc(-c3ccccc3)n2)=CN2B1c1cc(-c3ccccc3)cc(-c3ccccc3)c1-c1ccc3cccnc3c12 ; C1=Cc2cccnc2N2B1c1c(ccc(-c3ccccc3)c1-c1ccccc1)-c1ccc(-c3nc(-c4ccccc4)nc(-c4ccccc4)n3)cc12 ; C1=CC(c2nc(-c3ccccc3)cc(-c3ccccc3)n2)=CN2B1c1c(ccc(-c3ccccc3)c1-c1ccccc1)-c1ccc3cccnc3c12]

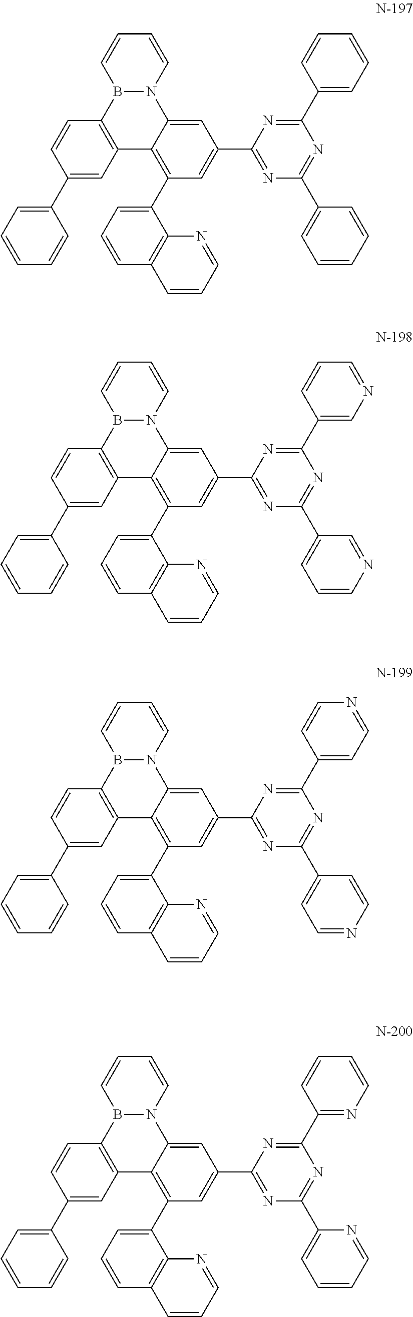 [SMILES: C1=CB2c3ccc(-c4ccccc4)cc3-c3c(-c4cccc5cccnc45)cc(-c4nc(-c5ccccc5)nc(-c5ccccc5)n4)cc3N2C=C1 ; C1=CB2c3ccc(-c4ccccc4)cc3-c3c(-c4cccc5cccnc45)cc(-c4nc(-c5cccnc5)nc(-c5cccnc5)n4)cc3N2C=C1 ; C1=CB2c3ccc(-c4ccccc4)cc3-c3c(-c4cccc5cccnc45)cc(-c4nc(-c5ccncc5)nc(-c5ccncc5)n4)cc3N2C=C1 ; C1=CB2c3ccc(-c4ccccc4)cc3-c3c(-c4cccc5cccnc45)cc(-c4nc(-c5ccccn5)nc(-c5ccccn5)n4)cc3N2C=C1]

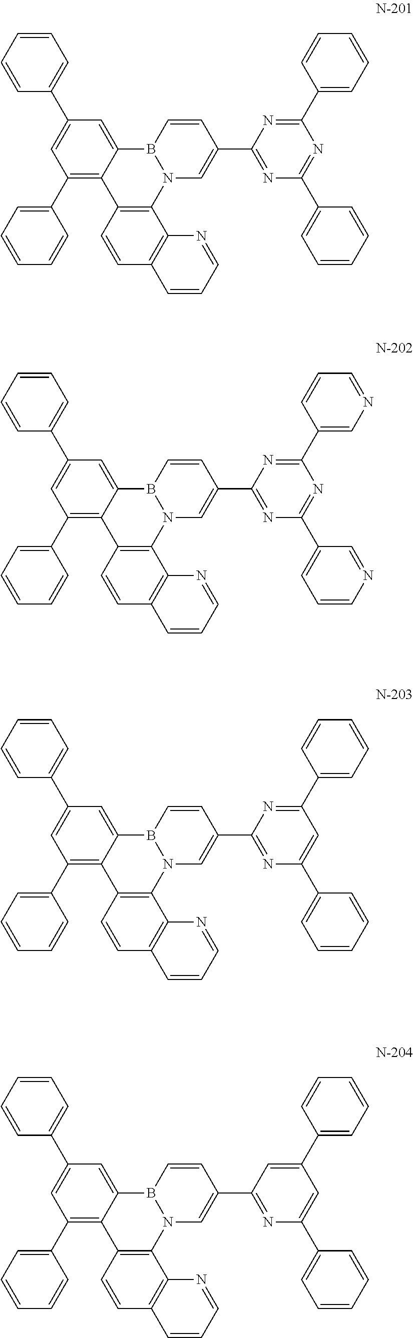 [SMILES: C1=CC(c2nc(-c3ccccc3)nc(-c3ccccc3)n2)=CN2B1c1cc(-c3ccccc3)cc(-c3ccccc3)c1-c1ccc3cccnc3c12 ; C1=CC(c2nc(-c3cccnc3)nc(-c3cccnc3)n2)=CN2B1c1cc(-c3ccccc3)cc(-c3ccccc3)c1-c1ccc3cccnc3c12 ; C1=CC(c2nc(-c3ccccc3)cc(-c3ccccc3)n2)=CN2B1c1cc(-c3ccccc3)cc(-c3ccccc3)c1-c1ccc3cccnc3c12 ; C1=CC(c2cc(-c3ccccc3)cc(-c3ccccc3)n2)=CN2B1c1cc(-c3ccccc3)cc(-c3ccccc3)c1-c1ccc3cccnc3c12]

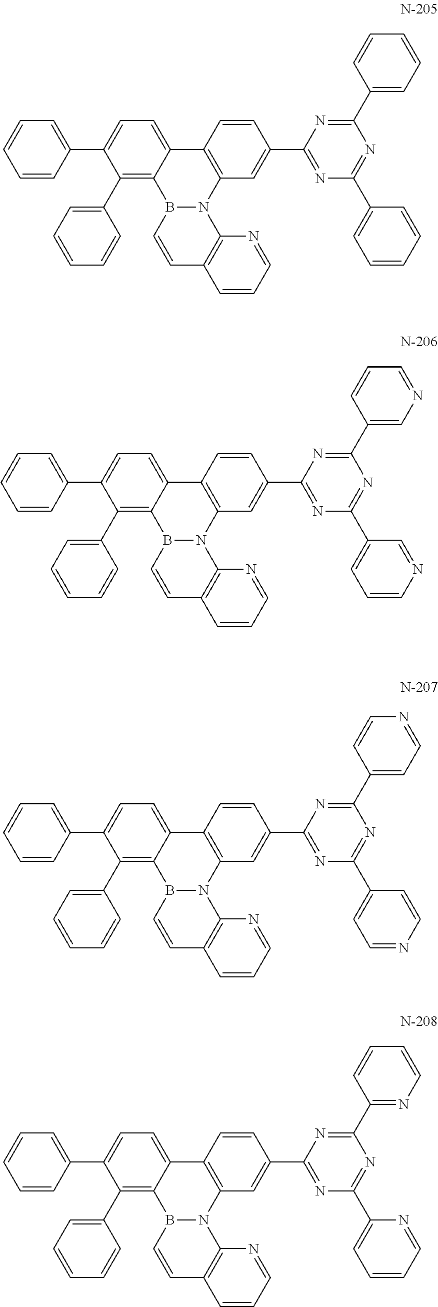 [SMILES: C1=Cc2cccnc2N2B1c1c(ccc(-c3ccccc3)c1-c1ccccc1)-c1ccc(-c3nc(-c4ccccc4)nc(-c4ccccc4)n3)cc12 ; C1=Cc2cccnc2N2B1c1c(ccc(-c3ccccc3)c1-c1ccccc1)-c1ccc(-c3nc(-c4cccnc4)nc(-c4cccnc4)n3)cc12 ; C1=Cc2cccnc2N2B1c1c(ccc(-c3ccccc3)c1-c1ccccc1)-c1ccc(-c3nc(-c4ccncc4)nc(-c4ccncc4)n3)cc12 ; C1=Cc2cccnc2N2B1c1c(ccc(-c3ccccc3)c1-c1ccccc1)-c1ccc(-c3nc(-c4ccccn4)nc(-c4ccccn4)n3)cc12]

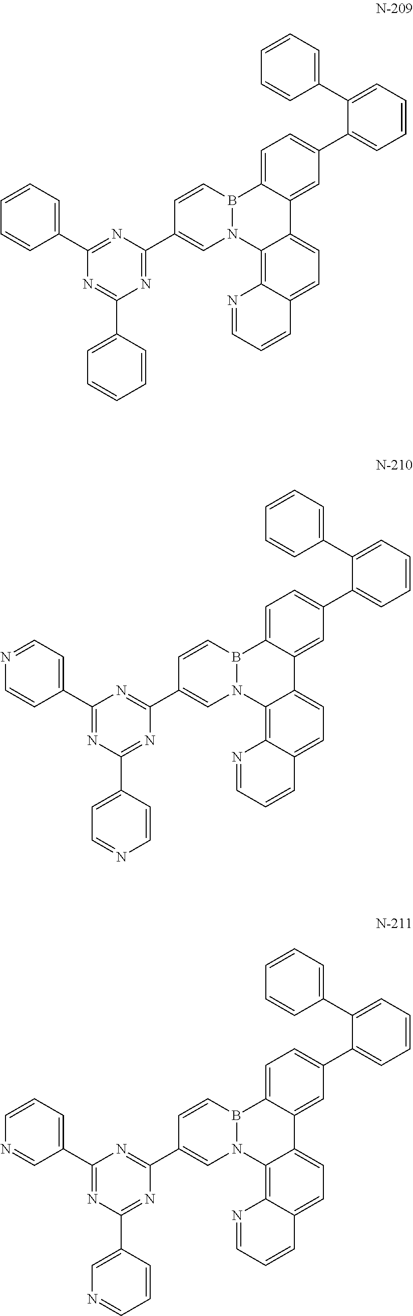 [SMILES: C1=CC(c2nc(-c3ccccc3)nc(-c3ccccc3)n2)=CN2B1c1ccc(-c3ccccc3-c3ccccc3)cc1-c1ccc3cccnc3c12 ; C1=CC(c2nc(-c3ccncc3)nc(-c3ccncc3)n2)=CN2B1c1ccc(-c3ccccc3-c3ccccc3)cc1-c1ccc3cccnc3c12 ; C1=CC(c2nc(-c3cccnc3)nc(-c3cccnc3)n2)=CN2B1c1ccc(-c3ccccc3-c3ccccc3)cc1-c1ccc3cccnc3c12]

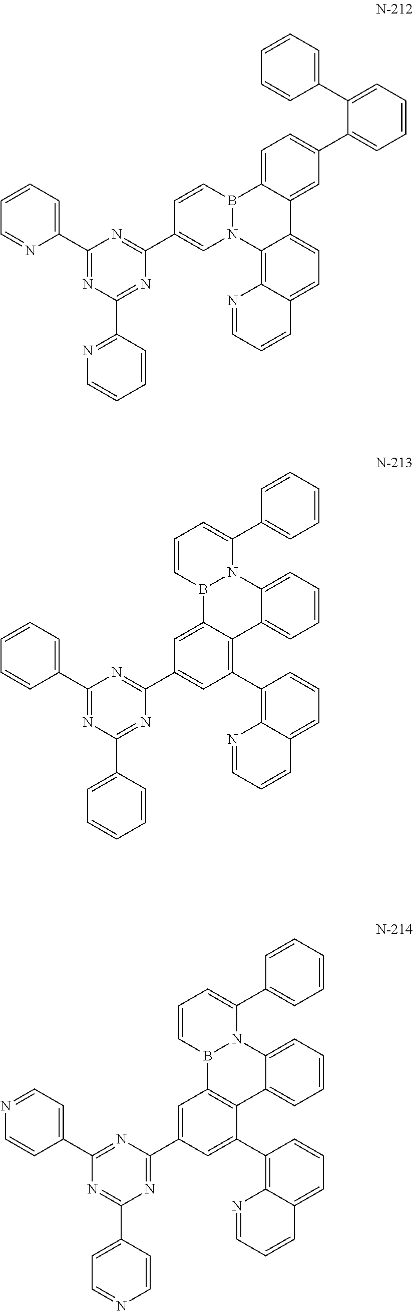 [SMILES: C1=CC(c2nc(-c3ccccn3)nc(-c3ccccn3)n2)=CN2B1c1ccc(-c3ccccc3-c3ccccc3)cc1-c1ccc3cccnc3c12 ; C1=CB2c3cc(-c4nc(-c5ccccc5)nc(-c5ccccc5)n4)cc(-c4cccc5cccnc45)c3-c3ccccc3N2C(c2ccccc2)=C1 ; C1=CB2c3cc(-c4nc(-c5ccncc5)nc(-c5ccncc5)n4)cc(-c4cccc5cccnc45)c3-c3ccccc3N2C(c2ccccc2)=C1]

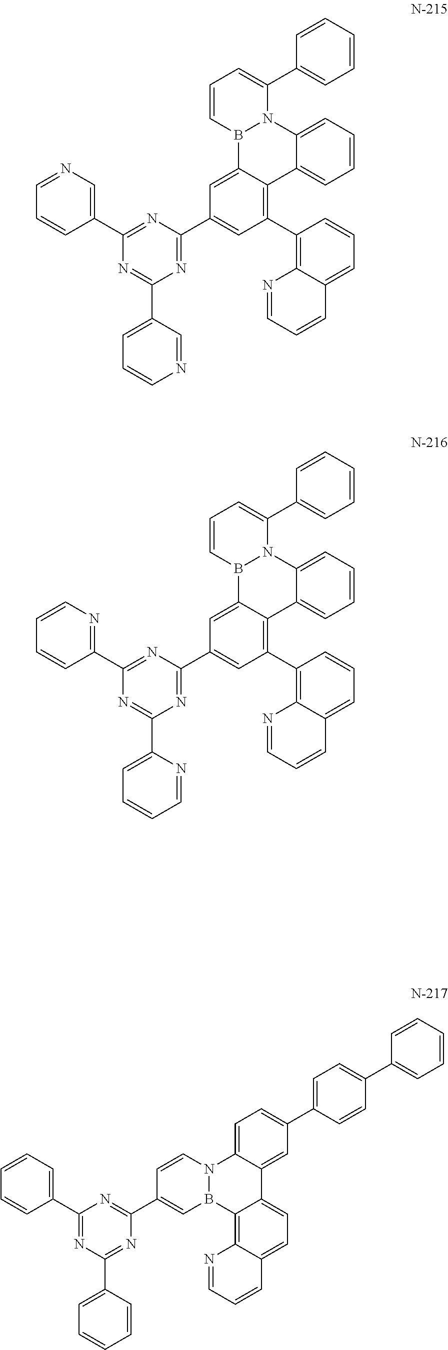 [SMILES: C1=CB2c3cc(-c4nc(-c5cccnc5)nc(-c5cccnc5)n4)cc(-c4cccc5cccnc45)c3-c3ccccc3N2C(c2ccccc2)=C1 ; C1=CB2c3cc(-c4nc(-c5ccccn5)nc(-c5ccccn5)n4)cc(-c4cccc5cccnc45)c3-c3ccccc3N2C(c2ccccc2)=C1 ; C1=CN2B(C=C1c1nc(-c3ccccc3)nc(-c3ccccc3)n1)c1c(ccc3cccnc13)-c1cc(-c3ccc(-c4ccccc4)cc3)ccc12]

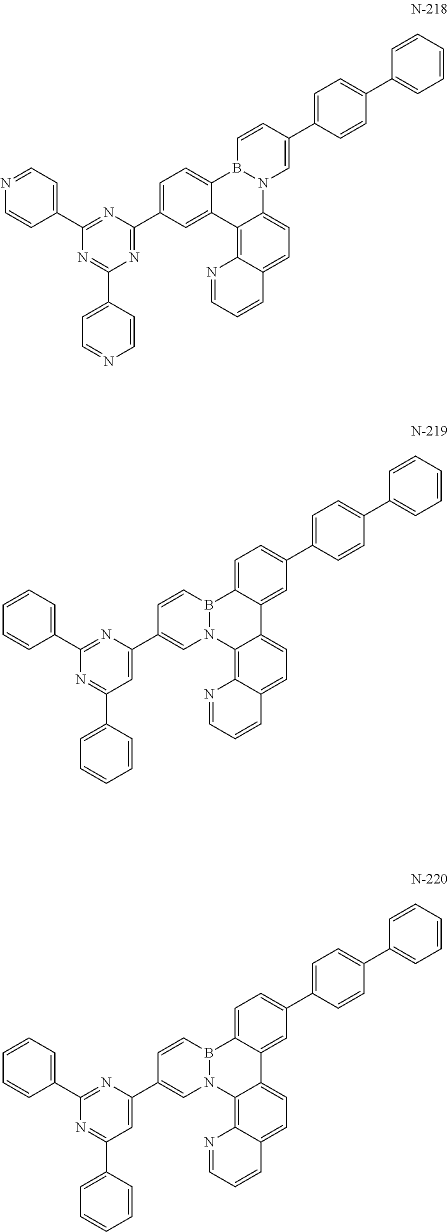 [SMILES: C1=CC(c2ccc(-c3ccccc3)cc2)=CN2B1c1ccc(-c3nc(-c4ccncc4)nc(-c4ccncc4)n3)cc1-c1c2ccc2cccnc12 ; C1=CC(c2cc(-c3ccccc3)nc(-c3ccccc3)n2)=CN2B1c1ccc(-c3ccc(-c4ccccc4)cc3)cc1-c1ccc3cccnc3c12 ; C1=CC(c2cc(-c3ccccc3)nc(-c3ccccc3)n2)=CN2B1c1ccc(-c3ccc(-c4ccccc4)cc3)cc1-c1ccc3cccnc3c12]

In some embodiments, the organic compound as described herein is used for evaporation-based OLEDs. For this purpose, the molecular weight of the organic compound 1000 g/mol, preferably ≤900 g/mol, more preferably ≤850 g/mol, further preferably ≤800 g/mol, and most preferably ≤750 g/mol.

In another aspect, the present disclosure also provides an organic mixture comprising a first compound H1 and a second compound H2, the first compound H1 comprises an organic compound as described herein, and min(LUMO(H1)−HOMO(H2), LUMO(H2)−HOMO(H1))≤min(E_{T}(H1), E_{T}(H2))+0.1 eV, where HOMO(H1), LUMO(H1), and E_{T}(H1) stand for the energy level of the highest occupied molecular orbital, the energy level of the lowest unoccupied molecular orbital, and the triplet energy level of the first compound H1. HOMO(H2), LUMO(H2), and E_{T}(H2) stand for the energy level of the highest occupied molecular orbital, the energy level of the lowest unoccupied molecular orbital, and the triplet energy level of the second compound H2.

In some embodiments, the first compound H1 and the second compound H2 of the organic mixture form a type II heterojunction energy structure.

In some embodiments, in the organic mixture as described herein, min(LUMO(H1)−HOMO(H2), LUMO(H2)−HOMO(H1))≤min(E_{T}(H1), E_{T}(H2)).

In some embodiments, in the organic mixture as described herein, min(LUMO(H1)−HOMO(H2), LUMO(H2)−HOMO(H1))≤min(E_{T}(H1), E_{T}(H2))−0.05 eV.

In some embodiments, in the organic mixture as described herein, min(LUMO(H1)−HOMO(H2), LUMO(H2)−HOMO(H1))≤min(E_{T}(H1), E_{T}(H2))−0.1 eV.

In some embodiments, in the organic mixture as described herein, min(LUMO(H1)−HOMO(H2), LUMO(H2)−HOMO(H1))≤min(E_{T}(H1), E_{T}(H2))−0.15 eV.

In some embodiments, in the organic mixture as described herein, min(LUMO(H1)−HOMO(H2), LUMO(H2)−HOMO(H1))≤min(E_{T}(H1), E_{T}(H2))−0.2 eV.

In the embodiments of the present disclosure, the energy level structure of the organic material, the singlet energy level E_{S}, triplet energy level E_{T}, HOMO, and LUMO play key roles. The determination of these energy levels is introduced as follows.

HOMO and LUMO energy levels can be measured by photoelectric effects, for example by XPS (X-ray photoelectron spectroscopy), UPS (UV photoelectron spectroscopy), or by cyclic voltammetry (hereinafter referred to as CV). Recently, quantum chemical methods, such as density functional theory (hereinafter referred to as DFT), are becoming effective methods for calculating the molecular orbital energy levels.

The singlet energy level E_{S} of the organic material can be determined by the emission spectrum, and the triplet energy level E_{T} of the organic material can be measured by low-temperature time-resolved spectroscopy. E_{S} and E_{T} can also be calculated by quantum simulation (for example, by Time-dependent DFT), for instance with the commercial software Gaussian 09W (Gaussian Inc.), the specific simulation method can be found in WO2011141110 or as described in the following embodiments.

It should be noted that the absolute values of HOMO, LUMO, E_{S}, and E_{T} may vary depending on the measurement method or calculation method used. Even for the same method, different evaluation ways, for example, using either the onset or peak value of a CV curve as reference, may result in different (HOMO/LUMO) values. Therefore, reasonable and meaningful comparison should be carried out by using the same measurement and evaluation methods. In the embodiments of the present disclosure, the values of HOMO, LUMO, E_{S}, and E_{T} are based on Time-dependent DFT simulation, which however should not exclude the applications of other measurement or calculation methods.

A potential advantage in the organic mixture as described herein is that the excited states of the system will preferentially occupy the lowest energy exciplex, or facilitate the energy transfer from the triplet excited states of the first compound H1/the second compound H2 to the exciplex, thereby increasing the concentration of the complex excited state.

In some embodiments, the organic mixture as described herein may be used as a phosphorescent host material.

In the present disclosure, (HOMO-1) is defined as the energy level of the second highest occupied molecular orbital, (HOMO-2) is defined as the energy level of the third highest occupied molecular orbital, and so on. (LUMO+1) is defined as the energy level of the second lowest unoccupied molecular orbital, (LUMO+2) is defined as the energy level of the third lowest occupied molecular orbital, and so on.

In some embodiments, in the organic mixture as described herein, min(LUMO_{H1}−HOMO_{H2}), (LUMO_{H2}−HOMO_{H1})) is less than or equal to the triplet excited states energy level of the first compound H1, while at the same time being less than or equal to the triplet excited energy level of the second compound H2. The energy of the exciplex formed by the first compound H1 and the second compound H2 is dependent on min((LUMO_{H1}−HOMO_{H2}), (LUMO_{H2}−HOMO_{H1})).

In some embodiments, in the organic mixture as described herein, at least one of the first compound H1 or the second compound H2 has (HOMO−(HOMO-1))≥0.2 eV, preferably ≥0.25 eV, more preferably ≥0.3 eV, even more preferably ≥0.35 eV, further preferably ≥0.4 eV, and most preferably ≥0.45 eV.

In some embodiments, the second compound H2 of the organic mixture as described herein comprises an organic compound containing an electron-donating group. One possible benefit of this combination is that the first compound H1 and the second compound H2 are easy to form a type II semiconductor heterojunction.

In some embodiments, the second compound H2 of the organic mixture has (HOMO−(HOMO-1))≥0.2 eV, preferably ≥0.25 eV, more preferably ≥0.3 eV, even more preferably ≥0.35 eV, further preferably ≥0.4 eV, and most preferably ≥0.45 eV.

In some embodiments, the second compound H2 of the organic mixture is selected from one of the following formulas (II-1)-(II-4):

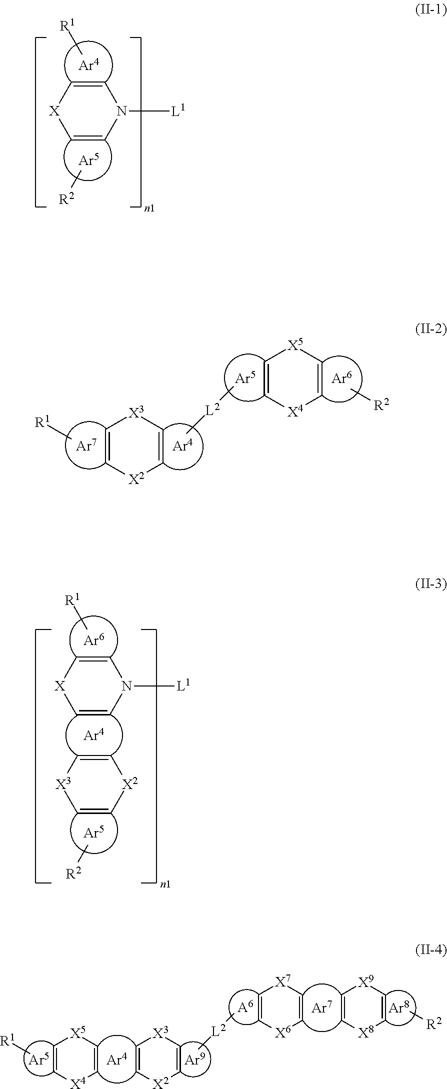

Where each L¹ represents an aromatic or a hetero-aromatic group containing 5 to 60 ring atoms; each L² represents a single bonded, an aromatic or a hetero-aromatic group containing 5 to 30 ring atoms, the attachment position of L¹ and L² can be on any carbon atom of the benzene ring; Ar⁴ to Ar⁹ are independently selected from an aromatic or a hetero-aromatic group containing 5 to 30 ring atoms; each X represents a single bond, N(R), C(R)₂, Si(R)₂, O, C═N(R), C═C(R)₂, P(R), P(═O)R, S, S═O, or SO₂; X² to X₉ are independently selected from a single bond, N(R), C(R)₂, Si(R)₂, O, C═N(R), C═C(R)₂, P(R), P(═O)R, S, S═O, or SO₂; and X² and X³ are not single bonds at the same time, X⁴ and X⁵ are not single bonds at the same time, X⁶ and X⁷ are not single bonds at the same time, X⁸ and X⁹ are not single bonds at the same time.

R¹, R², and R are independently selected from the group consisting of H, D, F, CN, an alkenyl group, an alkynyl group, a nitrile group, an amine group, a nitro group, an acyl group, an alkoxy group, a carbonyl group, a sulfone group, a C₁-C₃₀ alkyl group, a C₃-C₃₀ cycloalkyl group, a C₅-C₆₀ aromatic hydrocarbon group, and a C₅-C₆₀ aromatic heterocyclic group, where the attachment position of R¹ and R² can be on any carbon atom of the fused ring, and there can be any number of carbon atoms substituted with R¹ and R²; n1 is an integer from 1 to 4.

In some embodiments, the second compound H2 is selected from one of the following formulas (III-1)-(III-4):

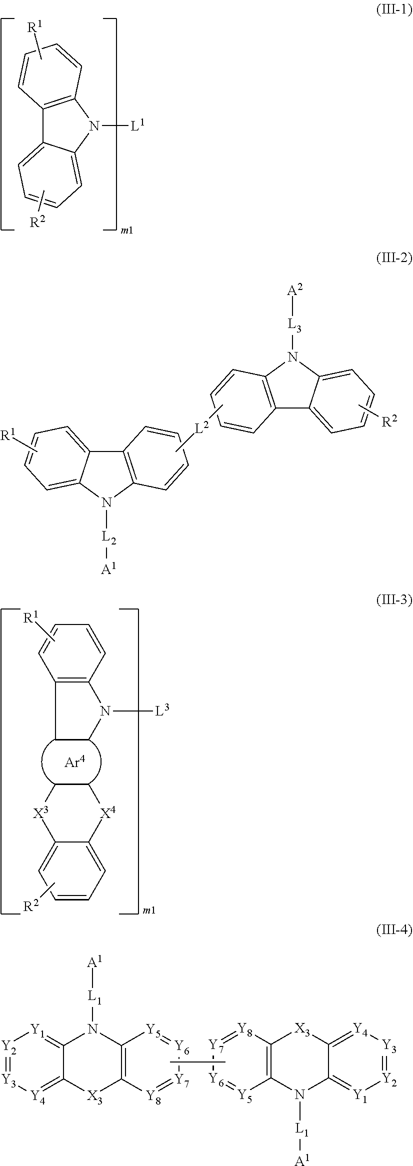

Where L³ is defined as the above-mentioned L¹; A¹ and A² are independently selected from an aromatic or a hetero-aromatic group containing 5 to 30 ring atoms; each of Yi to Ys is N or CR, and two adjacent Xs cannot be N at the same time.

Preferably, the second compound H2 of formulas (II-1)-(II-4) and (III-1)-(III-4) is selected from one of the following structural formulas:

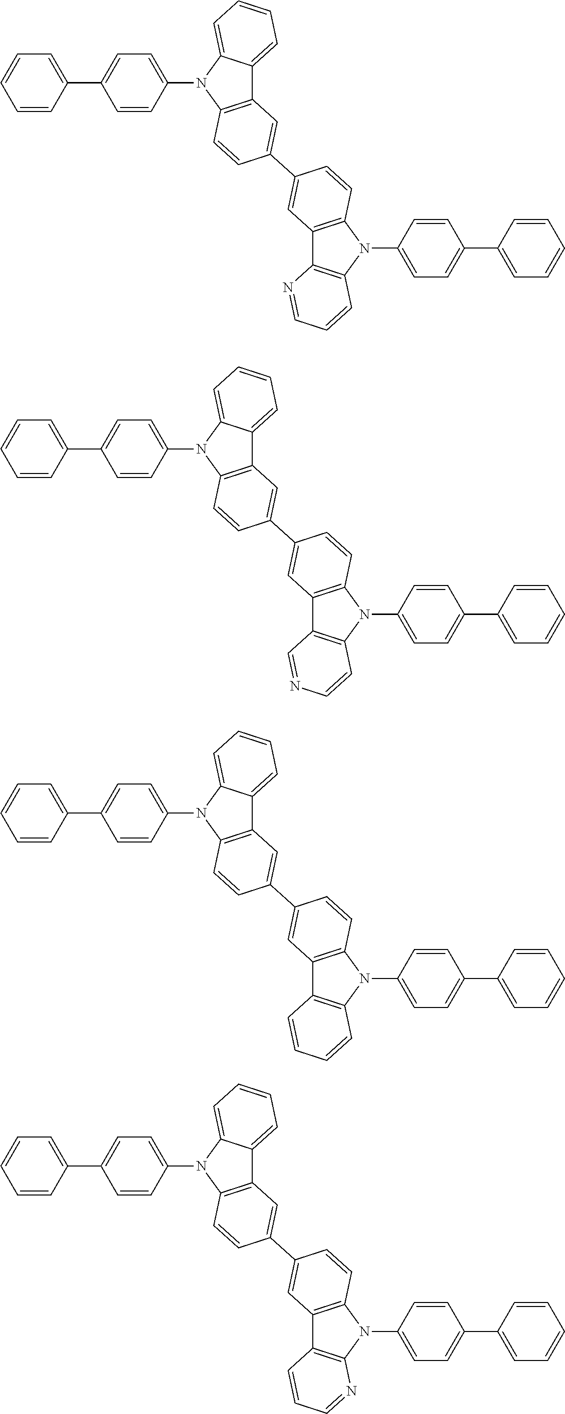 [SMILES: c1ccc(-c2ccc(-n3c4ccccc4c4cc(-c5ccc6c(c5)c5ncccc5n6-c5ccc(-c6ccccc6)cc5)ccc43)cc2)cc1 ; c1ccc(-c2ccc(-n3c4ccccc4c4cc(-c5ccc6c(c5)c5cnccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5cccnc5n6-c5ccc(-c6ccccc6)cc5)ccc43)cc2)cc1]

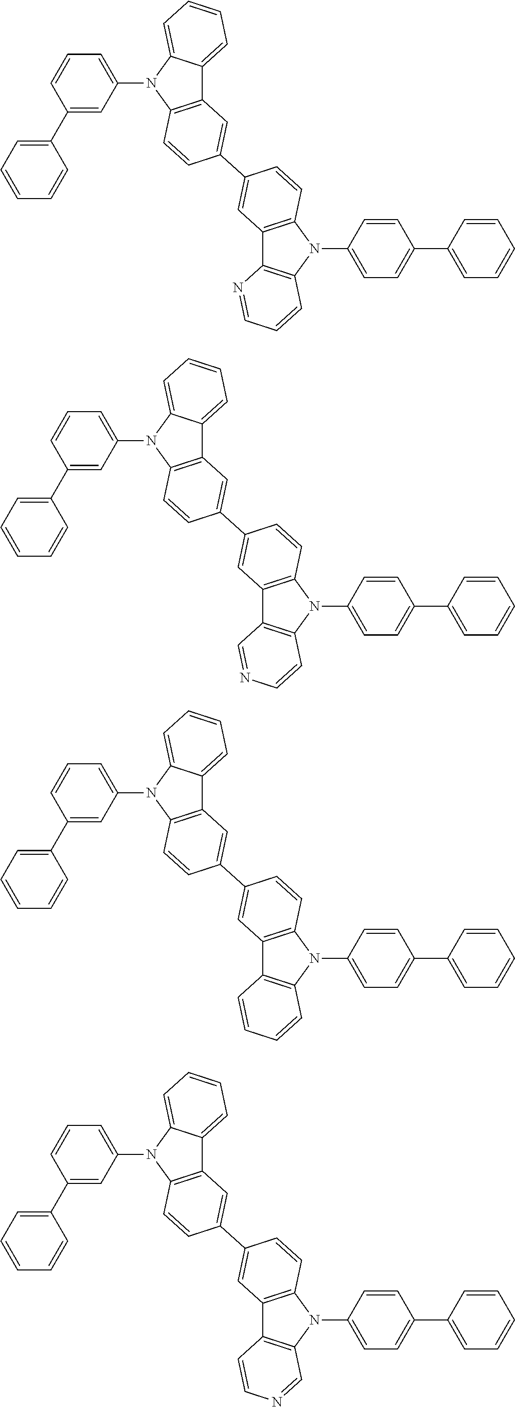 [SMILES: c1ccc(-c2ccc(-n3c4ccc(-c5ccc6c(c5)c5ccccc5n6-c5cccc(-c6ccccc6)c5)cc4c4ncccc43)cc2)cc1 ; c1ccc(-c2ccc(-n3c4ccncc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccc(-c5ccc6c(c5)c5ccccc5n6-c5cccc(-c6ccccc6)c5)cc4c4ccncc43)cc2)cc1]

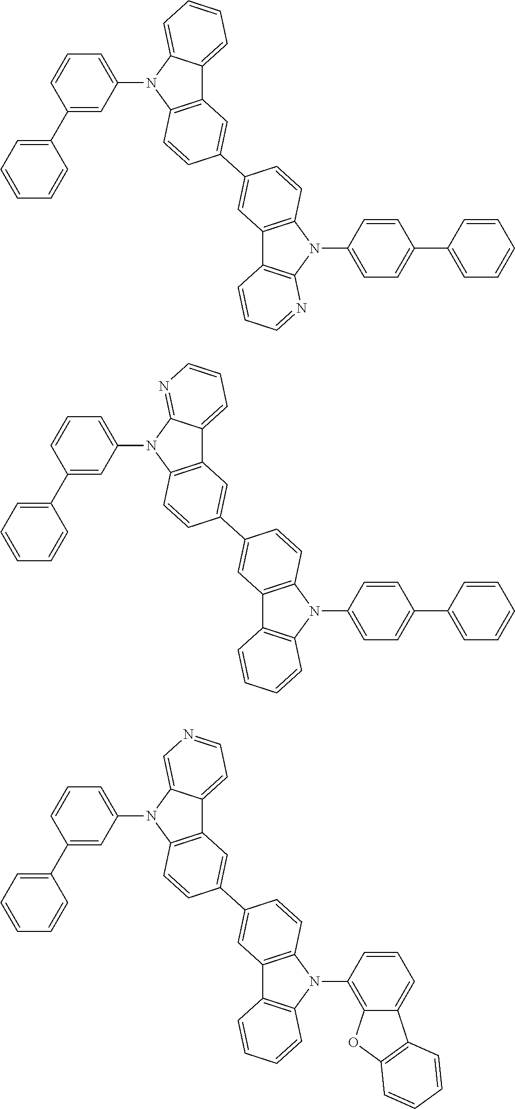 [SMILES: c1ccc(-c2ccc(-n3c4ccc(-c5ccc6c(c5)c5ccccc5n6-c5cccc(-c6ccccc6)c5)cc4c4cccnc43)cc2)cc1 ; c1ccc(-c2ccc(-n3c4ccccc4c4cc(-c5ccc6c(c5)c5cccnc5n6-c5cccc(-c6ccccc6)c5)ccc43)cc2)cc1 ; c1ccc(-c2cccc(-n3c4ccc(-c5ccc6c(c5)c5ccccc5n6-c5cccc6c5oc5ccccc56)cc4c4ccncc43)c2)cc1]

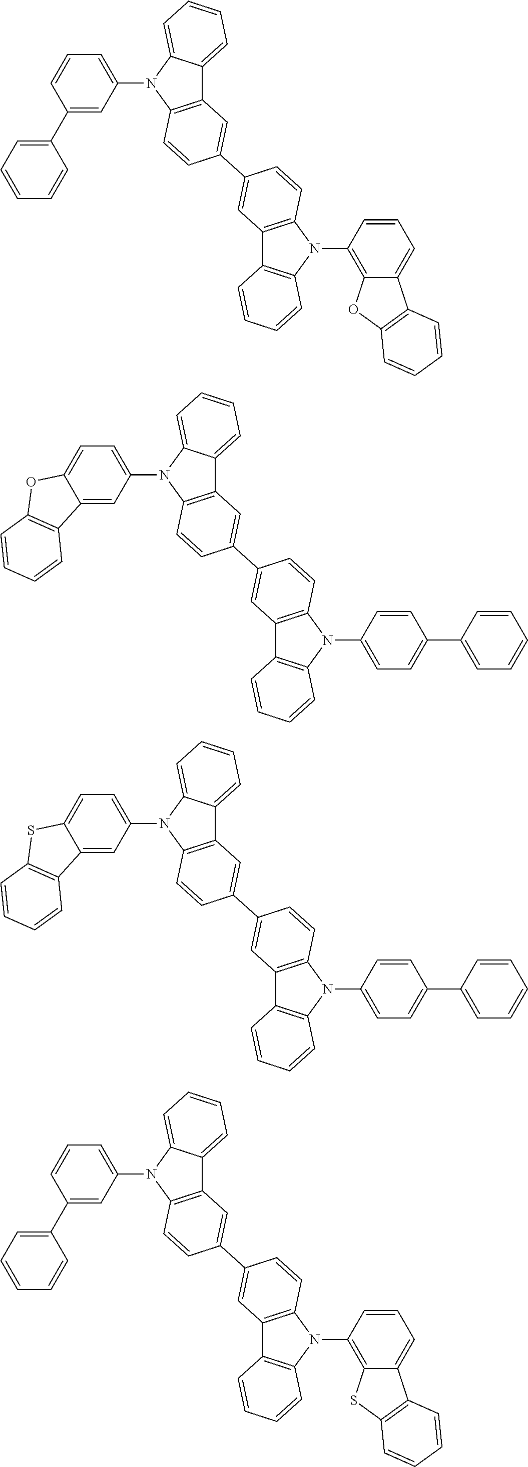 [SMILES: c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cccc(-n3c4ccccc4c4cc(-c5ccc6c(c5)c5ccccc5n6-c5cccc6c5sc5ccccc56)ccc43)c2)cc1]

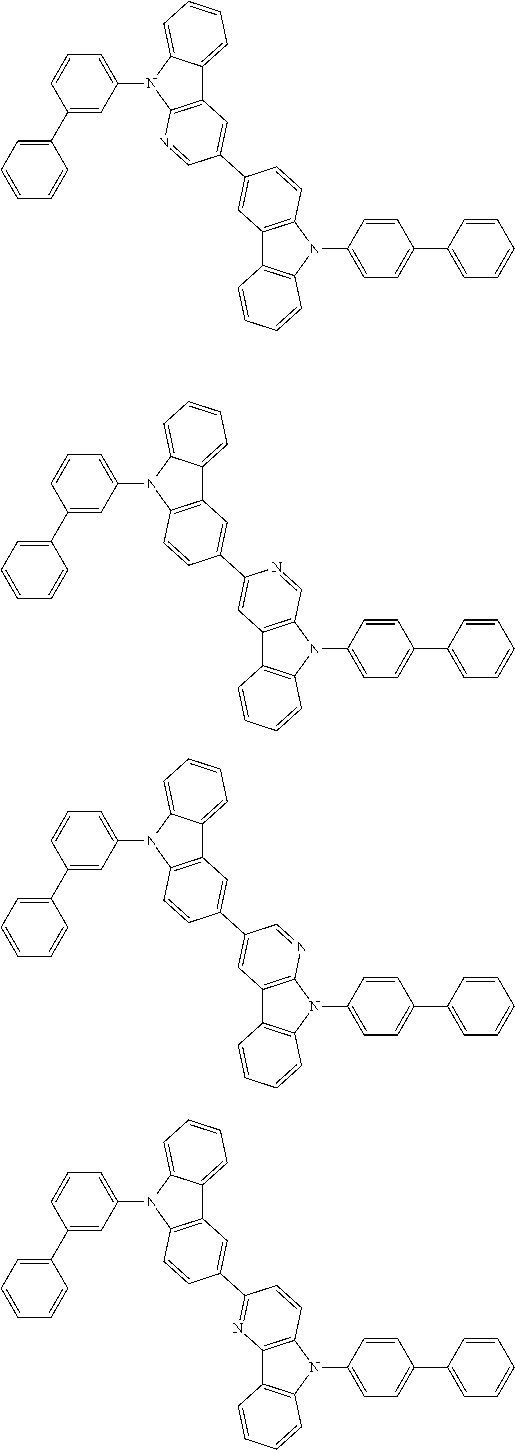 [SMILES: c1ccc(-c2ccc(-n3c4ccccc4c4cc(-c5cnc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cccc(-c6ccccc6)c5)ncc43)cc2)cc1 ; c1ccc(-c2ccc(-n3c4ccccc4c4cc(-c5ccc6c(c5)c5ccccc5n6-c5cccc(-c6ccccc6)c5)cnc43)cc2)cc1 ; c1ccc(-c2ccc(-n3c4ccccc4c4nc(-c5ccc6c(c5)c5ccccc5n6-c5cccc(-c6ccccc6)c5)ccc43)cc2)cc1]

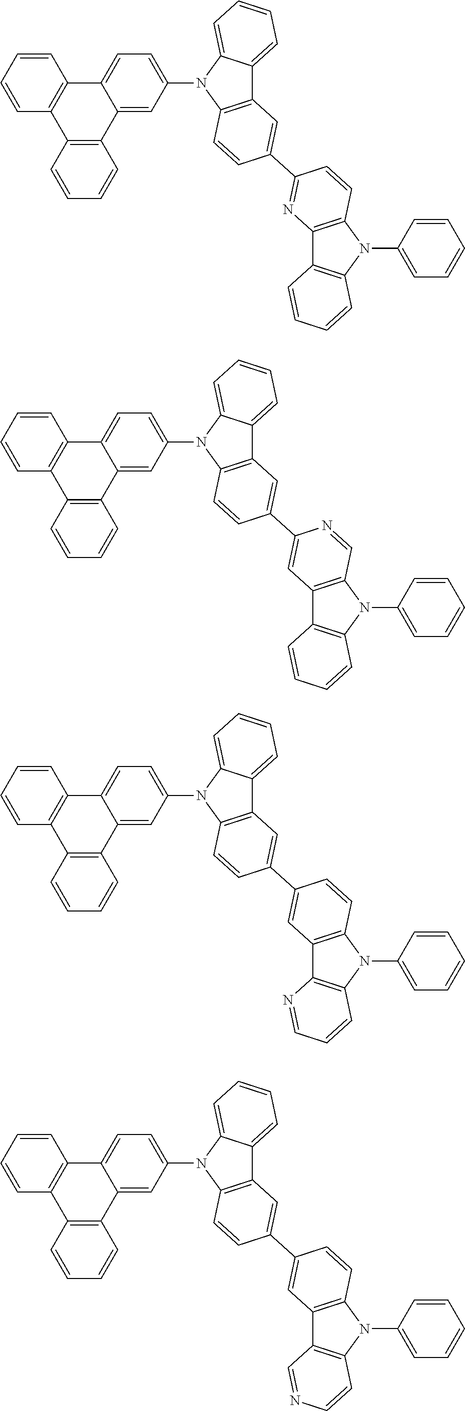 [SMILES: c1ccc(-n2c3ccccc3c3nc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4ccc5c6ccccc6c6ccccc6c5c4)ncc32)cc1 ; c1ccc(-n2c3ccc(-c4ccc5c(c4)c4ccccc4n5-c4ccc5c6ccccc6c6ccccc6c5c4)cc3c3ncccc32)cc1 ; c1ccc(-n2c3ccncc3c3cc(-c4ccc5c(c4)c4ccccc4n5-c4ccc5c6ccccc6c6ccccc6c5c4)ccc32)cc1]

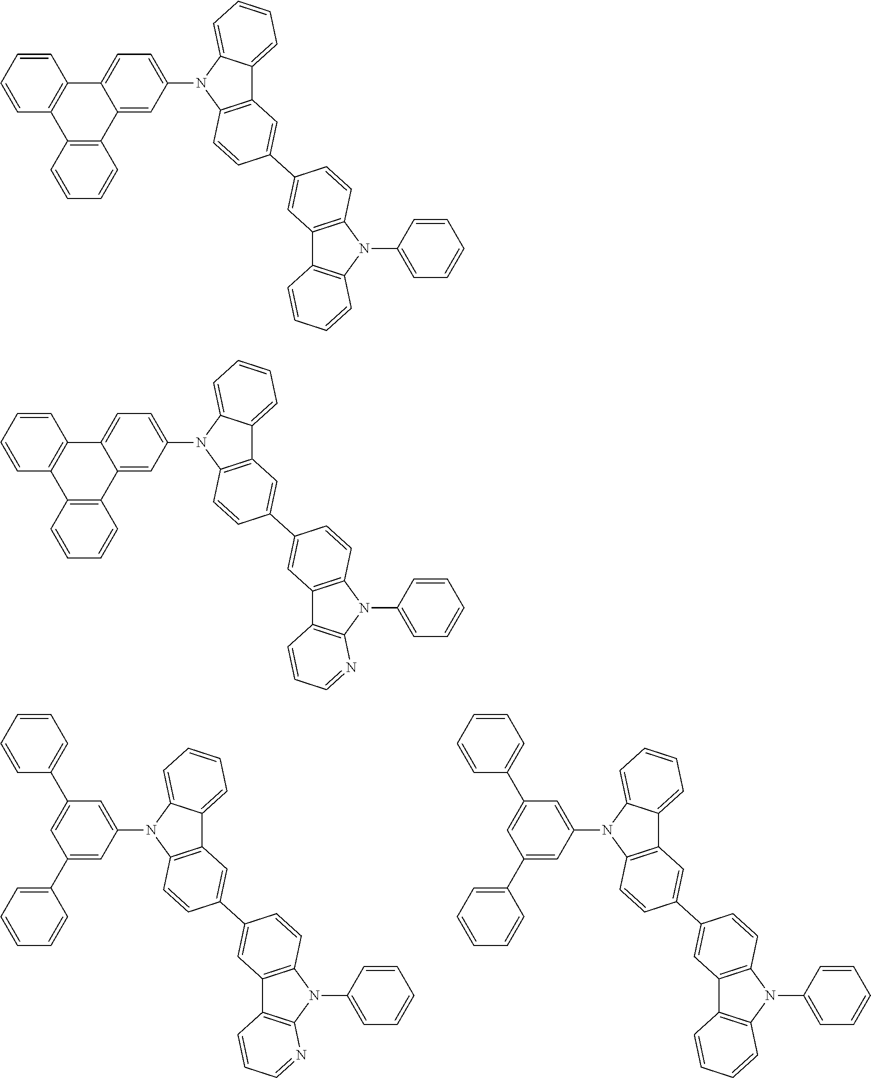 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccc(-c4ccc5c(c4)c4ccccc4n5-c4ccc5c6ccccc6c6ccccc6c5c4)cc3c3cccnc32)cc1 ; c1ccc(-c2cc(-c3ccccc3)cc(-n3c4ccccc4c4cc(-c5ccc6c(c5)c5cccnc5n6-c5ccccc5)ccc43)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

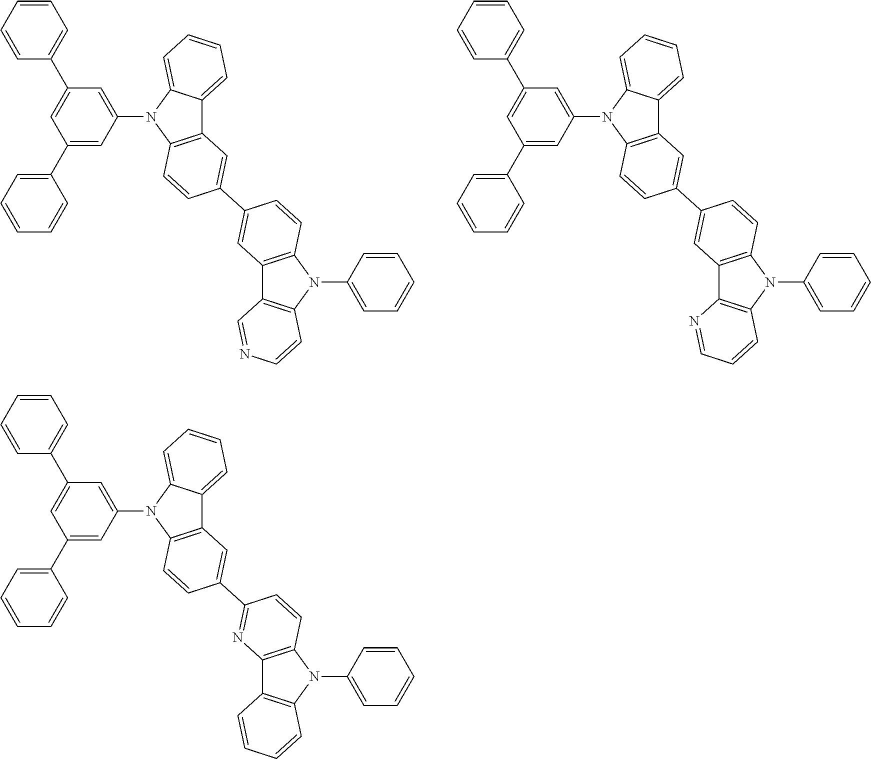 [SMILES: c1ccc(-c2cc(-c3ccccc3)cc(-n3c4ccccc4c4cc(-c5ccc6c(c5)c5cnccc5n6-c5ccccc5)ccc43)c2)cc1 ; c1ccc(-c2cc(-c3ccccc3)cc(-n3c4ccccc4c4cc(-c5ccc6c(c5)c5ncccc5n6-c5ccccc5)ccc43)c2)cc1 ; c1ccc(-c2cc(-c3ccccc3)cc(-n3c4ccccc4c4cc(-c5ccc6c(n5)c5ccccc5n6-c5ccccc5)ccc43)c2)cc1]

In some embodiments, the organic mixture as described herein can be used as a host material of the light-emitting layer in an electroluminescent device, where min((LUMO_{H1}−HOMO_{H2}), (LUMO_{H}2−HOMO_{H1})) is less than or equal to the triplet excited energy level of the first compound H1/the second compound H2.

When a single material with electron-biased or hole-biased characteristics is used to form the light-emitting layer, relatively more excitons can form at the interface between the light-emitting layer and the electron-transport layer or the hole-transport layer. Therefore, the excitons of the light-emitting layer may interact with the interfacial charge of the electron-transport layer or the hole-transport layer, thus causing a sharp roll-off in the device efficiency at high luminance and shortening device lifetime. In order to solve this problem, the first compound H1 as a first host material and the second compound H2 as a second host material are mixed and introduced into the light-emitting layer to balance the hole and electron mobility of the light-emitting layer, so that the middle region of the light-emitting layer emits light, increasing the device efficiency and improving the device lifetime. Mixing the first compound H1 and the second compound H2 to form an organic mixture, the range of mass ratios that can be used is preferably from 2:8 to 8:2, more preferably from 3:7 to 7:3, even more preferably from 4:6 to 6:4, further preferably from 4.5:5.5 to 5.5:4.5, and most preferably 5:5.

In some embodiments, the organic compound as described herein is a small molecule material, and the organic mixture as described herein is also a small molecule organic mixture.

The term “small molecule” herein refers to a molecule that is none of following: a polymer, an oligomer, a dendrimer, or a blend. In particular, there are no repeating structures in the small molecule. The molecular weight of the small molecule ≤4000 g/mol, preferably ≤3000 g/mol, more preferably ≤2000 g/mol, and most preferably ≤1500 g/mol.

The term of polymer comprises homopolymer, copolymer, and block copolymer. Also in the present disclosure, the term of polymer comprises dendrimer. For the synthesis and application of the dendrimers please refer to [Dendrimers and Dendrons, Wiley-VCH Verlag GmbH & Co. KGaA, 2002, Ed. George R. Newkome, Charles N. Moorefield, Fritz Vogtle.].

The term “conjugated polymer” refers to a polymer with backbone mainly comprising sp² hybrid orbitals of C atoms, well-known examples are polyacetylene and poly (phenylene vinylene).

The C atoms on the backbones can also be substituted with other non-C atoms. Moreover, the above-mentioned structure should still be considered as a conjugated polymer when the sp² hybridization on the backbone is interrupted by natural defects. Also in the present disclosure, the backbone of the conjugated polymer comprises aryl amines, aryl phosphines and other heteroaromatics, organometallic complexes, etc.

In some embodiments, in order to improve the material evaporation efficiency and material utilization, as well as to simplify the material evaporation process, the molecular weight difference between the first compound H1 (first host material) and the second compound H2 (second host material) is ≤100 Dalton, preferably ≤90 Dalton, more preferably ≤70 Dalton, even more preferably ≤60 Dalton, further preferably ≤50 Dalton, and most preferably ≤20 Dalton.

In some embodiments, in the organic mixture as described herein, the sublimation temperature difference between the first compound H1 and the second compound H2 ≤40 K, preferably ≤30 K, more preferably ≤25 K, even more preferably ≤20 K, further preferably ≤18 K, and most preferably ≤15 K.

In particular, the solubility of the small organic molecule compounds is ensured by the substituent R on the units of formulas (I), (I-1) to (I-3), (II-1) to (II-4), (III-1) to (III-4) and optionally on the additionally present unit, as well as by adjusting the linkage site between the core structure and the substituent.

Depending on substitution patterns, the structural unit of formulas (I), (I-1) to (I-3), (II-1) to (II-4), (III-1) to (III-4) can be modified to the various functions of the small organic molecule compounds. Thus, they can preferably be used as the core structure of the organic small molecule compound.

The preferred examples according to formulas (II-1) to (II-4), (III-1) to (III-4) are shown in the following structures. These structures may be substituted at all possible substitution sites.

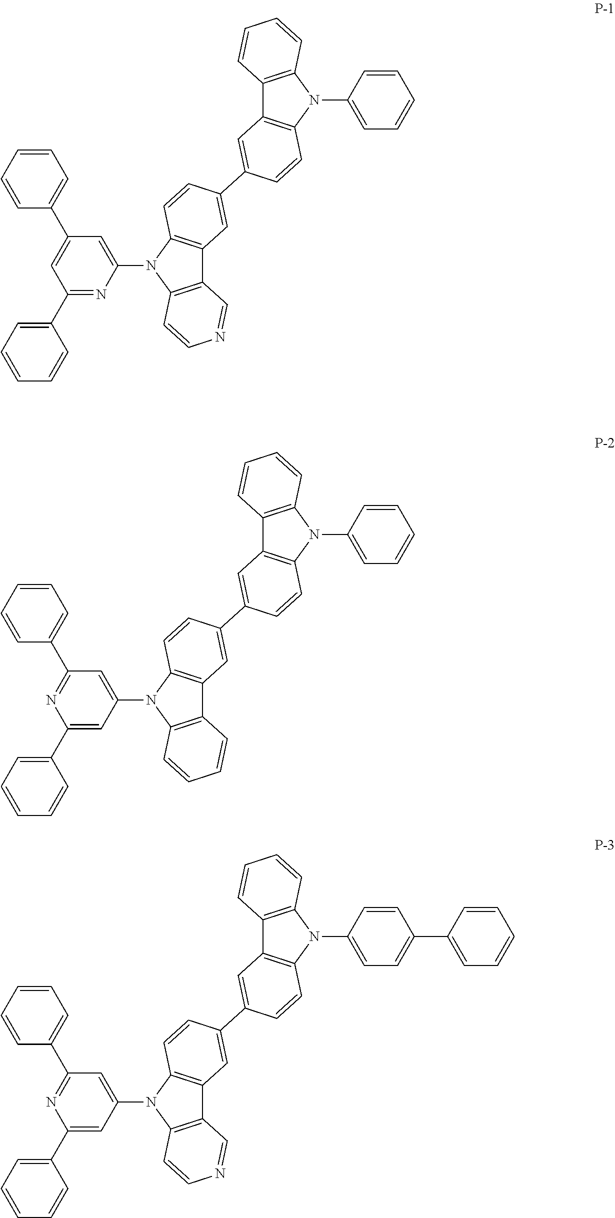 [SMILES: c1ccc(-c2cc(-c3ccccc3)nc(-n3c4ccncc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1 ; c1ccc(-c2ccc(-n3c4ccccc4c4cc(-c5ccc6c(c5)c5cnccc5n6-c5cc(-c6ccccc6)nc(-c6ccccc6)c5)ccc43)cc2)cc1]

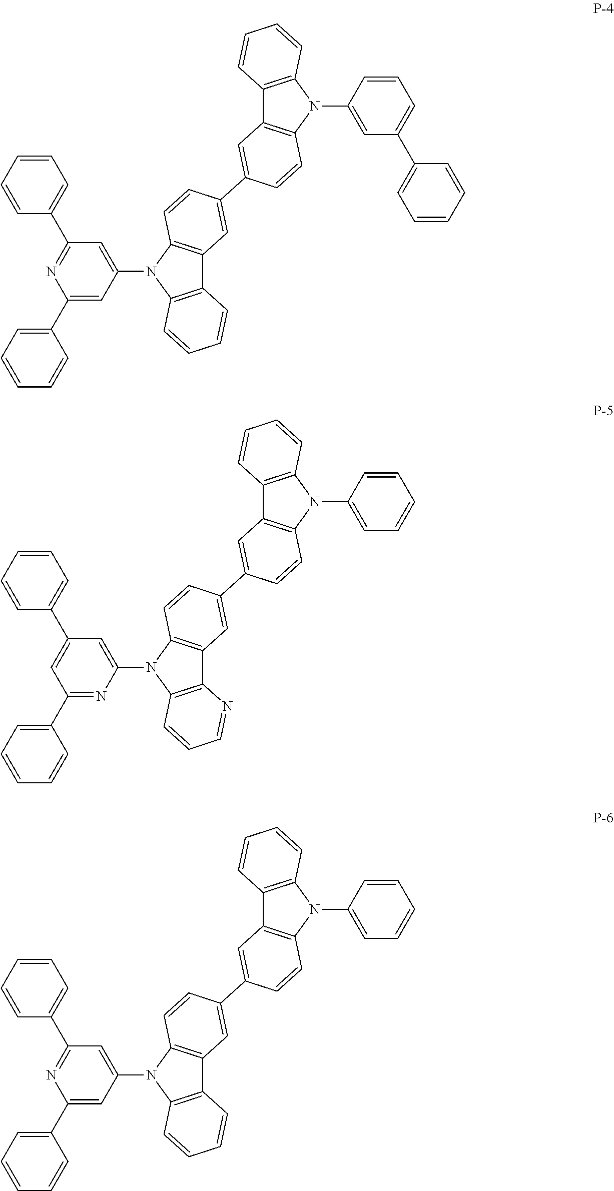 [SMILES: c1ccc(-c2cccc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)nc(-n3c4ccc(-c5ccc6c(c5)c5ccccc5n6-c5ccccc5)cc4c4ncccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1]

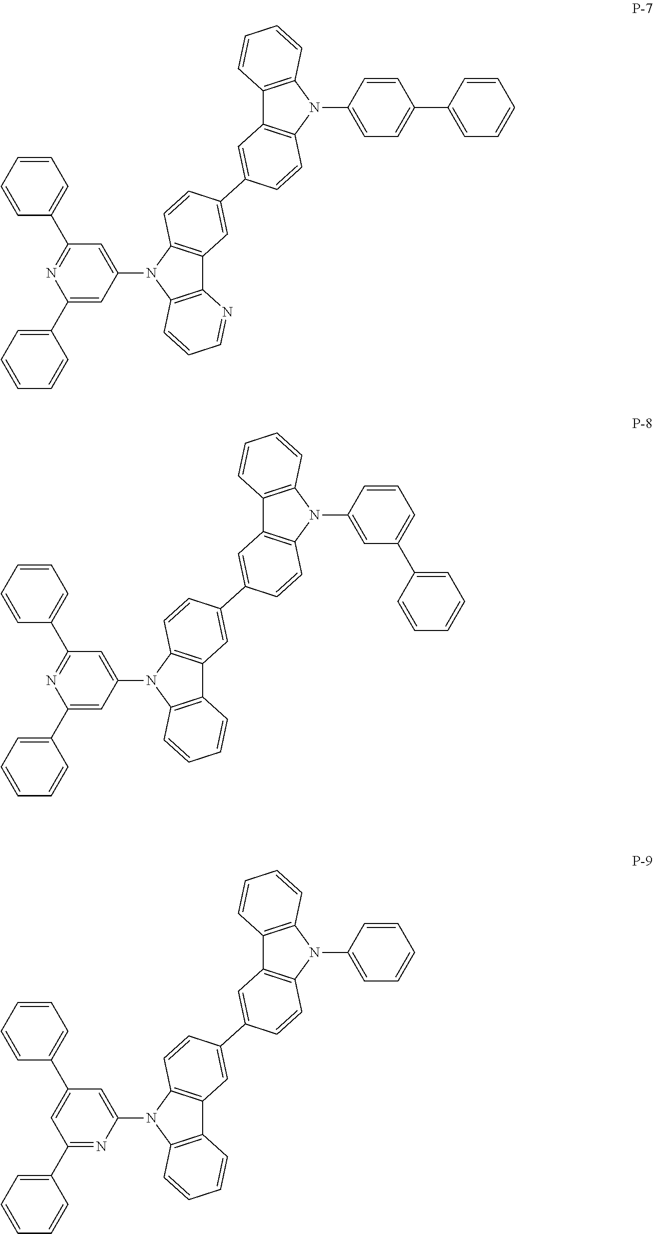 [SMILES: c1ccc(-c2ccc(-n3c4ccccc4c4cc(-c5ccc6c(c5)c5ncccc5n6-c5cc(-c6ccccc6)nc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)nc(-n3c4ccccc4c4cc(-c5ccc6c(c5)c5ccccc5n6-c5ccccc5)ccc43)c2)cc1]

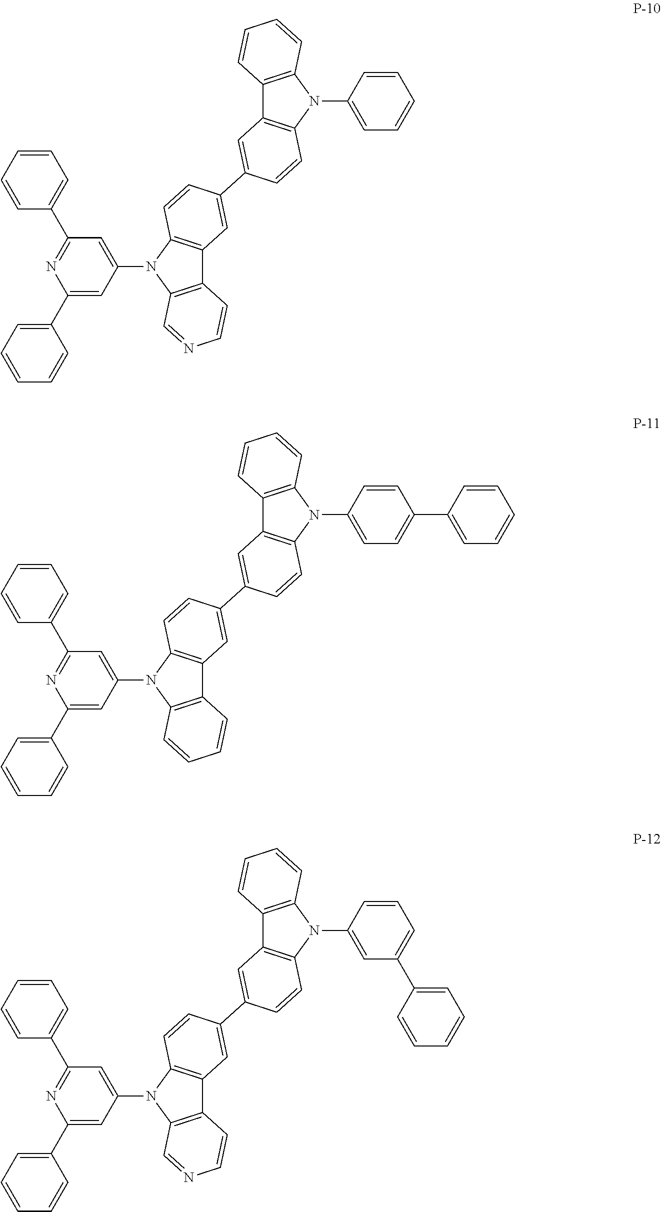 [SMILES: c1ccc(-c2cc(-n3c4ccc(-c5ccc6c(c5)c5ccccc5n6-c5ccccc5)cc4c4ccncc43)cc(-c3ccccc3)n2)cc1 ; c1ccc(-c2ccc(-n3c4ccccc4c4cc(-c5ccc6c(c5)c5ccccc5n6-c5cc(-c6ccccc6)nc(-c6ccccc6)c5)ccc43)cc2)cc1 ; c1ccc(-c2cccc(-n3c4ccccc4c4cc(-c5ccc6c(c5)c5ccncc5n6-c5cc(-c6ccccc6)nc(-c6ccccc6)c5)ccc43)c2)cc1]

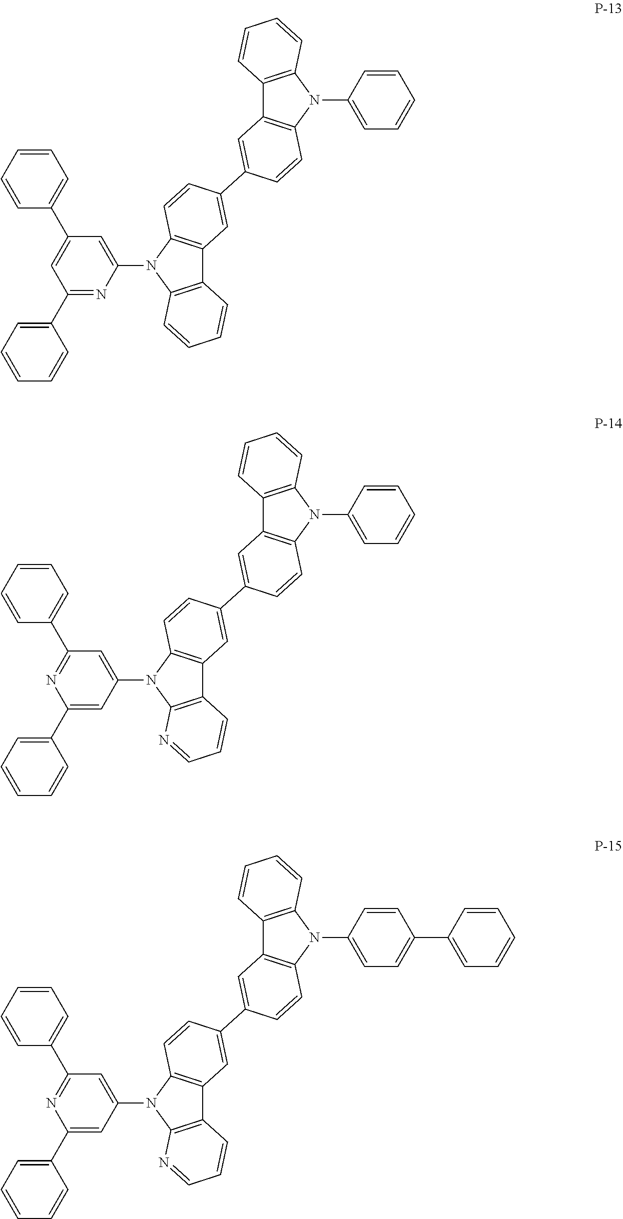 [SMILES: c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccc(-c5ccc6c(c5)c5ccccc5n6-c5ccccc5)cc4c4cccnc43)cc(-c3ccccc3)n2)cc1 ; c1ccc(-c2ccc(-n3c4ccccc4c4cc(-c5ccc6c(c5)c5cccnc5n6-c5cc(-c6ccccc6)nc(-c6ccccc6)c5)ccc43)cc2)cc1]

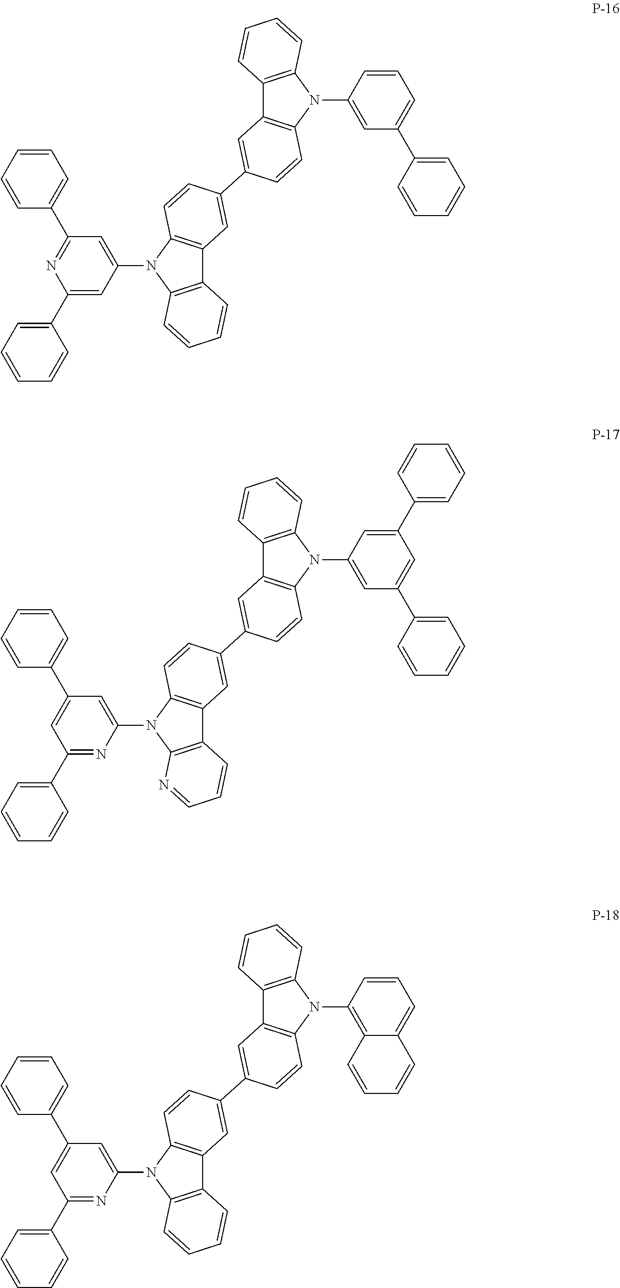 [SMILES: c1ccc(-c2cccc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)cc(-n3c4ccccc4c4cc(-c5ccc6c(c5)c5cccnc5n6-c5cc(-c6ccccc6)cc(-c6ccccc6)n5)ccc43)c2)cc1 ; c1ccc(-c2cc(-c3ccccc3)nc(-n3c4ccccc4c4cc(-c5ccc6c(c5)c5ccccc5n6-c5cccc6ccccc56)ccc43)c2)cc1]

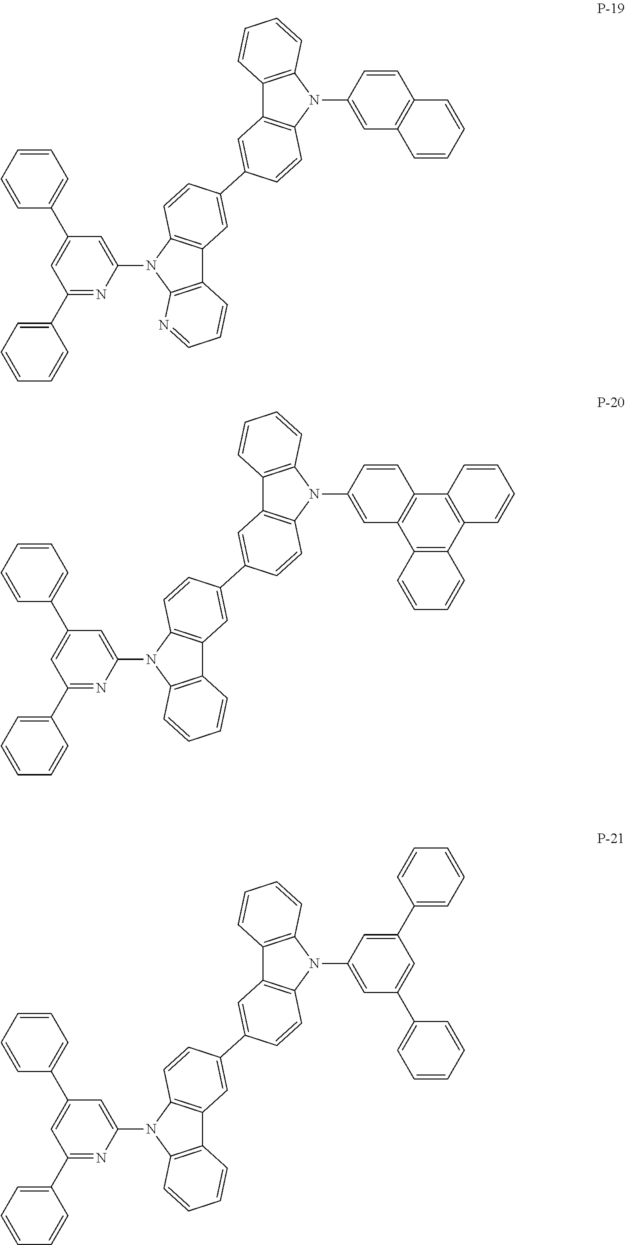 [SMILES: c1ccc(-c2cc(-c3ccccc3)nc(-n3c4ccc(-c5ccc6c(c5)c5ccccc5n6-c5ccc6ccccc6c5)cc4c4cccnc43)c2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1]

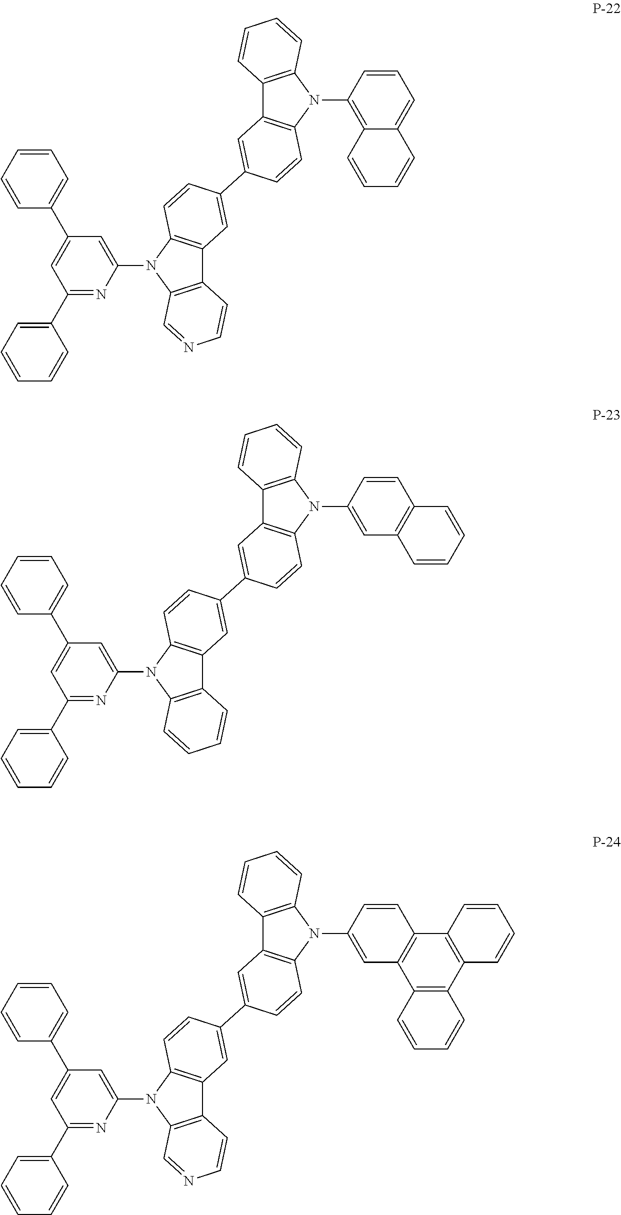 [SMILES: c1ccc(-c2cc(-c3ccccc3)nc(-n3c4ccc(-c5ccc6c(c5)c5ccccc5n6-c5cccc6ccccc56)cc4c4ccncc43)c2)cc1 ; c1ccc(-c2cc(-c3ccccc3)nc(-n3c4ccccc4c4cc(-c5ccc6c(c5)c5ccccc5n6-c5ccc6ccccc6c5)ccc43)c2)cc1 ; c1ccc(-c2cc(-c3ccccc3)nc(-n3c4ccc(-c5ccc6c(c5)c5ccccc5n6-c5ccc6c7ccccc7c7ccccc7c6c5)cc4c4ccncc43)c2)cc1]

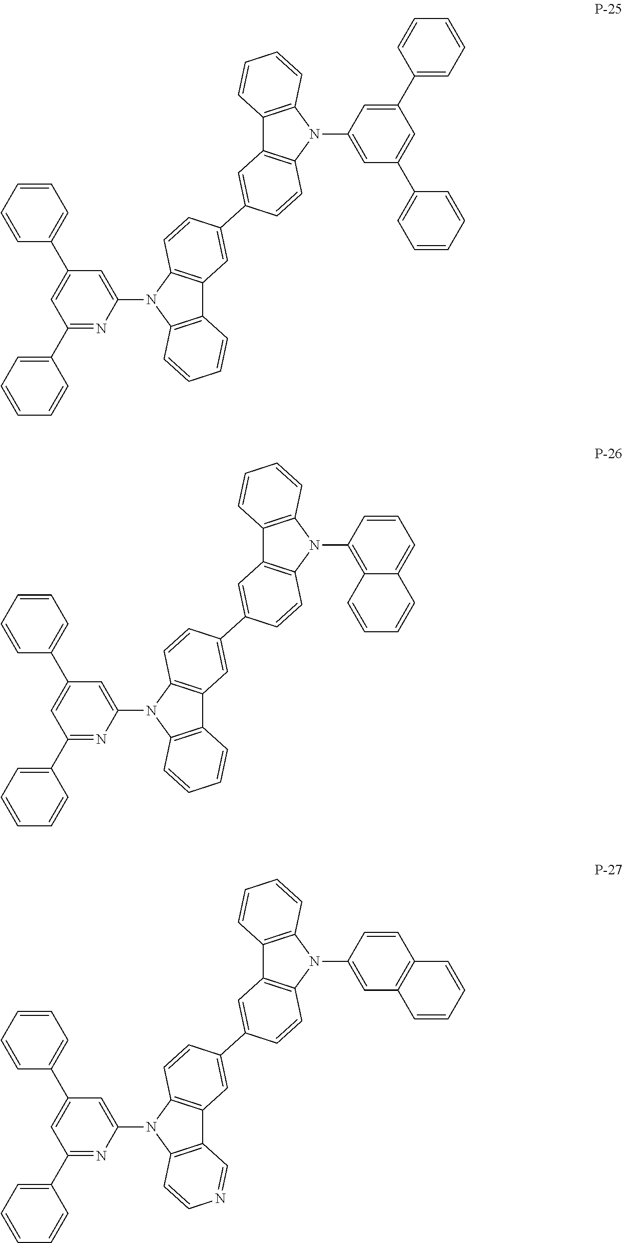 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)nc(-n3c4ccccc4c4cc(-c5ccc6c(c5)c5ccccc5n6-c5cccc6ccccc56)ccc43)c2)cc1 ; c1ccc(-c2cc(-c3ccccc3)nc(-n3c4ccncc4c4cc(-c5ccc6c(c5)c5ccccc5n6-c5ccc6ccccc6c5)ccc43)c2)cc1]

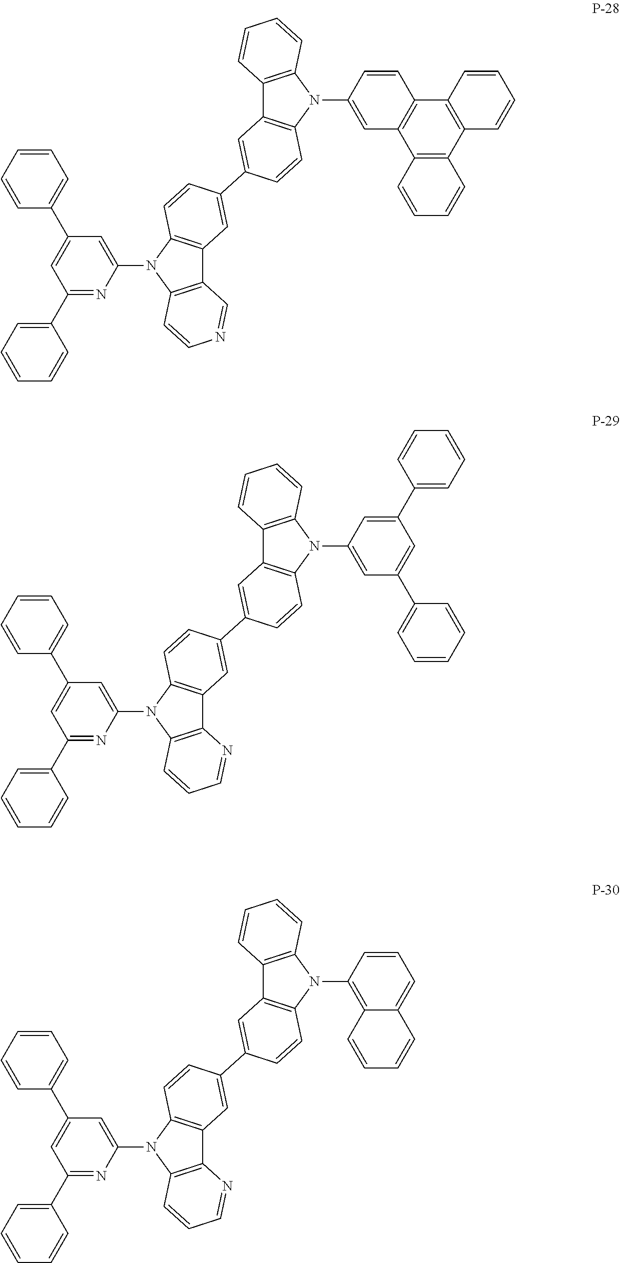 [SMILES: c1ccc(-c2cc(-c3ccccc3)nc(-n3c4ccncc4c4cc(-c5ccc6c(c5)c5ccccc5n6-c5ccc6c7ccccc7c7ccccc7c6c5)ccc43)c2)cc1 ; c1ccc(-c2cc(-c3ccccc3)cc(-n3c4ccccc4c4cc(-c5ccc6c(c5)c5ncccc5n6-c5cc(-c6ccccc6)cc(-c6ccccc6)n5)ccc43)c2)cc1 ; c1ccc(-c2cc(-c3ccccc3)nc(-n3c4ccc(-c5ccc6c(c5)c5ccccc5n6-c5cccc6ccccc56)cc4c4ncccc43)c2)cc1]

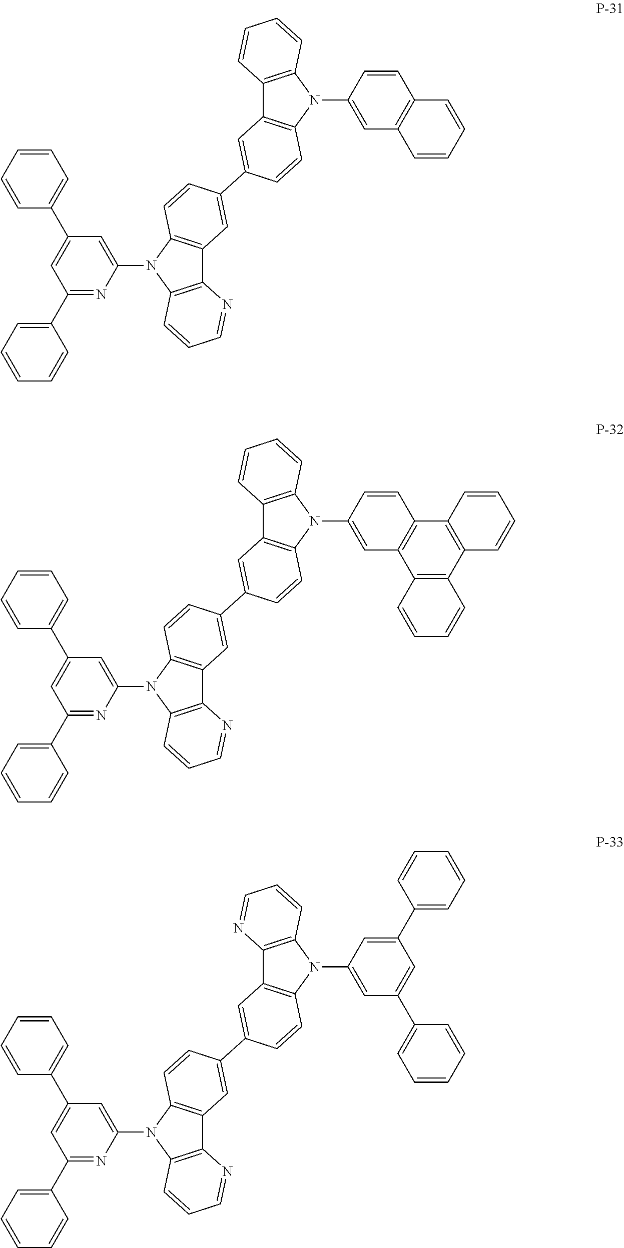 [SMILES: c1ccc(-c2cc(-c3ccccc3)nc(-n3c4ccc(-c5ccc6c(c5)c5ccccc5n6-c5ccc6ccccc6c5)cc4c4ncccc43)c2)cc1 ; c1ccc(-c2cc(-c3ccccc3)nc(-n3c4ccc(-c5ccc6c(c5)c5ccccc5n6-c5ccc6c7ccccc7c7ccccc7c6c5)cc4c4ncccc43)c2)cc1 ; c1ccc(-c2cc(-c3ccccc3)cc(-n3c4ccc(-c5ccc6c(c5)c5ncccc5n6-c5cc(-c6ccccc6)cc(-c6ccccc6)n5)cc4c4ncccc43)c2)cc1]

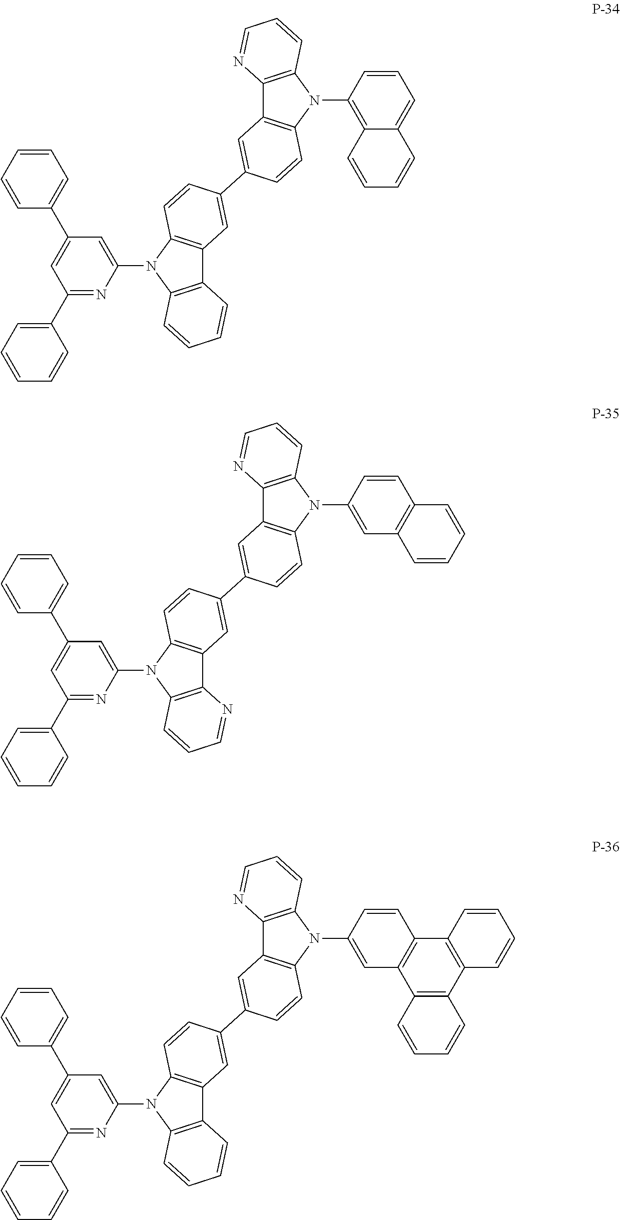 [SMILES: c1ccc(-c2cc(-c3ccccc3)nc(-n3c4ccccc4c4cc(-c5ccc6c(c5)c5ncccc5n6-c5cccc6ccccc56)ccc43)c2)cc1 ; c1ccc(-c2cc(-c3ccccc3)nc(-n3c4ccc(-c5ccc6c(c5)c5ncccc5n6-c5ccc6ccccc6c5)cc4c4ncccc43)c2)cc1 ; c1ccc(-c2cc(-c3ccccc3)nc(-n3c4ccccc4c4cc(-c5ccc6c(c5)c5ncccc5n6-c5ccc6c7ccccc7c7ccccc7c6c5)ccc43)c2)cc1]

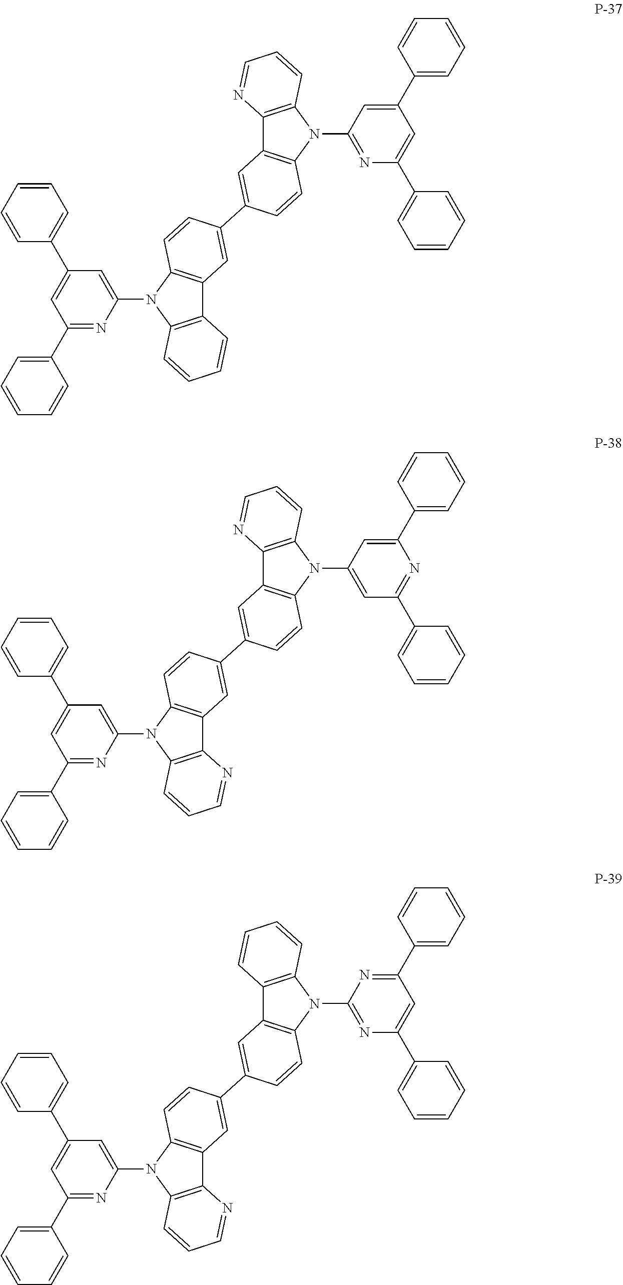 [SMILES: c1ccc(-c2cc(-c3ccccc3)nc(-n3c4ccccc4c4cc(-c5ccc6c(c5)c5ncccc5n6-c5cc(-c6ccccc6)cc(-c6ccccc6)n5)ccc43)c2)cc1 ; c1ccc(-c2cc(-c3ccccc3)nc(-n3c4ccc(-c5ccc6c(c5)c5ncccc5n6-c5cc(-c6ccccc6)nc(-c6ccccc6)c5)cc4c4ncccc43)c2)cc1 ; c1ccc(-c2cc(-c3ccccc3)nc(-n3c4ccc(-c5ccc6c(c5)c5ccccc5n6-c5nc(-c6ccccc6)cc(-c6ccccc6)n5)cc4c4ncccc43)c2)cc1]

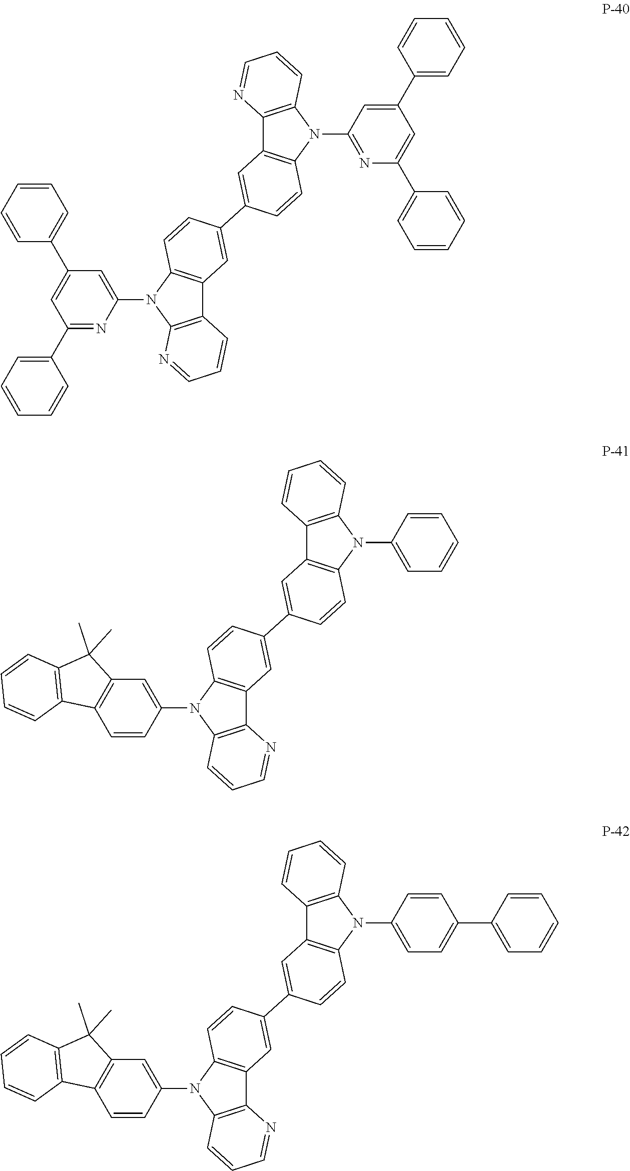 [SMILES: c1ccc(-c2cc(-c3ccccc3)nc(-n3c4ccc(-c5ccc6c(c5)c5cccnc5n6-c5cc(-c6ccccc6)cc(-c6ccccc6)n5)cc4c4ncccc43)c2)cc1 ; CC1(C)c2ccccc2-c2ccc(-n3c4ccc(-c5ccc6c(c5)c5ccccc5n6-c5ccccc5)cc4c4ncccc43)cc21 ; CC1(C)c2ccccc2-c2ccc(-n3c4ccc(-c5ccc6c(c5)c5ccccc5n6-c5ccc(-c6ccccc6)cc5)cc4c4ncccc43)cc21]

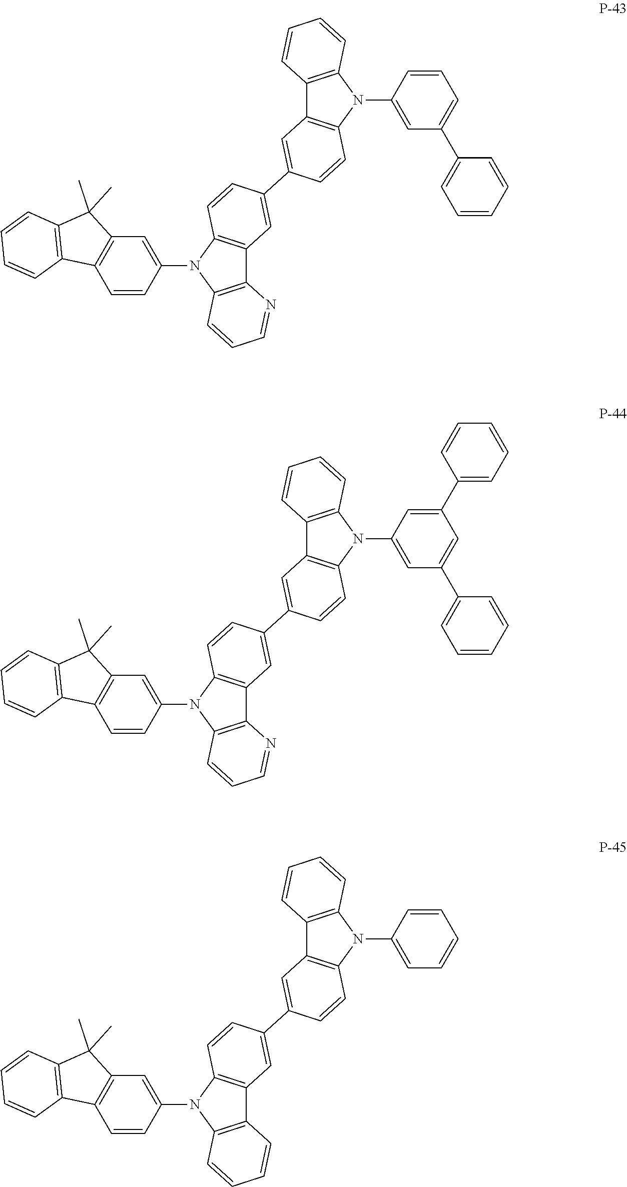 [SMILES: CC1(C)c2ccccc2-c2ccc(-n3c4ccc(-c5ccc6c(c5)c5ccccc5n6-c5cccc(-c6ccccc6)c5)cc4c4ncccc43)cc21 ; CC1(C)c2ccccc2-c2ccc(-n3c4ccc(-c5ccc6c(c5)c5ccccc5n6-c5cc(-c6ccccc6)cc(-c6ccccc6)c5)cc4c4ncccc43)cc21 ; CC1(C)c2ccccc2-c2ccc(-n3c4ccccc4c4cc(-c5ccc6c(c5)c5ccccc5n6-c5ccccc5)ccc43)cc21]

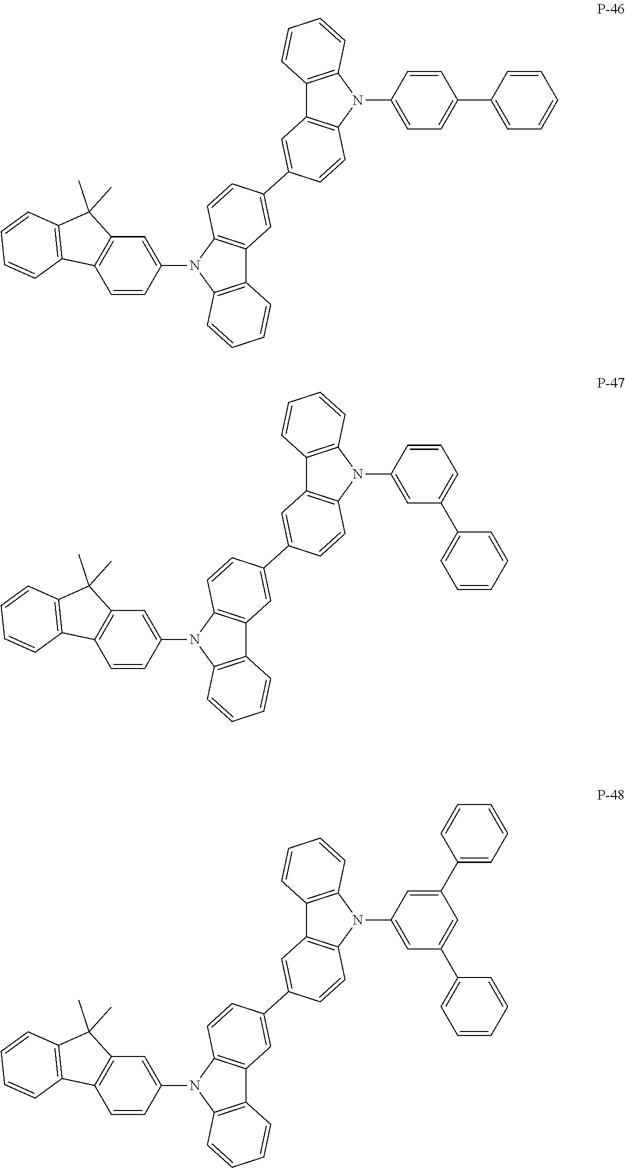 [SMILES: CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cccc(-c6ccccc6)c5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21]

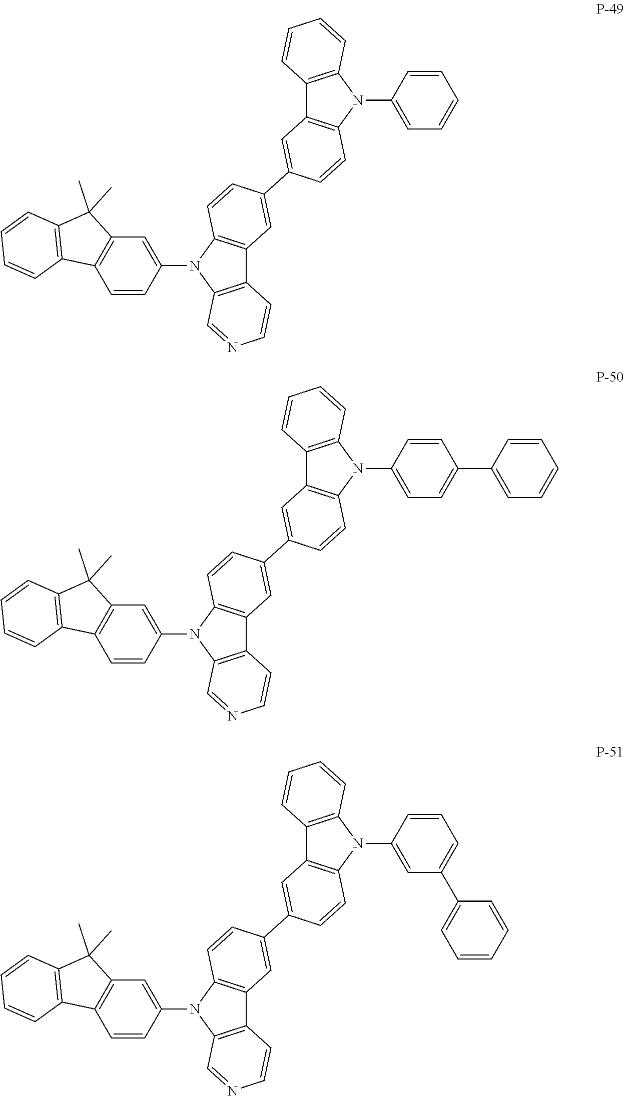 [SMILES: CC1(C)c2ccccc2-c2ccc(-n3c4ccc(-c5ccc6c(c5)c5ccccc5n6-c5ccccc5)cc4c4ccncc43)cc21 ; CC1(C)c2ccccc2-c2ccc(-n3c4ccc(-c5ccc6c(c5)c5ccccc5n6-c5ccc(-c6ccccc6)cc5)cc4c4ccncc43)cc21 ; CC1(C)c2ccccc2-c2ccc(-n3c4ccc(-c5ccc6c(c5)c5ccccc5n6-c5cccc(-c6ccccc6)c5)cc4c4ccncc43)cc21]

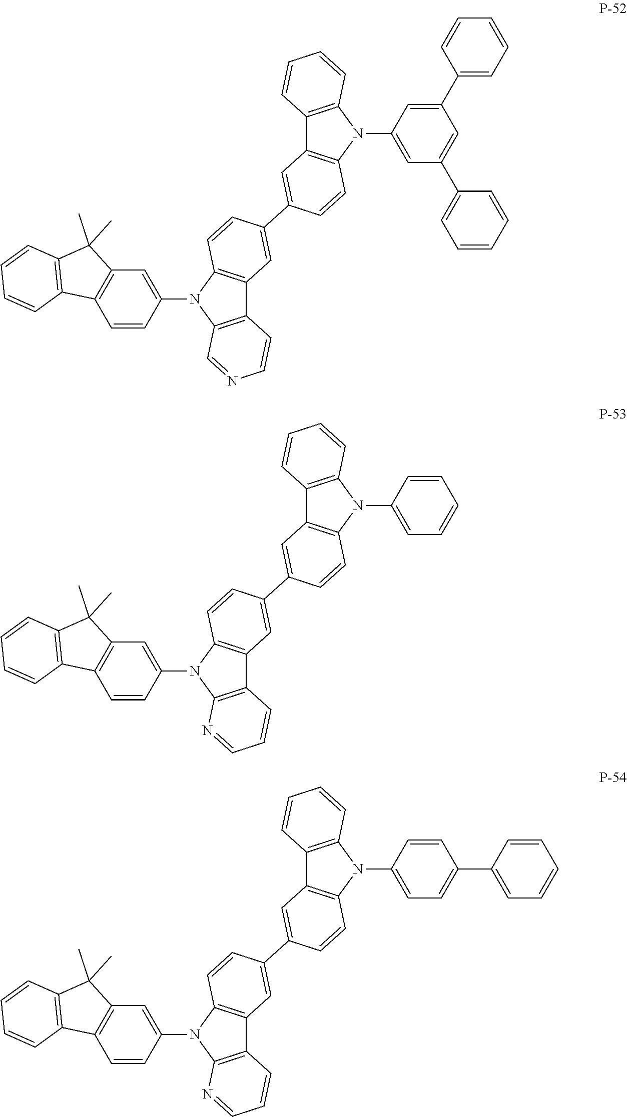 [SMILES: CC1(C)c2ccccc2-c2ccc(-n3c4ccc(-c5ccc6c(c5)c5ccccc5n6-c5cc(-c6ccccc6)cc(-c6ccccc6)c5)cc4c4ccncc43)cc21 ; CC1(C)c2ccccc2-c2ccc(-n3c4ccc(-c5ccc6c(c5)c5ccccc5n6-c5ccccc5)cc4c4cccnc43)cc21 ; CC1(C)c2ccccc2-c2ccc(-n3c4ccc(-c5ccc6c(c5)c5ccccc5n6-c5ccc(-c6ccccc6)cc5)cc4c4cccnc43)cc21]

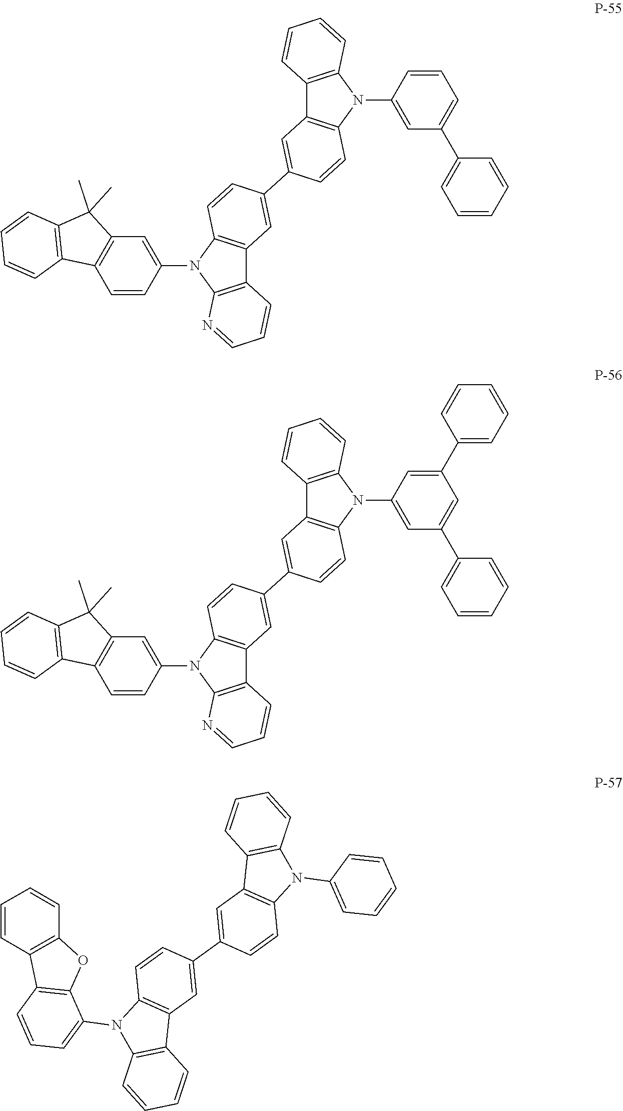 [SMILES: CC1(C)c2ccccc2-c2ccc(-n3c4ccc(-c5ccc6c(c5)c5ccccc5n6-c5cccc(-c6ccccc6)c5)cc4c4cccnc43)cc21 ; CC1(C)c2ccccc2-c2ccc(-n3c4ccc(-c5ccc6c(c5)c5ccccc5n6-c5cc(-c6ccccc6)cc(-c6ccccc6)c5)cc4c4cccnc43)cc21 ; c1ccc(-n2c3ccccc3c3cc(-c4ccc5c(c4)c4ccccc4n5-c4cccc5c4oc4ccccc45)ccc32)cc1]

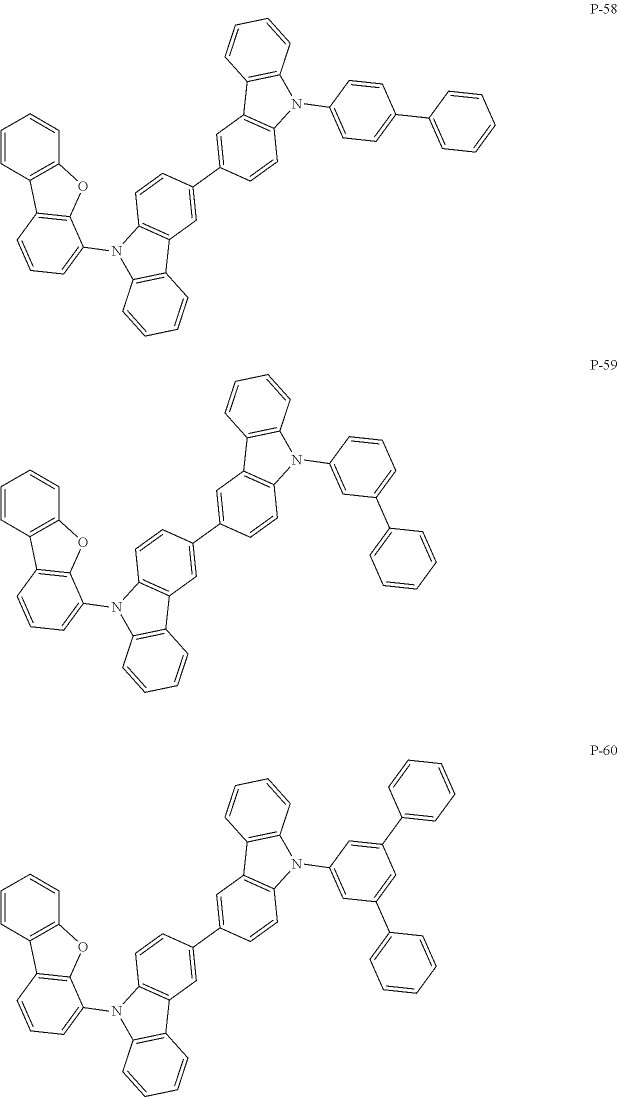 [SMILES: c1ccc(-c2ccc(-n3c4ccccc4c4cc(-c5ccc6c(c5)c5ccccc5n6-c5cccc6c5oc5ccccc56)ccc43)cc2)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1]

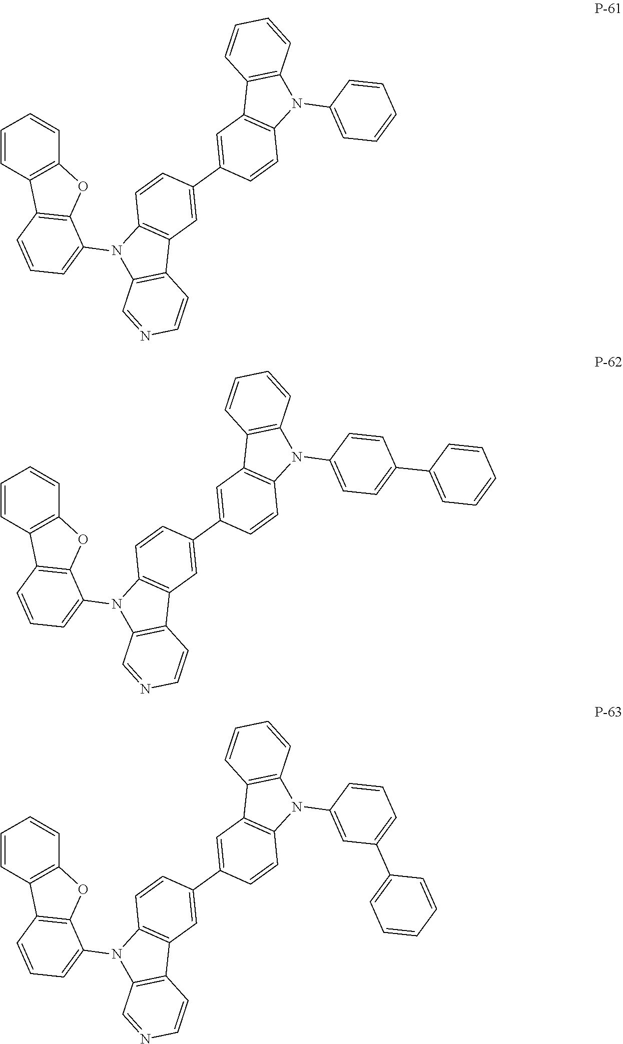 [SMILES: c1ccc(-n2c3ccccc3c3cc(-c4ccc5c(c4)c4ccncc4n5-c4cccc5c4oc4ccccc45)ccc32)cc1 ; c1ccc(-c2ccc(-n3c4ccccc4c4cc(-c5ccc6c(c5)c5ccncc5n6-c5cccc6c5oc5ccccc56)ccc43)cc2)cc1 ; c1ccc(-c2cccc(-n3c4ccccc4c4cc(-c5ccc6c(c5)c5ccncc5n6-c5cccc6c5oc5ccccc56)ccc43)c2)cc1]

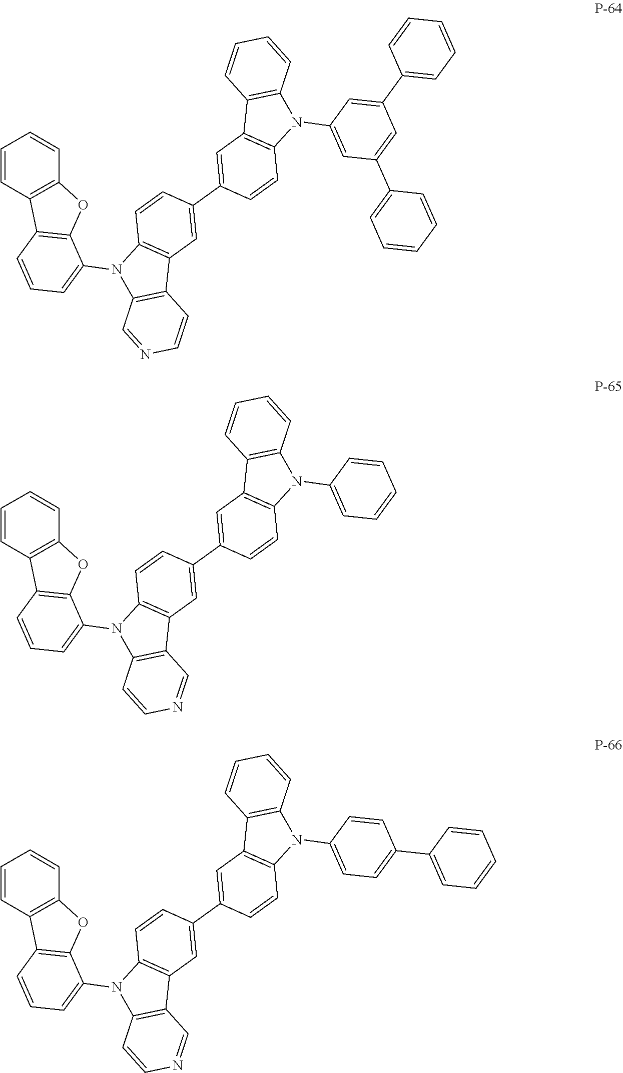 [SMILES: c1ccc(-c2cc(-c3ccccc3)cc(-n3c4ccccc4c4cc(-c5ccc6c(c5)c5ccncc5n6-c5cccc6c5oc5ccccc56)ccc43)c2)cc1 ; c1ccc(-n2c3ccccc3c3cc(-c4ccc5c(c4)c4cnccc4n5-c4cccc5c4oc4ccccc45)ccc32)cc1 ; c1ccc(-c2ccc(-n3c4ccccc4c4cc(-c5ccc6c(c5)c5cnccc5n6-c5cccc6c5oc5ccccc56)ccc43)cc2)cc1]

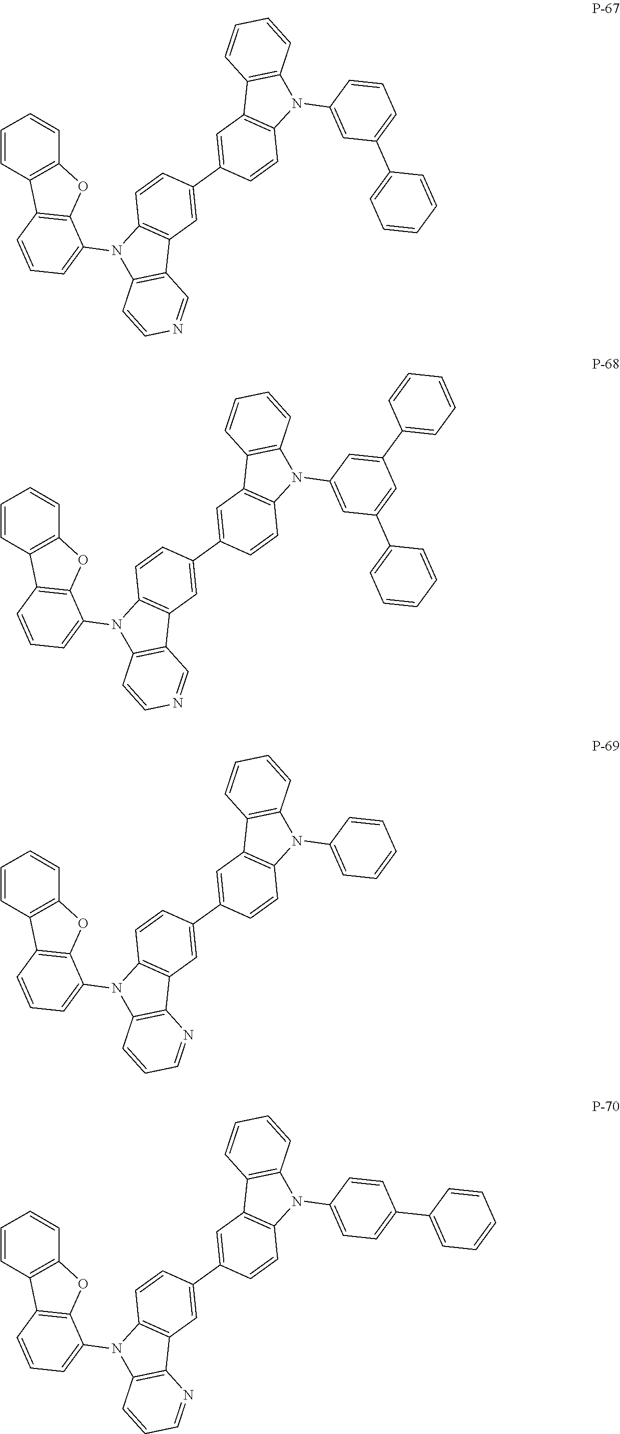 [SMILES: c1ccc(-c2cccc(-n3c4ccccc4c4cc(-c5ccc6c(c5)c5cnccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5cnccc5n6-c5cccc6c5oc5ccccc56)ccc43)c2)cc1 ; c1ccc(-n2c3ccccc3c3cc(-c4ccc5c(c4)c4ncccc4n5-c4cccc5c4oc4ccccc45)ccc32)cc1 ; c1ccc(-c2ccc(-n3c4ccccc4c4cc(-c5ccc6c(c5)c5ncccc5n6-c5cccc6c5oc5ccccc56)ccc43)cc2)cc1]

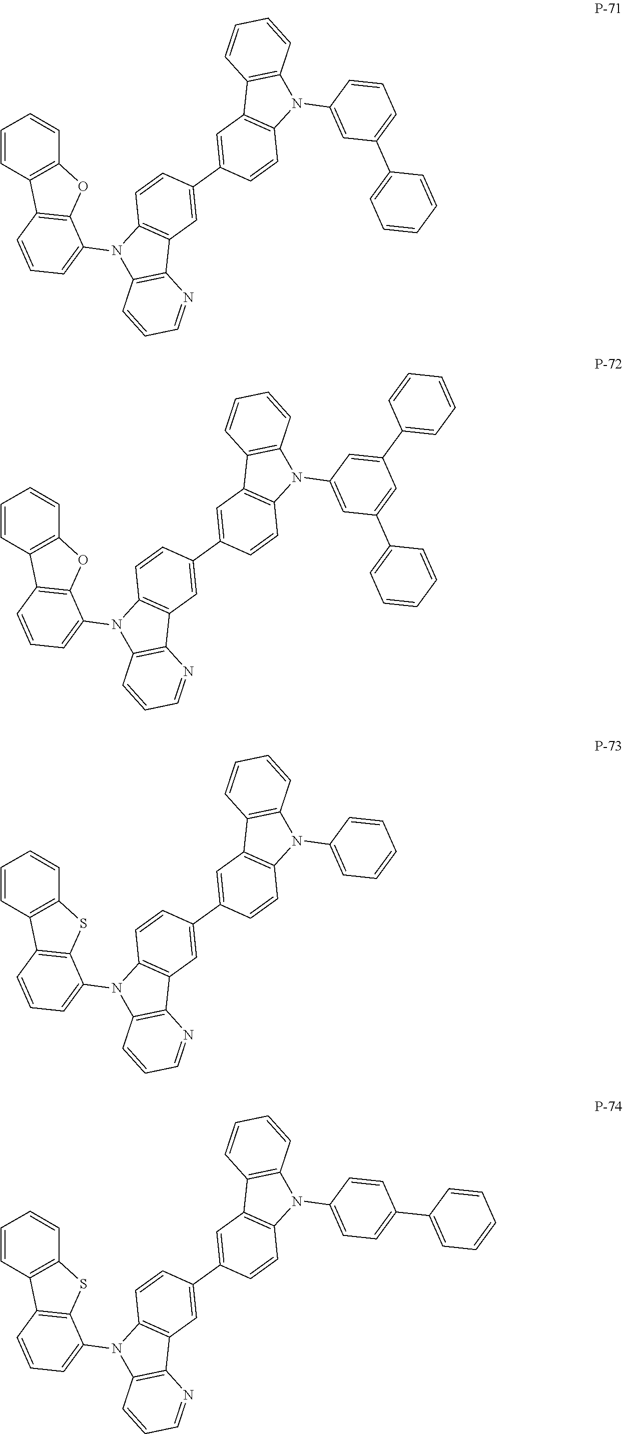 [SMILES: c1ccc(-c2cccc(-n3c4ccccc4c4cc(-c5ccc6c(c5)c5ncccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ncccc5n6-c5cccc6c5oc5ccccc56)ccc43)c2)cc1 ; c1ccc(-n2c3ccccc3c3cc(-c4ccc5c(c4)c4ncccc4n5-c4cccc5c4sc4ccccc45)ccc32)cc1 ; c1ccc(-c2ccc(-n3c4ccccc4c4cc(-c5ccc6c(c5)c5ncccc5n6-c5cccc6c5sc5ccccc56)ccc43)cc2)cc1]

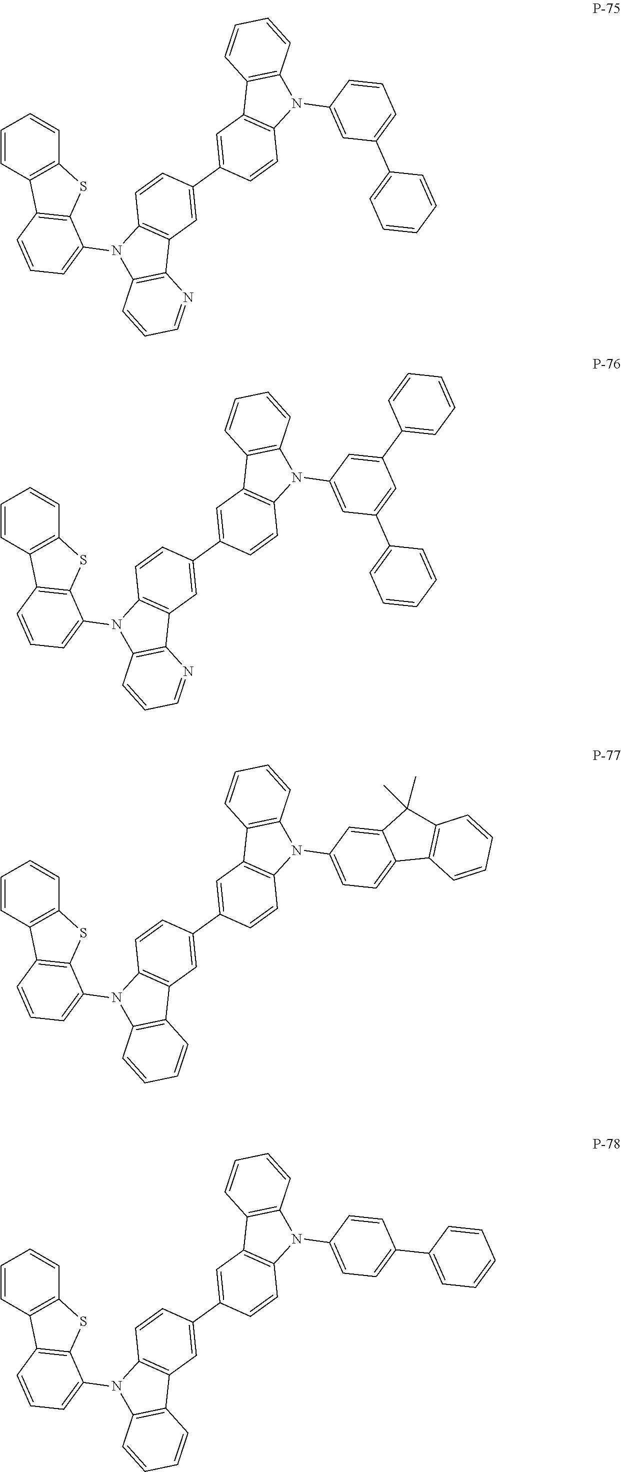 [SMILES: c1ccc(-c2cccc(-n3c4ccccc4c4cc(-c5ccc6c(c5)c5ncccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ncccc5n6-c5cccc6c5sc5ccccc56)ccc43)c2)cc1 ; CC1(C)c2ccccc2-c2ccc(-n3c4ccccc4c4cc(-c5ccc6c(c5)c5ccccc5n6-c5cccc6c5sc5ccccc56)ccc43)cc21 ; c1ccc(-c2ccc(-n3c4ccccc4c4cc(-c5ccc6c(c5)c5ccccc5n6-c5cccc6c5sc5ccccc56)ccc43)cc2)cc1]

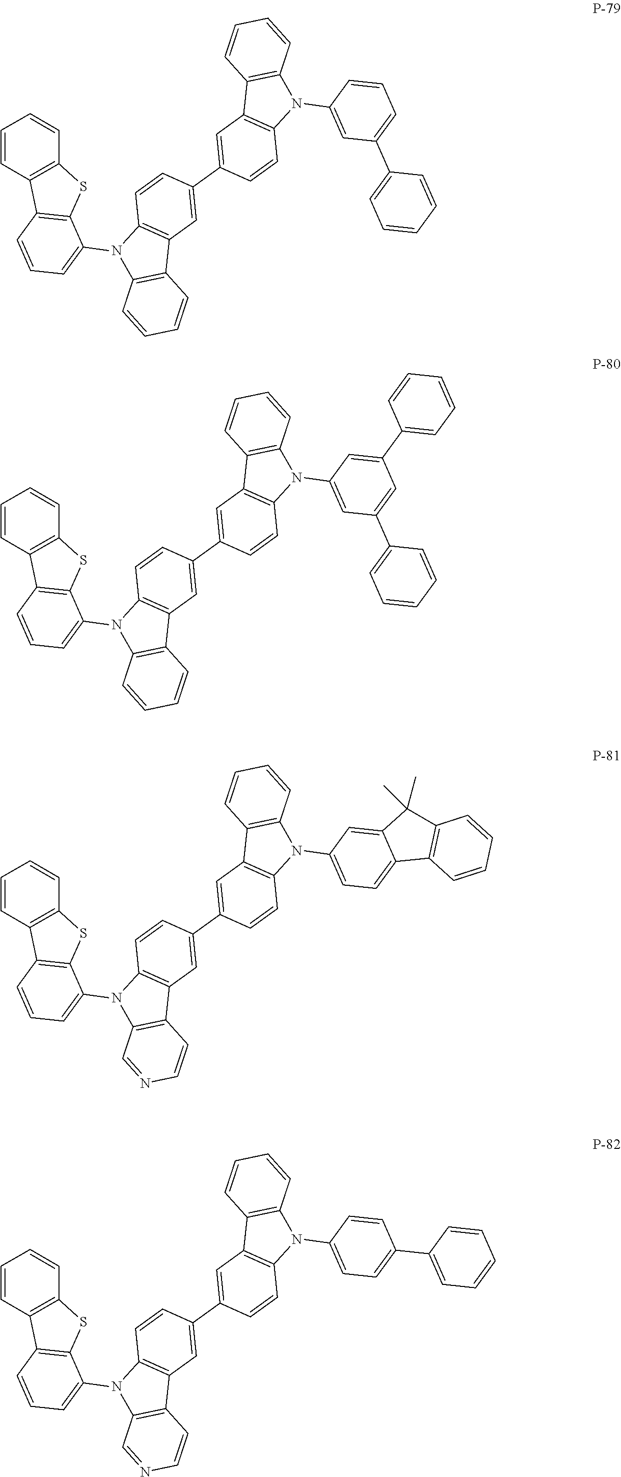 [SMILES: c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1 ; CC1(C)c2ccccc2-c2ccc(-n3c4ccccc4c4cc(-c5ccc6c(c5)c5ccncc5n6-c5cccc6c5sc5ccccc56)ccc43)cc21 ; c1ccc(-c2ccc(-n3c4ccccc4c4cc(-c5ccc6c(c5)c5ccncc5n6-c5cccc6c5sc5ccccc56)ccc43)cc2)cc1]

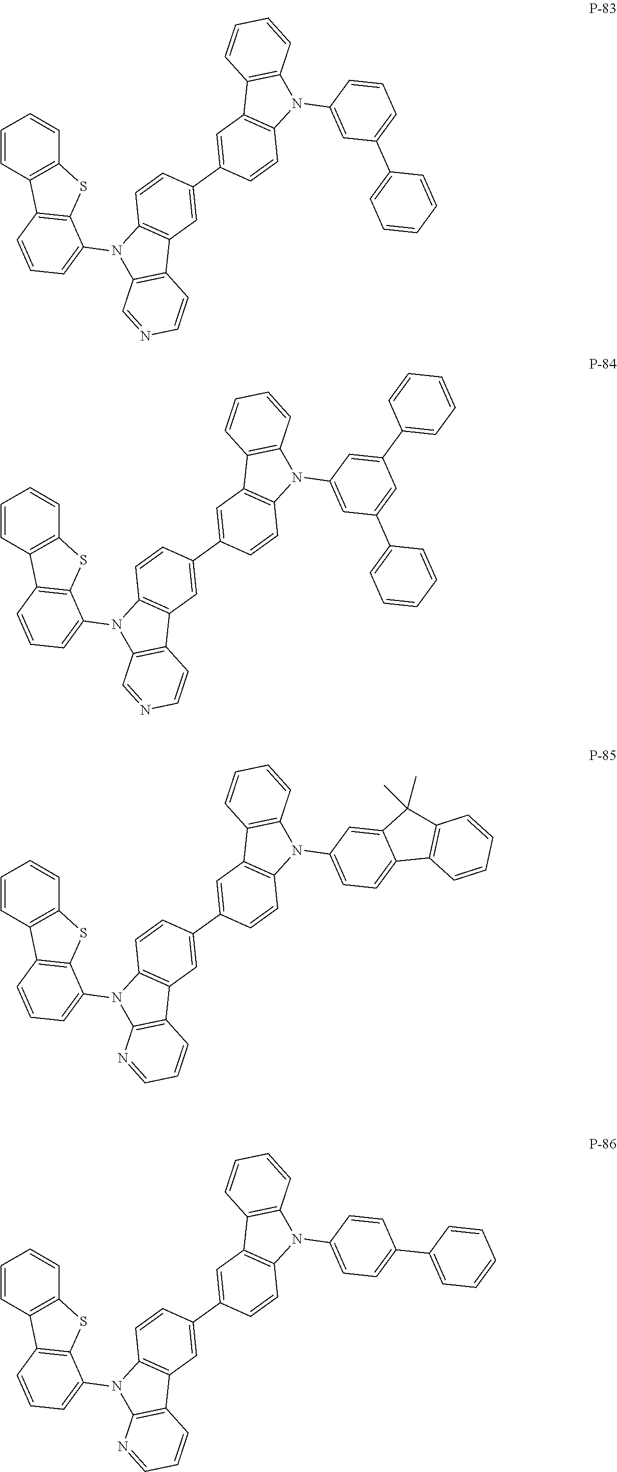 [SMILES: c1ccc(-c2cccc(-n3c4ccccc4c4cc(-c5ccc6c(c5)c5ccncc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccncc5n6-c5cccc6c5sc5ccccc56)ccc43)c2)cc1 ; CC1(C)c2ccccc2-c2ccc(-n3c4ccccc4c4cc(-c5ccc6c(c5)c5cccnc5n6-c5cccc6c5sc5ccccc56)ccc43)cc21 ; c1ccc(-c2ccc(-n3c4ccccc4c4cc(-c5ccc6c(c5)c5cccnc5n6-c5cccc6c5sc5ccccc56)ccc43)cc2)cc1]

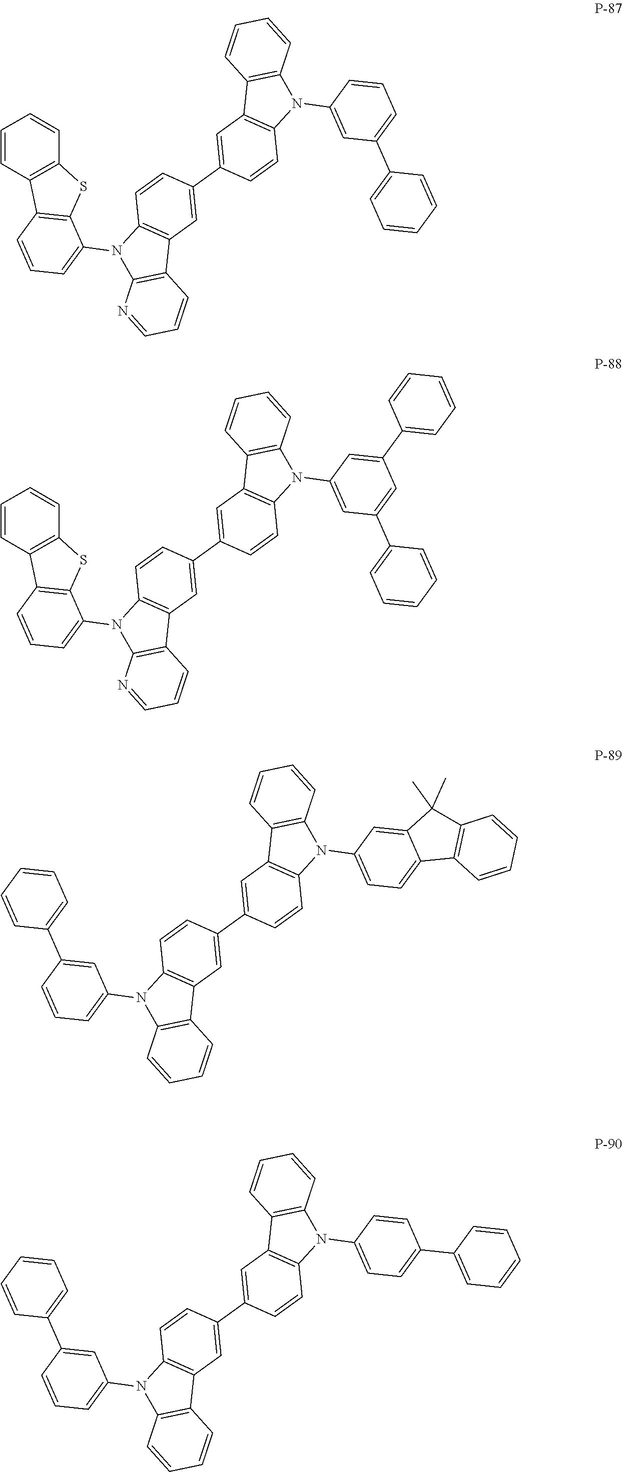 [SMILES: c1ccc(-c2cccc(-n3c4ccccc4c4cc(-c5ccc6c(c5)c5cccnc5n6-c5cccc6c5sc5ccccc56)ccc43)c2)cc1 ; c1ccc(-c2cc(-c3ccccc3)cc(-n3c4ccccc4c4cc(-c5ccc6c(c5)c5cccnc5n6-c5cccc6c5sc5ccccc56)ccc43)c2)cc1 ; CC1(C)c2ccccc2-c2ccc(-n3c4ccccc4c4cc(-c5ccc6c(c5)c5ccccc5n6-c5cccc(-c6ccccc6)c5)ccc43)cc21 ; c1ccc(-c2ccc(-n3c4ccccc4c4cc(-c5ccc6c(c5)c5ccccc5n6-c5cccc(-c6ccccc6)c5)ccc43)cc2)cc1]

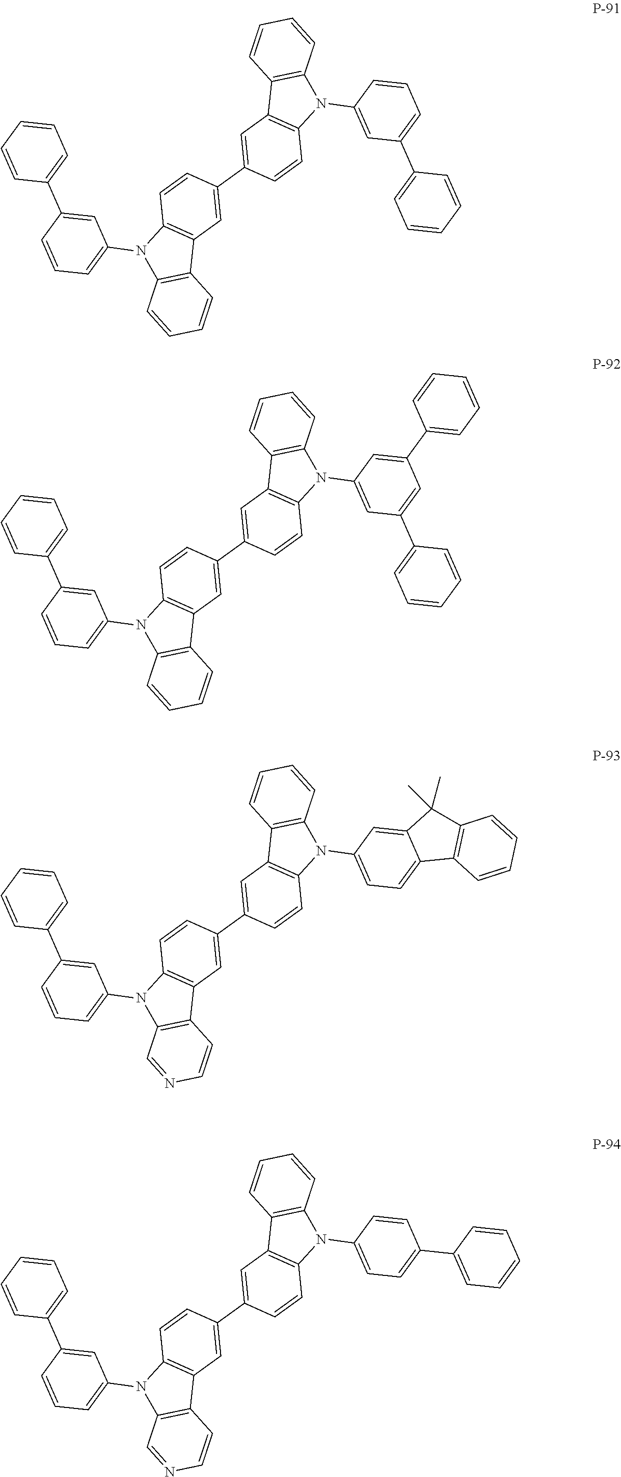 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2ccc(-n3c4ccccc4c4cc(-c5ccc6c(c5)c5ccncc5n6-c5cccc(-c6ccccc6)c5)ccc43)cc21 ; c1ccc(-c2ccc(-n3c4ccccc4c4cc(-c5ccc6c(c5)c5ccncc5n6-c5cccc(-c6ccccc6)c5)ccc43)cc2)cc1]

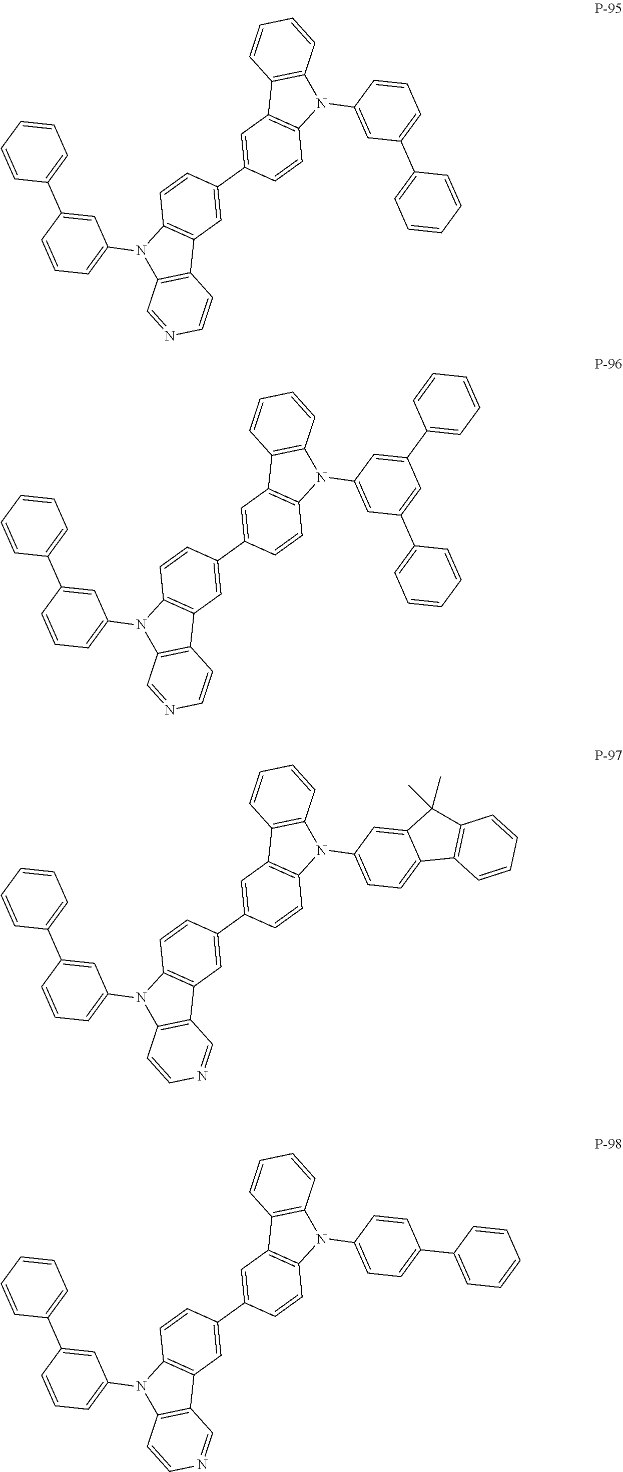 [SMILES: c1ccc(-c2cccc(-n3c4ccccc4c4cc(-c5ccc6c(c5)c5ccncc5n6-c5cccc(-c6ccccc6)c5)ccc43)c2)cc1 ; c1ccc(-c2cccc(-n3c4ccc(-c5ccc6c(c5)c5ccccc5n6-c5cc(-c6ccccc6)cc(-c6ccccc6)c5)cc4c4ccncc43)c2)cc1 ; CC1(C)c2ccccc2-c2ccc(-n3c4ccccc4c4cc(-c5ccc6c(c5)c5cnccc5n6-c5cccc(-c6ccccc6)c5)ccc43)cc21 ; c1ccc(-c2ccc(-n3c4ccccc4c4cc(-c5ccc6c(c5)c5cnccc5n6-c5cccc(-c6ccccc6)c5)ccc43)cc2)cc1]

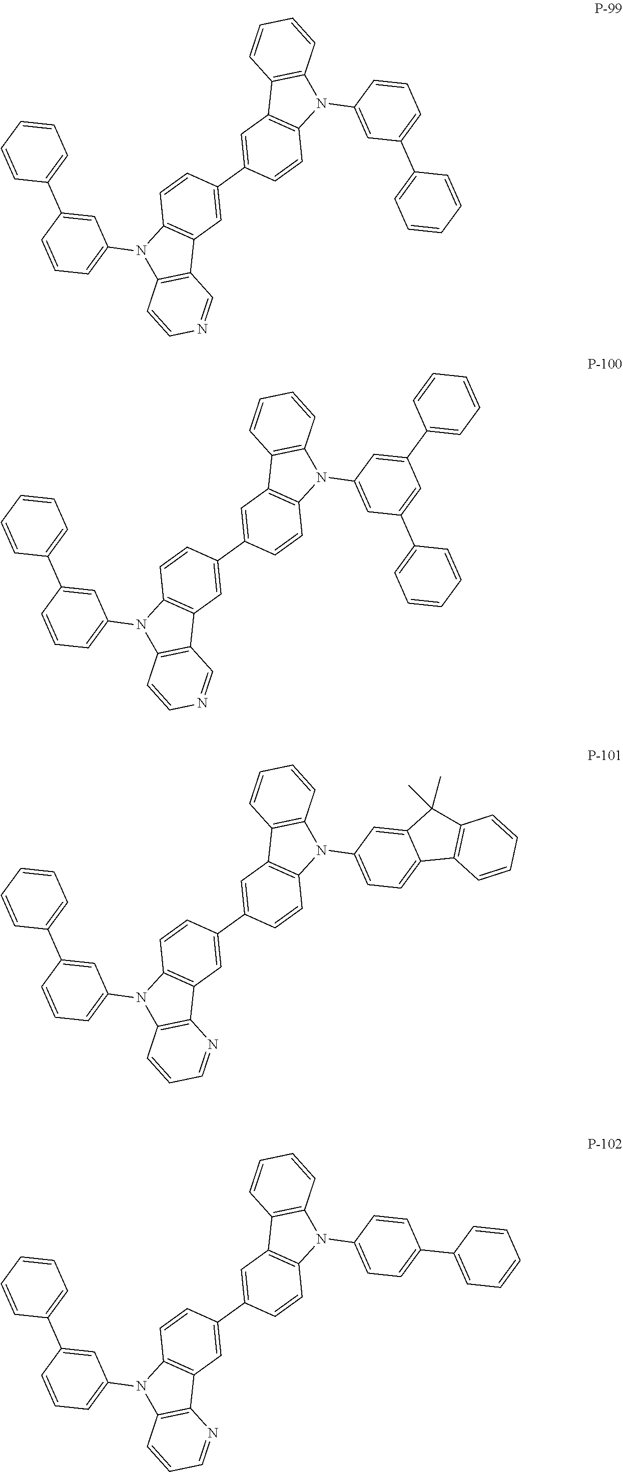 [SMILES: c1ccc(-c2cccc(-n3c4ccccc4c4cc(-c5ccc6c(c5)c5cnccc5n6-c5cccc(-c6ccccc6)c5)ccc43)c2)cc1 ; c1ccc(-c2cccc(-n3c4ccncc4c4cc(-c5ccc6c(c5)c5ccccc5n6-c5cc(-c6ccccc6)cc(-c6ccccc6)c5)ccc43)c2)cc1 ; CC1(C)c2ccccc2-c2ccc(-n3c4ccccc4c4cc(-c5ccc6c(c5)c5ncccc5n6-c5cccc(-c6ccccc6)c5)ccc43)cc21 ; c1ccc(-c2ccc(-n3c4ccccc4c4cc(-c5ccc6c(c5)c5ncccc5n6-c5cccc(-c6ccccc6)c5)ccc43)cc2)cc1]

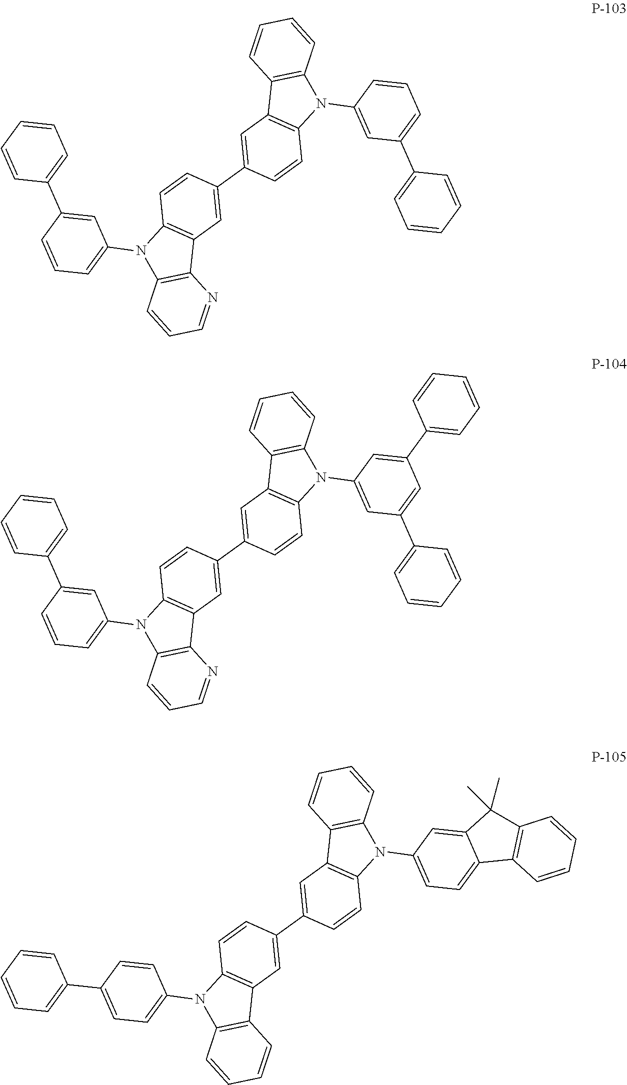 [SMILES: c1ccc(-c2cccc(-n3c4ccccc4c4cc(-c5ccc6c(c5)c5ncccc5n6-c5cccc(-c6ccccc6)c5)ccc43)c2)cc1 ; c1ccc(-c2cc(-c3ccccc3)cc(-n3c4ccccc4c4cc(-c5ccc6c(c5)c5ncccc5n6-c5cccc(-c6ccccc6)c5)ccc43)c2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21]

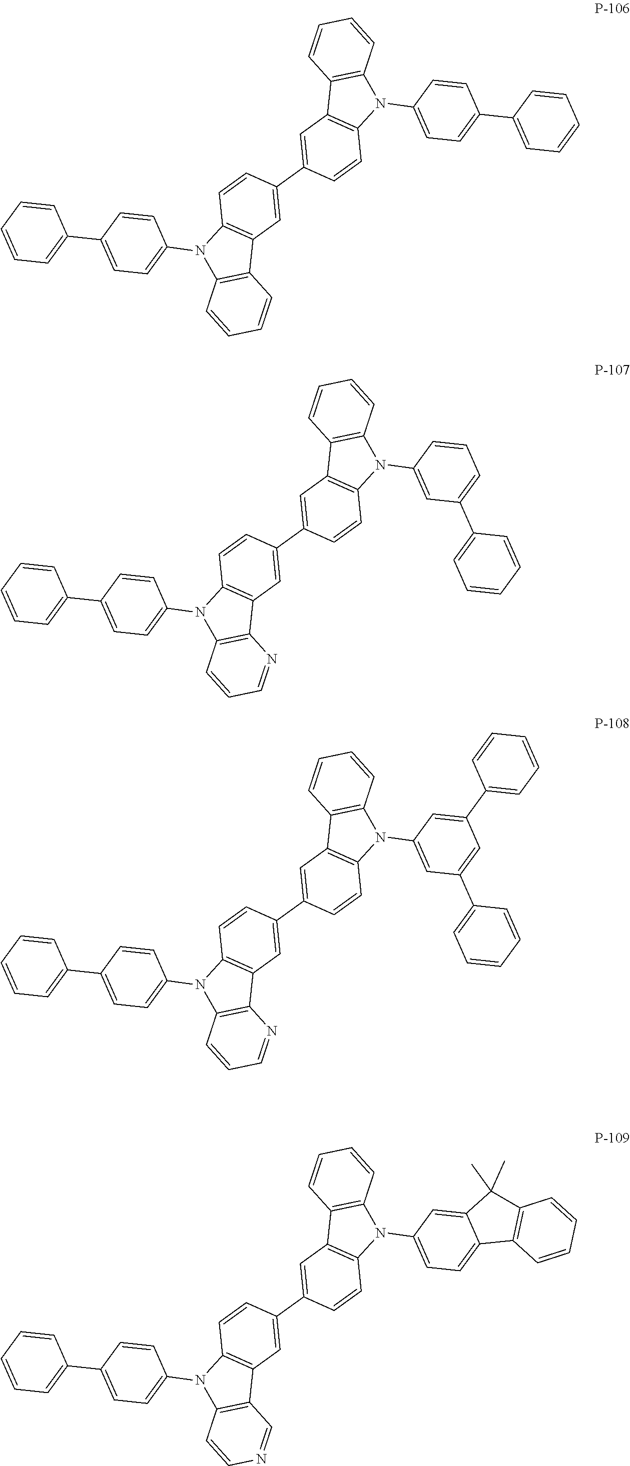 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccc(-c5ccc6c(c5)c5ccccc5n6-c5cccc(-c6ccccc6)c5)cc4c4ncccc43)cc2)cc1 ; c1ccc(-c2ccc(-n3c4ccc(-c5ccc6c(c5)c5ccccc5n6-c5cc(-c6ccccc6)cc(-c6ccccc6)c5)cc4c4ncccc43)cc2)cc1 ; CC1(C)c2ccccc2-c2ccc(-n3c4ccccc4c4cc(-c5ccc6c(c5)c5cnccc5n6-c5ccc(-c6ccccc6)cc5)ccc43)cc21]

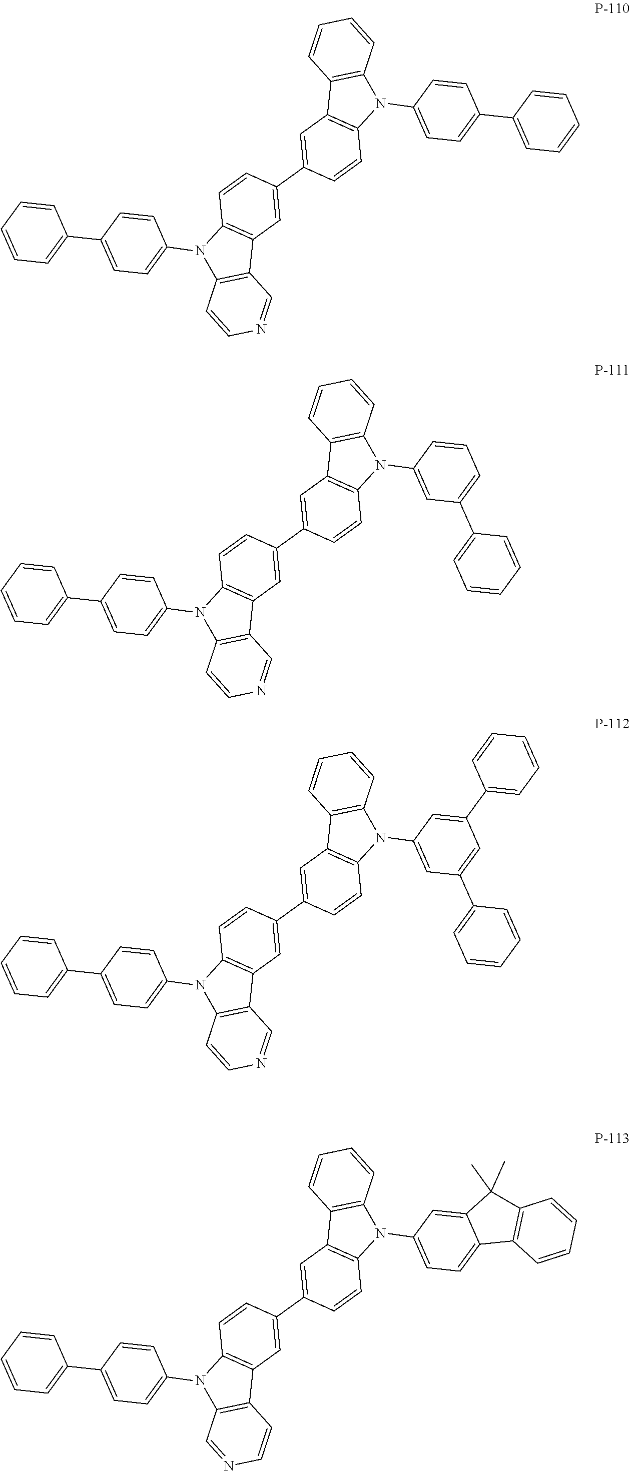 [SMILES: c1ccc(-c2ccc(-n3c4ccccc4c4cc(-c5ccc6c(c5)c5cnccc5n6-c5ccc(-c6ccccc6)cc5)ccc43)cc2)cc1 ; c1ccc(-c2ccc(-n3c4ccncc4c4cc(-c5ccc6c(c5)c5ccccc5n6-c5cccc(-c6ccccc6)c5)ccc43)cc2)cc1 ; c1ccc(-c2ccc(-n3c4ccncc4c4cc(-c5ccc6c(c5)c5ccccc5n6-c5cc(-c6ccccc6)cc(-c6ccccc6)c5)ccc43)cc2)cc1 ; CC1(C)c2ccccc2-c2ccc(-n3c4ccccc4c4cc(-c5ccc6c(c5)c5ccncc5n6-c5ccc(-c6ccccc6)cc5)ccc43)cc21]

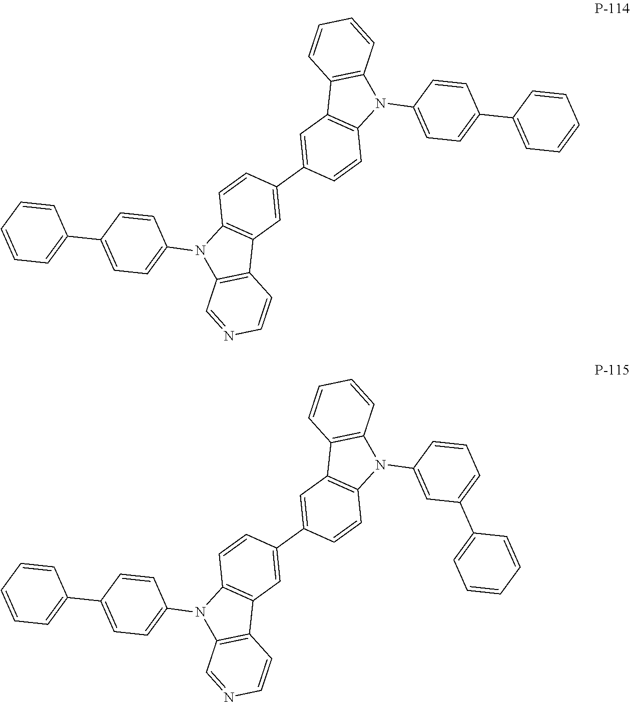 [SMILES: c1ccc(-c2ccc(-n3c4ccccc4c4cc(-c5ccc6c(c5)c5ccncc5n6-c5ccc(-c6ccccc6)cc5)ccc43)cc2)cc1 ; c1ccc(-c2ccc(-n3c4ccc(-c5ccc6c(c5)c5ccccc5n6-c5cccc(-c6ccccc6)c5)cc4c4ccncc43)cc2)cc1]

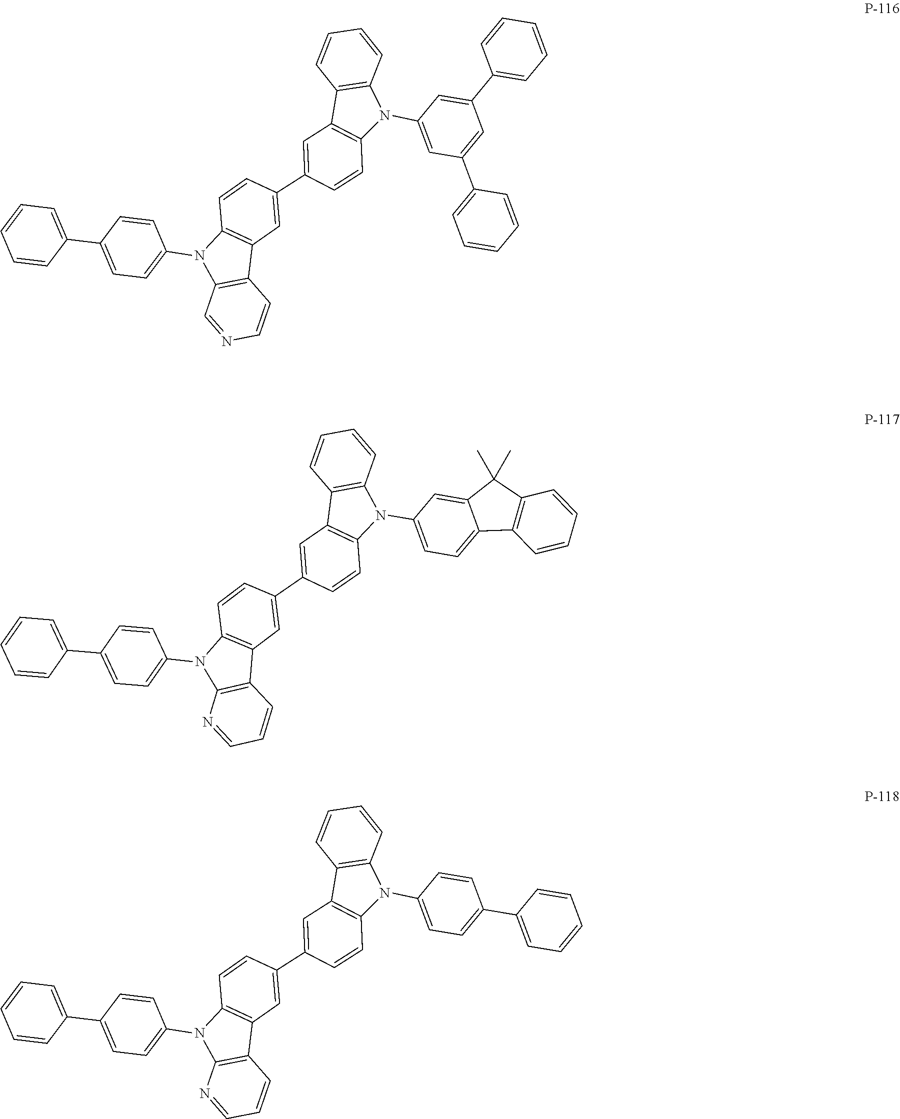 [SMILES: c1ccc(-c2ccc(-n3c4ccc(-c5ccc6c(c5)c5ccccc5n6-c5cc(-c6ccccc6)cc(-c6ccccc6)c5)cc4c4ccncc43)cc2)cc1 ; CC1(C)c2ccccc2-c2ccc(-n3c4ccccc4c4cc(-c5ccc6c(c5)c5cccnc5n6-c5ccc(-c6ccccc6)cc5)ccc43)cc21 ; c1ccc(-c2ccc(-n3c4ccccc4c4cc(-c5ccc6c(c5)c5cccnc5n6-c5ccc(-c6ccccc6)cc5)ccc43)cc2)cc1]

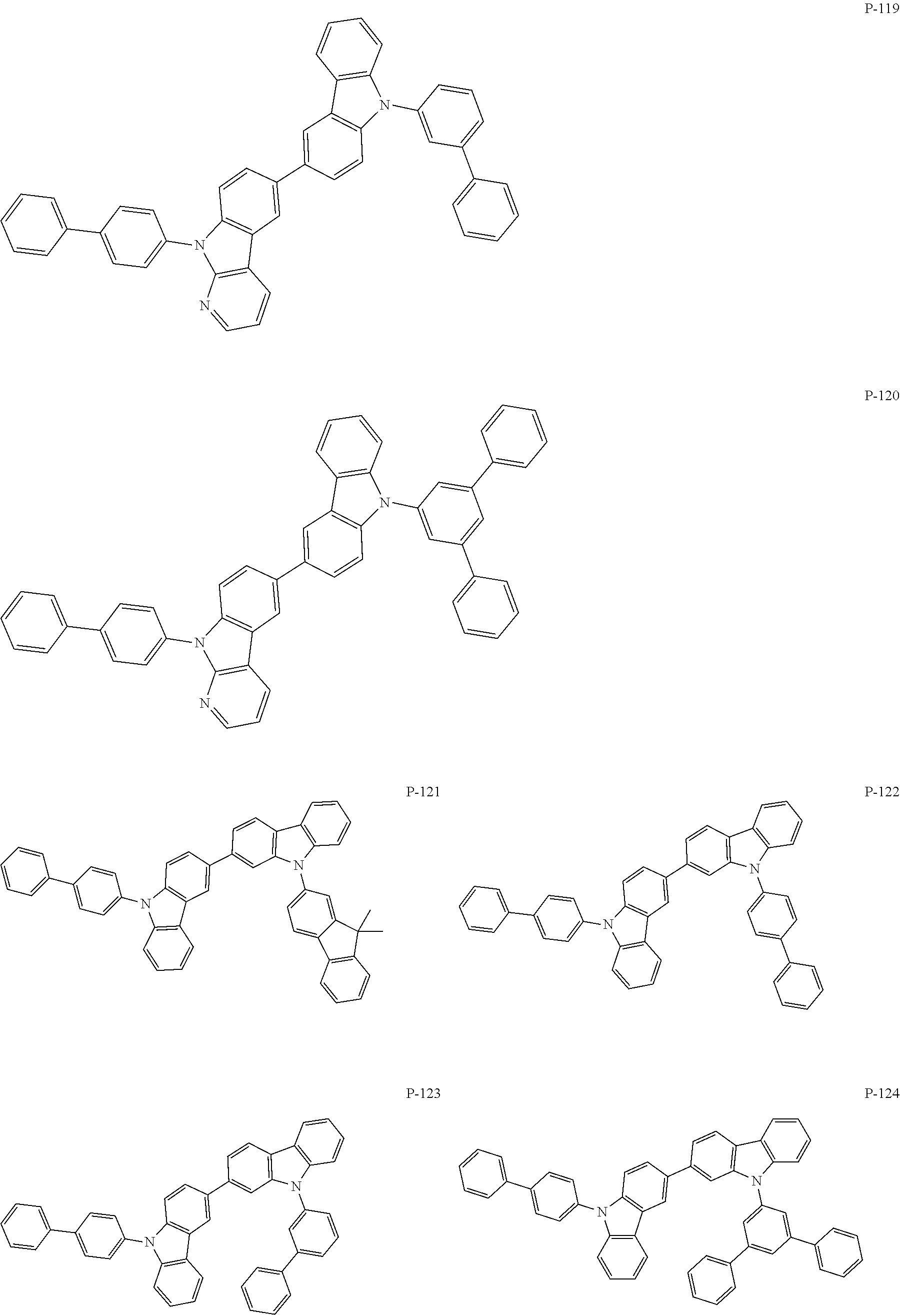

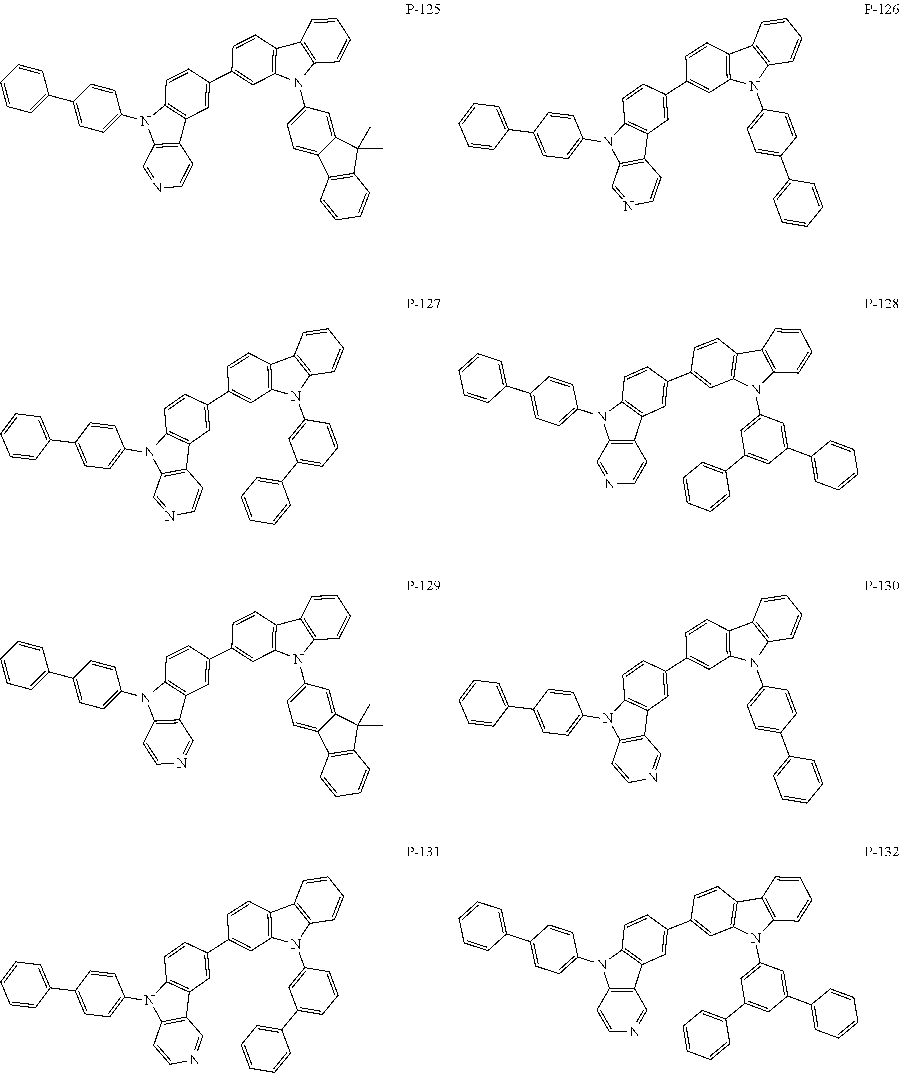

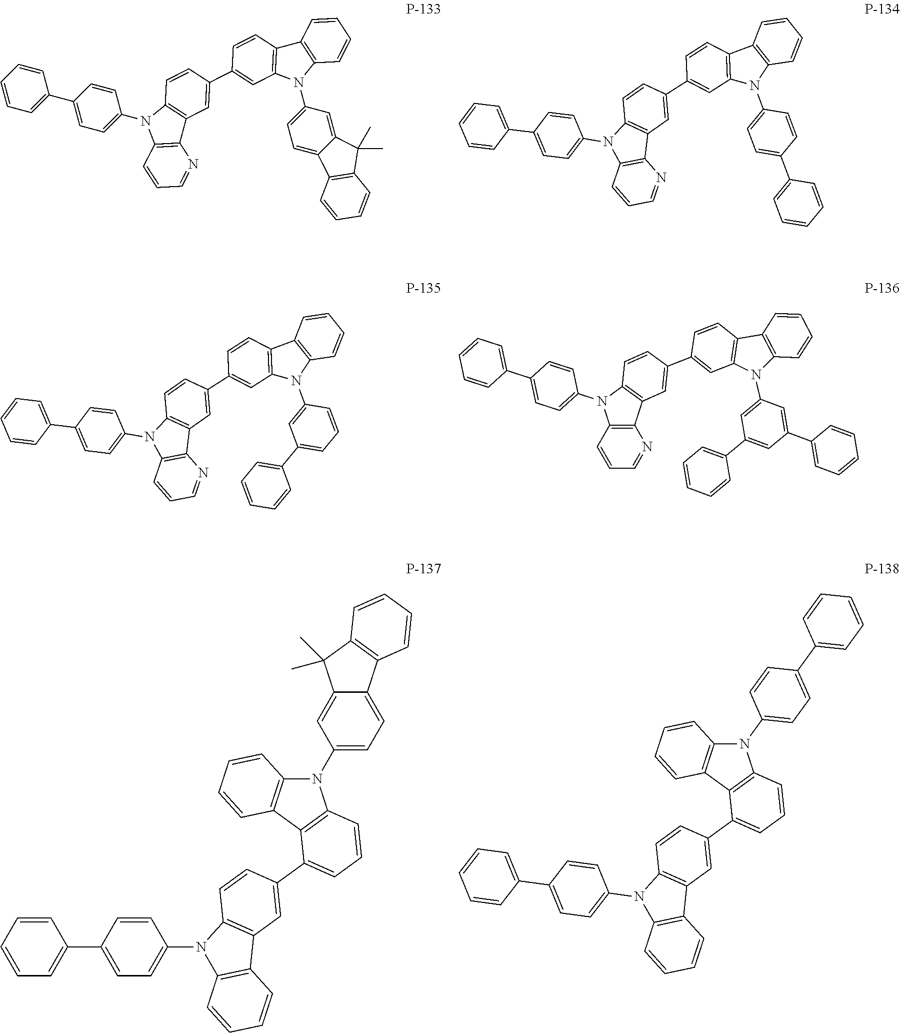

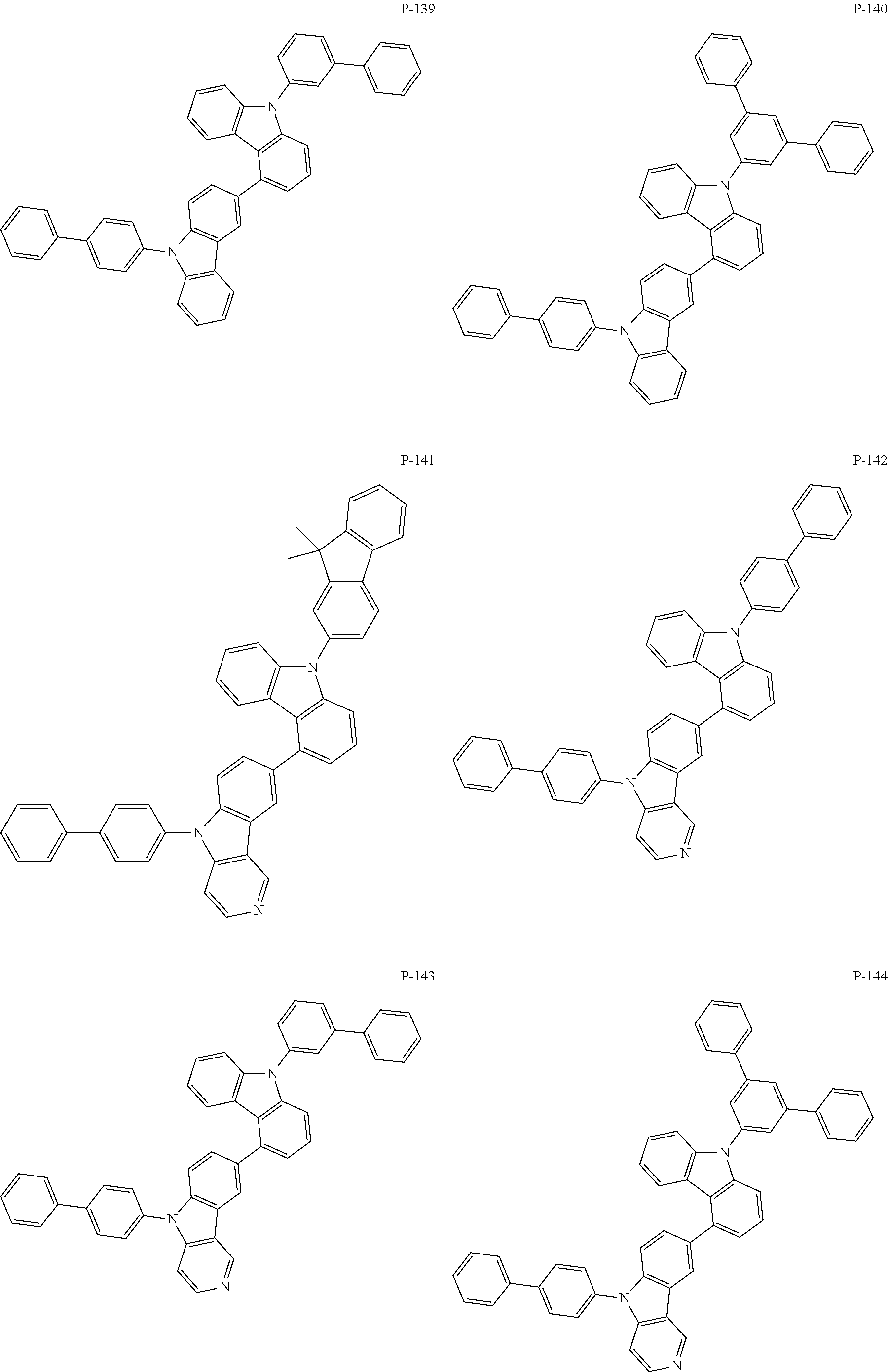

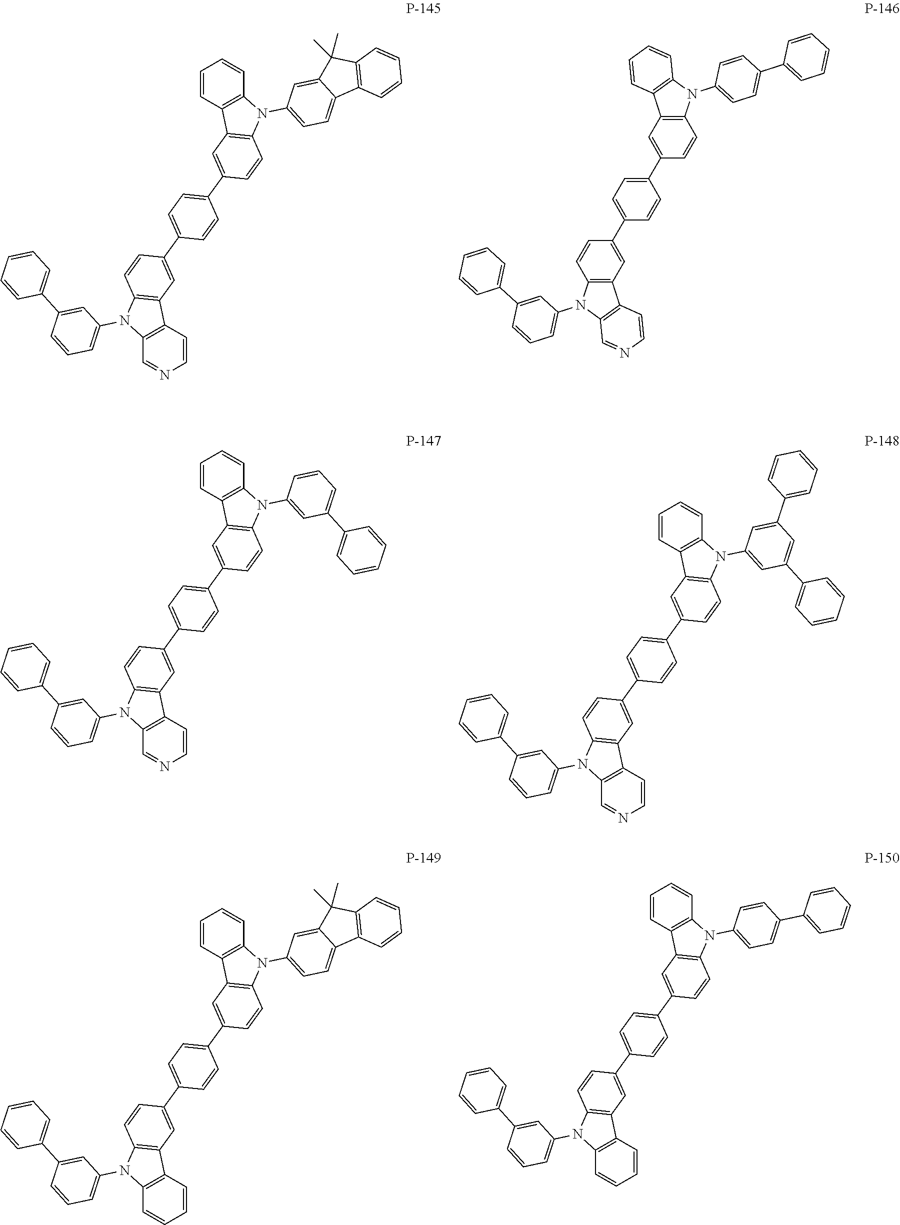

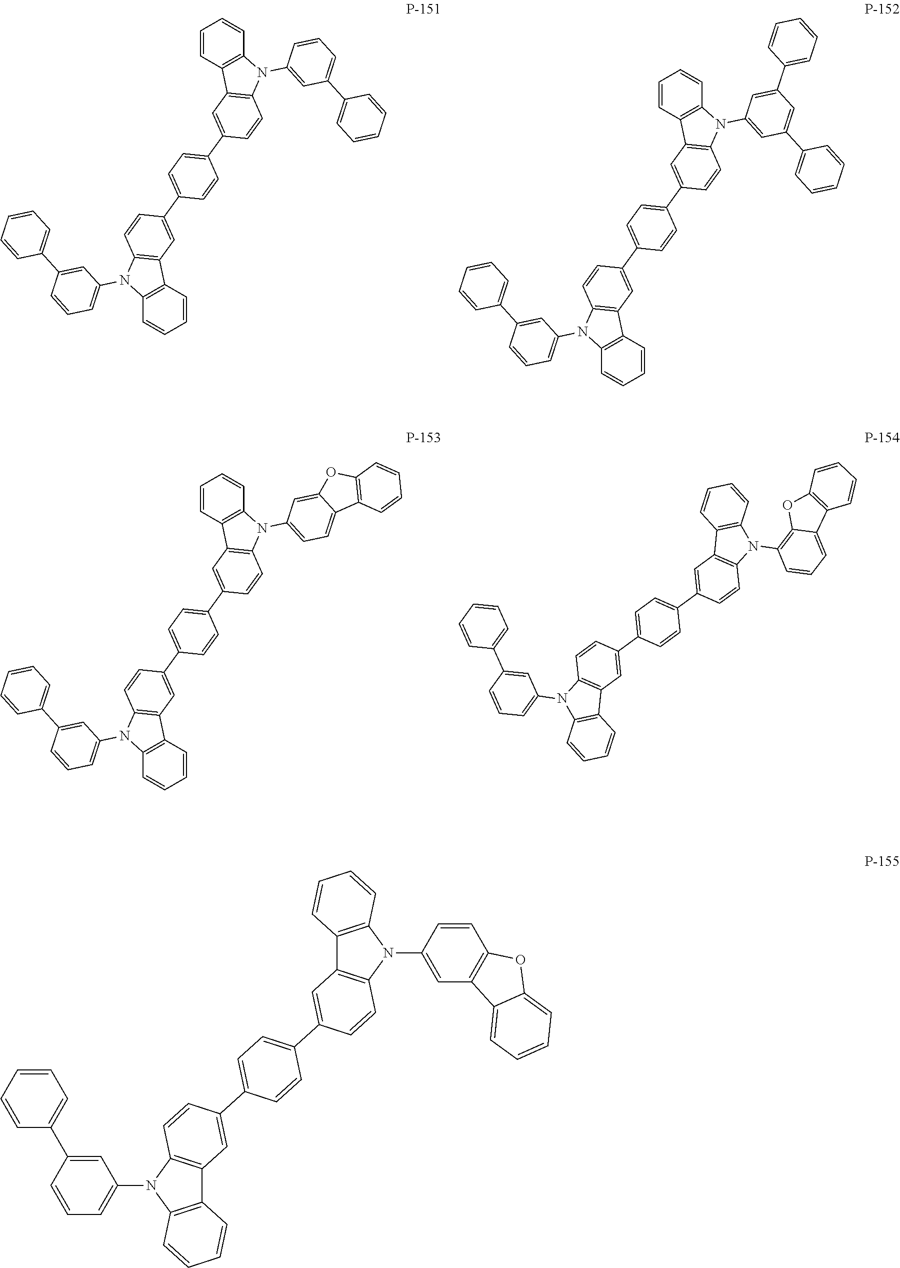

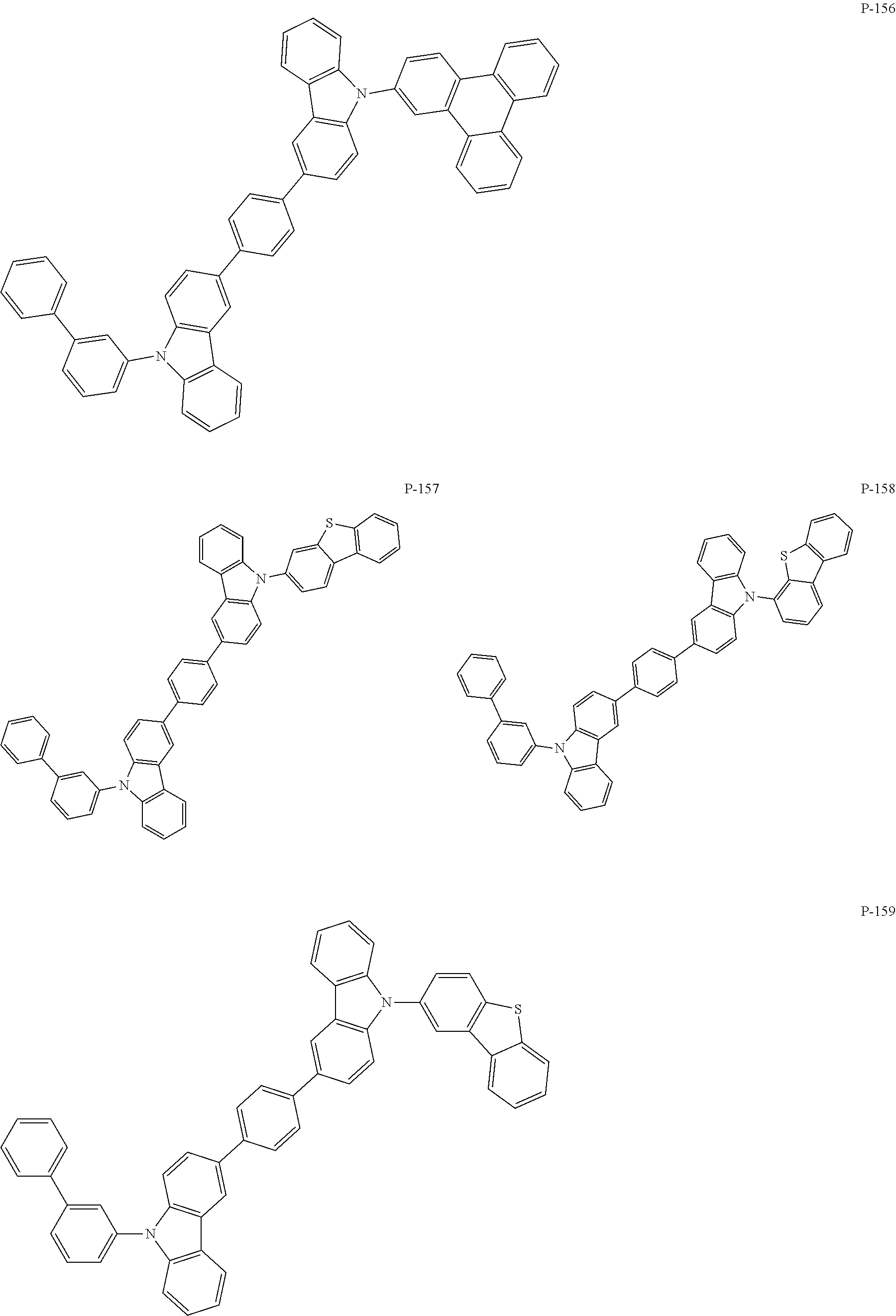 [SMILES: c1ccc(-c2cccc(-n3c4ccccc4c4cc(-c5ccc(-c6ccc7c(c6)c6ccccc6n7-c6ccc7c8ccccc8c8ccccc8c7c6)cc5)ccc43)c2)cc1 ; c1ccc(-c2cccc(-n3c4ccccc4c4cc(-c5ccc(-c6ccc7c(c6)c6ccccc6n7-c6ccc7c(c6)sc6ccccc67)cc5)ccc43)c2)cc1 ; c1ccc(-c2cccc(-n3c4ccccc4c4cc(-c5ccc(-c6ccc7c(c6)c6ccccc6n7-c6cccc7c6sc6ccccc67)cc5)ccc43)c2)cc1 ; c1ccc(-c2cccc(-n3c4ccccc4c4cc(-c5ccc(-c6ccc7c(c6)c6ccccc6n7-c6ccc7sc8ccccc8c7c6)cc5)ccc43)c2)cc1]

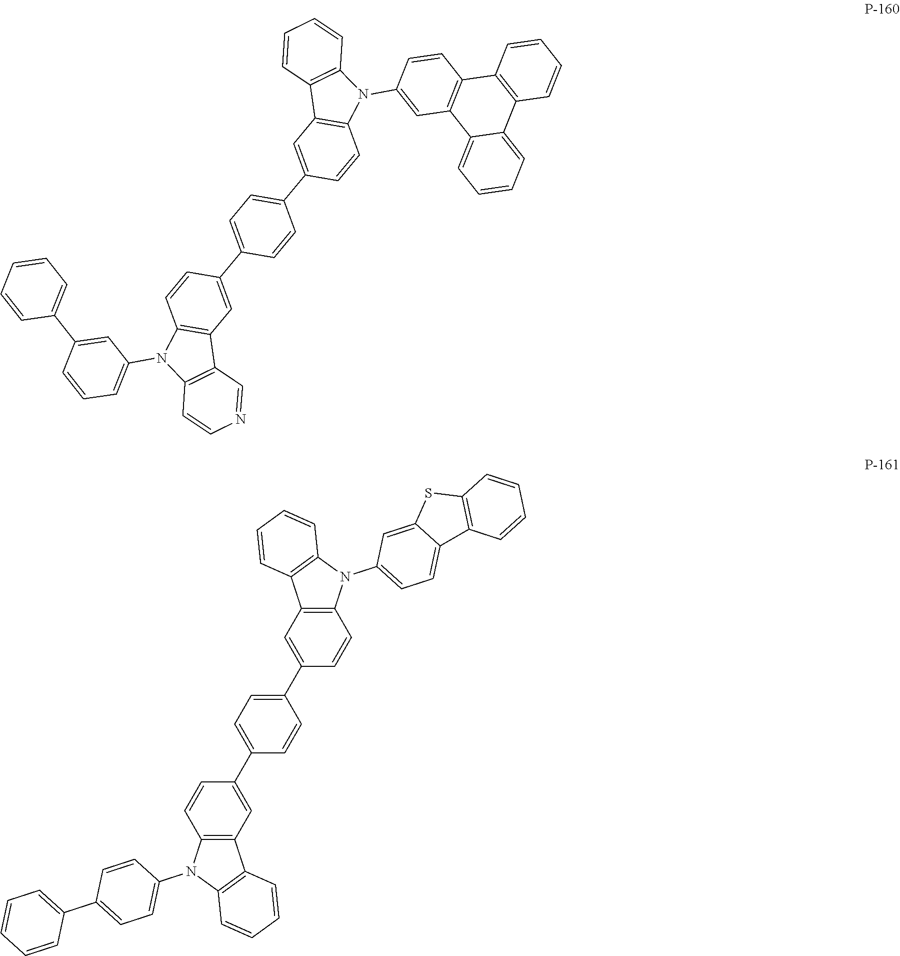 [SMILES: c1ccc(-c2cccc(-n3c4ccncc4c4cc(-c5ccc(-c6ccc7c(c6)c6ccccc6n7-c6ccc7c8ccccc8c8ccccc8c7c6)cc5)ccc43)c2)cc1 ; c1ccc(-c2ccc(-n3c4ccccc4c4cc(-c5ccc(-c6ccc7c(c6)c6ccccc6n7-c6ccc7c(c6)sc6ccccc67)cc5)ccc43)cc2)cc1]

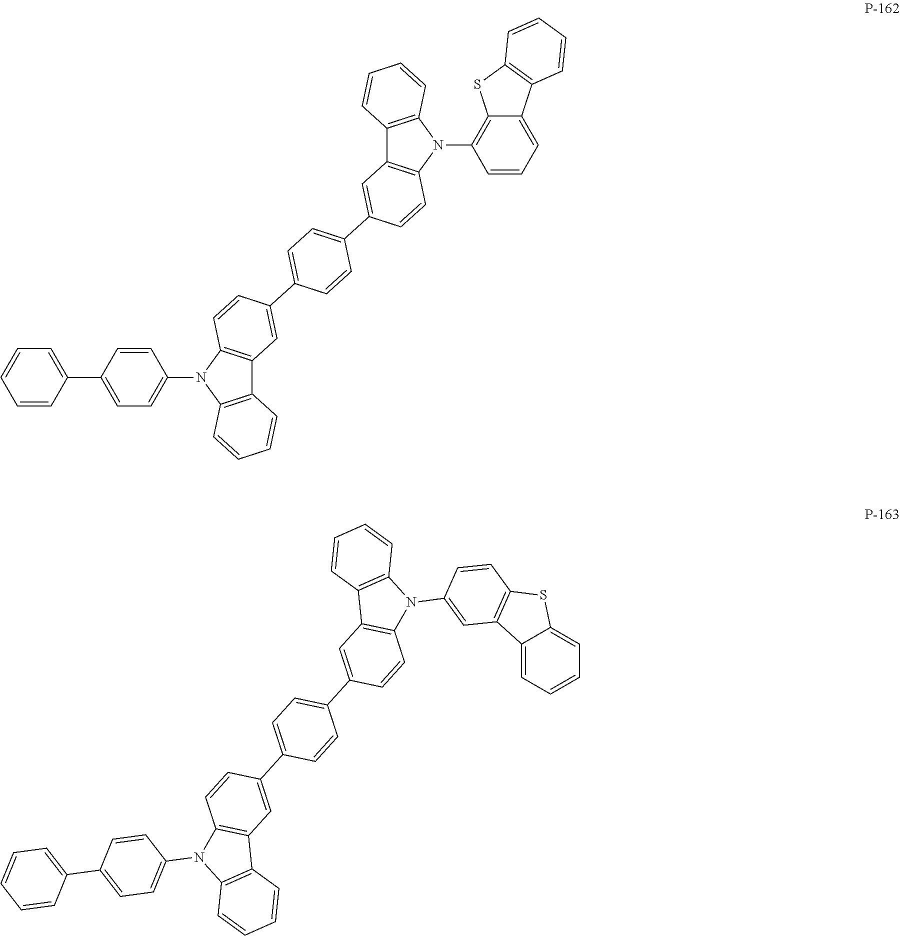 [SMILES: c1ccc(-c2ccc(-n3c4ccccc4c4cc(-c5ccc(-c6ccc7c(c6)c6ccccc6n7-c6cccc7c6sc6ccccc67)cc5)ccc43)cc2)cc1 ; c1ccc(-c2ccc(-n3c4ccccc4c4cc(-c5ccc(-c6ccc7c(c6)c6ccccc6n7-c6ccc7sc8ccccc8c7c6)cc5)ccc43)cc2)cc1]

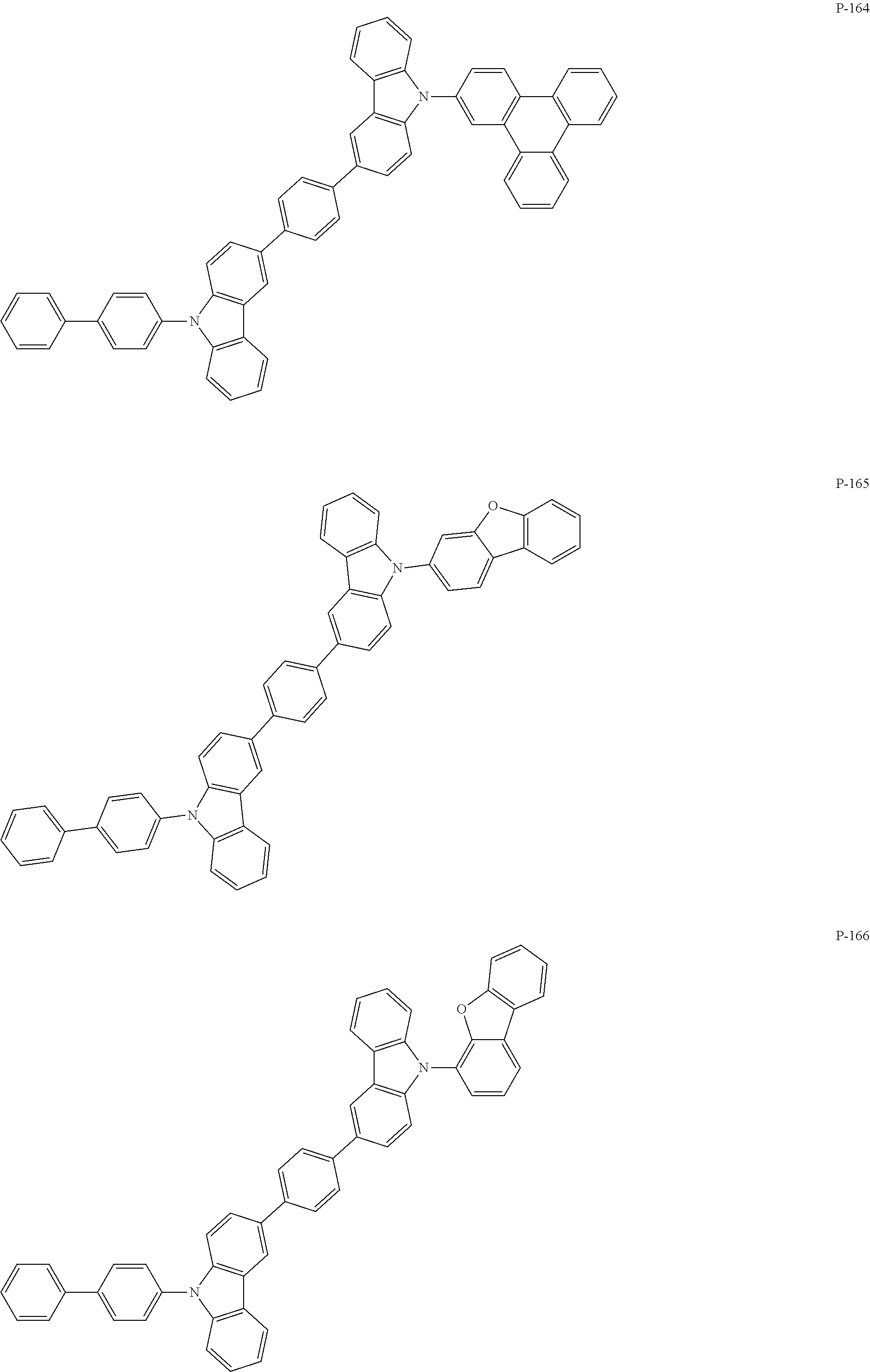 [SMILES: c1ccc(-c2ccc(-n3c4ccccc4c4cc(-c5ccc(-c6ccc7c(c6)c6ccccc6n7-c6ccc7c8ccccc8c8ccccc8c7c6)cc5)ccc43)cc2)cc1 ; c1ccc(-c2ccc(-n3c4ccccc4c4cc(-c5ccc(-c6ccc7c(c6)c6ccccc6n7-c6ccc7c(c6)oc6ccccc67)cc5)ccc43)cc2)cc1 ; c1ccc(-c2ccc(-n3c4ccccc4c4cc(-c5ccc(-c6ccc7c(c6)c6ccccc6n7-c6cccc7c6oc6ccccc67)cc5)ccc43)cc2)cc1]

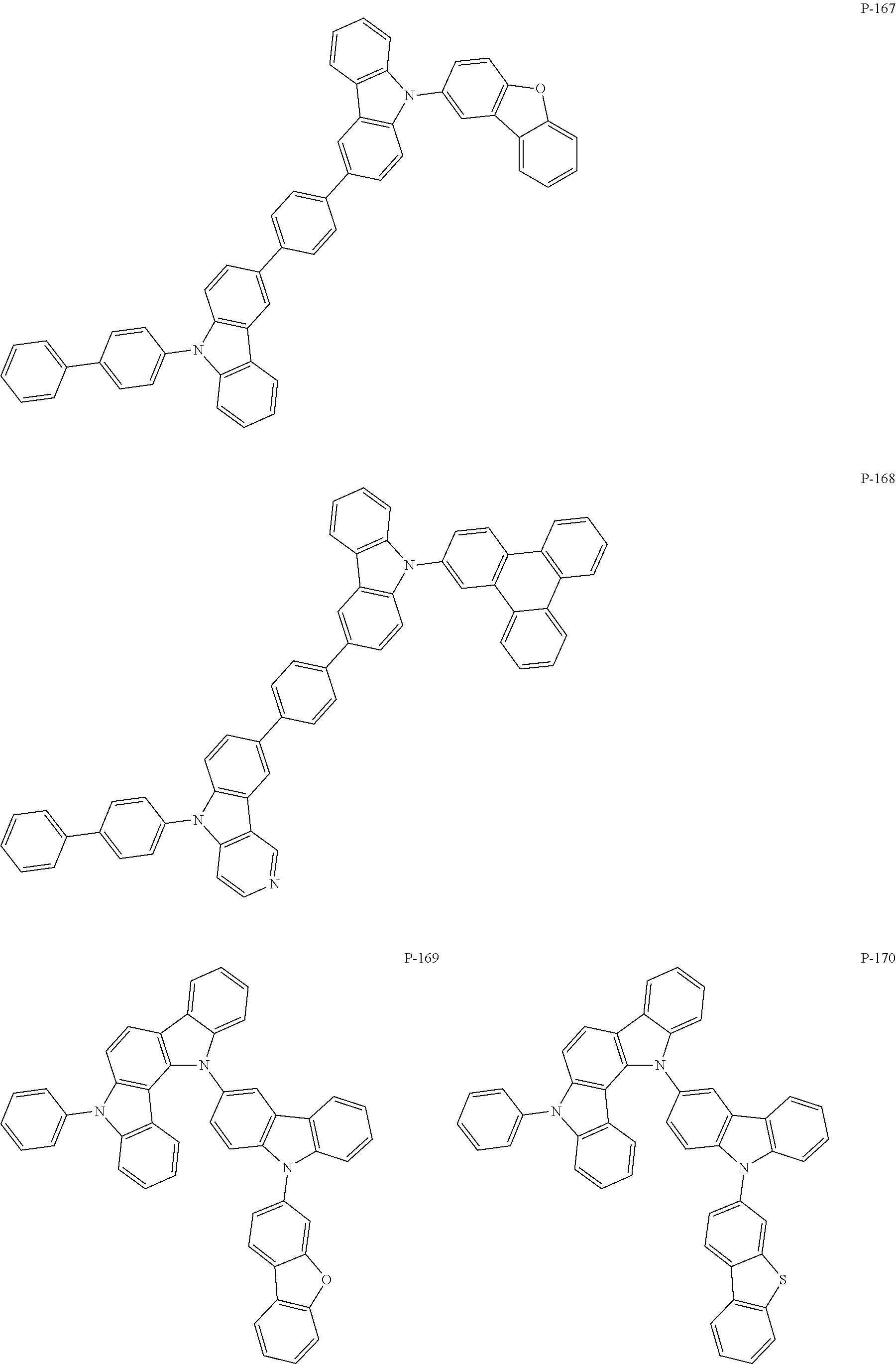 [SMILES: c1ccc(-c2ccc(-n3c4ccccc4c4cc(-c5ccc(-c6ccc7c(c6)c6ccccc6n7-c6ccc7oc8ccccc8c7c6)cc5)ccc43)cc2)cc1 ; c1ccc(-c2ccc(-n3c4ccncc4c4cc(-c5ccc(-c6ccc7c(c6)c6ccccc6n7-c6ccc7c8ccccc8c8ccccc8c7c6)cc5)ccc43)cc2)cc1 ; c1ccc(-n2c3ccccc3c3c2ccc2c4ccccc4n(-c4ccc5c(c4)c4ccccc4n5-c4ccc5c(c4)oc4ccccc45)c23)cc1 ; c1ccc(-n2c3ccccc3c3c2ccc2c4ccccc4n(-c4ccc5c(c4)c4ccccc4n5-c4ccc5c(c4)sc4ccccc45)c23)cc1]

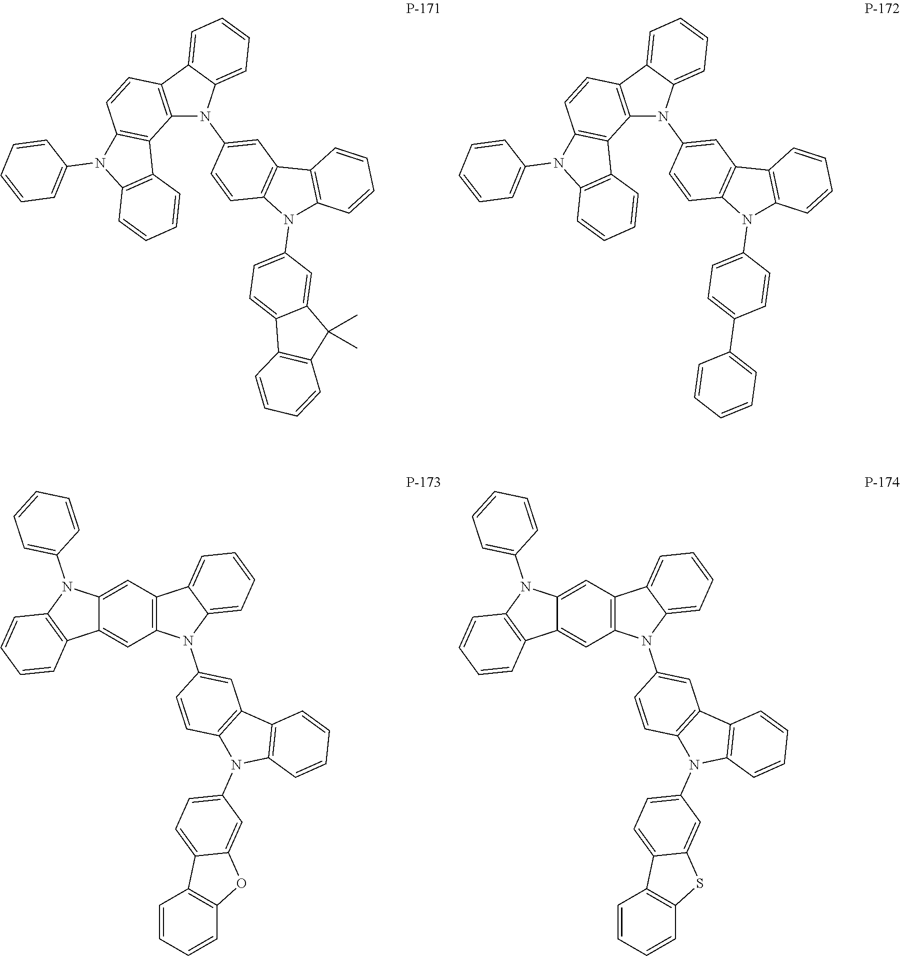 [SMILES: CC1(C)c2ccccc2-c2ccc(-n3c4ccccc4c4cc(-n5c6ccccc6c6ccc7c(c8ccccc8n7-c7ccccc7)c65)ccc43)cc21 ; c1ccc(-c2ccc(-n3c4ccccc4c4cc(-n5c6ccccc6c6ccc7c(c8ccccc8n7-c7ccccc7)c65)ccc43)cc2)cc1 ; c1ccc(-n2c3ccccc3c3cc4c(cc32)c2ccccc2n4-c2ccc3c(c2)c2ccccc2n3-c2ccc3c(c2)oc2ccccc23)cc1 ; c1ccc(-n2c3ccccc3c3cc4c(cc32)c2ccccc2n4-c2ccc3c(c2)c2ccccc2n3-c2ccc3c(c2)sc2ccccc23)cc1]

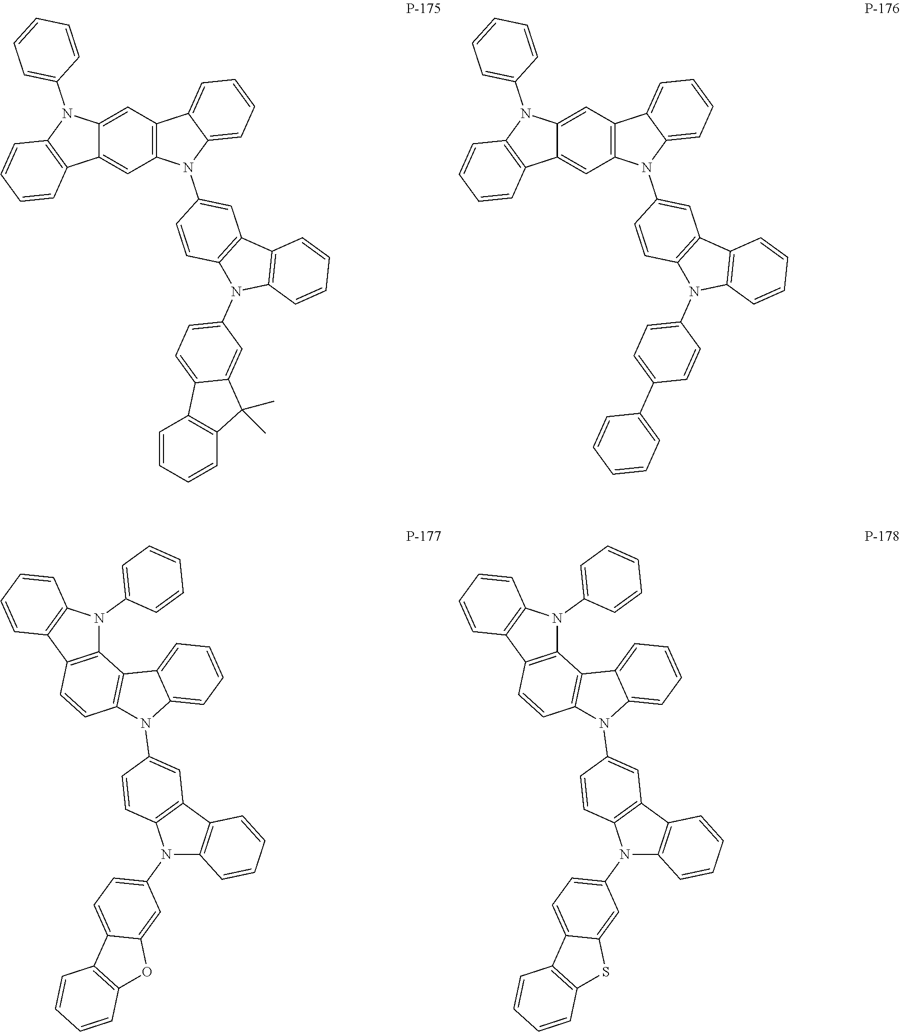 [SMILES: CC1(C)c2ccccc2-c2ccc(-n3c4ccccc4c4cc(-n5c6ccccc6c6cc7c(cc65)c5ccccc5n7-c5ccccc5)ccc43)cc21 ; c1ccc(-c2ccc(-n3c4ccccc4c4cc(-n5c6ccccc6c6cc7c(cc65)c5ccccc5n7-c5ccccc5)ccc43)cc2)cc1 ; c1ccc(-n2c3ccccc3c3ccc4c(c5ccccc5n4-c4ccc5c(c4)c4ccccc4n5-c4ccc5c(c4)oc4ccccc45)c32)cc1 ; c1ccc(-n2c3ccccc3c3ccc4c(c5ccccc5n4-c4ccc5c(c4)c4ccccc4n5-c4ccc5c(c4)sc4ccccc45)c32)cc1]

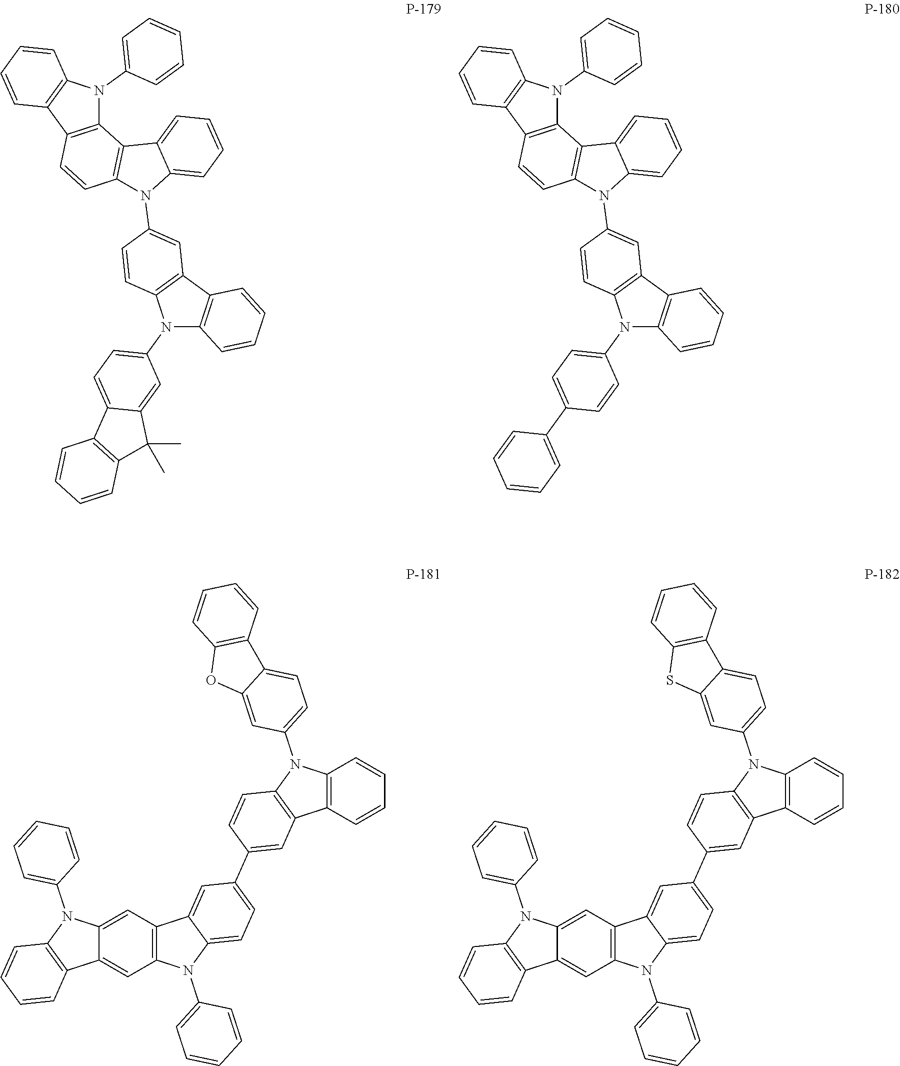 [SMILES: CC1(C)c2ccccc2-c2ccc(-n3c4ccccc4c4cc(-n5c6ccccc6c6c5ccc5c7ccccc7n(-c7ccccc7)c56)ccc43)cc21 ; c1ccc(-c2ccc(-n3c4ccccc4c4cc(-n5c6ccccc6c6c5ccc5c7ccccc7n(-c7ccccc7)c56)ccc43)cc2)cc1 ; c1ccc(-n2c3ccccc3c3cc4c(cc32)c2cc(-c3ccc5c(c3)c3ccccc3n5-c3ccc5c(c3)oc3ccccc35)ccc2n4-c2ccccc2)cc1 ; c1ccc(-n2c3ccccc3c3cc4c(cc32)c2cc(-c3ccc5c(c3)c3ccccc3n5-c3ccc5c(c3)sc3ccccc35)ccc2n4-c2ccccc2)cc1]

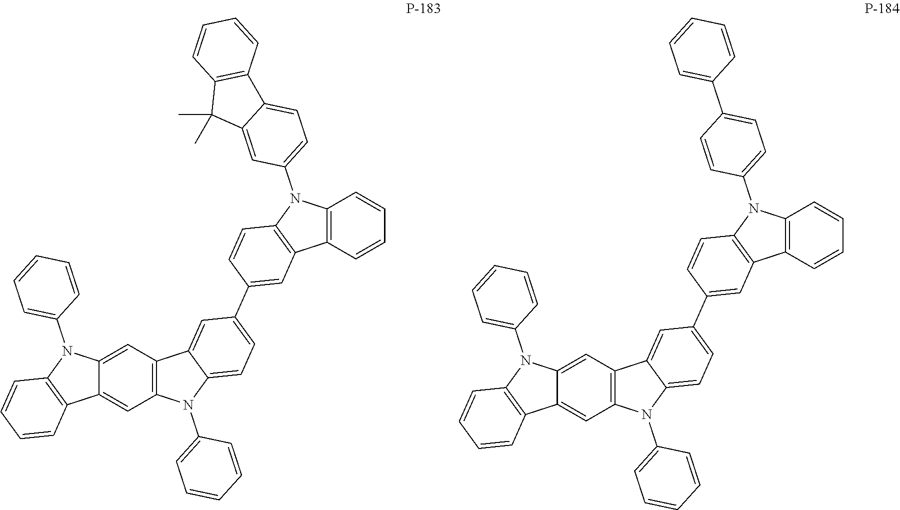 [SMILES: CC1(C)c2ccccc2-c2ccc(-n3c4ccccc4c4cc(-c5ccc6c(c5)c5cc7c(cc5n6-c5ccccc5)c5ccccc5n7-c5ccccc5)ccc43)cc21 ; c1ccc(-c2ccc(-n3c4ccccc4c4cc(-c5ccc6c(c5)c5cc7c(cc5n6-c5ccccc5)c5ccccc5n7-c5ccccc5)ccc43)cc2)cc1]

In yet another aspect, the present disclosure further provides a mixture comprising an organic compound, or an organic mixture as described herein, and at least one other organic functional material. The at least one other organic functional material may be selected from a hole-injection material (HIM), a hole-transport material (HTM), a hole-blocking material (HBM), an electron-injection material (EIM), an electron-transport material (ETM), an electron-blocking material (EBM), an emitting material, a host material (Host), a singlet emitting material (fluorescent emitting material), a triplet emitting material (phosphorescent emitting material), in particular a luminescent organometallic complex and a thermally activated delayed fluorescence material (TADF material). These organic functional materials are described in detail, for example, in WO2010135519A1, US20090134784A1 and WO2011110277A1. The entire contents of these three documents are incorporated herein by reference in their entirety. The organic functional material can be small molecule material and polymeric material.

In some embodiments, the mixture comprises an organic compound, or an organic mixture as described herein, and a phosphorescent emitter. Herein, the organic compound or the organic mixture as described herein can be used as a host, and the weight percentage of the phosphorescent emitter ≤20 wt %, preferably ≤15 wt %, more preferably ≤10 wt %.

In some embodiments, the mixture comprises an organic compound, or an organic mixture as described herein, and a fluorescent emitter. Herein, the organic compound or the organic mixture as described herein can be used as a fluorescent host material, the weight percentage of the fluorescent emitter ≤10 wt %, preferably ≤9 wt %, more preferably ≤8 wt %, further preferably ≤7 wt %, and most preferably ≤6 wt %.

In some embodiments, the mixture comprises an organic mixture as described herein, and a fluorescent host material. Herein, the organic mixture as described herein can be used as a fluorescent emitting material, the weight percentage of the fluorescent emitting material ≤10 wt %, preferably ≤9 wt %, more preferably ≤8 wt %, further particularly ≤7 wt %, and most preferably ≤6 wt %.

In some embodiments, the mixture comprises an organic mixture as described herein, a phosphorescent emitter, and a host material. Herein, the organic mixture as described herein can be used as an auxiliary emitting material, and the weight ratio of the organic mixture to the phosphorescent emitter is from 1:2 to 2:1. In some embodiments, the triplet energy level T₁ of the organic compound as described herein is higher than the phosphorescent emitter.

In some embodiments, the mixture comprises an organic mixture as described herein, and a TADF material. Herein, the organic compound as described herein can be used as a host material of the TADF, the weight percentage of the TADF material ≤15 wt %, preferably ≤10 wt %, more preferably ≤8 wt %.

The detailed description of the host material, phosphorescent emitting material, fluorescent host material, fluorescent emitting material, and TADF material is described in detail in WO2018095395. The entire contents of the patent document are hereby incorporated herein for reference.

Hereinafter, either the organic mixture or the mixture is referred to a mixture.

Another object of the present disclosure is to provide a material for the printed OLEDs.

For this purpose, the molecular weight of the organic compound as described herein ≥700 g/mol, preferably ≥800 g/mol, more preferably ≥900 g/mol, further preferably ≥1000 g/mol, and most preferably ≥1100 g/mol.

In some embodiments, the organic compound or mixture as described herein has a solubility of ≥10 mg/mL in toluene at 25° C., preferably ≥15 mg/mL, and most preferably ≥20 mg/mL.

In yet another aspect, the present disclosure further provides a formulation or ink comprising an organic compound, or a mixture as described herein, and at least one organic solvent.

The viscosity and surface tension of the ink are important parameters of the printing process. A suitable surface tension is required for the specific substrates and specific printing methods.

In some embodiments, the surface tension of the ink as described herein at 25° C. is in the range of 19 dyne/cm to 50 dyne/cm; more preferably in the range of 22 dyne/cm to 35 dyne/cm; and most preferably in the range of 25 dyne/cm to 33 dyne/cm.

In some embodiments, the viscosity of the ink as described herein at 25° C. is in the range of from about 1 cps to 100 cps; particularly in the range of 1 cps to 50 cps; more particularly in the range of 1.5 cps to 20 cps; and most particularly in the range of 4.0 cps to 20 cps. The resulting formulation will be particularly suitable for ink-jet printing.

The viscosity can be adjusted by different methods, such as by the selection of appropriate organic solvent and the concentration of the functional materials in the ink. In the ink comprising the above-mentioned metal-organic complexes or polymers as described herein facilitate the adjustment of the printing ink in the appropriate range according to the printing method used.

Generally, in the formulation comprising the functional material as described herein, the weight ratio of the functional material ranges from 0.3 wt % to 30 wt %, preferably in the range of 0.5 wt % to 20 wt %, more preferably in the range of 0.5 wt % to 15 wt %, even more preferably in the range of 0.5 wt % to 10 wt %, and most preferably in the range of 1 wt % to 5 wt %.

In some embodiments, the at least one organic solvent of the ink as described herein is selected from aromatic-based or heteroaromatic-based solvents, particular in aliphatic chain/ring substituted aromatic solvents, aromatic ketone solvents, or aromatic ether solvents.

Examples of solvents suitable for the present disclosure include, but not limited to aromatic-based or heteroaromatic-based solvents, such as p-diisopropylbenzene, amylbenzene, tetralin, cyclohexylbenzene, chloronaphtalene, 1,4-dimethylnaphthalene, 3-isopropylbenzene, p-methylisopropylbenzene, dipentylbenzene, tripentylbenzene, pentyltoluene, o-xylene, m-xylene, p-xylene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diiisopropylbenzene, 1-methoxynaphthalene, cyclohexylbenzene, dimethylnaphthalene, 3-isopropylbiphenyl, p-methylisopropylbenzene, 1-methylnaphthalene, 1,2,4-trichlorobenzene, 1,3-dipropoxybenzene, 4,4-difluorobenzenemethane, 1,2-dimethoxy-4-(1-propenyl) benzene, diphenylmethane, 2-phenylpyridine, 3-phenylpyridine, N-methyldiphenylamine, 4-isopropylbipheny, 1,1,1-bis(3,4-dimethylphenyl) ethane, 2-isopropylnaphthalene, dibenzyl ether, etc; ketone-based solvents, such as 1-tetrahydronaphthalone, 2-tetrahydronaphthalone, 2-(phenylepoxy)tetrahydronaphthalone, 6-(methoxy)tetrahydronaphthalone, acetophenone, phenylacetone, benzophenone, and derivatives thereof such as 4-methyl acetophenone, 3-methyl acetophenone, 2-methyl acetophenone, 4-methyl propanone, 3-methyl propanone, 2-methyl propanone, isophorone, 2,6,8-trimethyl-4-nonanone, fenchone, 2-nonanone, 3-nonanone, 5-nonanone, 2-decanone, 2,5-hexanedione, phoron, di-n-amyl ketone, etc; aromatic ether solvents, such as 3-phenoxytoluene, butoxybenzene, benzylbutylbenzene, p-anisaldehyde dimethyl acetal, tetrahydro-2-phenoxy-2H-pyran, 1,2-dimethoxy-4-(1-propenyl)benzene, 1,4-benzodioxane, 1,3-dipropylbenzene, 2,5-dimethoxytoluene, 4-ethylphenyl ether, 1,2,4-trimethoxybenzene, 4-(1-propenyl)-1,2-dimethoxybenzene, 1,3-dimethoxybenzene, glycidyl phenyl ether, dibenzyl ether, 4-tert-butyl anisole, trans-anethole, 1,2-dimethoxybenzene, 1-methoxynaphthalene, diphenyl ether, 2-phenoxymethyl ether, 2-phenoxytetrahydrofuran, ethyl-2-naphthyl ether, dipentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc; ester solvent, such as alkyl octanoate, alkyl sebacate, alkyl stearate, alkyl benzoate, alkyl phenylacetate, alkyl cinnamate, alkyl oxalate, alkyl maleate, alkyl lactone, alkyl oleate, etc.

Further, the at least one organic solvent of the ink as described herein can be selected from aliphatic ketones, such as 2-nonanone, 3-nonanone, 5-nonanone, 2-decanone, 2,5-hexanedione, 2,6,8-trimethyl-4-nonanone, phoron, di-n-amyl ketone, etc; and the at least one organic solvent as described herein can be selected from aliphatic, ethers, such as, dipentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc.

In some embodiments, the printing ink further comprises another organic solvent. Examples of the another organic solvent include, but not limited to: methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4 dioxane, acetone, methyl ethyl ketone, 1,2 dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetrahydronaphthalene, decalin, indene, and/or mixtures thereof.

In some embodiments, the formulation as described herein is a solution.

In some embodiments, the formulation as described herein is a dispersion.

The organic formulations in the embodiments as described herein may comprise the organic mixture of 0.01 wt % to 20 wt %, preferably 0.1 wt % to 15 wt %, more preferably 0.2 wt % to 10 wt %, and most preferably 0.25 wt % to 5 wt %.

The present disclosure further provides the use of the formulation as a coating or printing ink in the preparation of organic electronic devices, particularly preferably by printing or coating processing methods.

Where suitable printing or coating techniques include, but not limited to gravure printing, ink-jet printing, nozzle printing, typographic printing, screen printing, dip coating, spin coating, blade coating, roller printing, torsion roll printing, planographic printing, flexographic printing, rotary printing, spray coating, brush coating, pad printing, slit die coating, and so on. Preferred techniques are gravure printing, nozzle printing, and ink-jet printing. The solution or dispersion may additionally comprise one or more components, such as surface active compounds, lubricants, wetting agents, dispersing agents, hydrophobic agents, binders, etc., which are used to adjust the viscosity and film forming properties, or to improve adhesion, etc. For more information on printing technologies and their requirements for solutions, such as solvent, concentration, viscosity, etc, please refer to Handbook of Print Media: Technologies and Production Methods, edited by Helmut Kipphan, ISBN 3-540-67326-1.

Based on the above-mentioned organic compound or mixture, the present disclosure also provides an application of the organic compound or mixture as described herein, i.e., the organic compound or mixture is applied to an organic electronic device, and the organic electronic device may be selected from, but not limited to an organic light emitting diode (OLED), an organic photovoltaic cell (OPV), an organic light emitting electrochemical cell (OLEEC), an organic field effect transistor (OFET), an organic light emitting field effect transistor, an organic laser, an organic spintronic electronic device, a photodiode, an organic sensor, or an organic plasmon emitting diode (OPED) etc, particular an OLED. In the embodiments of the present disclosure, it is preferred to use the organic compound for the light-emitting layer of the OLED.

In yet another aspect, the present disclosure further provides an organic electronic device comprising an organic compound, or a mixture as described herein. In some embodiments, the organic electronic device comprises a first electrode, a second electrode, and at least one functional layer disposed between the first electrode and the second electrode, where at least one of the functional layer is selected from a light-emitting layer, an electron-transport layer, or a hole-transport layer. Preferably, the light-emitting layer comprises an organic compound, or a mixture as described herein. In some embodiments, the electron-transport layer comprises an organic compound as described herein. In some embodiments, the hole-transport layer comprises an organic compound as described herein.

The organic electronic device may be selected from, but not limited to, an organic light emitting diode (OLED), an organic photovoltaic cell (OPV), an organic light emitting electrochemical cell (OLEEC), an organic field effect transistor (OFET), an organic light emitting field effect transistor, an organic laser, an organic spintronic device, an organic sensor, or an organic plasmon emitting diode (OPED), etc, particular in an organic electroluminescent device, such as an OLED, an OLEEC, an organic light emitting field effect transistor.

In some embodiments, the light-emitting layer of the electroluminescent device comprises an organic compound, or a mixture as described herein; or comprises an organic compound, or a mixture as described herein, and a phosphorescent emitter; or comprises an organic compound, or a mixture as described herein, and a host material; or comprises an organic compound, or a mixture as described herein, a phosphorescent emitter, and a host material.

In some embodiments, the electron-transport layer of the electroluminescent device comprises an organic compound as described herein.

In the organic electroluminescent devices as described herein, in particular an OLED, which comprises a substrate, an anode, at least one light-emitting layer, and a cathode.

The substrate should be opaque or transparent. A transparent substrate could be used to produce a transparent light-emitting device (for example: Bulovic et al., Nature 1996, 380, p 29, and Gu et al., Appl. Phys. Lett. 1996, 68, p 2606). Substrate may be either rigid or elastic. The substrate can be rigid/flexible, e.g., it can be plastic, metal, semiconductor wafer, or glass.

Preferably, the substrate has a smooth surface. Particularly desirable are substrates without surface defects. In some embodiments, the substrate is flexible and can be selected from a polymer film or plastic with a glass transition temperature Tg over 150° C., preferably over 200° C., more preferably over 250° C., and most preferably over 300° C. Examples of the suitable flexible substrate includes poly (ethylene terephthalate) (PET) and polyethylene glycol (2,6-naphthalene) (PEN).

The choice of anodes may include a conductive metal, a metal oxide, or a conductive polymer. The anode should be able to easily inject holes into a hole-injection layer (HIL), a hole-transport layer (HTL), or a light-emitting layer. In some embodiments, the absolute value of the difference between the work function of the anode and the HOMO energy level of the emitter of the light-emitting layer, or the HOMO energy level/valence band energy level of the p-type semiconductor material for the hole-injection layer (HIL)/hole-transport layer (HTL)/electron-blocking layer (EBL) is less than 0.5 eV, preferably less than 0.3 eV, more preferably less than 0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by one of ordinary skill in the art. The anode material can be deposited using any suitable technique, such as a suitable physical vapor deposition method, including RF magnetron sputtering, vacuum thermal evaporation, e-beam, etc. In some embodiments, the anode is patterned. Patterned conductive ITO substrates are commercially available and can be used to produce the devices as described herein.

The choice of cathode may include a conductive metal and a metal oxide. The cathode should be able to easily inject electrons into the EIL, the ETL, or the directly into the emitting layer. In some embodiments, the absolute value of the difference between the work function of the cathode and the LUMO energy level of the emitter of the light-emitting layer, or the LUMO energy level/conduction band energy level of the n-type semiconductor material for electron-injection layer (EIL)/electron-transport layer (ETL)/hole-blocking layer (HBL) is less than 0.5 eV, preferably less than 0.3 eV, most preferably less than 0.2 eV. In principle, all materials that may be used as cathodes for OLEDs are possible to apply as cathode materials for the present disclosure.

Examples of cathode materials include, but not limited to: Al, Au, Ag, Ca, Ba, Mg, LiF/Al, MgAg alloys, BaF₂/Al, Cu, Fe, Co, Ni, Mn, Pd, Pt, ITO, etc. The cathode material can be deposited using any suitable technique, such as the suitable physical vapor deposition method, including RF magnetron sputtering, vacuum thermal evaporation, e-beam, etc.

The OLED may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in detail above and in WO2010135519A1, US20090134784A1 and WO2011110277A1, the entire contents of these three documents are hereby incorporated herein for reference.

In some embodiments, the electron-transport layer (ETL) or hole-blocking layer (HBL) of the electroluminescent device as described herein comprises an organic compound, or a polymer as described herein. In some embodiments, the light-emitting layer of the light-emitting device as described herein comprises a light-emitting layer, which is formed by using the formulation as described herein.

In some embodiments, the light-emitting layer of the light-emitting device as described herein comprises an organic compound, or a mixture as described herein. The light-emitting layer may preferably be formed by one of the following two methods:
(1) The mixture comprising the first compound H1 and the second compound H2 is deposited as a source. This can be prepared by printing using a formulation as described herein, or by vacuum evaporation deposition using the mixture as a source.
(2) The first compound H1 and the second compound H2 are independently used as two separate sources, and are deposited by vacuum evaporation.

In some embodiments, the molecular weight difference between the first compound H1 and the second compound H2 is small, and its sublimation temperature is also small. In order to simplify the vapor deposition process of the material and reduce the production cost of the OLED display devices, the mixture can be used to prepare the light-emitting layer by mixing two host materials (i.e., the first compound H1 and the second compound H2) in a certain ratio and then evaporating them through a vapor deposition heat source.

The light-emitting device as described herein has a light-emitting wavelength between 300 nm and 1000 nm, preferably between 350 nm and 900 nm, more preferably between 400 nm and 800 nm.

In yet another aspect, the present disclosure further provides the applications of organic electronic devices in various electronic equipment, including, but not limited to, display devices, lighting equipment, light sources, sensors, etc.

In yet another aspect, the present disclosure further provides electronic devices comprising organic electronic devices as described herein, including, but not limited to, display devices, lighting equipment, light sources, sensors, etc.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### SPECIFIC EMBODIMENT

### Synthesis of the First Host Material (i.e., the First Compound H1)

### Example 1

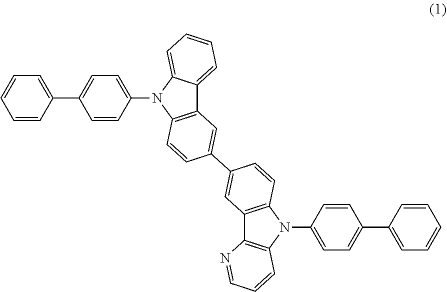 [SMILES: c1ccc(-c2ccc(-n3c4ccccc4c4cc(-c5ccc6c(c5)c5ncccc5n6-c5ccc(-c6ccccc6)cc5)ccc43)cc2)cc1]

### 5-([1,1′-diphenyl]-4-yl)-8-(9-([1,1′-diphenyl]-4-yl)-9H-carbazole-3-yl)-5H-pyrido[3,2-b]indole

(9-([1,1′-diphenyl]-4-yl)-9H-carbazole-3-yl)boric acid (3.63 g, 10 mmol), 5-([1,1′-diphenyl]-4-yl)-8-bromo-5H-pyrido[3,2-b]indole (3.98 g, 10 mmol), potassium carbonate (6.9 g, 50 mmol), Pd(PPh₃)₄ (0.58 g, 0.5 mmol), 100 mL of toluene, 25 mL of water, and 25 mL of ethanol were added to a 250 mL three-necked flask under N2 atmosphere, and reacted at 110° C., meanwhile TLC was used to follow the reaction process. After the reaction was completed, the reaction solution was cooled to room temperature, then was poured into water, washed to remove K₂CO₃. After the suction filtration, a solid product was obtained and washed with dichloromethane. The crude product was recrystallized with dichloromethane and methanol to give 5.8 g of 5-([1,1′-diphenyl]-4-yl)-8-(9-([1,1′-diphenyl]-4-yl)-9H-carbazole-3-yl)-5H-pyrido[3,2-b]indole, MS(ASAP)=637.4.

### Example 2

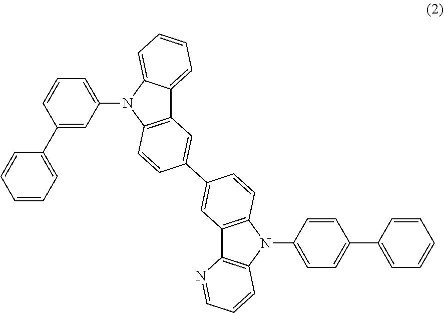 [SMILES: c1ccc(-c2ccc(-n3c4ccc(-c5ccc6c(c5)c5ccccc5n6-c5cccc(-c6ccccc6)c5)cc4c4ncccc43)cc2)cc1]

### 8-(9-([1,1′-diphenyl]-3-yl)-9H-carbazole-3-yl)-5-([1,1′-diphenyl]-4-yl)-5H-pyrido[3,2-b]indole

(9-([1,1′-diphenyl]-3-yl)-9H-carbazole-3-yl)boric acid (3.63 g, 10 mmol), 5-([1,1′-diphenyl]-4-yl)-8-bromo-5H-pyrido[3,2-b]indole (3.98 g, 10 mmol), potassium carbonate (6.9 g, 50 mmol), Pd(PPh₃)₄ (0.58 g, 0.5 mmol), 100 mL of toluene, 25 mL of water, and 25 mL of ethanol were added to a 250 mL three-necked flask under N2 atmosphere, and reacted at 110° C., meanwhile TLC was used to follow the reaction process. After the reaction was completed, the reaction solution was cooled to room temperature, then was poured into water, washed to remove K₂CO₃. After the suction filtration, a solid product was obtained and washed with dichloromethane. The crude product was recrystallized with dichloromethane and methanol to give 5.4 g of 8-(9-([1,1′-diphenyl]-3-yl)-9H-carbazole-3-yl)-5-([1,1′-diphenyl]-4-yl)-5H-pyrido[3,2-b]indole, MS(ASAP)=637.8.

### Example 3

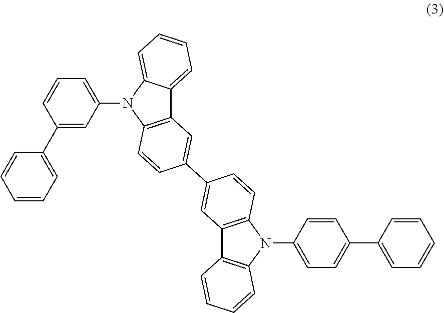 [SMILES: c1ccc(-c2ccc(-n3c4ccccc4c4cc(-c5ccc6c(c5)c5ccccc5n6-c5cccc(-c6ccccc6)c5)ccc43)cc2)cc1]

### 6-(9-([1,1′-diphenyl]-3-yl)-9H-carbazole-3-yl)-9-([1,1′-diphenyl]-4-yl)-9H-pyrido[2,3-b]indole

(9-([1,1′-diphenyl]-3-yl)-9H-carbazole-3-yl)boric acid (3.63 g, 10 mmol), 9-([1,1′-diphenyl]-4-yl)-3-bromo-9H-carbazole (3.98 g, 10 mmol), potassium carbonate (6.9 g, 50 mmol), Pd(PPh3)4 (0.58 g, 0.5 mmol), 100 mL of toluene, 25 mL of water and 25 mL of ethanol were added to a 250 mL three-necked flask under N2 atmosphere, and reacted at 110° C., meanwhile TLC was used to follow the reaction process. After the reaction was completed, the reaction solution was cooled to room temperature, then was poured into water, washed to remove K₂CO₃. After the suction filtration, a solid product was obtained and washed with dichloromethane. The crude product was recrystallized with dichloromethane and methanol to give 5.4 g of 6-(9-([1,1′-diphenyl]-3-yl)-9H-carbazole-3-yl)-9-([1,1′-diphenyl]-4-yl)-9H-pyrido[2,3-b]indole, MS(ASAP)=636.6.

### Example 4

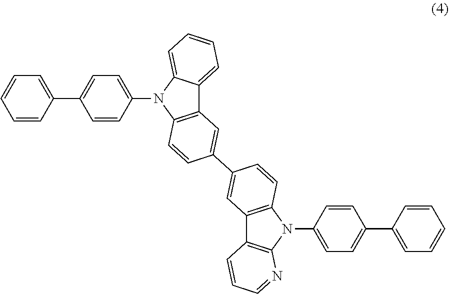 [SMILES: c1ccc(-c2ccc(-n3c4ccccc4c4cc(-c5ccc6c(c5)c5cccnc5n6-c5ccc(-c6ccccc6)cc5)ccc43)cc2)cc1]

### 9-([1,1′-diphenyl]-4-yl)-6-(9-([1,1′-diphenyl]-4-yl)-9H-carbazole-3-yl)-9H-pyrido[2,3-b]indole

(9-([1,1′-diphenyl]-4-yl)-9H-carbazole-3-yl)boric acid (3.63 g, 10 mmol), 9-([1,1′-diphenyl]-4-yl)-6-bromo-9H-pyrido[2,3-b]indole (3.98 g, 10 mmol), potassium carbonate (6.9 g, 50 mmol), Pd(PPh₃)₄ (0.58 g, 0.5 mmol), 100 mL of toluene, 25 mL of water, and 25 mL of ethanol were added to a 250 mL three-necked flask under N2 atmosphere, and reacted at 110° C., meanwhile TLC was used to follow the reaction process. After the reaction was completed, the reaction solution was cooled to room temperature, then was poured into water, washed to remove K₂CO₃. After the suction filtration, a solid product was obtained and washed with dichloromethane. The crude product was recrystallized with dichloromethane and methanol to give 5.5 g of 9-([1,1′-diphenyl]-4-yl)-6-(9-([1,1′-diphenyl]-4-yl)-9H-carbazole-3-yl)-9H-pyrido[2,3-b]indole, MS(ASAP)=637.4.

### Example 5

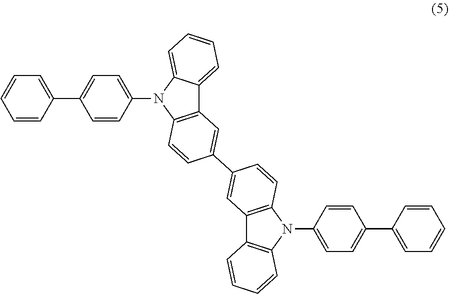 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

### 9,9′-bis([1,1′-diphenyl]-4-yl)-9H,9′H-3,3′-connected dicarbazole

(9-([1,1′-diphenyl]-4-yl)-9H-carbazole-3-yl)boric acid (3.63 g, 10 mmol), (9-([1,1′-diphenyl]-4-yl)-9H-3-bromo-substituted)carbazole (3.98 g, 10 mmol), potassium carbonate (6.9 g, 50 mmol), Pd(PPh₃)₄ (0.58 g, 0.5 mmol), 100 mL of toluene, 25 mL of water and 25 mL of ethanol were added to a 250 mL three-necked flask under N2 atmosphere, and reacted at 110° C., meanwhile TLC was used to follow the reaction process. After the reaction was completed, the reaction solution was cooled to room temperature, then was poured into water, washed to remove K₂CO₃. After the suction filtration, a solid product was obtained and washed with dichloromethane. The crude product was recrystallized with dichloromethane and methanol to give 6.2 g of 9,9′-bis([1,1′-diphenyl]-4-yl)-9H,9′H-3,3′-lien dicarbazole, MS(ASAP)=636.2.

### Synthesis of Second Organic Host Material (i.e., the Second Compound H2)

The three intermediates shown in the following formulas a, b and c were synthesized according to the synthetic method in the literature (JACS, 1964, 86(6), 1125-1127) under the harsh synthetic conditions. Firstly, the amine hydrochloride was reacted with LiBH₄ at high temperature (300° C.) to form nonaromatic aza-boron tritylene, which then was dehydrogenated by Pd—C to form aromatic aza-boron tritylene.

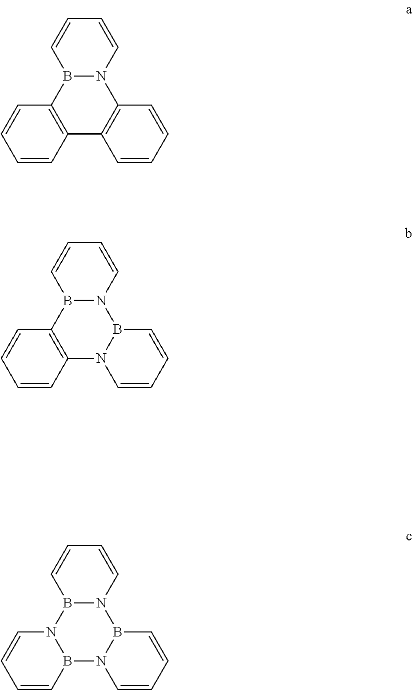 [SMILES: C1=CB2c3ccccc3-c3ccccc3N2C=C1 ; C1=CB2c3ccccc3N3C=CC=CB3N2C=C1 ; C1=CB2N3C=CC=CB3N3C=CC=CB3N2C=C1]

### Example 6

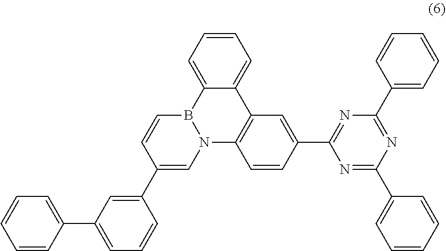 [SMILES: C1=CC(c2cccc(-c3ccccc3)c2)=CN2B1c1ccccc1-c1cc(-c3nc(-c4ccccc4)nc(-c4ccccc4)n3)ccc12]

### 7-([1,1′-diphenyl]-3-yl)-2-(4,6-diphenyl-1,3,5-triazin-2-yl)-[1,2]borazo[1,2-a]diphenyl[c,e][1,2]borazotriphenylene

The synthetic route is shown as follows:

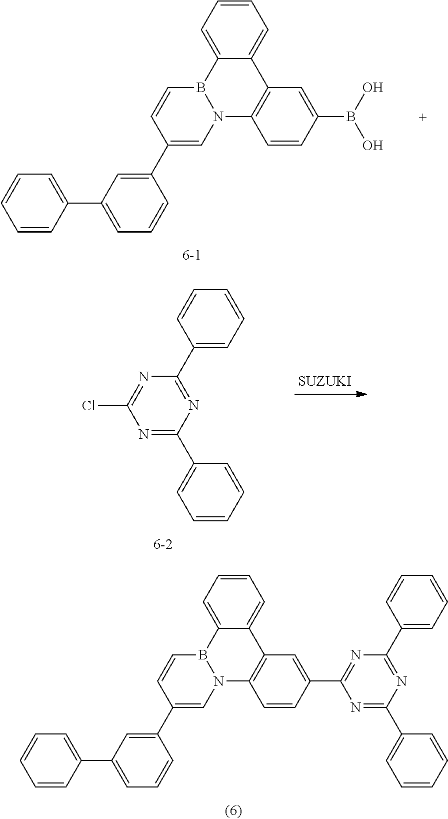 [SMILES: OB(O)c1ccc2c(c1)-c1ccccc1B1C=CC(c3cccc(-c4ccccc4)c3)=CN12 ; Clc1nc(-c2ccccc2)nc(-c2ccccc2)n1 ; C1=CC(c2cccc(-c3ccccc3)c2)=CN2B1c1ccccc1-c1cc(-c3nc(-c4ccccc4)nc(-c4ccccc4)n3)ccc12]

Synthesis of compound (6) by the classical SUZUKI coupling reaction is as follows: intermediate 6-1 (2.74 g, 10 mmol), 11 mmol of intermediate 6-2, potassium carbonate (6.9 g, 50 mmol), Pd(PPh₃)₄ (0.58 g, 0.5 mmol), 100 mL of toluene, 25 mL of water and 25 mL of ethanol were added to a 250 mL three-necked flask under N2 atmosphere, and reacted at 110° C., meanwhile TLC was used to follow the reaction process. After the reaction was completed, the reaction solution was cooled to room temperature, then was poured into water, washed to remove K₂CO₃. After the suction filtration, a solid product was obtained and washed with dichloromethane. The crude product was recrystallized with dichloromethane and ethanol to give 80.3% yield of 7-([1,1′-diphenyl]-3-yl)-2-(4,6-diphenyl-1,3,5-triazin-2-yl)-[1,2]borazo[1,2-a]diphenyl[c,e][1,2]borazotriphenylene, MS(ASAP)=612.2.

### Example 7

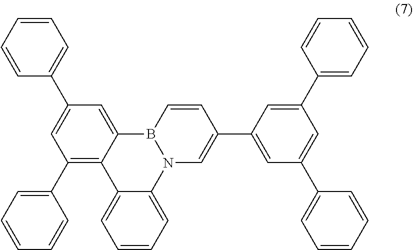 [SMILES: C1=CC(c2cc(-c3ccccc3)cc(-c3ccccc3)c2)=CN2B1c1cc(-c3ccccc3)cc(-c3ccccc3)c1-c1ccccc12]

### 8-([1,1′:3′,1″-triphenyl]-5′-yl)-12,14-diphenyl-[1,2]borazo[1,2-a]diphenyl[c,e][1,2]borazotriphenylene

The synthetic route is shown as follows:

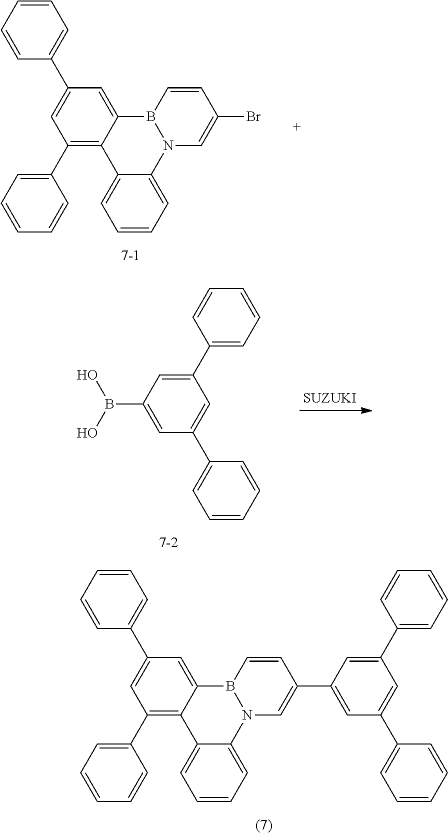 [SMILES: BrC1=CN2B(C=C1)c1cc(-c3ccccc3)cc(-c3ccccc3)c1-c1ccccc12 ; OB(O)c1cc(-c2ccccc2)cc(-c2ccccc2)c1 ; C1=CC(c2cc(-c3ccccc3)cc(-c3ccccc3)c2)=CN2B1c1cc(-c3ccccc3)cc(-c3ccccc3)c1-c1ccccc12]

The synthesis of compound (7) was similar to that of compound (6), both using the classical SUZUKI coupling reaction as follows:

intermediate 7-1 (2.74 g, 10 mmol), 11 mmol of intermediate 7-2, potassium carbonate (6.9 g, 50 mmol), Pd(PPh₃)₄ (0.58 g, 0.5 mmol), 100 mL of toluene, 25 mL of water and 25 mL of ethanol were added to a 250 mL three-necked flask under N2 atmosphere, and reacted at 110° C., meanwhile TLC was used to follow the reaction process. After the reaction was completed, the reaction solution was cooled to room temperature, then was poured into water, washed to remove K₂CO₃. After the suction filtration, a solid product was obtained and washed with dichloromethane. The crude product was recrystallized with dichloromethane and petroleum ether to give 78.8% yield of 8-([1,1′:3′,1″-triphenyl]-5′-yl)-12,14-diphenyl-[1,2]borazo[1,2-a]diphenyl[c,e][1,2]borazotriphenylene, MS(ASAP)=609.4.

### Example 8

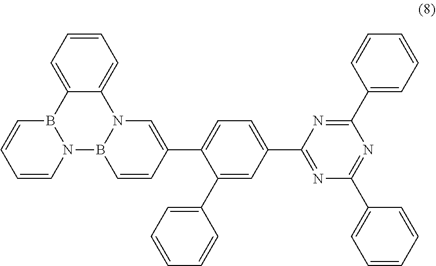 [SMILES: C1=CB2c3ccccc3N3C=C(c4ccc(-c5nc(-c6ccccc6)nc(-c6ccccc6)n5)cc4-c4ccccc4)C=CB3N2C=C1]

### 9-(5-(4,6-diphenyl-1,3,5-triazin-2-yl)-[1,1′-diphenyl]-2-yl)bis([1,2]azaboronophenyl)[1,2-a:1′,2′-c]phenyl[e][1,3,2,4]diazaboronylidene-3-benzene

The synthetic route is shown as follows:

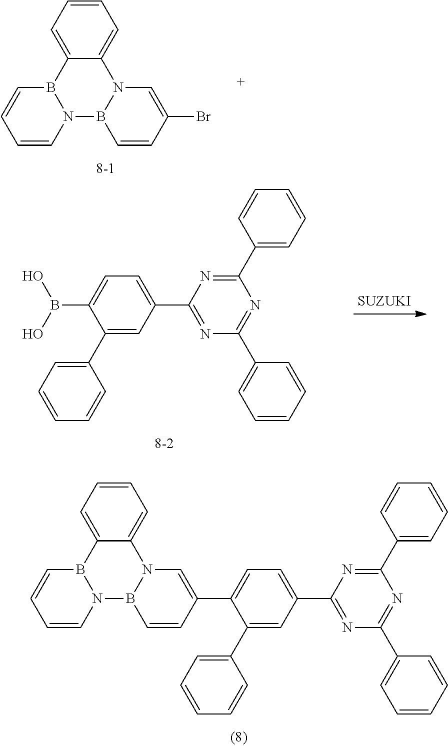 [SMILES: BrC1=CN2B(C=C1)N1C=CC=CB1c1ccccc12 ; OB(O)c1ccc(-c2nc(-c3ccccc3)nc(-c3ccccc3)n2)cc1-c1ccccc1 ; C1=CB2c3ccccc3N3C=C(c4ccc(-c5nc(-c6ccccc6)nc(-c6ccccc6)n5)cc4-c4ccccc4)C=CB3N2C=C1]

The synthesis of compound (8) was similar to that of compound (6), both using the classical SUZUKI coupling reaction as follows:

intermediate 8-1 (2.73 g, 10 mmol), 11 mmol of intermediate 8-2, potassium carbonate (6.9 g, 50 mmol), Pd(PPh₃)₄ (0.58 g, 0.5 mmol), 100 mL of toluene, 25 mL of water, and 25 mL of ethanol were added to a 250 mL three-necked flask under N2 atmosphere, and reacted at 110° C., meanwhile TLC was used to follow the reaction process. After the reaction was completed, the reaction solution was cooled to room temperature, then was poured into water, washed to remove K₂CO₃. After the suction filtration, a solid product was obtained and washed with dichloromethane. The crude product was recrystallized by heating with o-xylene to give 78.4% yield of 9-(5-(4,6-diphenyl-1,3,5-triazin-2-yl)-[1,1′-diphenyl]-2-yl)bis([1,2]azaboronophenyl)[1,2-a:1′,2′-c]phenyl[e][1,3,2,4]diazaboronylidene-3-benzene, MS(ASAP)=613.4.

### Example 9

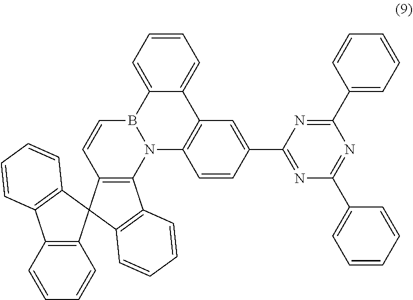 [SMILES: C1=CC2=C(c3ccccc3C23c2ccccc2-c2ccccc23)N2B1c1ccccc1-c1cc(-c3nc(-c4ccccc4)nc(-c4ccccc4)n3)ccc12]

### 16-(4,6-diphenyl-1,3,5-triazin-2-yl)spiro[diphenyl[c,e]indeno[2′,1′:5,6][1,2]azaboribenz[1,2-a][1,2] azaborotritylidene-8,9′-fluorene]

The synthetic route is shown as follows:

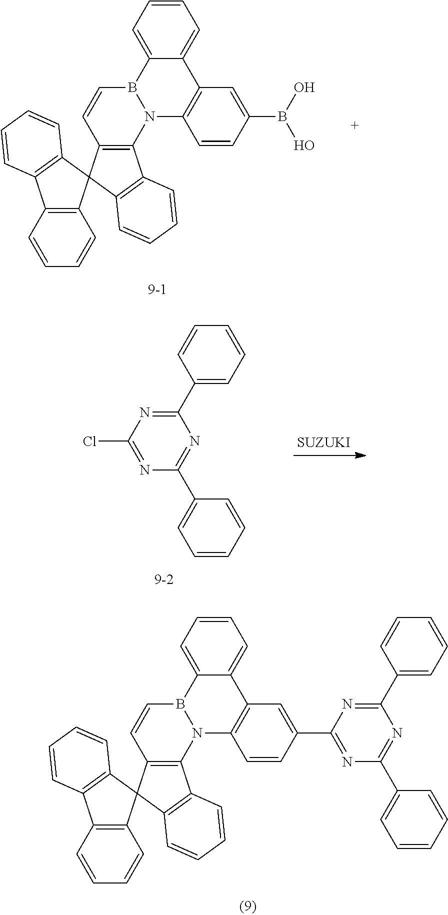 [SMILES: OB(O)c1ccc2c(c1)-c1ccccc1B1C=CC3=C(c4ccccc4C34c3ccccc3-c3ccccc34)N12 ; Clc1nc(-c2ccccc2)nc(-c2ccccc2)n1 ; C1=CC2=C(c3ccccc3C23c2ccccc2-c2ccccc23)N2B1c1ccccc1-c1cc(-c3nc(-c4ccccc4)nc(-c4ccccc4)n3)ccc12]

The synthesis of compound (9) was similar to that of compound (6), both using the classical SUZUKI coupling reaction as follows:

10 mmol of intermediate 9-1, 11 mmol of intermediate 9-2, potassium carbonate (6.9 g, 50 mmol), Pd(PPh₃)₄ (0.58 g, 0.5 mmol), 100 mL of toluene, 25 mL of water, and 25 mL of ethanol were added to a 250 mL three-necked flask under N2 atmosphere, and reacted at 110° C., meanwhile TLC was used to follow the reaction process After the reaction was completed, the reaction solution was cooled to room temperature, then was poured into water, washed to remove K₂CO₃. After the suction filtration, a solid product was obtained and washed with dichloromethane. The crude product was recrystallized with dioxane to give 80.1% yield of solid powder, MS(ASAP)=698.7.

### Example 10

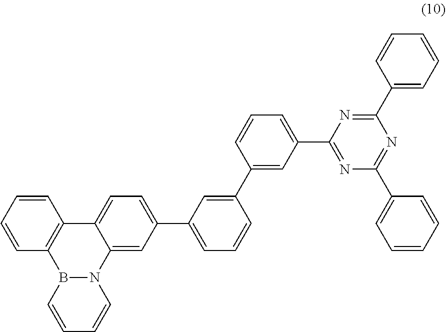 [SMILES: C1=CB2c3ccccc3-c3ccc(-c4cccc(-c5cccc(-c6nc(-c7ccccc7)nc(-c7ccccc7)n6)c5)c4)cc3N2C=C1]

### 3-(3′-(4,6-diphenyl-1,3,5-triazin-2-yl)-[1,1′-diphenyl]-3-yl)-[1,2]azaboronatophenyl[1,2-a]diphenyl[c,e][1,2]azaboronatritylenzene

The synthetic route is shown as follows:

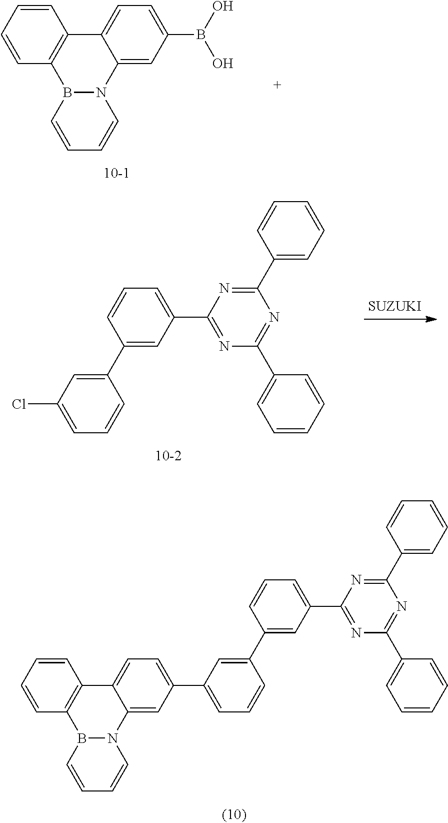 [SMILES: OB(O)c1ccc2c(c1)N1C=CC=CB1c1ccccc1-2 ; Clc1cccc(-c2cccc(-c3nc(-c4ccccc4)nc(-c4ccccc4)n3)c2)c1 ; C1=CB2c3ccccc3-c3ccc(-c4cccc(-c5cccc(-c6nc(-c7ccccc7)nc(-c7ccccc7)n6)c5)c4)cc3N2C=C1]

The synthesis of compound (10) was similar to that of compound (6), both using the classical SUZUKI coupling reaction, with a final reaction yield of 81.5%. MS(ASAP)=612.2.

### Example 11

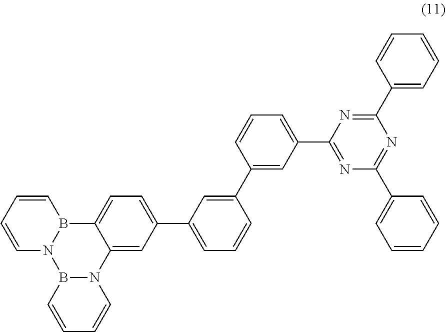 [SMILES: C1=CB2c3ccc(-c4cccc(-c5cccc(-c6nc(-c7ccccc7)nc(-c7ccccc7)n6)c5)c4)cc3N3C=CC=CB3N2C=C1]

### 13-(3′-(4,6-diphenyl-1,3,5-triazin-2-yl)-[1,1′-diphenyl]-3-yl)bis([1,2]azaboronophenyl)[1,2-a:1′,2′-c]phenyl[e][1,3,2,4]diazaboronylidenebenzene

The synthetic route is shown as follows:

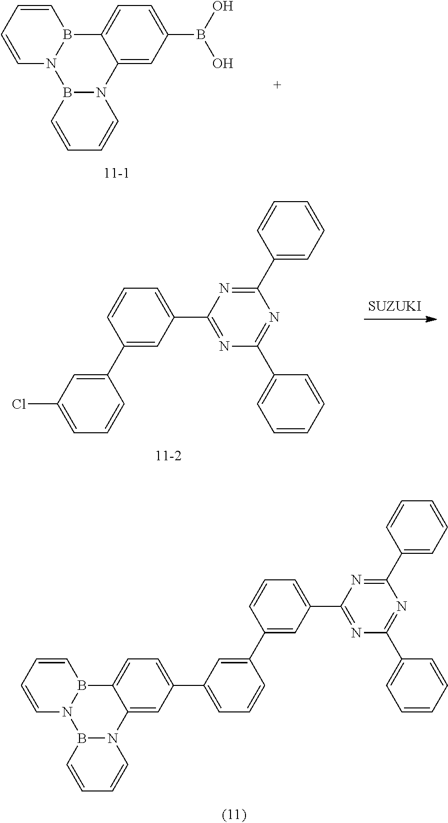 [SMILES: OB(O)c1ccc2c(c1)N1C=CC=CB1N1C=CC=CB21 ; Clc1cccc(-c2cccc(-c3nc(-c4ccccc4)nc(-c4ccccc4)n3)c2)c1 ; C1=CB2c3ccc(-c4cccc(-c5cccc(-c6nc(-c7ccccc7)nc(-c7ccccc7)n6)c5)c4)cc3N3C=CC=CB3N2C=C1]

The synthesis of compound (11) was similar to that of compound (6), both using the classical SUZUKI coupling reaction, with a final reaction yield of 84.4%, MS(ASAP)=613.3.

### Example 12

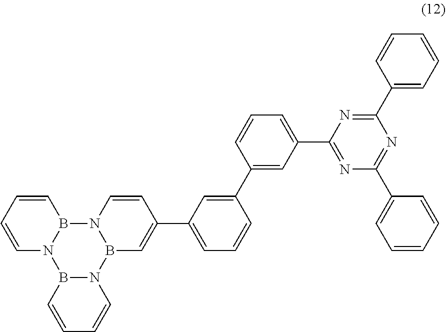 [SMILES: C1=CB2N3C=CC=CB3N3C=CC(c4cccc(-c5cccc(-c6nc(-c7ccccc7)nc(-c7ccccc7)n6)c5)c4)=CB3N2C=C1]

### 2-(3′-(4,6-diphenyl-1,3,5-triazin-2-yl)-[1,1′-diphenyl]-3-yl)tris([1,2]azaboribenzyl)[1,2-a:1′,2′-c:1″,2″-e][1,3,5,2,4,6]triazatriboronitritylenzene

The synthetic route is shown as follows:

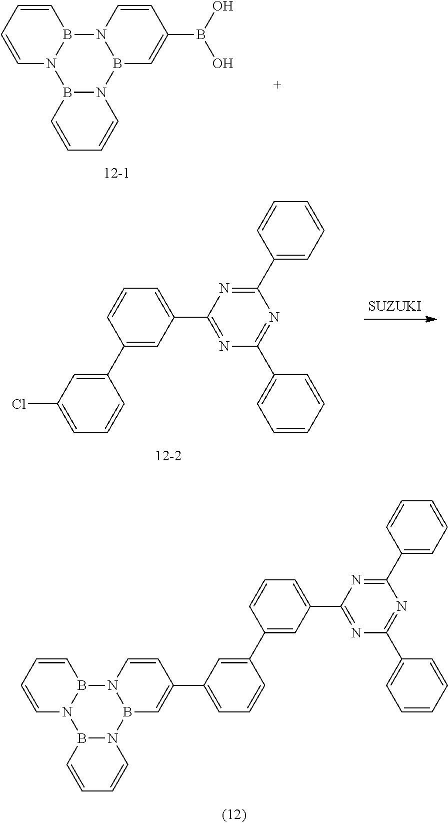 [SMILES: OB(O)C1=CB2N3C=CC=CB3N3C=CC=CB3N2C=C1 ; Clc1cccc(-c2cccc(-c3nc(-c4ccccc4)nc(-c4ccccc4)n3)c2)c1 ; C1=CB2N3C=CC=CB3N3C=CC(c4cccc(-c5cccc(-c6nc(-c7ccccc7)nc(-c7ccccc7)n6)c5)c4)=CB3N2C=C1]

The synthesis of compound (12) was similar to that of compound (6), both using the classical SUZUKI coupling reaction, with a final reaction yield of 79.5%, MS(ASAP)=614.3.

Preparation process of the organic mixture: the first host material and the second host material with the mass ratio of 1:1 were firstly mixed as well as possible, then the mixture was placed in a vacuum environment of less than or equal to 10-3 Torr, and the temperature in the vacuum environment was increased to make the two host materials melt completely. After the mixture was mixed well, it was allowed to cool to room temperature so that the mixture solidified, and then it was grounded into a powder with a ball mill for use.

The energy level of the organic material can be calculated by quantum computation, for example, using TD-DFT (time-dependent density functional theory) by Gaussian09W (Gaussian Inc.), the specific simulation methods of which can be found in WO2011141110. Firstly, the molecular geometry is optimized by semi-empirical method “Ground State/Semi-empirical/Default Spin/AM1” (Charge 0/Spin Singlet), and then the energy structure of organic molecules is calculated by TD-DFT (time-dependent density functional theory) “TD-SCF/DFT/Default Spin/B3PW91” and the basis set “6-31G (d)” (Charge 0/Spin Singlet). The HOMO and LUMO levels are calculated using the following calibration formula, where S₁, T₁, and resonance factor f(S₁) are used directly.

HOMO(eV)=((HOMO(G)×27.212)−0.9899)/1.1206

LUMO(eV)=((LUMO(G)×27.212)−2.0041)/1.385

Where HOMO (G) and LUMO (G) are the direct calculation results of Gaussian 09W, in units of Hartree. The results are shown in Table 1:

TABLE 1

| | HOMO | LUMO | T₁ | S₁ | ΔHOMO |
|---|---|---|---|---|---|
| Material | [eV] | [eV] | [eV] | [eV] | [eV] |
| (1) | −5.48 | −2.33 | 2.90 | 3.03 | 0.43 |
| (2) | −5.51 | −2.33 | 2.91 | 3.34 | 0.43 |
| (3) | −5.43 | −2.24 | 2.90 | 3.11 | 0.41 |
| (4) | −5.54 | −2.31 | 2.90 | 3.10 | 0.40 |
| (5) | −5.43 | −2.25 | 2.95 | 3.13 | 0.18 |
| (6) | −6.02 | −2.94 | 2.61 | 3.18 | 0.37 |
| (7) | −5.91 | −2.44 | 2.71 | 3.33 | 0.12 |
| (8) | −6.03 | −2.91 | 2.66 | 3.11 | 0.11 |
| (9) | −5.80 | −2.94 | 2.19 | 3.06 | 0.47 |
| (10) | −6.10 | −2.88 | 2.78 | 3.13 | 0.03 |
| (11) | −6.02 | −2.86 | 2.87 | 3.02 | 0.18 |
| (12) | −6.07 | −2.89 | 2.76 | 2.96 | 0.05 |

Among them, materials (1) to (4) can be applied to the first host material, and materials (5) to (8) can be applied to the second host material. For the numbering and formulation of the mixtures see Table 2, the mass ratio of the first host material to the second host material in all mixtures is 1:1.

TABLE 2

| | | | | | | | | | | | | |
|---|---|---|---|---|---|---|---|---|---|---|---|---|
| Mixture Number | (1) | (2) | (3) | (4) | (5) | (6) | (7) | (8) | (9) | (10) | (11) | (12) |

| | | | | | | | | | | | |
|---|---|---|---|---|---|---|---|---|---|---|---|
| A-1 | ∘ | | | | | | | | | ∘ | |
| A-2 | | ∘ | | | | ∘ | | | | | |
| A-3 | | ∘ | | | | | ∘ | | | | |
| A-4 | | | ∘ | | | | | ∘ | | | |
| A-5 | | | | ∘ | | | | ∘ | | | |
| A-6 | ∘ | | | | | | | ∘ | | | |
| A-7 | | | ∘ | | | | ∘ | | | | |
| A-8 | | ∘ | | | | | | ∘ | | | |
| A-9 | | | | | ∘ | | | | ∘ | | |
| A-10 | | | | | ∘ | | | | | ∘ | |
| A-11 | | | | | ∘ | | | | | | ∘ |
| A-12 | ∘ | | | | | | | | | ∘ | |
| A-13 | ∘ | | | | | | | | | | ∘ |
| A-14 | | | | ∘ | | | | | ∘ | | |
| A-15 | | | ∘ | | | | | | | ∘ | |

In comparison with the hybrid phosphorescent host materials, the host materials of the carbazole-based material architectures commonly used at present are marked with Ref1:

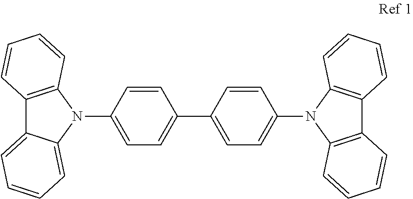 [SMILES: c1ccc2c(c1)c1ccccc1n2-c1ccc(-c2ccc(-n3c4ccccc4c4ccccc43)cc2)cc1]

Preparation of OLED: The structure of the OLED is as follows: ITO/HATCN (10 nm)/NPB (35 nm)/TCTA (5 nm)/(A-1)-(A-15): 5% Ir (ppy)₃/B3PYMPM (40 nm)/LiF (1 nm)/Al (150 nm).

The preparation steps are as follows:
a. Cleaning of the conductive glass substrate: prior to first-time use, the substrates are washed with various solvents (such as: chloroform, ketone, or isopropyl alcohol), and then treated with UV and ozone.
b. HIL (35 nm), EML (15 nm), and ETL (65 nm) were formed by thermal evaporation in a high vacuum (1×10⁻⁶ mbar).
c. Cathode: LiF/Al (1 nm/150 nm) were formed by thermal evaporation in a high vacuum (1×10⁻⁶ mbar).
d. Encapsulation: encapsulating the device in a nitrogen-regulated glove box with UV curable resin.

The current-voltage (J-V) characteristics of each OLED were studied with characterization equipment, while important parameters such as efficiency, lifetime, and external quantum efficiency were recorded in Table 3.

TABLE 3

| | | Current efficiency | Device Lifetime |
|---|---|---|---|
| | Device | @ 1000 nits | (T9 5@ 1000 nits) |
| A-1 | OLED1 | 71.23 | 855% |
| A-2 | OLED2 | 75.44 | 784% |
| A-3 | OLED3 | 78.98 | 779% |
| A-4 | OLED4 | 82.55 | 1050% |
| A-5 | OLED5 | 84.34 | 967% |
| A-6 | OLED6 | 81.22 | 932% |
| A-7 | OLED7 | 85.44 | 1010% |
| A-8 | OLED8 | 81.56 | 955% |
| A-9 | OLED9 | 78.99 | 678% |
| A-10 | OLED10 | 77.98 | 775% |
| A-11 | OLED11 | 76.48 | 758% |
| A-12 | OLED12 | 70.11 | 988% |
| A-13 | OLED13 | 78.44 | 588% |
| A-14 | OLED14 | 83.57 | 644% |
| A-15 | OLED15 | 74.33 | 555% |
| (6) | OLED16 | 49.84 | 288% |
| (8) | OLED17 | 51.22 | 234% |
| (11) | OLED18 | 46.78 | 267% |
| (12) | OLED19 | 45.22 | 255% |
| Ref1 | OLED REF | 30.11 | 100% |

It can be seen that the OLEDs prepared by the mixture as described herein have greatly improved luminescence efficiency and lifetime, and the external quantum efficiency has also been significantly improved.

In addition, compounds (6), (8), (11), and (12) are also used as single hosts for the preparation of OLEDs with more than 2.3 times efficiency and lifetime compared to Ref1. However, the co-host can achieve better performance in this device structure.

It is to be understood that the application of the disclosure is not limited to the above-described examples and that those skilled in the art would understand that it may be modified or changed in accordance with the above description, all of which are within the scope of the claims appended hereto.

What is claimed is:

1. An organic compound, comprising a structure of formula (I): 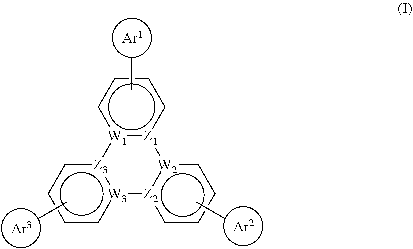
wherein:
each of Z₁ to Z₃ is independently N or C, and at least one of Z₁ to Z₃ is N;
each of W₁ to W₃ is independently B or C, and at least one of W₁ to W₃ is B;
Ar¹ to Ar³ at multiple occurrences, are independently selected from the group consisting of null, a C₅-C₃₀ aromatic group, a C₅-C₃₀ heteroaromatic group, a C₅-C₃₀ non-aromatic ring system, and can be substituted with one or more R⁸s;
R⁸ at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic group containing 6 to 40 ring atoms, a substituted/unsubstituted heteroaromatic group containing 5 to 40 ring atoms, an aryloxy or heteroaryloxy group containing 6 to 40 ring atoms, and any combination thereof, wherein one or more R⁸s may form a monocyclic or polycyclic aliphatic or aromatic ring system with each other and/or with the rings bonded thereto.

2. The organic compound according to claim 1, wherein Ar¹ to Ar³ at multiple occurrences are independently selected from null, one or combinations of more than one of the following groups: 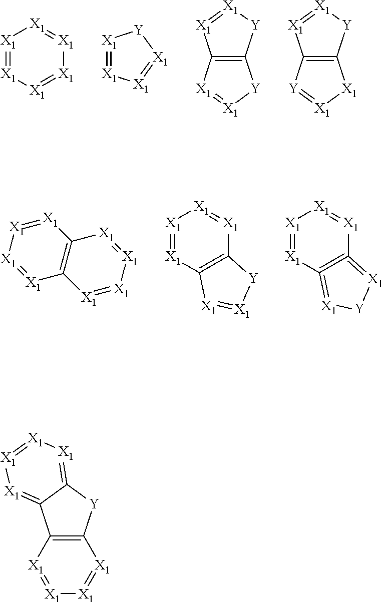
wherein:
each X₁ is CR¹⁰ or N;
each Y is selected from CR¹¹R¹², SiR¹³R¹⁴, NR¹⁵, C(═O), S, or O;
each of R¹⁰ to R¹⁵ is independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R¹⁰-R¹⁵ may form a monocyclic or polycyclic aliphatic or aromatic ring system with each other and/or with the rings bonded thereto.

3. The organic compound according to claim 1, wherein the organic compound is selected from one of formulas (I-1)-(I-3): 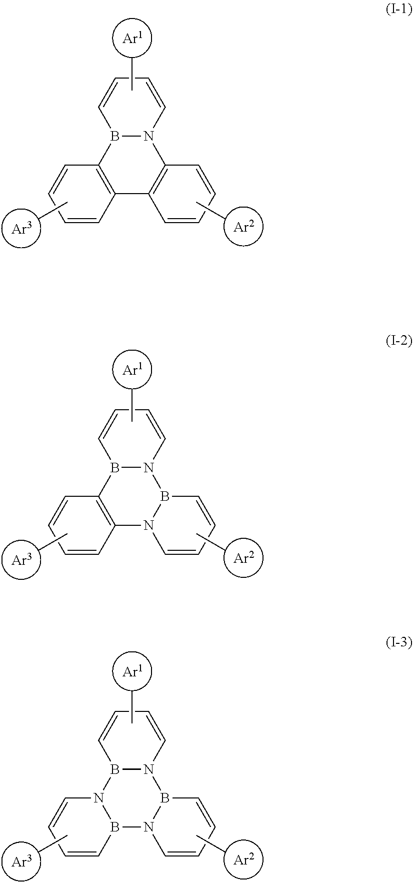
wherein, Ar¹ to Ar³ at multiple occurrences, are independently selected from the group consisting of null, a C₅-C₃₀ aromatic group, a C₅-C₃₀ heteroaromatic group, a C₅-C₃₀ non-aromatic ring system, and can be substituted with one or more R⁸s.

4. The organic compound according to claim 1, wherein Ar¹ to Ar³ at multiple occurrences, are independently selected from one or combinations of more than one of the following groups: 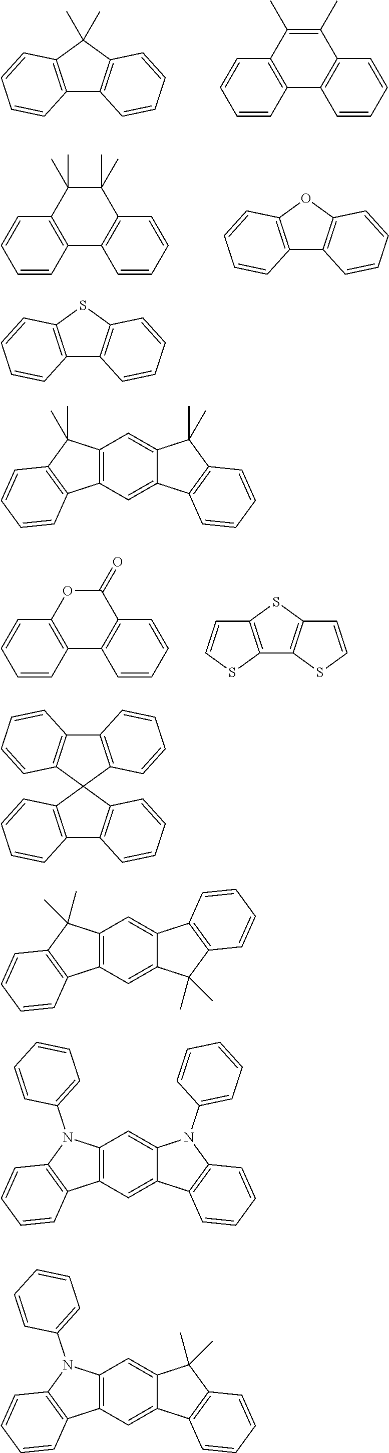 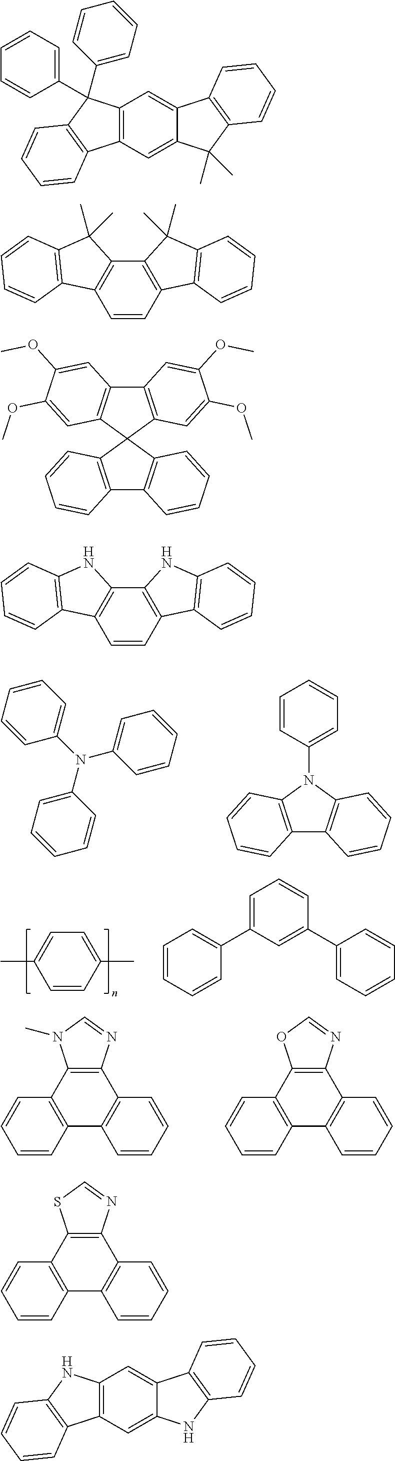 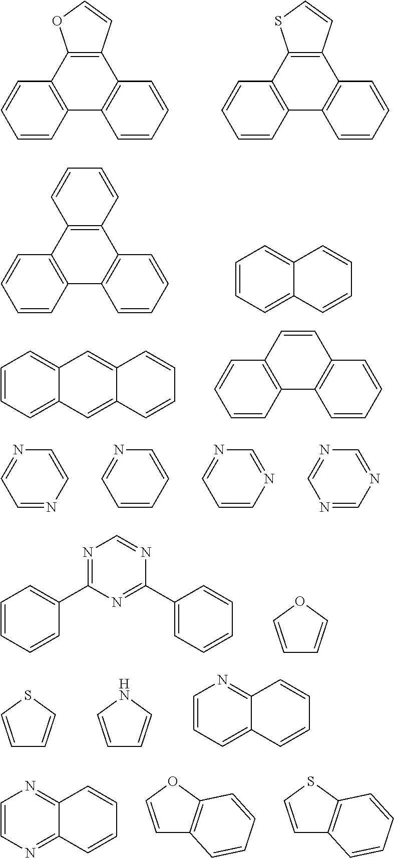
wherein, u is 1, or 2, or 3, or 4.

5. The organic compound according to claim 1, wherein at multiple occurrences, at least one of Ar¹ to Ar³ comprises an electron-donating group, and/or at least one of Ar¹ to Ar³ comprises an electron-accepting group.

6. The organic compound according to claim 5, wherein the electron-donating group can be selected from formulas D1-D10.

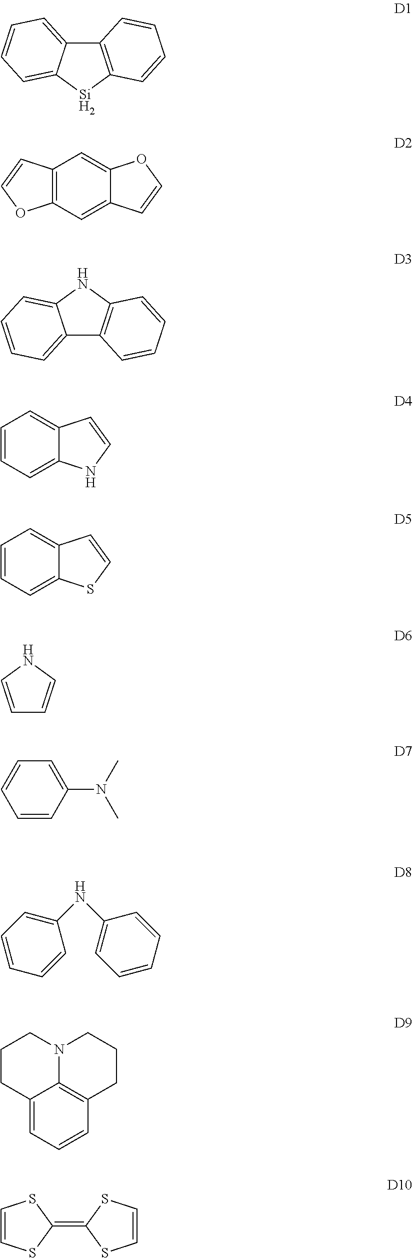

7. The organic compound according to claim 5, wherein the electron-accepting group can be selected from F, a cyano group, or comprise the following groups: 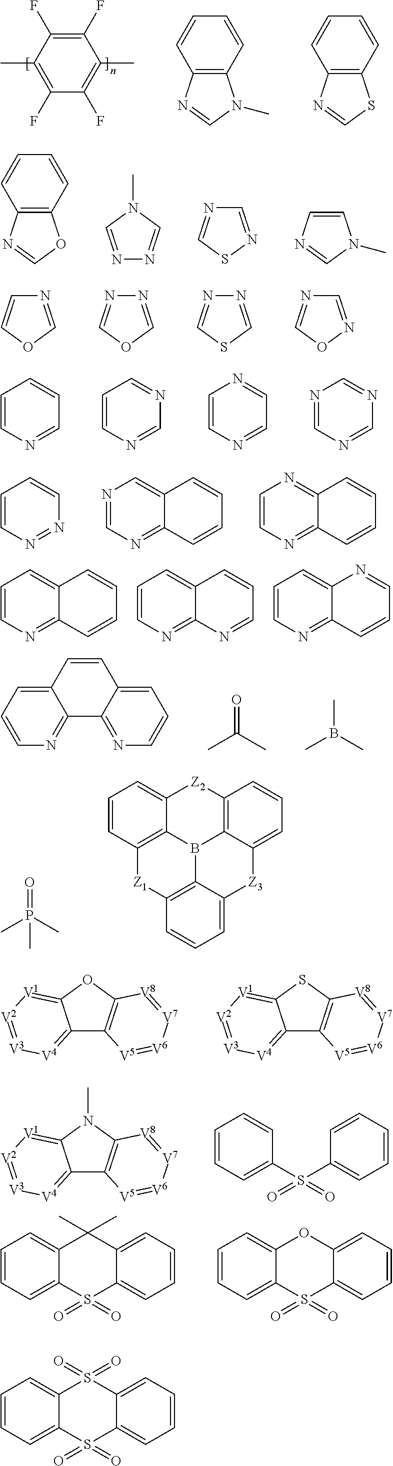
wherein
n is an integer from 1 to 3;
each of V¹ to V⁸ is CR¹⁶ or N, and at least one of them is N;
each of Z₁ to Z₃ is a single bond, or C(R¹⁶)₂, or O, or S;
R¹⁶ at multiple occurrences, is independently selected from the following groups: H, alkyl, alkoxy, amino, alkene, alkyne, aralkyl, heteroalkyl, aryl, or heteroaryl.

8. The organic compound according to claim 1, wherein the organic compound is selected from one of the following formulas.

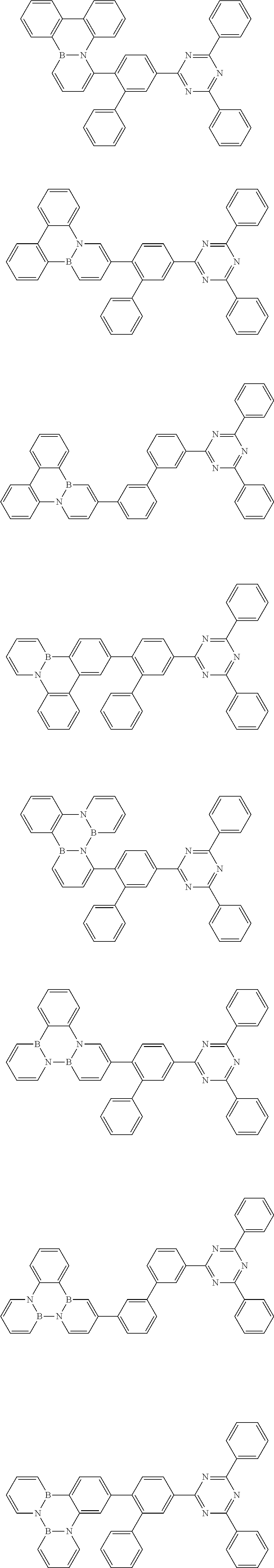

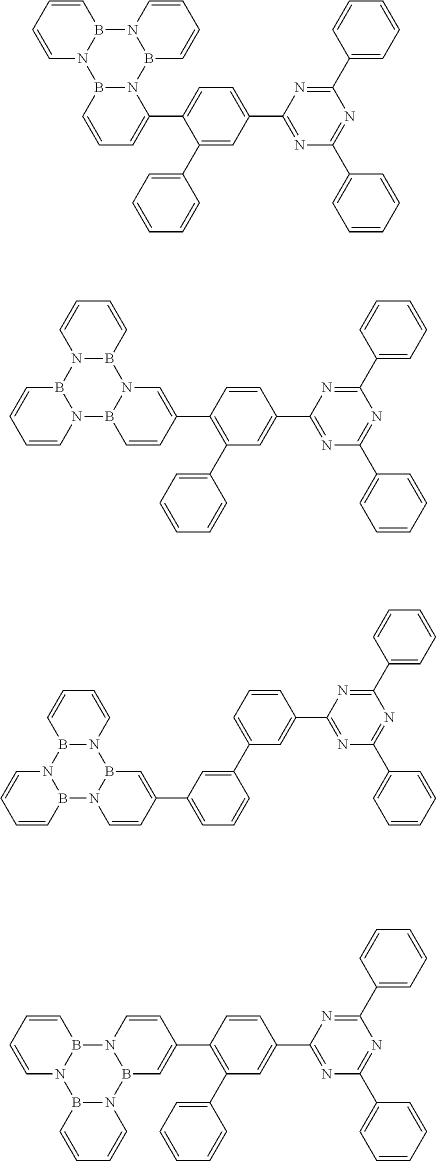 [SMILES: C1=CB2N3C=CC=CB3N3B(C=CC=C3c3ccc(-c4nc(-c5ccccc5)nc(-c5ccccc5)n4)cc3-c3ccccc3)N2C=C1 ; C1=CB2N3C=CC=CB3N3C=C(c4ccc(-c5nc(-c6ccccc6)nc(-c6ccccc6)n5)cc4-c4ccccc4)C=CB3N2C=C1 ; C1=CB2N3C=CC=CB3N3C=CC(c4cccc(-c5cccc(-c6nc(-c7ccccc7)nc(-c7ccccc7)n6)c5)c4)=CB3N2C=C1 ; C1=CB2N3C=CC=CB3N3C=CC(c4ccc(-c5nc(-c6ccccc6)nc(-c6ccccc6)n5)cc4-c4ccccc4)=CB3N2C=C1]

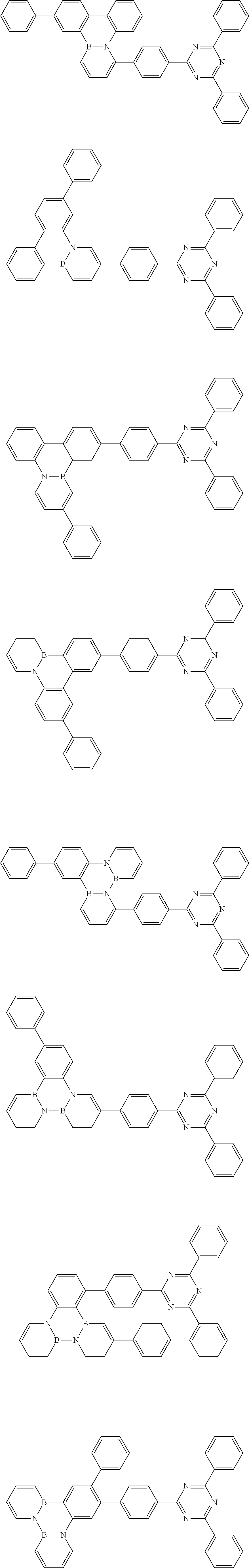

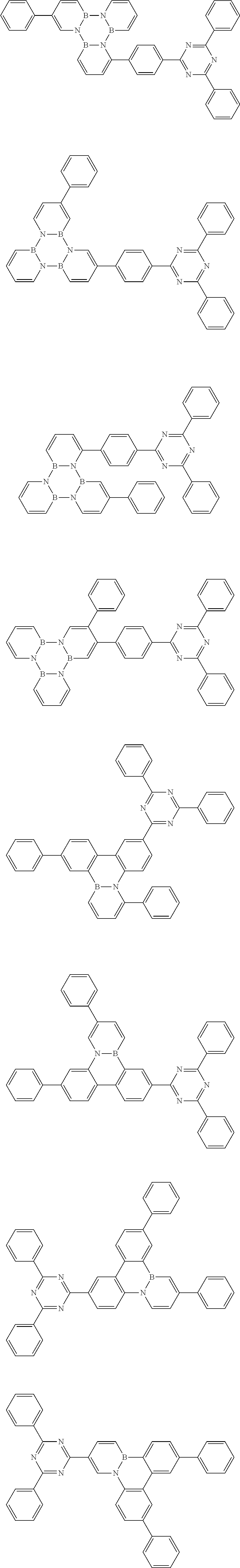

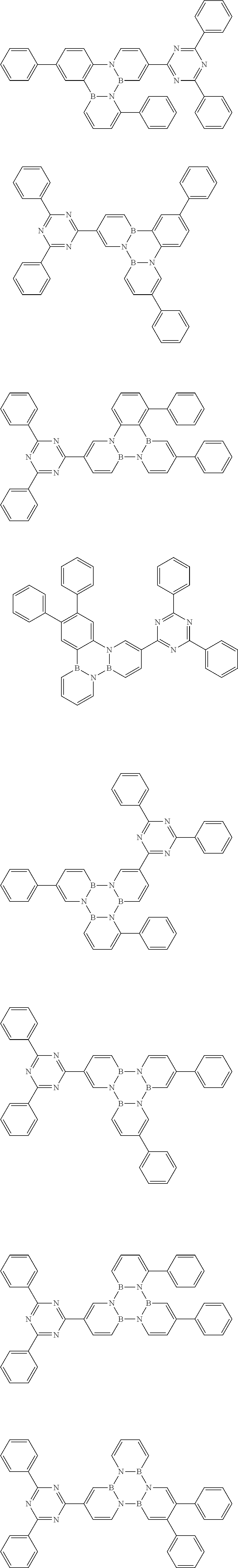

9. An organic mixture, comprising a first compound H1 and a second compound H2, wherein the first compound H1 comprises an organic compound according to claim 1, and min(LUMO(H1)−HOMO(H2), LUMO(H2)−HOMO(H1))≤min(E_{T}(H1), E_{T}(H2))+0.1 eV, LUMO(H1), HOMO(H1), and E_{T}(H1) stand for the energy level of the highest occupied molecular orbital, the energy level of the lowest unoccupied molecular orbital, and the triplet energy level of the first compound H1, LUMO(H2), HOMO(H2), and E_{T}(H2) stand for the energy level of the highest occupied orbital, the energy level of the lowest unoccupied orbital, and the triplet energy level of the second compound H2.

10. The organic mixture according to claim 9, wherein the second compound H2 is selected from one of formulas (II-1)-(II-4): 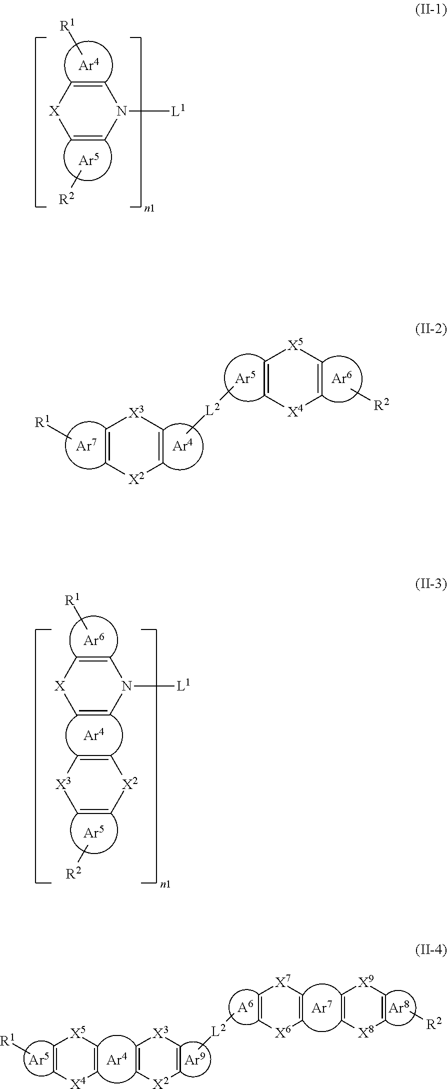
wherein,
each L¹ represents an aromatic or hetero-aromatic group containing 5 to 60 ring atoms;
each L² represents a single bonded, an aromatic or hetero-aromatic group containing 5 to 30 ring atoms, the attachment position of L¹ and L² can be on any carbon atom of the benzene ring;
Ar⁴ to Ar⁹ are independently selected from an aromatic or hetero-aromatic group containing 5 to 30 ring atoms;
each X represents a single bond, N(R), C(R)₂, Si(R)₂, O, C═N(R), C═C(R)₂, P(R), P(═O)R, S, S═O, or SO₂;
X₂ to X₉ are independently selected from a single bond, N(R), C(R)₂, Si(R)₂, O, C═N(R), C═C(R)₂, P(R), P(═O)R, S, S═O, or SO₂, and X² and X³ are not single bonds at the same time, X⁴ and X⁵ are not single bonds at the same time, X⁶ and X⁷ are not single bonds at the same time, X⁸ and X⁹ are not single bonds at the same time;
R¹, R², and R are independently selected from the group consisting of H, D, F, CN, an alkenyl group, an alkynyl group, a nitrile group, an amine group, a nitro group, an acyl group, an alkoxy group, a carbonyl group, a sulfone group, a C₁-C₃₀ alkyl group, a C₃-C₃₀ cycloalkyl group, a C₅-C₆₀ aromatic hydrocarbon group, and a C₅-C₆₀ aromatic heterocyclic group, wherein the attachment position of R¹ and R² can be on any carbon atom of the fused ring, and there can be any number of carbon atoms substituted with R¹ and R²;
n1 is an integer from 1 to 4.

11. The organic mixture according to claim 9, wherein the second compound H2 is selected from one of formulas (III-1)-(III-4): 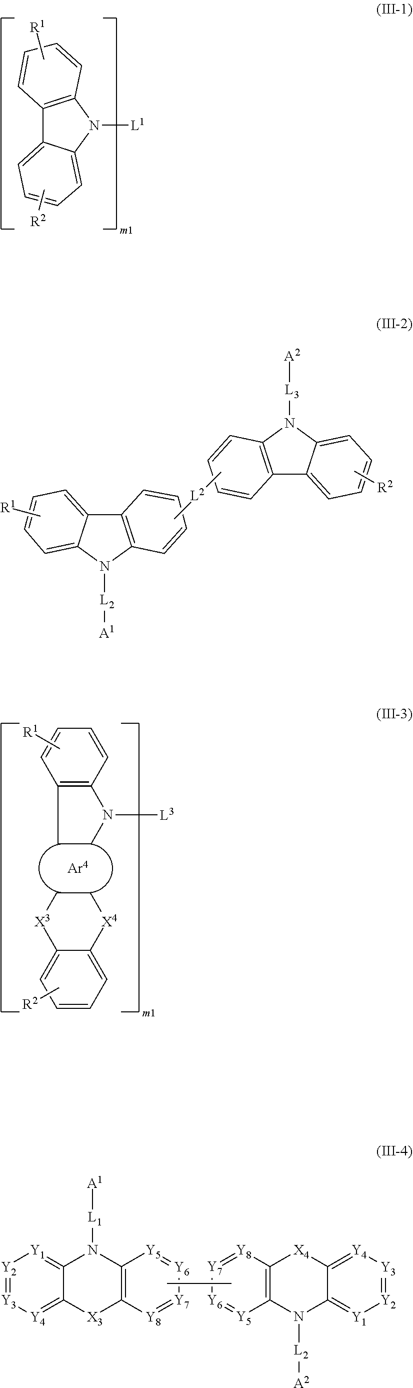
wherein, L³ is defined as the above-mentioned L¹; A¹ and A² are independently selected from an aromatic or hetero-aromatic group containing 5 to 30 ring atoms; each of Yi to Ys is N or CR, and two adjacent Xs cannot be Ns at the same time.

12. The organic mixture according to claim 9, wherein the molecular weight difference between the first compound H1 and the second compound ≤100 Dalton.

13. The organic mixture according to claim 9, wherein the second compound H2 is selected from one of the following formulas.

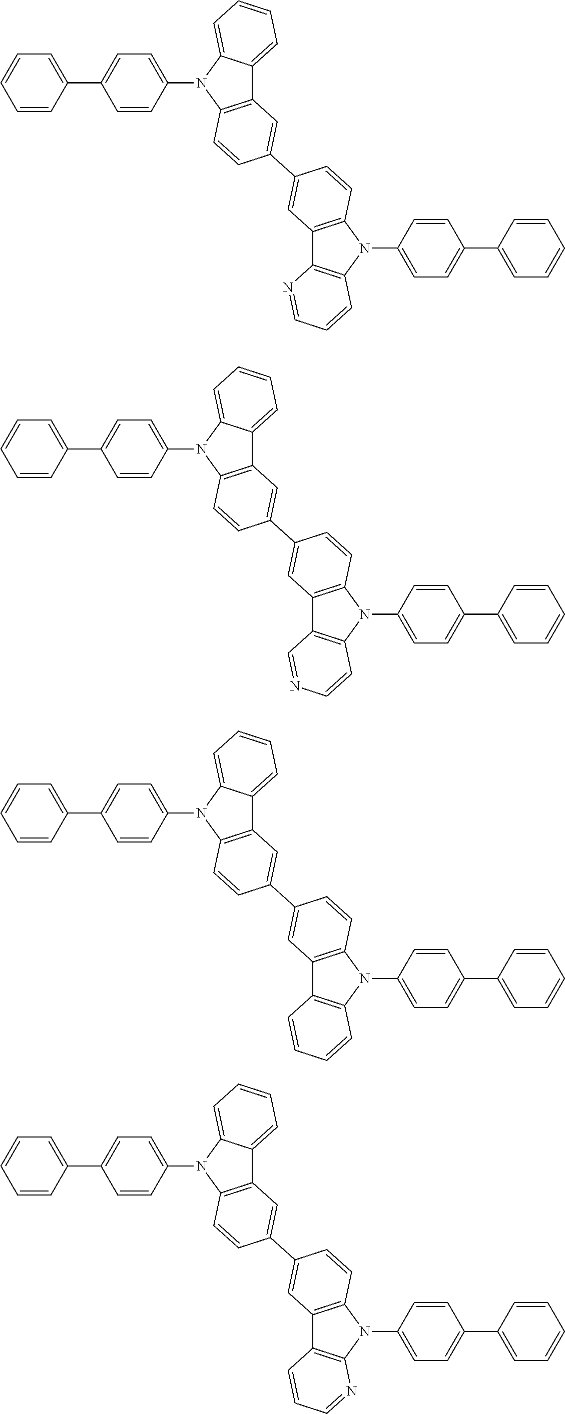 [SMILES: c1ccc(-c2ccc(-n3c4ccccc4c4cc(-c5ccc6c(c5)c5ncccc5n6-c5ccc(-c6ccccc6)cc5)ccc43)cc2)cc1 ; c1ccc(-c2ccc(-n3c4ccccc4c4cc(-c5ccc6c(c5)c5cnccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5cccnc5n6-c5ccc(-c6ccccc6)cc5)ccc43)cc2)cc1]

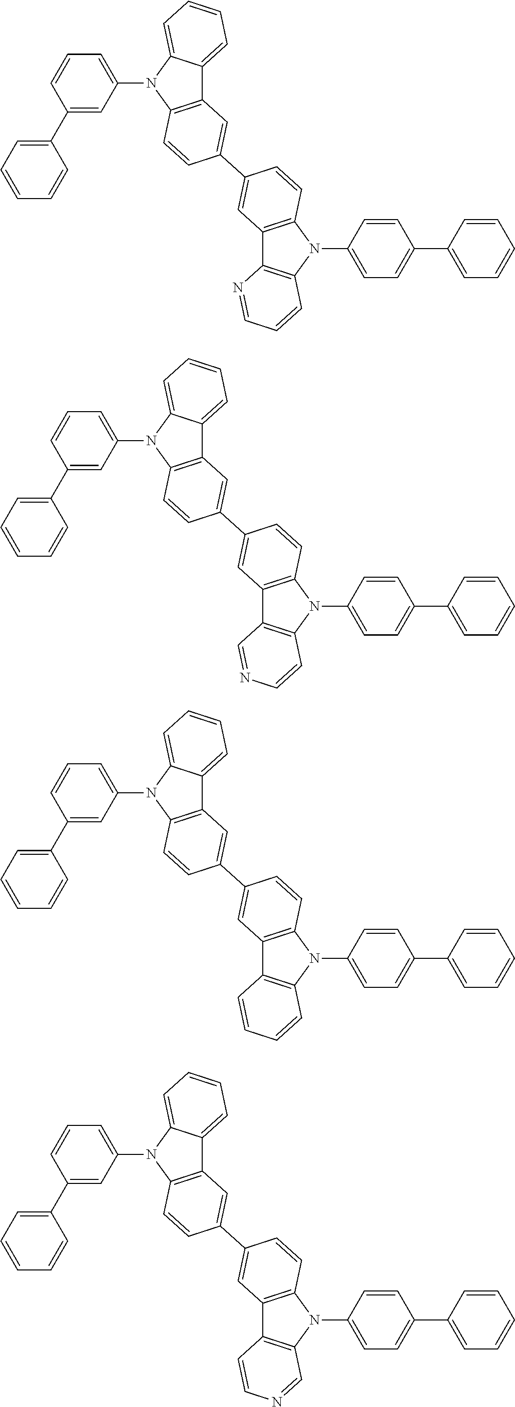 [SMILES: c1ccc(-c2ccc(-n3c4ccc(-c5ccc6c(c5)c5ccccc5n6-c5cccc(-c6ccccc6)c5)cc4c4ncccc43)cc2)cc1 ; c1ccc(-c2ccc(-n3c4ccncc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccc(-c5ccc6c(c5)c5ccccc5n6-c5cccc(-c6ccccc6)c5)cc4c4ccncc43)cc2)cc1]

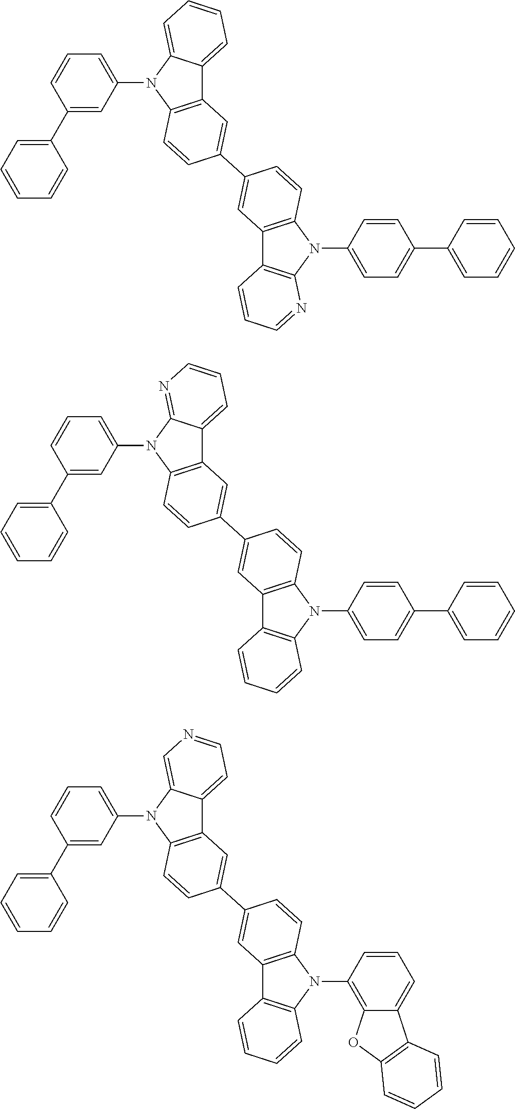 [SMILES: c1ccc(-c2ccc(-n3c4ccc(-c5ccc6c(c5)c5ccccc5n6-c5cccc(-c6ccccc6)c5)cc4c4cccnc43)cc2)cc1 ; c1ccc(-c2ccc(-n3c4ccccc4c4cc(-c5ccc6c(c5)c5cccnc5n6-c5cccc(-c6ccccc6)c5)ccc43)cc2)cc1 ; c1ccc(-c2cccc(-n3c4ccc(-c5ccc6c(c5)c5ccccc5n6-c5cccc6c5oc5ccccc56)cc4c4ccncc43)c2)cc1]

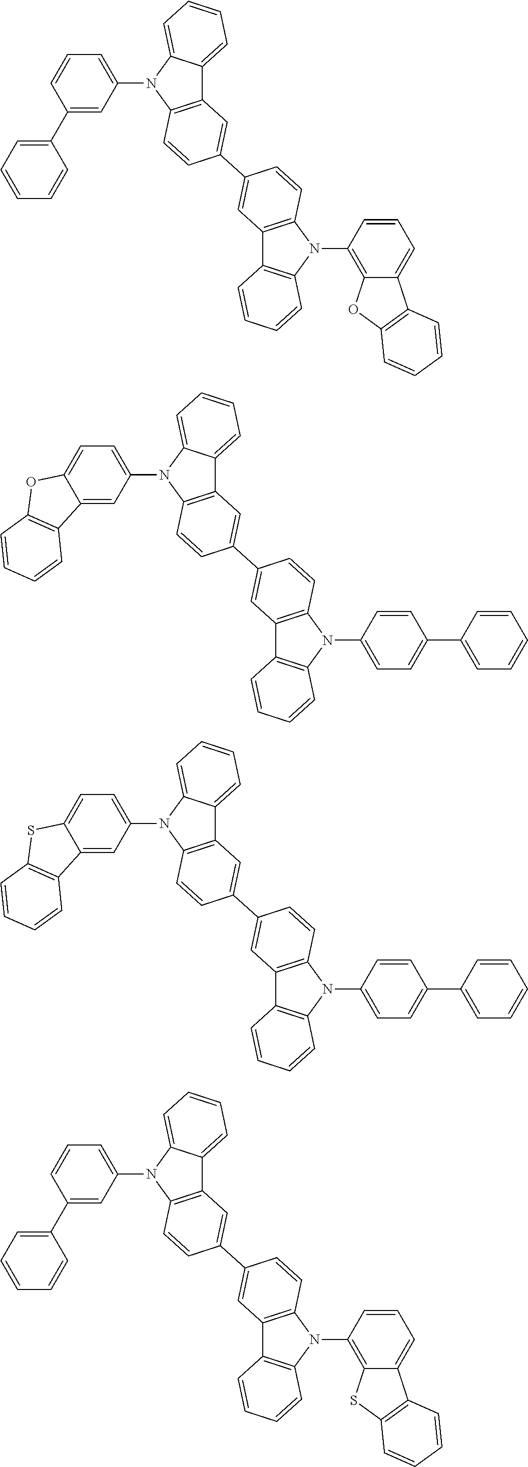 [SMILES: c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cccc(-n3c4ccccc4c4cc(-c5ccc6c(c5)c5ccccc5n6-c5cccc6c5sc5ccccc56)ccc43)c2)cc1]

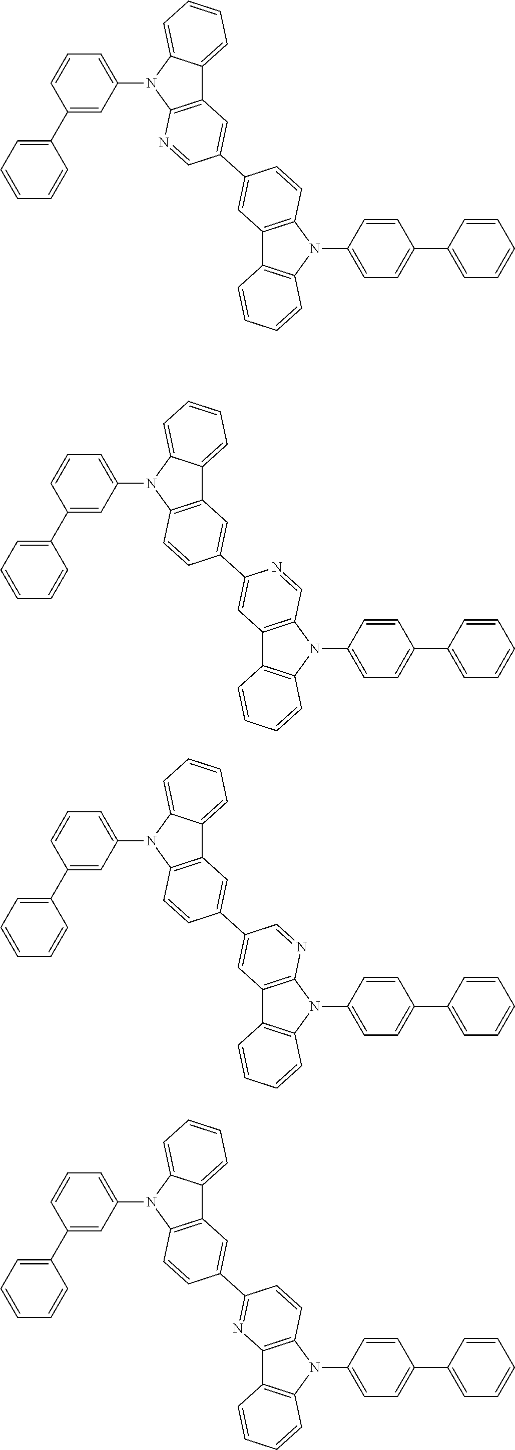 [SMILES: c1ccc(-c2ccc(-n3c4ccccc4c4cc(-c5cnc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cccc(-c6ccccc6)c5)ncc43)cc2)cc1 ; c1ccc(-c2ccc(-n3c4ccccc4c4cc(-c5ccc6c(c5)c5ccccc5n6-c5cccc(-c6ccccc6)c5)cnc43)cc2)cc1 ; c1ccc(-c2ccc(-n3c4ccccc4c4nc(-c5ccc6c(c5)c5ccccc5n6-c5cccc(-c6ccccc6)c5)ccc43)cc2)cc1]

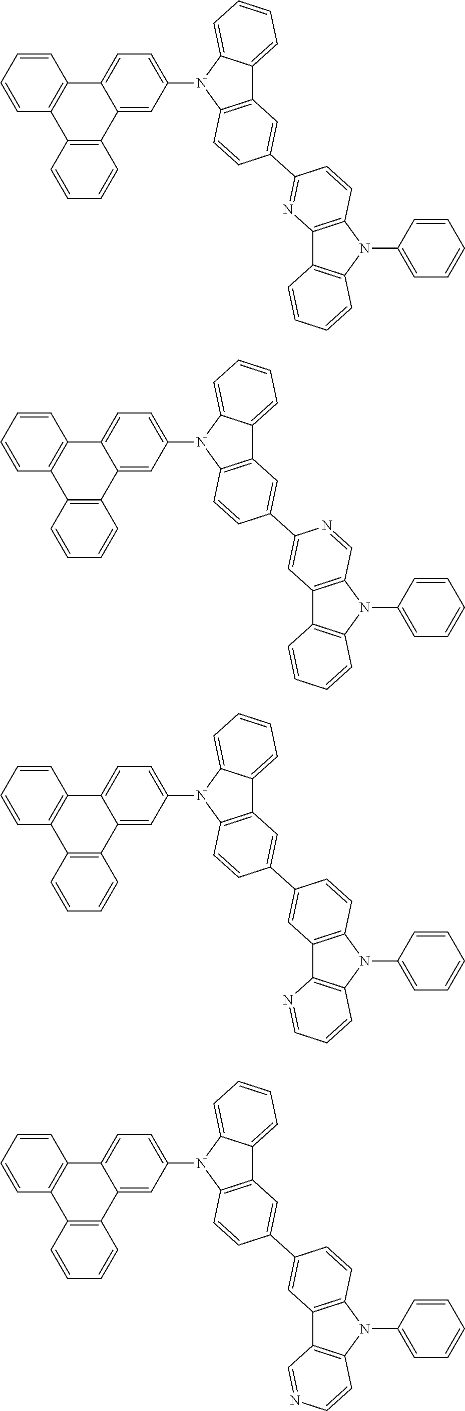 [SMILES: c1ccc(-n2c3ccccc3c3nc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4ccc5c6ccccc6c6ccccc6c5c4)ncc32)cc1 ; c1ccc(-n2c3ccc(-c4ccc5c(c4)c4ccccc4n5-c4ccc5c6ccccc6c6ccccc6c5c4)cc3c3ncccc32)cc1 ; c1ccc(-n2c3ccncc3c3cc(-c4ccc5c(c4)c4ccccc4n5-c4ccc5c6ccccc6c6ccccc6c5c4)ccc32)cc1]

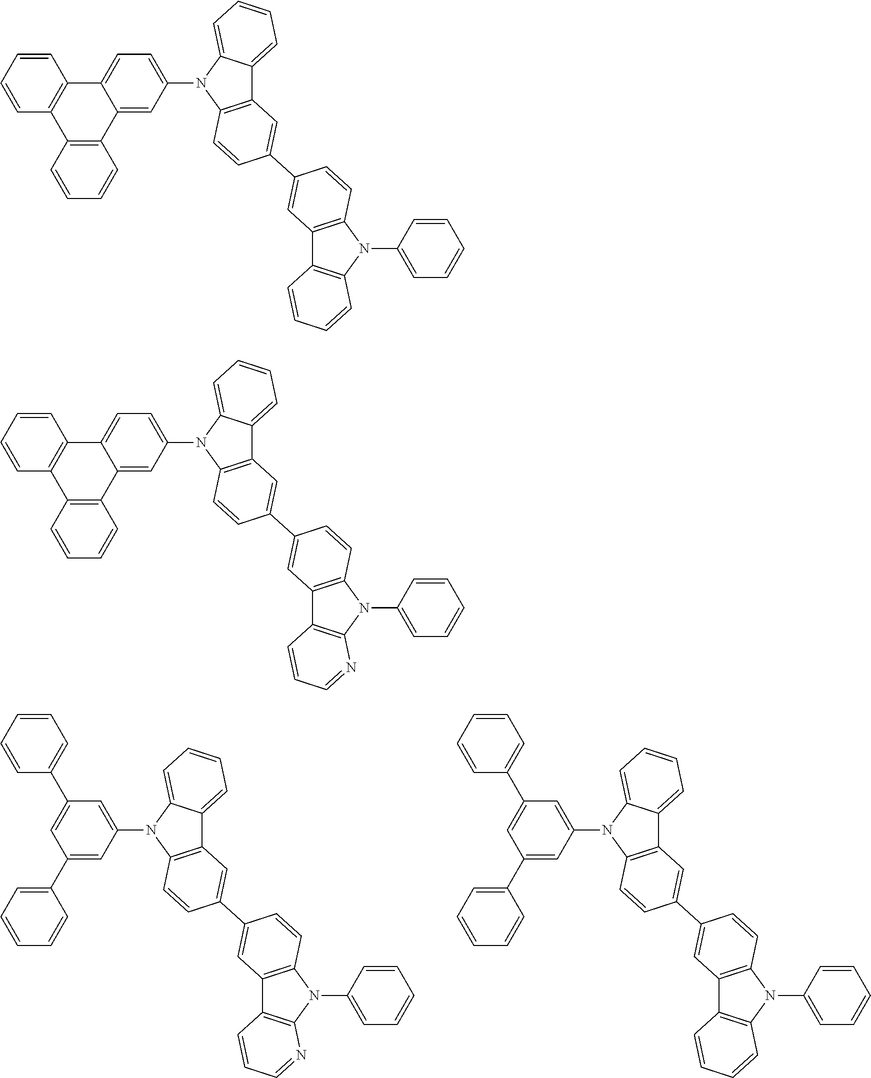 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccc(-c4ccc5c(c4)c4ccccc4n5-c4ccc5c6ccccc6c6ccccc6c5c4)cc3c3cccnc32)cc1 ; c1ccc(-c2cc(-c3ccccc3)cc(-n3c4ccccc4c4cc(-c5ccc6c(c5)c5cccnc5n6-c5ccccc5)ccc43)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

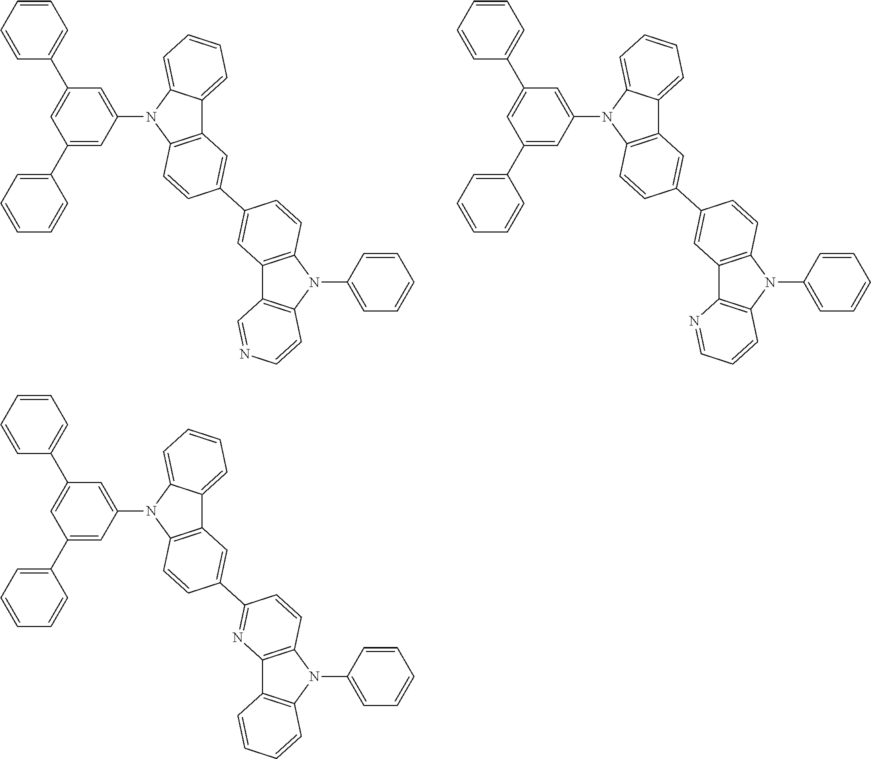 [SMILES: c1ccc(-c2cc(-c3ccccc3)cc(-n3c4ccccc4c4cc(-c5ccc6c(c5)c5cnccc5n6-c5ccccc5)ccc43)c2)cc1 ; c1ccc(-c2cc(-c3ccccc3)cc(-n3c4ccccc4c4cc(-c5ccc6c(c5)c5ncccc5n6-c5ccccc5)ccc43)c2)cc1 ; c1ccc(-c2cc(-c3ccccc3)cc(-n3c4ccccc4c4cc(-c5ccc6c(n5)c5ccccc5n6-c5ccccc5)ccc43)c2)cc1]

14. An organic electronic device, comprising the organic compound according to claim 1.

15. The organic electronic device according to claim 14, wherein the organic electronic device can be selected from an organic light emitting diode, an organic photovoltaic cell, an organic light emitting electrochemical cell, an organic field effect transistor, an organic light emitting field effect transistor, an organic laser, an organic spintronic device, an organic sensor, or an organic plasmon emitting diode.

16. The organic electronic device according to claim 15, comprising a first electrode, a second electrode and at least one functional layer disposed between the first electrode and the second electrode, wherein at least one of the functional layer is selected from a light-emitting layer, an electron-transport layer; the light-emitting layer comprises the organic compound; the electron-transport layer comprises the organic compound.

17. An organic electronic device, comprising the organic mixture according to claim 9.

18. The organic electronic device according to claim 17, wherein the organic electronic device can be selected from an organic light emitting diode, an organic photovoltaic cell, an organic light emitting electrochemical cell, an organic field effect transistor, an organic light emitting field effect transistor, an organic laser, an organic spintronic device, an organic sensor, or an organic plasmon emitting diode.

19. The organic electronic device according to claim 18, comprising a first electrode, a second electrode and at least one functional layer disposed between the first electrode and the second electrode, wherein at least one of the functional layer is selected from a light-emitting layer, an electron-transport layer; the light-emitting layer comprises the organic mixture; the electron-transport layer comprises the organic compound.
